SEQUENCE LISTING (226 sequences)

SEQ ID NO: 1 - protein, 272 aa, Human cytomegalovirus
MLFRYITFHREKVLYLTAACIFGVYISLHDACIPVVGKIGTNVTLNAVDVLPPRDQVRWSYGPGGQGYMLCIFTGTSTTTFNNTRFNFSCLSNYSLLLINVTTQYSTTYRTMTSLDHWLHQRHNHGSRWTLDTCYNLTVNENGTFPTTTTKKPTTTTRTTTTTTQRTTTTRTTTTAKKTTISTTHHKHPSPKKSTTPNSHVEHHVGFEATAAETPLQPSPQHQHLATHALWVLAVVIVIIIIIIFYFRIPQKLWLLWQHDKHGIVLIPQTDL

SEQ ID NO: 2 - DNA, 820 bp, Artificial Sequence
  Description of Artificial Sequence: Synthetic polynucleotide
atgttgttcaggtacatcactttccatagagagaaggtgctatacctgaccgccgcctgcatattcggggtgtatatctccctgcacgacgcgtgtatccccgtggtaggcaaaattggtacgaacgttaccctgaatgcggtggacgtgctcccccctcgtgaccaagtgcggtggagctatgggccgggcgggcagggatatatgctctgcatctttactggcacatcaaccactactttcaataatacccgcttcaatttcagctgcctgagcaattattctctcctgttgattaatgtgaccacccaatactcaacaacttatagaacaatgacctctctggaccactggctgcatcagaggcataaccacgggagtcgctggacactggacacttgttacaatctaaccgttaacgaaaatggcactttccctacaaccaccacaaagaaacccactactacaacacgaactaccacaactactacgcagcgaactaccactacccggaccaccaccacagctaagaagacaacaataagcactactcaccacaagcaccctagcccaaagaaaagcactactcctaactcacatgttgagcatcatgtgggttttgaagctacggccgcagagacacccctgcaaccctctccgcagcatcagcacctcgctacccacgccctttgggttcttgcagttgtgatcgtcatcattatcataatcattttttattttaggattcctcagaagctgtggttgctttggcagcacgacaagcatggcattgtgcttattcctcaaacggacctggtaa

SEQ ID NO: 3 - protein, 428 aa, Human adenovirus D37
MNTVIRIVLLSLLVAFSQAGFHTINATWWANITLVGPPDTPVTWYDTQGLWFCNGSRVKNPQIRHTCNDQNLTLIHVNKTYERTYMGYNRQGTKKEDYKVVVIPPPPATVKPQPEPEYVFVYMGENKTLEGPPGTPVTWFNQDGKKFCEGEKVLHPEFNHTCDKQNLILLFVNFTHDGAYLGYNHQGTQRTHYEVTVLDLFPDSGQMKIENHSEETEQKNDEHHNWQKQGGQKQGGQKTNQTKVNDRRKTAQKRPSKLKPATIEAMLVTVTAGSNLTLVGPKAEGKVTWFDGDLKRPCEPNYRLRHECNNQNLTLINVTKDYEGTYYGTNDKDEGKRYRVKVNTTNSQSVKIQPYTRQTTPDQEHKFELQFETNGNYDSKIPSTTVAIVVGVIAGFITLIIVFICYICCRKRPRAYNHMVDPLLSFSY

SEQ ID NO: 4 - DNA, 1287 bp, Artificial Sequence
  Description of Artificial Sequence: Synthetic polynucleotide
atgaacacggtgatccgcatagtccttctgtctctgctggtggctttctcccaggccggcttccacacaattaatgccacctggtgggctaacattactctcgtaggccccccggatacccccgtgacttggtacgacactcagggtctgtggttctgtaacgggagtcgagtgaaaaatcctcaaattcgccatacctgtaacgaccaaaatctgaccttgatccacgtgaacaagacatacgagcgtacatatatgggctacaataggcagggtacaaagaaagaggactataaagtggtagtgattccgcctccccccgcaacagtcaagccccaaccagagcctgagtatgtcttcgtgtatatgggcgagaacaagaccctggaaggacctccaggaacacccgttacctggtttaaccaggatggaaagaagttttgcgaaggggagaaagtgcttcaccccgagttcaatcatacctgcgacaagcagaacctgatcctgctttttgtgaatttcacccatgacggtgcgtacctcggttataaccatcaaggcacccagcggacccattatgaggttactgtcctcgatctcttccccgacagtggtcagatgaaaatcgaaaaccatagtgaggaaactgagcagaaaaatgacgagcatcacaactggcagaaacaaggcggacaaaagcagggcggccagaagacaaatcagacaaaagtcaatgatcgacgcaaaaccgcccagaaacgtcctagcaaactaaagccagcaactattgaggcaatgctggtgacagtaactgctggaagtaacctgaccctcgtggggcccaaggcggaggggaaagtaacctggttcgacggcgatctaaaacgcccctgtgaaccaaactacagacttagacacgaatgcaacaaccagaacctgactctgattaacgtgaccaaggactacgaaggaacatactacgggacgaatgataaggatgagggaaaacggtaccgggttaaggttaacaccacaaactcccagagtgtcaaaattcagccttacaccaggcagactactcctgaccaggaacacaaattcgaattacagtttgagactaacggtaactatgactccaagattccatctacaacggtcgcgatcgtagtgggcgtgattgcaggcttcatcacattgatcatcgtgttcatctgctatatctgctgtaggaagcgccctcgggcgtacaaccacatggtggaccctctgttgagtttctcatattaa

SEQ ID NO: 5 - protein, 394 aa, Artificial Sequence
  Description of Artificial Sequence: Synthetic polypeptide
MYRMQLLSCIALSLALVTNSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKSSGSGSPTTTPAPRPPTPAPTIASQPLSLRPEACRPAAGGAVHTRGLDFAPRKIEVMYPPPYLDNEKSNGTIIHVKGKHLCPSPLFPGPSKPTLIALVTSGALLAVLGITGYFL

SEQ ID NO: 6 - DNA, 1185 bp, Artificial Sequence
  Description of Artificial Sequence: Synthetic polynucleotide
atgtacaggatgcaactcctgtcttgcattgcactaagtcttgcacttgtcacaaacagtcaggttcagctggtggaatcaggaggtggcctggtgcagcctggaggatccctgaaactctcctgtgcagcctcaggattcgatttcagtagatactggatgagttgggtccggcaggctccagggaaagggctagaatggattggagagattaatccaactagcagtacgataaactttacgccatctctaaaggataaagtcttcatctccagagacaacgccaaaaatacgctgtacctgcaaatgagcaaagtgagatccgaggacacagccctttattactgtgcaagagggaactactataggtacggagatgctatggactactggggtcaaggaacctcagtcaccgtgagcaagatctctggtggcggtggctcgggcggtggtgggtcgggtggcggcggctcgggtggtggtgggtcgggcggcggcggctcgagcgacatcgtgctgacccagtctcctgcttccttagctgtatctctgggacagagggccaccatctcatgcagggccagcaaaagtgtcagtacatctggctatagttatctgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaagagctctggctctggttcgcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccaccctgattgcactggtcacctcgggagccctgctggctgtcttgggcatcactggctatttcctgtaa

SEQ ID NO: 7 - protein, 266 aa, Artificial Sequence
  Description of Artificial Sequence: Synthetic polypeptide
  MOD_RES (28)..(28): Any amino acid
  MOD_RES (36)..(36): Any amino acid
  MOD_RES (60)..(60): Any amino acid
  MOD_RES (93)..(93): Any amino acid
  MOD_RES (251)..(251): Any amino acid
QVQLVESGGGLVQPGGSLKLSCAASGFXFSRYWMSXVRQAPGKGLEWIGEINPTSSTINXTPSLKDKVFISRDNAKNTLYLQMSKVRSEDTAXYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTXGSGTKLEIKSSGSGS

SEQ ID NO: 8 - protein, 122 aa, Artificial Sequence
  Description of Artificial Sequence: Synthetic polypeptide
  MOD_RES (28)..(28): Any amino acid
  MOD_RES (36)..(36): Any amino acid
  MOD_RES (60)..(60): Any amino acid
  MOD_RES (93)..(93): Any amino acid
QVQLVESGGGLVQPGGSLKLSCAASGFXFSRYWMSXVRQAPGKGLEWIGEINPTSSTINXTPSLKDKVFISRDNAKNTLYLQMSKVRSEDTAXYYCARGNYYRYGDAMDYWGQGTSVTVSKI

SEQ ID NO: 9 - protein, 111 aa, Artificial Sequence
  Description of Artificial Sequence: Synthetic polypeptide
  MOD_RES (102)..(102): Any amino acid
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTXGSGTKLEIK

SEQ ID NO: 10 - DNA, 258 bp, Artificial Sequence
  Description of Artificial Sequence: Synthetic polynucleotide
cccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagccc

SEQ ID NO: 11 - protein, 86 aa, Artificial Sequence
  Description of Artificial Sequence: Synthetic polypeptide
PTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 12 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 13 - protein, 44 aa, Homo sapiens
PTTTPAPRPPTPAPTIASQPLSLRPEACRPAAGGAVHTRGLDFA

SEQ ID NO: 14 - DNA, 708 bp, Homo sapiens
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 15 - DNA, 132 bp, Homo sapiens
cccaccacgacgccagcgccgcgaccaccaacaccggcgcccaccatcgcgtcgcagcccctgtccctgcgcccagaggcgtgccggccagcggcggggggcgcagtgcacacgagggggctggacttcgcc

SEQ ID NO: 16 - protein, 235 aa, Homo sapiens; identical to SEQ ID NO: 12

SEQ ID NO: 17 - protein, 70 aa, Homo sapiens
PTTTPAPRPPTPAPTIASQPLSLRPEACRPAAGGAVHTRGLDFACDIYIWAPLAGTCGVLLLSLVITLYC

SEQ ID NO: 18 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 19 - DNA, 663 bp, Homo sapiens
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctga

SEQ ID NO: 20 - protein, 40 aa, Homo sapiens
KIEVMYPPPYLDNEKSNGTIIHVKGKHLCPSPLFPGPSKP

SEQ ID NO: 21 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 22 - protein, 21 aa, Homo sapiens
LIALVTSGALLAVLGITGYFL

SEQ ID NO: 23 - DNA, 1158 bp, Homo sapiens
atgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtga

SEQ ID NO: 24 - DNA, 66 bp, Homo sapiens
accctgattgcactggtcacctcgggagccctgctggctgtcttgggcatcactggctatttcctg

SEQ ID NO: 25 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 26 - protein, 21 aa, Homo sapiens
ALIAFLAFLIIVTSIALLVVL

SEQ ID NO: 27 - DNA, 3921 bp, Homo sapiens
atgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcatag

SEQ ID NO: 28 - protein, 220 aa, Homo sapiens; identical to SEQ ID NO: 18

SEQ ID NO: 29 - protein, 27 aa, Homo sapiens
FWVLVVVGGVLACYSLLVTVAFIIFWV

SEQ ID NO: 30 - DNA, 663 bp, Homo sapiens; identical to SEQ ID NO: 19

SEQ ID NO: 31 - protein, 235 aa, Homo sapiens; identical to SEQ ID NO: 12

SEQ ID NO: 32 - protein, 21 aa, Homo sapiens
IYIWAPLAGTCGVLLLSLVIT

SEQ ID NO: 33 - DNA, 708 bp, Homo sapiens; identical to SEQ ID NO: 14

SEQ ID NO: 34 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 35 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 36 - protein, 20 aa, Homo sapiens
MYRMQLLSCIALSLALVTNS

SEQ ID NO: 37 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 38 - protein, 23 aa, Homo sapiens
MKYTSYILAFQLCIVLGSLGCYC

SEQ ID NO: 39 - DNA, 501 bp, Homo sapiens
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 40 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 41 - protein, 21 aa, Homo sapiens
MDSYLLMWGLLTFIMVPGCQA

SEQ ID NO: 42 - DNA, 819 bp, Homo sapiens
atggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctag

SEQ ID NO: 43 - protein, 266 aa, Artificial Sequence; identical to SEQ ID NO: 7
  Description of Artificial Sequence: Synthetic polypeptide
  MOD_RES (28)..(28): Any amino acid
  MOD_RES (36)..(36): Any amino acid
  MOD_RES (60)..(60): Any amino acid
  MOD_RES (93)..(93): Any amino acid
  MOD_RES (251)..(251): Any amino acid

SEQ ID NO: 44-47 - skipped (4 entries)

SEQ ID NO: 48 - DNA, 131 bp, Homo sapiens
ccaccacgacgccagcgccgcgaccaccaacaccggcgcccaccatcgcgtcgcagcccctgtccctgcgcccagaggcgtgccggccagcggcggggggcgcagtgcacacgagggggctggacttcgcc

SEQ ID NO: 49 - DNA, 131 bp, Homo sapiens; identical to SEQ ID NO: 48

SEQ ID NO: 50-51 - skipped (2 entries)

SEQ ID NO: 52 - DNA, 120 bp, Homo sapiens
aaaattgaagttatgtatcctcctccttacctagacaatgagaagagcaatggaaccattatccatgtgaaagggaaacacctttgtccaagtcccctatttcccggaccttctaagccc

SEQ ID NO: 53 - DNA, 120 bp, Homo sapiens; identical to SEQ ID NO: 52

SEQ ID NO: 54 - protein, 393 aa, Artificial Sequence
  Description of Artificial Sequence: Synthetic polypeptide
MYRMQLLSCIALSLALVTNSQVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSSASGGGGSGGGGSGGGGSGGGGSGGGGSSDIVLTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIKSSGSGSTGPTTTPAPRPPTPAPTIASQPLSLRPEACRPAAGGAVHTRGLDFAPRKIEVMYPPPYLDNEKSNGTIIHVKGKHLCPSPLFPGPSKPTLIALVTSGALLAVLGITGYFL

SEQ ID NO: 55 - DNA, 1182 bp, Artificial Sequence
  Description of Artificial Sequence: Synthetic polynucleotide
atgtacagaatgcagctgctgagctgcatcgccctgagcctggccctggtgaccaacagccaggtgcagctgcagcagctgggcgccgagctggccagacccggcgccagcgtgaagatgagctgcaaggccagcggctacaccttcaccagctacagcatccagtgggtgaagcagagacccggccagggcctggagtggatcggctacatcaaccccagcagcggctacatcaagtacaaccagcacttcagagacagagccaccctgaccgccgacagaagcagcagcaccgcctacatgcagctgagcagcctgaccagcgaggacagcgccgtgtactactgcgccagaggcaacagcggcagcttcgactactggggccagggcaccaccctgaccgtgagcagcgccagcggcggcggcggcagcggcggcggcggcagcggcggcggcggcagcggcggcggcggcagcggcggcggcggcagcagcgacatcgtgctgacccaggccgcccccagcgtgcccgtgacccccggcgagagcctgagcatcagctgcagaagcagcaagagcctgctgcacagcagcggcatcacctacctgtactggttcctgcagagacccggccagagcccccagctgctgatctacagaatgagcaacctggccagcggcgtgcccgacagattcagcggcagcggcagcggcaccgccttcaccctgagaatcagcagagtggaggccgaggacgtgggcgtgtactactgcatgcagcacctggagtaccccttcaccttcggcggcggcaccaagctggagatcaagagcagcggcagcggcagcaccggtcccaccaccacccccgcccccagaccccccacccccgcccccaccatcgccagccagcccctgagcctgagacccgaggcctgcagacccgccgccggcggcgccgtgcacaccagaggcctggacttcgcccccagaaagatcgaggtgatgtacccccccccctacctggacaacgagaagagcaacggcaccatcatccacgtgaagggcaagcacctgtgccccagccccctgttccccggccccagcaagcccaccctgatcgccctggtgaccagcggcgccctgctggccgtgctgggcatcaccggctacttcctgtaa

SEQ ID NO: 56 - protein, 402 aa, Artificial Sequence
  Description of Artificial Sequence: Synthetic polypeptide
MYRMQLLSCIALSLALVTNSEVQLQLQQSGPELVKTGASVKISCKASGYSFTGYFIHWVKQSHGKSLEWIGYISCYNGATSYNQKFKGKATFTVDTSSSTAYMQFNSVTSEDSAVYYCVRNYYGNLDAMDYWGQGTSVTVSSASGGGGSGGGGSGGGGSGGGGSGGGGSSDIVMTQSHKFMSTSVGDRVSITCKASQDVSTAVAWYQQKPGQSPKILIYSASYRYTGVPDRFTGSGSGTDFTFTISSVQAEDLAVYYCQQHYSTPRTFGGGTKLEIKRADAAQTCISSGSGSTGPTTTPAPRPPTPAPTIASQPLSLRPEACRPAAGGAVHTRGLDFAPRKIEVMYPPPYLDNEKSNGTIIHVKGKHLCPSPLFPGPSKPTLIALVTSGALLAVLGITGYFL

SEQ ID NO: 57 - DNA, 1209 bp, Artificial Sequence
  Description of Artificial Sequence: Synthetic polynucleotide
atgtacagaatgcagctgctgagctgcatcgccctgagcctggccctggtgaccaacagcgaggtgcagctgcagctgcagcagagcggccccgagctggtgaagaccggcgccagcgtgaagatcagctgcaaggccagcggctacagcttcaccggctacttcatccactgggtgaagcagagccacggcaagagcctggagtggatcggctacatcagctgctacaacggcgccaccagctacaaccagaagttcaagggcaaggccaccttcaccgtggacaccagcagcagcaccgcctacatgcagttcaacagcgtgaccagcgaggacagcgccgtgtactactgcgtgagaaactactacggcaacctggacgccatggactactggggccagggcaccagcgtgaccgtgagcagcgccagcggcggcggcggcagcggcggcggcggcagcggcggcggcggcagcggcggcggcggcagcggcggcggcggcagcagcgacatcgtgatgacccagagccacaagttcatgagcaccagcgtgggcgacagagtgagcatcacctgcaaggccagccaggacgtgagcaccgccgtggcctggtaccagcagaagcccggccagagccccaagatcctgatctacagcgccagctacagatacaccggcgtgcccgacagattcaccggcagcggcagcggcaccgacttcaccttcaccatcagcagcgtgcaggccgaggacctggccgtgtactactgccagcagcactacagcacccccagaaccttcggcggcggcaccaagctggagatcaagagagccgacgccgcccagacctgcatcagcagcggcagcggcagcaccggtcccaccaccacccccgcccccagaccccccacccccgcccccaccatcgccagccagcccctgagcctgagacccgaggcctgcagacccgccgccggcggcgccgtgcacaccagaggcctggacttcgcccccagaaagatcgaggtgatgtacccccccccctacctggacaacgagaagagcaacggcaccatcatccacgtgaagggcaagcacctgtgccccagccccctgttccccggccccagcaagcccaccctgatcgccctggtgaccagcggcgccctgctggccgtgctgggcatcaccggctacttcctgtaa

SEQ ID NO: 58 - protein, 399 aa, Artificial Sequence
  Description of Artificial Sequence: Synthetic polypeptide
MYRMQLLSCIALSLALVTNSQVQLKESGPGLVKPSLTLSLTCTVSGFSLNSYGVIWVRQPPGKGLEWLGVKWGYGNTNYNSALKSRLNINRDTSKSQVFLKMDNVQTEDTAMYFCARSRFNYGGPLDYWGQGVMVTVSSASGGGGSGGGGSGGGGSGGGGSGGGGSSDIVLTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRADAAPTVSSSGSGSTGPTTTPAPRPPTPAPTIASQPLSLRPEACRPAAGGAVHTRGLDFAPRKIEVMYPPPYLDNEKSNGTIIHVKGKHLCPSPLFPGPSKPTLIALVTSGALLAVLGITGYFL

SEQ ID NO: 59 - DNA, 1200 bp, Artificial Sequence
  Description of Artificial Sequence: Synthetic polynucleotide
atgtacagaatgcagctgctgagctgcatcgccctgagcctggccctggtgaccaacagccaggtgcagctgaaggagagcggccccggcctggtgaagcccagcctgaccctgagcctgacctgcaccgtgagcggcttcagcctgaacagctacggcgtgatctgggtgagacagccccccggcaagggcctggagtggctgggcgtgaagtggggctacggcaacaccaactacaacagcgccctgaagagcagactgaacatcaacagagacaccagcaagagccaggtgttcctgaagatggacaacgtgcagaccgaggacaccgccatgtacttctgcgccagaagcagattcaactacggcggccccctggactactggggccagggcgtgatggtgaccgtgagcagcgccagcggcggcggcggcagcggcggcggcggcagcggcggcggcggcagcggcggcggcggcagcggcggcggcggcagcagcgacatcgtgctgacccagagccccaagagcatgagcatgagcgtgggcgagagagtgaccctgacctgcaaggccagcgagaacgtggtgacctacgtgagctggtaccagcagaagcccgagcagagccccaagctgctgatctacggcgccagcaacagatacaccggcgtgcccgacagattcaccggcagcggcagcgccaccgacttcaccctgaccatcagcagcgtgcaggccgaggacctggccgactaccactgcggccagggctacagctacccctacaccttcggcggcggcaccaagctggagatcaagagagccgacgccgcccccaccgtgagcagcagcggcagcggcagcaccggtcccaccaccacccccgcccccagaccccccacccccgcccccaccatcgccagccagcccctgagcctgagacccgaggcctgcagacccgccgccggcggcgccgtgcacaccagaggcctggacttcgcccccagaaagatcgaggtgatgtacccccccccctacctggacaacgagaagagcaacggcaccatcatccacgtgaagggcaagcacctgtgccccagccccctgttccccggccccagcaagcccaccctgatcgccctggtgaccagcggcgccctgctggccgtgctgggcatcaccggctacttcctgtaa

SEQ ID NO: 60 - protein, 402 aa, Artificial Sequence
  Description of Artificial Sequence: Synthetic polypeptide
MYRMQLLSCIALSLALVTNSQVQLKESGAELARPGASVKMSCKASGYTFTSYTMQWVKQRPGQGLEWIGYINPSSGYIKYNQKFKDKVTLTADKSSTTAYMQLSRLTSEDSAVYYCARRGSYFFDFWGQGTSVTVSSASGGGGSGGGGSGGGGSGGGGSGGGGSSDIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGGGTKLEVKRADAAPTVSSSGSGSTGPTTTPAPRPPTPAPTIASQPLSLRPEACRPAAGGAVHTRGLDFAPRKIEVMYPPPYLDNEKSNGTIIHVKGKHLCPSPLFPGPSKPTLIALVTSGALLAVLGITGYFL

SEQ ID NO: 61 - DNA, 1209 bp, Artificial Sequence
  Description of Artificial Sequence: Synthetic polynucleotide
atgtacagaatgcagctgctgagctgcatcgccctgagcctggccctggtgaccaacagccaggtgcagctgaaggagagcggcgccgagctggccagacccggcgccagcgtgaagatgagctgcaaggccagcggctacaccttcaccagctacaccatgcagtgggtgaagcagagacccggccagggcctggagtggatcggctacatcaaccccagcagcggctacatcaagtacaaccagaagttcaaggacaaggtgaccctgaccgccgacaagagcagcaccaccgcctacatgcagctgagcagactgaccagcgaggacagcgccgtgtactactgcgccagaagaggcagctacttcttcgacttctggggccagggcaccagcgtgaccgtgagcagcgccagcggcggcggcggcagcggcggcggcggcagcggcggcggcggcagcggcggcggcggcagcggcggcggcggcagcagcgacatcgtgatcacccaggacgagctgagcaaccccgtgaccagcggcgagagcgtgagcatcagctgcagaagcagcaagagcctgctgtacaaggacggcaagacctacctgaactggttcctgcagagacccggccagagcccccagctgctgatctacctgatgagcaccagagccagcggcgtgagcgacagattcagcggcagcggcagcggcaccgacttcaccctggagatcagcagagtgaaggccgaggacgtgggcgtgtactactgccagcagctggtggagtaccccttcaccttcggcggcggcaccaagctggaggtgaagagagccgacgccgcccccaccgtgagcagcagcggcagcggcagcaccggtcccaccaccacccccgcccccagaccccccacccccgcccccaccatcgccagccagcccctgagcctgagacccgaggcctgcagacccgccgccggcggcgccgtgcacaccagaggcctggacttcgcccccagaaagatcgaggtgatgtacccccccccctacctggacaacgagaagagcaacggcaccatcatccacgtgaagggcaagcacctgtgccccagccccctgttccccggccccagcaagcccaccctgatcgccctggtgaccagcggcgccctgctggccgtgctgggcatcaccggctacttcctgtaa

SEQ ID NO: 62 - protein, 391 aa, Artificial Sequence
  Description of Artificial Sequence: Synthetic polypeptide
MYRMQLLSCIALSLALVTNSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKSGGGGSGGGGSGGGGSGGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRSSGSGSTGPTTTPAPRPPTPAPTIASQPLSLRPEACRPAAGGAVHTRGLDFAPRKIEVMYPPPYLDNEKSNGTIIHVKGKHLCPSPLFPGPSKPTLIALVTSGALLAVLGITGYFL

SEQ ID NO: 63 - DNA, 1176 bp, Artificial Sequence
  Description of Artificial Sequence: Synthetic polynucleotide
atgtacagaatgcagctgctgagctgcatcgccctgagcctggccctggtgaccaacagccaggtgcagctgcagcagagcggcgccgagctggccagacccggcgccagcgtgaagatgagctgcaaggccagcggctacaccttcaccagatacaccatgcactgggtgaagcagagacccggccagggcctggagtggatcggctacatcaaccccagcagaggctacaccaactacaaccagaagttcaaggacaaggccaccctgaccaccgacaagagcagcagcaccgcctacatgcagctgagcagcctgaccagcgaggacagcgccgtgtactactgcgccagatactacgacgaccactactgcctggactactggggccagggcaccaccctgaccgtgagcagcgccaagagcggcggcggcggcagcggcggcggcggcagcggcggcggcggcagcggcggcggcggcagcggcggcggcggcagcagccagatcgtgctgacccagagccccgccatcatgagcgccagccccggcgagaaggtgaccatgacctgcagcgccagcagcagcgtgagctacatgaactggtaccagcagaagagcggcaccagccccaagagatggatctacgacaccagcaagctggccagcggcgtgcccgcccacttcagaggcagcggcagcggcaccagctacagcctgaccatcagcggcatggaggccgaggacgccgccacctactactgccagcagtggagcagcaaccccttcaccttcggcagcggcaccaagctggagatcaacagaagcagcggcagcggcagcaccggtcccaccaccacccccgcccccagaccccccacccccgcccccaccatcgccagccagcccctgagcctgagacccgaggcctgcagacccgccgccggcggcgccgtgcacaccagaggcctggacttcgcccccagaaagatcgaggtgatgtacccccccccctacctggacaacgagaagagcaacggcaccatcatccacgtgaagggcaagcacctgtgccccagccccctgttccccggccccagcaagcccaccctgatcgccctggtgaccagcggcgccctgctggccgtgctgggcatcaccggctacttcctgtaa

SEQ ID NO: 64 - protein, 395 aa, Artificial Sequence
  Description of Artificial Sequence: Synthetic polypeptide
MYRMQLLSCIALSLALVTNSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGRTEVATPGAYWGQGTMVTVSSASGGGGSGGGGSGGGGSGGGGSGGGGSSQAVLTQPSSVSGAPGQRVTISCTGSSSNIGAGYDVHWYQQLPGTAPKLLIYGNSNRPSGVPDRFSGSKSGTSASLAVTGLQAEDEADYYCQSYDSSLSDVVFGGGTKLTVLSSGSGSTGPTTTPAPRPPTPAPTIASQPLSLRPEACRPAAGGAVHTRGLDFAPRKIEVMYPPPYLDNEKSNGTIIHVKGKHLCPSPLFPGPSKPTLIALVTSGALLAVLGITGYFL

SEQ ID NO: 65 - DNA, 1188 bp, Artificial Sequence
  Description of Artificial Sequence: Synthetic polynucleotide
atgtacagaatgcagctgctgagctgcatcgccctgagcctggccctggtgaccaacagcgaggtgcagctgctggagagcggcggcggcctggtgcagcccggcggcagcctgagactgagctgcgccgccagcggcttcaccttcagcagctacgccatgagctgggtgagacaggcccccggcaagggcctggagtgggtgagcgccatcagcggcagcggcggcagcacctactacgccgacagcgtgaagggcagattcaccatcagcagagacaacagcaagaacaccctgtacctgcagatgaacagcctgagagccgaggacaccgccgtgtactactgcgccagaggcagaaccgaggtggccacccccggcgcctactggggccagggcaccatggtgaccgtgagcagcgccagcggcggcggcggcagcggcggcggcggcagcggcggcggcggcagcggcggcggcggcagcggcggcggcggcagcagccaggccgtgctgacccagcccagcagcgtgagcggcgcccccggccagagagtgaccatcagctgcaccggcagcagcagcaacatcggcgccggctacgacgtgcactggtaccagcagctgcccggcaccgcccccaagctgctgatctacggcaacagcaacagacccagcggcgtgcccgacagattcagcggcagcaagagcggcaccagcgccagcctggccgtgaccggcctgcaggccgaggacgaggccgactactactgccagagctacgacagcagcctgagcgacgtggtgttcggcggcggcaccaagctgaccgtgctgagcagcggcagcggcagcaccggtcccaccaccacccccgcccccagaccccccacccccgcccccaccatcgccagccagcccctgagcctgagacccgaggcctgcagacccgccgccggcggcgccgtgcacaccagaggcctggacttcgcccccagaaagatcgaggtgatgtacccccccccctacctggacaacgagaagagcaacggcaccatcatccacgtgaagggcaagcacctgtgccccagccccctgttccccggccccagcaagcccaccctgatcgccctggtgaccagcggcgccctgctggccgtgctgggcatcaccggctacttcctgtaa

SEQ ID NO: 66 - protein, 207 aa, Artificial Sequence
  Description of Artificial Sequence: Synthetic polypeptide
MNTVIRIVLLSLLVAFSQAGFHTINATWWANITLVGPPDTPVTWYDTQGLWFCNGSRVKNPQIRHTCNDQNLTLIHVNKTYERTYMGYNRQGTKKEDYKVVVIGGGGSDEGKRYRVKVIPPNTTNSQSVKIQPYTRQTTPDQEHKFELQFETNGNYDSKIPSTTVAIVVGVIAGFITLIIVFICYICCRKRPRAYNHMVDPLLSFSY

SEQ ID NO: 67 - DNA, 624 bp, Artificial Sequence
  Description of Artificial Sequence: Synthetic polynucleotide
atgaacacggtgatccgcatagtccttctgtctctgctggtggctttctcccaggccggcttccacacaattaatgccacctggtgggctaacattactctcgtaggccccccggatacccccgtgacttggtacgacactcagggtctgtggttctgtaacgggagtcgagtgaaaaatcctcaaattcgccatacctgtaacgaccaaaatctgaccttgatccacgtgaacaagacatacgagcgtacatatatgggctacaataggcagggtacaaagaaagaggactataaagtggtagtgattggcggcggcggcagcgatgagggaaaacggtaccgggttaaggttattccgcctaacaccacaaactcccagagtgtcaaaattcagccttacaccaggcagactactcctgaccaggaacacaaattcgaattacagtttgagactaacggtaactatgactccaagattccatctacaacggtcgcgatcgtagtgggcgtgattgcaggcttcatcacattgatcatcgtgttcatctgctatatctgctgtaggaagcgccctcgggcgtacaaccacatggtggaccctctgttgagtttctcatattaa

SEQ ID NO: 68 - protein, 201 aa, Artificial Sequence
  Description of Artificial Sequence: Synthetic polypeptide
MNTVIRIVLLSLLVAFSQAGFHTINATWWANITLVGGGGSVTVTAGSNLTLVGPKAEGKVTWFDGDLKRPCEPNYRLRHECNNQNLTLINVTKDYEGTYYGTNDKDEGKRYRVKVNTTNSQSVKIQPYTRQTTPDQEHKFELQFETNGNYDSKIPSTTVAIVVGVIAGFITLIIVFICYICCRKRPRAYNHMVDPLLSFSY

SEQ ID NO: 69 - DNA, 606 bp, Artificial Sequence
  Description of Artificial Sequence: Synthetic polynucleotide
atgaacacggtgatccgcatagtccttctgtctctgctggtggctttctcccaggccggcttccacacaattaatgccacctggtgggctaacattactctcgtaggcggcggcggcagcgtgacagtaactgctggaagtaacctgaccctcgtggggcccaaggcggaggggaaagtaacctggttcgacggcgatctaaaacgcccctgtgaaccaaactacagacttagacacgaatgcaacaaccagaacctgactctgattaacgtgaccaaggactacgaaggaacatactacgggacgaatgataaggatgagggaaaacggtaccgggttaaggttaacaccacaaactcccagagtgtcaaaattcagccttacaccaggcagactactcctgaccaggaacacaaattcgaattacagtttgagactaacggtaactatgactccaagattccatctacaacggtcgcgatcgtagtgggcgtgattgcaggcttcatcacattgatcatcgtgttcatctgctatatctgctgtaggaagcgccctcgggcgtacaaccacatggtggaccctctgttgagtttctcatattaa

SEQ ID NO: 70 - DNA, 360 bp, Artificial Sequence
  Description of Artificial Sequence: Synthetic polynucleotide
atggcccaggtgcagctggtggagagcggcggcggcctggtgcaccccggcgacagcctgagactgagctgcgccgccagcggcagcgtgttcaacagcgccaccatgggctggtacagacagagccccggcagccagagagagctggtggccaccatcgtggtgggcacccccacctacgccgacagcgtgaagggcagattcaccatcagcagagacaacgccaagaacatcgtgtacctgcagatgaacagcctgaagcccgaggacaccgccgtgtactactgcaactacagagccacctacaccagcggctacagcagagactactggggccagggcacccaggtgaccgtgagc

SEQ ID NO: 71 - protein, 120 aa, Artificial Sequence
  Description of Artificial Sequence: Synthetic polypeptide
MAQVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVS

SEQ ID NO: 72 - DNA, 360 bp, Artificial Sequence
  Description of Artificial Sequence: Synthetic polynucleotide
atggcccaggtgcagctggtgcagagcggcggcggcctggtgcagcccggcggcagcctgagactgagctgcgccgccagcggcagagccttcaacagcgccgccatgggctggtacagacaggcccccggcagccagagagagctggtggccagcatcagcgccggcaccgccagctacgccgacgccgtgaagggcagattcaccatcagcagagactacgccaagaacatcatctacctgcagatgaacagcctgaagcccgacgacaccgccgtgtacttctgcaactacagaaccacctacaccagcggctacagcgaggactactggggccagggcacccaggtgaccgtgagc

SEQ ID NO: 73 - protein, 120 aa, Artificial Sequence
  Description of Artificial Sequence: Synthetic polypeptide
MAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVS

SEQ ID NO: 74 - protein, 130 aa, Artificial Sequence
  Description of Artificial Sequence: Synthetic polypeptide
EVQLVESGGGLVQPGGSLRLSCAASERAYRNRLLGWFRQVPGKEREFVAWIRPIDSSTNYADSVRGRFTISRDNARSTLHLQMNSLKPEDTAVYYCVKGNGLTSTRASDYWGQGTQVTVLSAHHSEDPIS

SEQ ID NO: 75 - DNA, 390 bp, Artificial Sequence
  Description of Artificial Sequence: Synthetic polynucleotide
gaggtgcagctggtggagtctggcggaggcttggtgcagcctggggggtctctgagactctcctgtgcagcctctgaacgcgcctacaggaaccgtcttcttggctggttccgccaggttccagggaaggagcgtgaatttgtggcatggatcagacccattgatagcagcacaaattatgcagactccgtgaggggccgattcaccatctccagagataacgccaggagcacgctgcatctgcaaatgaacagcctgaaacctgaggacacggccgtgtattactgtgtaaaggggaacggacttacttctactcgcgcgagtgactactggggccaggggacccaggtcaccgtcttgtcagcgcaccacagcgaagaccctattagt

SEQ ID NO: 76 - protein, 135 aa, Artificial Sequence
  Description of Artificial Sequence: Synthetic polypeptide
EVQLLESGGGLVQAGDSLRLSCAASGLTNPERRLAWFRQAPGKEREFVASIRWSGGPNTHYGDSVKGRFTISRDNGKNTVALQMNNLKPEDTAVYFCAAAVRLTAPLNFDTSYDYWGQGTQVTISSEPKTPKPQT

SEQ ID NO: 77 - DNA, 405 bp, Artificial Sequence
  Description of Artificial Sequence: Synthetic polynucleotide
gaggtgcagctgctggagtctgggggaggattggtgcaggctggggactctctgagactctcctgtgcagcttctggactgactaaccctgaaagacgcttggcctggttccgccaggctccagggaaggagcgtgagtttgtagcgtccattcgctggagtggtggtcccaacacacactatggcgactccgtgaagggccgattcaccatctccagagacaacggcaagaacacggtggctctacaaatgaacaacctgaaacctgaggacacggccgtttatttctgtgcagcggctgtgcgtctaactgcgcctctcaattttgacacctcgtatgactactggggccaggggacccaggtcaccatctcctcagaacccaagacaccaaaaccacaaact

SEQ ID NO: 78 - protein, 132 aa, Artificial Sequence
  Description of Artificial Sequence: Synthetic polypeptide
EVQLEESGGGLVQPGGSLRLSCAASGFTFSNQVMSWVRQAPGKGPERVAVIGSVGGATGATSYADSVRGRFTISRDNARSTLHLQMNSLKPEDTAVYYCAARVRGSTGDFGSWGQGTQVTVSSEPKTPKPQT

SEQ ID NO: 79 - DNA, 396 bp, Artificial Sequence
  Description of Artificial Sequence: Synthetic polynucleotide
gaggtgcagctggaggagtctgggggaggcttggtgcagcctggggggtctctgagactctcctgtgcagcctctggattcaccttcagtaaccaagtcatgagctgggtccgccaggctccaggaaaggggcccgagcgggtcgcagttatcggcagtgtcggaggtgccacaggtgccacaagttatgcagactccgtgaggggccgattcaccatctccagagataacgccaggagcacgctgcatctgcaaatgaacagcctgaaacctgaggacacggccgtgtattactgtgcagcgagggtacgcggcagcacaggggactttggttcctggggccaggggacccaggtcaccgtctcctcggaacccaagacaccaaaaccacaaact

SEQ ID NO: 80 - protein, 134 aa, Artificial Sequence
  Description of Artificial Sequence: Synthetic polypeptide
EVQLVESGGGLVETGGSLRLSCAGSGRTFSSRHVGWFRQTPGKEREFVASIRWSGGHTYYADSVKGRFTISRDNGKNTVALQMNNLKPEDTAVYFCAAAVRLTAPLNFDTSYDYWGQGTQVTISSEPKTPKPQT

SEQ ID NO: 81 - DNA, 402 bp, Artificial Sequence
  Description of Artificial Sequence: Synthetic polynucleotide
gaggtgcagctggtggagtctgggggaggattggtcgaaactgggggttctctgagactctcctgtgcaggttctggacgcaccttcagtagccggcacgtgggctggttccgccagactccagggaaggagcgtgagtttgtagcatccattaggtggagtggcggtcacacatactatgcagactccgtgaagggccgattcaccatctccagagacaacggcaagaacacggtggctctacaaatgaacaacctgaaacctgaggacacggccgtttatttctgtgcagcggctgtgcgtctaactgcgcctctcaattttgacacctcgtatgactactggggccaggggacccaggtcaccatctcctcagaacccaagacaccaaaaccacaaact

SEQ ID NO: 82 - protein, 133 aa, Artificial Sequence
  Description of Artificial Sequence: Synthetic polypeptide
EVQLVESGGGLVQPGGSLRLSCAASGFTFSNQVMSWVRQAPGKGPERVAVIGSVGGATGATSYADSVRGRFTISRDNARSTLHLQMNSLKPEDTAVYYCVKGNGLTSTRASDYWGQGTQVTVSSEPKTPKPQT

SEQ ID NO: 83 - DNA, 399 bp, Artificial Sequence
  Description of Artificial Sequence: Synthetic polynucleotide
gaggtgcagctggtggagtctgggggaggcttggtgcagcctggggggtctctgagactctcctgtgcagcctctggattcaccttcagtaaccaagtcatgagctgggtccgccaggctccaggaaaggggcccgagcgggtcgcagttatcggcagtgtcggaggtgccacaggtgccacaagttatgcagactccgtgaggggccgattcaccatctccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 84 - protein, 133 aa, Artificial Sequence
  Description of Artificial Sequence: Synthetic polypeptide
EVQLQESGGGLVQPGGSLRLSCVASGFTFSIYAMSWVRQAPGKGPERVAVIGSVGGATGVTSYADSVKDRFTISRDNARSTLHLQMNSLKPEDTAVYYCVKGNGLTSTRASDYWGQGTQVTVSSEPKTPKPQT

SEQ ID NO: 85 - DNA, 399 bp, Artificial Sequence
  Description of Artificial Sequence: Synthetic polynucleotide
gaggtgcagctgcaggagtctgggggaggcttggtgcagcctggggggtctctgagactctcctgtgtagcctctggattcaccttcagtatctacgccatgagctgggtccgccaggctccaggaaaggggcccgagcgggtcgcagttatcggcagtgtcggaggtgccacaggtgtcacaagttatgcagactccgtgaaggaccgattcaccatctccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 86 - protein, 120 aa, Artificial Sequence
  Description of Artificial Sequence: Synthetic polypeptide
EVQLVESGGGLVQAGGSLKLSCAASGRTLTYYTAWFRQAPGKEREFVASLGWSGDVTYYADSVKGRFTISGDNAKNTVYLQMNSLKPEDTATYYCNVMQAWGQGTQVTVSSEPKTPKPQT

SEQ ID NO: 87 - DNA, 360 bp, Artificial Sequence
  Description of Artificial Sequence: Synthetic polynucleotide
gaggtgcagctggtggagtctgggggaggattggtgcaggcggggggctctctgaaactctcctgtgcagcctccggacgcaccctcacttattatactgcctggttccgccaggctccagggaaggagcgtgagtttgtagcatcgctagggtggagtggcgatgtcacatactatgcagactccgtgaagggccgattcaccatctccggcgacaacgccaagaacacggtatatctgcaaatgaacagcctgaaacccgaggacacggccacttattactgtaatgtcatgcaggcttggggtcaggggacccaggtcaccgtctcctcagaacccaagacaccaaaaccacaaact

SEQ ID NO: 88 - protein, 135 aa, Artificial Sequence; identical to SEQ ID NO: 76
  Description of Artificial Sequence: Synthetic polypeptide

SEQ ID NO: 89 - DNA, 405 bp, Artificial Sequence
  Description of Artificial Sequence: Synthetic polynucleotide
gaggtgcagctgctggagtctgggggaggattggtgcaggctggggactctctgagactctcctgtgcagcttctggactgaccaaccctgaaagacgcttggcctggttccgccaggctccagggaaggagcgtgagtttgtagcgtccattcgctggagtggtggtcccaacacacactatggggactccgtgaagggccgattcaccatctccagagacaacggcaagaacacggtggctctacaaatgaacaacctgaaacctgaggacacggccgtttatttctgtgcagcggctgtgcgtctaactgcgcctctcaattttgacacctcgtatgactactggggccaggggacccaggtcaccatctcctcagaacccaagacaccaaaaccacaaact

SEQ ID NO: 90 - protein, 133 aa, Artificial Sequence
  Description of Artificial Sequence: Synthetic polypeptide
EVQLLESGGGLVQAGGSLRLSCAASGRTLTFYTGWFRQAPGKEREFVASIRWSGGHTYYADSVKGRFTISGDNAKNTVYLQMNSLKPEDTAIYYCAALRSWTTTPQREDLYDVWGQGTQVTVSSEPKTPKPQT

SEQ ID NO: 91 - DNA, 399 bp, Artificial Sequence
  Description of Artificial Sequence: Synthetic polynucleotide
gaggtgcagctgctggagtctgggggaggattggtgcaggcggggggctctctgagactctcctgtgcagcctccggacgcaccctcactttttatactggctggttccgccaggctccagggaaggagcgtgagtttgtagcatccattaggtggagtggcggtcacacatactatgcagactccgtgaagggccgattcaccatctccggagacaacgccaagaacacggtgtatctacaaatgaacagcctgaaacccgaggacacggccatttattactgcgcagcacttagatcttggactactacacctcagagggaggacctctatgatgtctggggccaggggacccaggtcaccgtctcctcagaacccaagacaccaaaaccacaaact

SEQ ID NO: 92 - protein, 133 aa, Artificial Sequence
  Description of Artificial Sequence: Synthetic polypeptide
EVQLQESGGGLVQAGGSLRLSCAASGRTLTFYTGWFRQAPGKEREFVASIRWSGGNTYYADSVKGRFTITGDNAKNTVYLQMNSLKPEDTAIYYCAALRSWTTTPQREVLYDNWGHGTQVTVSSAHHSEDPIS

SEQ ID NO: 93 - DNA, 399 bp, Artificial Sequence
  Description of Artificial Sequence: Synthetic polynucleotide
gaggtgcagctgcaggagtctgggggaggattggtgcaggcggggggctctctgagactctcctgtgcagcctccggacgcaccctcactttttatactggctggttccgccaggctccagggaaggagcgtgagtttgtagcatctattaggtggagtggcggtaacacatactatgcagactccgtgaagggccgattcaccatcaccggagacaacgccaagaacacggtgtatctgcaaatgaacagcctgaaacccgaggacacggccatttattactgcgcagcacttagatcttggactactacacctcagagggaggtcctctatgacaactggggccacgggacccaggtcaccgtctcctcagcgcaccacagcgaagaccctattagt

SEQ ID NO: 94 - protein, 132 aa, Artificial Sequence
  Description of Artificial Sequence: Synthetic polypeptide
EVQLEESGGGLVQAGDSLRLSCACSERAYRNRLLGWFRQAPGKEREFVANIRPIDSASDYAGSVKGRFTISRDIAKRTVYLQMNSLKPEDTAVYYCASTYMFDSVREDEYDYWGQGTQVTVSSEPKTPKPQT

SEQ ID NO: 95 - DNA, 396 bp, Artificial Sequence
  Description of Artificial Sequence: Synthetic polynucleotide
gaggtgcagctggaggagtctgggggaggattggtgcaggctggggactctctgagactctcctgtgcttgctctgaacgcgcctataggaaccgtcttcttggctggttccgccaggctccaggaaaggagcgtgaatttgtagcaaatatcagacccattgatagcgcctccgattatgcaggctccgtgaagggccgattcaccatctctagagacatcgccaagagaacggtgtatctgcaaatgaacagcctgaaacctgaggacacggccgtttattattgtgcgtccacatacatgttcgatagtgtccgggaggatgaatatgactactggggccagggaacccaggtcaccgtctcctcagaacccaagacaccaaaaccacaaact

SEQ ID NO: 96 - protein, 129 aa, Artificial Sequence
  Description of Artificial Sequence: Synthetic polypeptide
EVQLVESGGGLVQAGGSLRLSCVVSGRTLTFYTGWFRQAPGKEREFVASIRWSGGNTYYADSVKGRFTITRDNARSTLHLQMNSLKPEDTAVYYCVKGNGLTSTRASDYWGQGTQVTISSEPKTPKPQT

SEQ ID NO: 97 - DNA, 387 bp, Artificial Sequence
  Description of Artificial Sequence: Synthetic polynucleotide
gaggtgcagctggtggagtctgggggaggattggtgcaggctgggggctctctgagactctcgtgtgtagtctctggacgcaccctcactttttatactggctggttccgccaggctccagggaaggagcgtgagtttgtagcatctattaggtggagtggcggtaacacatactatgcagactccgtgaagggccgattcaccatcaccagagataacgccaggagcacgctgcatcttcaaatgaacagcctgaaacctgaggacacggccgtgtattactgtgtaaaggggaacggacttacttctactcgcgcgagtgactactggggccaggggacccaggtcaccatctcctcagaacccaagacaccaaaaccacaaact

SEQ ID NO: 98 - protein, 129 aa, Artificial Sequence
  Description of Artificial Sequence: Synthetic polypeptide
EVQLLESGGGLVQAGGSLRLSCVASGRGFSRYDMGWFRQASGKEREFVAAISWSNSTTAYADSVKGRFAISRDNNKNMVYLQMNSLKPEDTAVYYCAARVRGSTGDFGSWGQGTQVTVSSEPKTPKPQT

SEQ ID NO: 99 - DNA, 387 bp, Artificial Sequence
  Description of Artificial Sequence: Synthetic polynucleotide
gaggtgcagctgctggagtctgggggaggcttggtgcaggctgggggctctctgagactctcctgtgtagcctctggacggggcttcagtaggtatgacatgggctggttccgccaggcttcagggaaggagcgtgagtttgtagcagcaattagctggagtaatagtaccacggcctatgcagactccgtgaagggccgattcgccatctcaagagacaacaacaagaatatggtgtatctgcaaatgaacagcctgaaaccggaggacacggccgtgtattactgtgcagcgagggtacgcggcagcacaggggactttggttcctggggccaggggacccaggtcaccgtctcctcggaacccaagacaccaaaaccacaaact

SEQ ID NO: 100 - protein, 137 aa, Artificial Sequence
  Description of Artificial Sequence: Synthetic polypeptide
EVQLVESGGGLVQAGGSLSLSCAASGRTFSTGAMGWFRQAPGKEREFLARITLIGHGTYYADALKGRFTISRDHAKNTVYLQMNSLKPEDTAVYYCVARDSPCVGNCWYENAGDYNYWGQGTQVTVSSEPKTPKPQT

SEQ ID NO: 101 - DNA, 411 bp, Artificial Sequence
  Description of Artificial Sequence: Synthetic polynucleotide
gaggtgcagctggtggagtctgggggaggattggtgcaggctgggggctctctgagtctctcctgtgcagcctctggacgcaccttcagtaccggtgccatgggctggttccgccaggctccagggaaggagcgtgagtttctggcacgaattactctgattggccacggcacatactatgcagatgccttgaagggccgattcaccatttccagagaccacgctaagaacacggtgtatctgcaaatgaacagcctgaaacctgaggacacggccgtatattactgtgtagcgcgagacagcccgtgcgtgggtaattgttggtacgagaatgcgggcgactataattactggggccaggggacccaggtcaccgtctcctcagaacccaagacaccaaaaccacaaact

SEQ ID NO: 102 - protein, 119 aa, Artificial Sequence
  Description of Artificial Sequence: Synthetic polypeptide
EVQLLESGGGLVQAGGSLRLSCVSSGDSISGVVVRWYRQVPGKQREWIGGIGTSDNPEYADSVWGRFVLSRDNAGSRVNLQMNNLKLEDTATYYCNAVHKWGPGTQVTVSSEPKTPKPQ

SEQ ID NO: 103 - DNA, 359 bp, Artificial Sequence
  Description of Artificial Sequence: Synthetic polynucleotide
gaggtgcagctgctggagtctgggggaggcctggtgcaggctggggggtctctgagactctcctgtgtaagttctggagacagtatcagtggagtggtcgtccgttggtaccgccaggttccagggaagcagcgcgagtggatcggaggtattggtactagtgataacccagaatatgcggactccgtctggggccgattcgtcctctccagagacaatgccgggagccgcgtaaatctgcaaatgaacaacctgaaacttgaggacacggccacctattactgcaatgcagtgcacaaatggggcccgggtacccaggtcaccgtctcttctgaacccaagacaccaaaaccacaaac

SEQ ID NO: 104 - protein, 134 aa, Artificial Sequence
  Description of Artificial Sequence: Synthetic polypeptide
EVQLLESGGGLVQPGGSLRLSCAASGFTFSNAVMSWVRQAPGKEREFVASIRWSGGNTYYADSVKGRFTITGDNAKNTVYLQMNSLKPEDTAIYYCAALRSWTTTPQREVLYDNWGHGTQVTVSSEPKTPKPQT

SEQ ID NO: 105 - DNA, 402 bp, Artificial Sequence
  Description of Artificial Sequence: Synthetic polynucleotide
gaggtgcagctgctggagtctgggggaggcttggtgcagcctggggggtctctgagactctcctgtgcagcctctggattcaccttcagtaacgccgtcatgagctgggtccgccaggctccagggaaggagcgtgagtttgtagcatctattaggtggagtggcggtaacacatactatgcagactccgtgaagggccgattcaccatcaccggagacaacgccaagaacacggtgtatctgcaaatgaacagcctgaaacccgaggacacggccatttattactgcgcagcacttagatcttggactactacacctcagagggaggtcctctatgacaactggggccacgggacccaggtcaccgtctcctcagaacccaagacaccaaaaccacaaact

SEQ ID NO: 106 - protein, 128 aa, Artificial Sequence
  Description of Artificial Sequence: Synthetic polypeptide
EVQLEESGGGLVQAGGSLRLSCAASGRTFSSYRLGWFRQAPGKEREFVAGWSGGSTYYADSVKGRFTISRDNAKNTVYLQMNSLKPEDTAVYYCVKGNGLTSTRASDYWGQGTQVTVSSEPKTPKPQT

SEQ ID NO: 107 - DNA, 384 bp, Artificial Sequence
  Description of Artificial Sequence: Synthetic polynucleotide
gaggtgcagctggaggagtctgggggaggattggtgcaggctgggggctctctgagactctcctgtgcagcctctggacgcaccttcagtagctatcgactgggctggttccgccaggctccagggaaggagcgtgagtttgtagcaggctggagtggtggtagcacatactatgcagactccgtgaagggccgattcaccatctccagagacaacgccaagaacacggtgtatctgcaaatgaacagcctcaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 108 - protein, 135 aa, Artificial Sequence
  Description of Artificial Sequence: Synthetic polypeptide
EVQLVESGGGLVQAGDSLRLSCAASGLTNPERRLAWFRQAPGKEREFVASIRWSGGPNTHYGDSVKGRFTISRDNGKNTVALQMNNLKPEDTAVYFCAAAVRLTAPLNFDTSYDYWGQGTQVTISSEPKTPKPQT

SEQ ID NO: 109 - DNA, 405 bp, Artificial Sequence
  Description of Artificial Sequence: Synthetic polynucleotide
gaggtgcagctggtggagtctgggggaggattggtgcaggctggggactctctgagactctcctgtgcagcttctggactgaccaaccctgaaagacgcttggcctggttccgccaggctccagggaaggagcgtgagtttgtagcgtccattcgctggagtggtggtcccaacacacactatggagactccgtgaagggccgattcaccatctccagagacaacggcaagaacacggtggctctacaaatgaacaacctgaaacctgaggacacggccgtttatttctgtgcagcggctgtgcgtctaactgcgcctctcaattttgacacctcgtatgactactggggccaggggacccaggtcaccatctcctcagaacccaagacaccaaaaccacaaact

SEQ ID NO: 110 - protein, 133 aa, Artificial Sequence
  Description of Artificial Sequence: Synthetic polypeptide
EVQLLESGGGLVQPGGSLRLSCAASGFTFSNSVMSWVRQAPGKGPERVSIIGSVGGTSGVTSYADSVKGRFTITRDNARSTLHLQMNSLKPEDTAVYYCVKGNGLTSTRASDYWGQGTQVTVSSEPKTPKPQT

SEQ ID NO: 111 - DNA, 399 bp, Artificial Sequence
  Description of Artificial Sequence: Synthetic polynucleotide
gaggtgcagctgctggagtctgggggaggcttggtgcagcctggggggtctctgagactctcctgtgcagcctctggattcaccttcagtaacagcgtcatgagctgggtccgccaggctccaggaaaggggcccgagcgggtctcaattatcggcagtgtcggaggtacctcaggtgtcacaagttatgcagactccgtgaagggccgattcaccatcaccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 112 - protein, 133 aa, Artificial Sequence
  Description of Artificial Sequence: Synthetic polypeptide
EVQLEESGGGLVQAGDSLRLSCVVSGSISSIYAMGWVREDPGKERVVVAGINSGAIRWYADSVKGRFTISGDNAKNTVYLQMNSLKPEDTAVYFCAAAVRLTAPLNFDTSYDYWGQGTQVTVSSEPKTPKPQT

SEQ ID NO: 113 - DNA, 399 bp, Artificial Sequence
  Description of Artificial Sequence: Synthetic polynucleotide
gaggtgcagctggaggagtctgggggaggattggtgcaggctggggactctctgagactctcctgtgtagtctctggaagcatctccagtatctatgccatgggatgggtccgcgaggatccagggaaggagcgcgtagtggttgcaggtattaatagcggagctatcagatggtacgcagactctgtgaagggccgattcaccatctccggagacaacgccaagaacacggtgtatctgcaaatgaacagcctgaaacctgaggacacggccgtttatttctgtgcagcggctgtgcgtctaactgcgcctctcaattttgacacctcgtatgactactggggccaggggacccaggtcaccgtctcctcagaacccaagacaccaaaaccacaaact

SEQ ID NO: 114 - protein, 133 aa, Artificial Sequence
  Description of Artificial Sequence: Synthetic polypeptide
EVQLLESGGGLVQPGGSLRLSCAASGFTFSNYAMSWVRQAPGKGPERVSIIGSVGGTSGVTSYADSVKGRFTITRDNARSTLHLQMNSLKPEDTAVYYCVKGNGLTSTRASDYWGQGTQVTVSAEPKTPKPQT

SEQ ID NO: 115 - DNA, 399 bp, Artificial Sequence
  Description of Artificial Sequence: Synthetic polynucleotide
gaggtgcagctgctggagtctgggggaggcttggtgcagcctggggggtctctgagactctcctgtgcagcctctggattcaccttcagtaactacgccatgagctgggtccgccaggctccaggaaaggggcccgagcgggtctcaattatcggcagtgtcggaggtacctcaggtgtcacaagttatgcagactccgtgaagggccgattcaccatcaccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctccgcagaacccaagacaccaaaaccacaaact

SEQ ID NO: 116 - protein, 133 aa, Artificial Sequence
  Description of Artificial Sequence: Synthetic polypeptide
EVQLEESGGGLVQPGGSLRLSCAASGFTFSNAVMSWVRQAPGKGPERVSIIGSVGGTSGVTSYADSVKGRFTITRDNARSTLHLQMNSLKPEDTAVYYCVKGNGLTSTRASDYWGQGTQVTVSSEPKTPKPQT

SEQ ID NO: 117 - DNA, 399 bp, Artificial Sequence
  Description of Artificial Sequence: Synthetic polynucleotide
gaggtgcagctggaggagtctgggggaggcttggtgcagcctggggggtctctgagactctcctgtgcagcctctggattcaccttcagtaacgccgtcatgagctgggtccgccaggctccaggaaaggggcccgagcgggtctcaattatcggcagtgtcggaggtacctcaggtgtcacaagttatgcagactccgtgaagggccgattcaccatcaccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 118 - protein, 133 aa, Artificial Sequence
  Description of Artificial Sequence: Synthetic polypeptide
EVQLEESGGGLVQPGGSLRLSCAASGFTFSNQVMSWVRQAPGKGPERVSVIGSVGGATGATSYADSVRGRFTISRDNARSTLHLQMNSLKPEDTAVYYCVKGNGLTSTRASDYWGQGTQVTVSSEPKTPKPQT

SEQ ID NO: 119 - DNA, 399 bp, Artificial Sequence
  Description of Artificial Sequence: Synthetic polynucleotide
gaggtgcagctggaggagtctgggggaggcttggtgcagcctggggggtctctgagactctcctgtgcagcctctggattcaccttcagtaaccaagtcatgagctgggtccgccaggctccaggaaaggggcccgagcgggtctcagttatcggcagtgtcggaggtgccacaggtgccacaagttatgcagactccgtgaggggccgattcaccatctccagagataacgccaggagcacgctgcatctgcaaatgaacagcctcaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 120 - protein, 135 aa, Artificial Sequence
  Description of Artificial Sequence: Synthetic polypeptide
EVQLEESGGGLVETGDSLRLSCSASGGGFSFNAIGWYRQGPGKGRELVAAGTSGSTTYYAPSVKGRFIFSRDSAKNTVYLQMNNLNPEDTAIYYCATPALGQMEYDVVSGDGLAHWGKGTLVIVSSAHHSEDPNS

SEQ ID NO: 121 - DNA, 405 bp, Artificial Sequence
  Description of Artificial Sequence: Synthetic polynucleotide
gaggtgcagctggaggagtctgggggaggcctggtggagactggggattctctgagactctcctgctctgcctctgggggcggttttagtttcaatgccataggctggtaccggcaggggccgggaaaggggcgcgaattggtcgcagcaggtactagtggaagtaccacatattacgcgccctctgtgaagggccgattcatcttctccagagacagtgccaaaaacaccgtctatctgcaaatgaacaacctgaaccctgaagacacggccatctattactgtgccacaccggcacttggacaaatggagtatgacgtagtgagcggcgacggcttggcccactggggcaaagggaccctggtcatcgtctcttcagcgcaccacagcgaagaccctaatagt

SEQ ID NO: 122 - protein, 133 aa, Artificial Sequence
  Description of Artificial Sequence: Synthetic polypeptide
EVQLVESGGGLVQPGGSLRLSCAASGFTFSNQVMSWVRQAPGKGPERVSVIGSVGGATGATSYADSVRGRFTISRDNARSTLHLQMNSLKPEDTAVYYCVKGNGLTSTRASDYWGQGTQVTVSSEPKTPKPQT

SEQ ID NO: 123 - DNA, 399 bp, Artificial Sequence
  Description of Artificial Sequence: Synthetic polynucleotide
gaggtgcagctggtggagtctgggggaggcttggtgcagcctggggggtctctgagactctcctgtgcagcctctggattcaccttcagtaaccaagtcatgagctgggtccgccaggctccaggaaaggggcccgagcgggtctcagttatcggcagtgtcggaggtgccacaggtgccacaagttatgcagactccgtgaggggccgattcaccatctccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 124 - protein, 133 aa, Artificial Sequence
  Description of Artificial Sequence: Synthetic polypeptide
EVQLVESGGGLVQPGGSLRLSCAASGFTFSNHVMSWVRQAPGKGPERVSIIGSVGGTSGVTSYADSVKGRFTITRDNARSTLHLQMNSLKPEDTAVYYCVKGNGLTSTRASDYWGQGTQVTVSSEPKTPKPQT

SEQ ID NO: 125 - DNA, 399 bp, Artificial Sequence
  Description of Artificial Sequence: Synthetic polynucleotide
gaggtgcagctggtggagtctgggggaggcttggtgcagcctggggggtctctgagactctcctgtgcagcctctggattcaccttcagtaaccacgtcatgagctgggtccgccaggctccaggaaaggggcccgagcgggtctcaattatcggcagtgtcggaggtacctcaggtgtcacaagttatgcagactccgtgaagggccgattcaccatcaccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 126 - protein, 138 aa, Artificial Sequence
  Description of Artificial Sequence: Synthetic polypeptide
EVQLVESGGGLVQPGGSLRLSCATSGLTNPERRLAWFRQEPGKEREFVASIRWSGGPNTHYGDSVKGRFTISRDNGKNTVALQMNNLKPEDTAVYYCAARDSPCVGNCWYENAGDYEYWGQGTQVTVSSEPKTPKPQT

SEQ ID NO: 127 - DNA, 414 bp, Artificial Sequence
  Description of Artificial Sequence: Synthetic polynucleotide
gaggtgcagctggtggagtctgggggaggcttggtgcagcctggggggtctctgagactctcctgtgcaacttctggactgaccaaccctgaaagacgcttggcctggttccgccaggaaccagggaaggagcgtgagtttgtagcgtccattcgctggagtggtggtcccaacacacactatggggactccgtgaagggccgattcaccatctccagagacaacggcaagaacacggtggctctacaaatgaacaacctgaaacctgaggacacggccgtttattactgtgcagcgcgagacagcccgtgcgtgggtaattgttggtacgagaatgcgggcgactatgagtactggggccaggggacccaggtcaccgtctcctcagaacccaagacaccaaaaccacaaact

SEQ ID NO: 128 - protein, 137 aa, Artificial Sequence; identical to SEQ ID NO: 100
  Description of Artificial Sequence: Synthetic polypeptide

SEQ ID NO: 129 - DNA, 411 bp, Artificial Sequence; identical to SEQ ID NO: 101
  Description of Artificial Sequence: Synthetic polynucleotide

SEQ ID NO: 130 - protein, 130 aa, Artificial Sequence
  Description of Artificial Sequence: Synthetic polypeptide
EVQLVESGGGLVQAGDSLTLSCAASERAYRNRLLGWFRQVPGKEREFVAWIRPIDSSTNYADSVKGRFTITRDNARSTLHLQMNSLKPEDTAVYYCVKGNGLTSTRASDYWGQGTQVTVSSEPKTPKPQT

SEQ ID NO: 131 - DNA, 390 bp, Artificial Sequence
  Description of Artificial Sequence: Synthetic polynucleotide
gaggtgcagctggtggagtctgggggaggattggtgcaggctggggactcgctgacactctcctgtgcagcctctgaacgcgcctacaggaaccgtcttcttggctggttccgccaggttccagggaaggagcgtgaatttgtggcatggatcagacccattgatagcagcacaaattatgcagactccgtgaagggccgattcaccatcaccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 132 - protein, 133 aa, Artificial Sequence
  Description of Artificial Sequence: Synthetic polypeptide
EVQLEESGGGSVQAGGSLRLSCAASGFTFSNSVMSWVRQAPGKGPERVSIIGSVGGTSGVTSYADSVKGRFTITRDNARSTLHLQMNSLKPEDTAVYYCVKGNGLTSTRASDYWGQGTQVTVSSEPKTPKPQT

SEQ ID NO: 133 - DNA, 399 bp, Artificial Sequence
  Description of Artificial Sequence: Synthetic polynucleotide
gaggtgcagctggaggagtctgggggaggatcggtgcaggctggggggtctctgagactctcctgtgcagcctctggattcaccttcagtaactccgtcatgagctgggtccgccaggctccaggaaaggggcccgagcgggtctcaattatcggcagtgtcggaggtacctcaggtgtcacaagttatgcagactccgtgaagggccgattcaccatcaccagagataacgccaggagcacgctgcatctacaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 134 - protein, 133 aa, Artificial Sequence
  Description of Artificial Sequence: Synthetic polypeptide
EVQLEESGGGLVQPGGSLRLSCAASGFTFSNSVMSWVRQAPGKGPERVSIIGSVGGTSGVTSYADSVKGRFTITRDNARSTLHLQMNSLKPEDTAVYYCVKGNGLTSTRASDYWGQGTQVTVSSEPKTPKPQT

SEQ ID NO: 135 - DNA, 399 bp, Artificial Sequence
  Description of Artificial Sequence: Synthetic polynucleotide
gaggtgcagctggaggagtctgggggaggcttggtgcagcctggggggtctctgagactctcctgtgcagcctctggattcaccttcagtaacagcgtcatgagctgggtccgccaggctccaggaaaggggcccgagcgggtctcaattatcggcagtgtcggaggtacctcaggtgtcacaagttatgcagactccgtgaagggccgattcaccatcaccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 136 - protein, 133 aa, Artificial Sequence
  Description of Artificial Sequence: Synthetic polypeptide
EVQLLESGGGLVQAGGSLRLSCAASGRTLTFYTGWFRQAPGKEREFVASIRWSGGNTYYADSVKGRFTISGDNAKNTVYLQMNSLKPEDTAIYYCAALRSWTTTPQREVLYDNWGQGTQVTVSSAHHSEDPIS

SEQ ID NO: 137 - DNA, 399 bp, Artificial Sequence
  Description of Artificial Sequence: Synthetic polynucleotide
gaggtgcagctgctggagtctgggggaggattggtgcaggcggggggctctctgagactctcctgtgcagcctccggacgcaccctcactttttatactggctggttccgccaggctccagggaaggagcgtgagtttgtagcttctattaggtggagtggcggtaacacatactatgcagactccgtgaagggccgattcaccatctccggagacaacgccaagaacacggtgtatctgcaaatgaacagcctgaaacccgaggacacggccatttattactgcgcagcacttagatcttggactactacacctcagagggaggtcctctatgacaactggggccaggggacccaggtcaccgtctcctcagcgcaccacagcgaagaccctattagt

SEQ ID NO: 138 - protein, 133 aa, Artificial Sequence
  Description of Artificial Sequence: Synthetic polypeptide
EVQLVESGGGLVQAGDSLRLSCAASGLTNPERRLAWFRQAPGKEREFVASIRWSGGPNTHYGDSVKGRFTISRDNAKNMVYLQMDNIKPEDTARYFCASSYTFSSVREDDYDYWGQGTQVTVLSAHHSEDPIS

SEQ ID NO: 139 - DNA, 399 bp, Artificial Sequence
  Description of Artificial Sequence: Synthetic polynucleotide
gaggtgcagctggtggagtctgggggaggattggtgcaggctggggactctctgagactctcctgtgcagcttctggactgaccaaccctgaaagacgcttggcctggttccgccaggctccagggaaggagcgtgagtttgtagcgtccattcgctggagtggtggtcccaacacacactatggagactccgtgaagggccgatttaccatctctcgagataacgccaagaacatggtgtacctgcaaatggacaacataaaacctgaagacacggcccgttatttctgtgcgtcctcatacaccttcagcagtgtccgggaggatgactatgactactggggccaggggacccaggtcaccgtcttgtcagcgcaccacagcgaagaccctattagt

SEQ ID NO: 140 - protein, 128 aa, Artificial Sequence
  Description of Artificial Sequence: Synthetic polypeptide
EVQLVESGGGLVQAGGSLRLSCAASGRTVSRYDMGWFRQAPGAERVVVAISWSGGSTYYVDSVKGRFTMSRDNSKNTVYLQMNSLKPEDTAVYYCAVRTERSSLDFHSWGQGTQVTVSSEPKTPKPQT

SEQ ID NO: 141 - DNA, 384 bp, Artificial Sequence
  Description of Artificial Sequence: Synthetic polynucleotide
gaggtgcagctggtggagtctgggggaggattggtgcaggctgggggctctctgagactctcctgtgcagcctctggacgcaccgtcagtagatatgacatgggctggttccgccaggctccaggggcggagcgtgtcgttgtagctattagctggagcggtggtagtacatactatgtagactccgtgaagggccgattcaccatgtccagagacaacagcaagaacacggtatatctgcaaatgaacagcctgaaacctgaggacacggccgtttattactgtgcagtcagaaccgaacgctccagtcttgactttcattcctggggccaggggacccaggtcaccgtctcctcggaacccaagacaccaaaaccacaaact

SEQ ID NO: 142 - protein, 132 aa, Artificial Sequence
  Description of Artificial Sequence: Synthetic polypeptide
EVQLEESGGGLVQAGDSLRLSCAASERAYRNRLLGWFRQVPGKEREFVAWIRPIDSSTNYADSVKGRFTISRDNDKNTVYLQMDNMKPEDTALYYCASTYYYSSIREDDYDYWGQGTQVTVSSEPKTPKPQT

SEQ ID NO: 143 - DNA, 396 bp, Artificial Sequence
  Description of Artificial Sequence: Synthetic polynucleotide
gaggtgcagctggaggagtctgggggaggattggtgcaggctggggactctctgagactctcctgtgcagcctctgaacgcgcctacaggaaccgtcttcttggctggttccgccaggttccagggaaggagcgtgaatttgtggcatggatcagacccattgatagcagcacaaattatgcagactccgtgaagggccgattcaccatctctagagataacgacaagaacacggtgtatttgcaaatggacaatatgaaacctgaggacacggccctctattattgtgcgtccacatactactacagtagtatccgggaggatgactatgactactggggccaggggacccaggtcaccgtctcctcagaacccaagacaccaaaaccacaaact

SEQ ID NO: 144 - protein, 132 aa, Artificial Sequence
  Description of Artificial Sequence: Synthetic polypeptide
EVQLVESGGGLVQAGGSLRLSCAASGRAFSNRALGWFRQAPGKEREFVAWIRGIGSSTNYAGSVQGRFTISRDNAKNTLYLQMDKLKPEDTAVYYCASTYMFDSVREDEYDYWGQGTQVTVSSEPKTPKPQT

SEQ ID NO: 145 - DNA, 396 bp, Artificial Sequence
  Description of Artificial Sequence: Synthetic polynucleotide
gaggtgcagctggtggagtctgggggaggattggtgcaggctgggggctctctgagactctcctgtgcagcctctggacgcgccttcagtaaccgtgcacttggctggttccgccaggctccagggaaggagcgtgagtttgtagcgtggattagaggcatcggtagcagcacaaattatgcaggctccgtacagggccgattcaccatctccagagacaacgccaagaacacgctgtatctgcagatggacaagctgaaacctgaggacacggccgtttattattgtgcgtccacatacatgttcgatagtgtgcgggaggatgaatatgactactggggccaggggacccaggtcaccgtctcctcagaacccaagacaccaaaaccacaaact

SEQ ID NO: 146 - protein, 135 aa, Artificial Sequence
  Description of Artificial Sequence: Synthetic polypeptide
EVQLQESGGGLLQTGDSLRLACEASEIVVENYVMAWFRQAPGKEREWLARIIWNTGGTHLQEFVKGRLTISRDIAKKTVYLQMNSLKPEDTAVYYCAGGSFDAIADPFSARRYGFWGQGTQVTVSSEPKTPKPQT

SEQ ID NO: 147 - DNA, 405 bp, Artificial Sequence
  Description of Artificial Sequence: Synthetic polynucleotide
gaggtgcagctgcaggagtctgggggaggattgctgcagactggggactcactgagactcgcctgtgaagcctctgaaatcgtcgtcgaaaattatgtcatggcctggttccgccaggctccagggaaggagcgtgagtggctagcgcgtattatttggaataccggtggcacacatcttcaagaatttgtgaagggccgactcaccatctctagagacatcgccaagaaaacggtgtatctgcaaatgaacagcctgaaacctgaggacacggccgtttattactgtgccggtggaagttttgacgctatagccgatcccttctcggcccgccggtatgggttctggggccaggggacccaggtcaccgtctcctcagaacccaagacaccaaaaccacaaact

SEQ ID NO: 148 - protein, 120 aa, Artificial Sequence
  Description of Artificial Sequence: Synthetic polypeptide
EVQLQESGGGLVQAGGSLRLSCVSSGDSISGVVVRWYRQVPGKQREWIGGIGTSDNPEYADSVWGRFVLSRDNAGSRVNLQMNNLKLEDTATYYCNAVHKWGPGTQVTVSSEPKTPKPQT

SEQ ID NO: 149 - DNA, 360 bp, Artificial Sequence
  Description of Artificial Sequence: Synthetic polynucleotide
gaggtgcagctgcaggagtctgggggaggcctggtgcaggctggggggtctctgagactctcctgtgtaagttctggagacagtatcagtggagtggtcgtccgttggtaccgccaggttccagggaagcagcgcgagtggatcggaggtattggtactagtgataacccagaatatgcggactccgtctggggccgattcgtcctctccagagacaatgccgggagccgcgtaaatctgcaaatgaacaacctgaaacttgaggacacggccacctattactgcaatgcagtgcacaaatggggcccgggtacccaggtcaccgtctcttctgaacccaagacaccaaaaccacaaact

SEQ ID NO: 150 - protein, 132 aa, Artificial Sequence
  Description of Artificial Sequence: Synthetic polypeptide
EVQLVESGGGLVQPGGSLRLSCAASGFTFSNQVMSWVRQAPGKEREFVAWIRGIGGSTHYAGSVEGRFTISRDSAKNTLYLQMDNVKPEDTAVYYCASTYMFDSVREDEYDYWGQGTEVTVSSAHHSEDPNS

SEQ ID NO: 151 - DNA, 396 bp, Artificial Sequence
  Description of Artificial Sequence: Synthetic polynucleotide
gaggtgcagctggtggagtctgggggaggcttggtgcagcctggggggtctctgagactctcctgtgcagcctctggattcaccttcagtaaccaagtcatgagctgggtccgccaggctccagggaaggagcgtgagtttgtagcgtggattagaggcatcggtggcagcacacattatgcaggctccgtggagggccgattcaccatctccagagacagcgccaagaacacgttgtatctacagatggacaacgtgaaacccgaggacacggccgtttattattgtgcgtccacatacatgttcgatagtgtccgggaggatgaatatgactactggggccaggggaccgaggtcaccgtctcctcagcgcaccacagcgaagaccctaatagt

SEQ ID NO: 152 - protein, 135 aa, Artificial Sequence
  Description of Artificial Sequence: Synthetic polypeptide
EVQLLESGGGLVQPGGSLRLSCAASGFTFSNHVMSWVRQAPGKGPERVSVIGSVGGATGATSYADSVRGRFTISRDSAKNTLYLQMDNVKPEDTAVYYCASTYMFDSVREDEYDYWGQGTEVTVSSEPKTPKPQT

SEQ ID NO: 153 - DNA, 405 bp, Artificial Sequence
  Description of Artificial Sequence: Synthetic polynucleotide
gaggtgcagctgctggagtctgggggaggcttggtgcagcctggggggtctctgagactctcctgtgcagcctctggattcaccttcagtaaccacgtcatgagttgggtccgccaggctccaggaaaggggcccgagcgggtctcagttatcggcagtgtcggaggtgccacaggtgccacaagttatgcagactccgtgaggggccgattcaccatctccagagacagcgccaagaacacgttgtatctacagatggacaacgtgaaacccgaggacacggccgtttattattgtgcgtccacatacatgttcgatagtgtccgggaggatgaatatgactactggggccaggggaccgaggtcaccgtctcctcagaacccaagacaccaaaaccacaaact

SEQ ID NO: 154 - protein, 132 aa, Artificial Sequence
  Description of Artificial Sequence: Synthetic polypeptide
EVQLEESGGGLVQTGGSLRLSCAASGGTFSSYVMGWFRQAPGKEREFVAWIRPIDSSTNYADSVKGRFTISRDDAKNSLYLQMDNMKPEDTALYYCASTYYYSSIREDDYDYWGRGTQVTVLSAHHSEDPNS

SEQ ID NO: 155 - DNA, 396 bp, Artificial Sequence
  Description of Artificial Sequence: Synthetic polynucleotide
gaggtgcagctggaggagtctgggggaggattggtacagaccgggggatctttgagactctcctgtgcagcctctggcggcaccttcagtagctatgtcatgggctggttccgccaggctccagggaaggagcgtgaatttgtggcatggatcagacccattgatagcagcacaaattatgcagactccgtgaagggccgattcaccatctctagggatgacgccaagaactcgctgtatctgcaaatggacaatatgaaacctgaggacacggccctctattattgtgcgtccacatactactacagtagtatccgggaggatgactatgactactggggccgggggacccaggtcaccgtcttgtcagcgcaccacagcgaagaccctaatagt

SEQ ID NO: 156 - protein, 133 aa, Artificial Sequence
  Description of Artificial Sequence: Synthetic polypeptide
EVQLVESGGGLVQPGGSLRLSCATSGFTFSNNVMSWVRQAPGKGPERVAVIGSVGGTTGATSYADSVKGRFTITRDNARSTLHLQMNSLKPEDTAVYYCVKGNGLTSTRASDYWGQGTQVTVSSEPKTPKPQT

SEQ ID NO: 157 - DNA, 399 bp, Artificial Sequence
  Description of Artificial Sequence: Synthetic polynucleotide
gaggtgcagctggtggagtctgggggaggcttggtgcagcctggggggtctctgagactctcctgtgcaacctctggattcaccttcagtaacaacgtcatgagctgggtccgccaggctccaggaaaggggcccgagcgggtcgcagttatcggcagtgtcggaggtaccacgggtgccacaagttatgcagactccgtgaagggccgattcaccatcaccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 158 - protein, 129 aa, Artificial Sequence
  Description of Artificial Sequence: Synthetic polypeptide
EVQLVESGGGLVQARGSLRLSCVASGRTLTYYTGWFRQAPGKEREFVASFAWSGGNTYYADSVKGRFTISRDNARSTLHLQMNSLKPEDTAVYYCVKGNGLTSTRASDYWGQGTQVTVSSEPKTPKPQT

SEQ ID NO: 159 - DNA, 387 bp, Artificial Sequence
  Description of Artificial Sequence: Synthetic polynucleotide
gaggtgcagctggtggagtctgggggaggattggtgcaggcgaggggctctctgagactctcctgtgtagcctccggccgcaccctcacttactatactggctggttccgccaggctccaggaaaggagcgtgagtttgtagcatcttttgcgtggagtggcggtaacacatactatgcagactccgtgaagggccgattcaccatctccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 160 - protein, 133 aa, Artificial Sequence
  Description of Artificial Sequence: Synthetic polypeptide
EVQLVESGGGLVQPGGSLRLSCAASGFTFSNQVMSWVRQAPGKGPERVSIIGSVGGTSGVTSYADSVKGRFTITRDNARSTLHLQMNSLKPEDTAVYYCVKGNGLTSTRASDYWGQGTQVTVSSEPKTPKPQT

SEQ ID NO: 161 - DNA, 399 bp, Artificial Sequence
  Description of Artificial Sequence: Synthetic polynucleotide
gaggtgcagctggtggagtctgggggaggcttggtgcagcctggggggtctctgagactctcctgtgcagcctctggattcaccttcagtaaccaagtcatgagctgggtccgccaggctccaggaaaggggcccgagcgggtctcaattatcggcagtgtcggaggtacctcaggtgtcacaagttatgcagactccgtgaagggccgattcaccatcaccagagataacgccaggagcacgctgcatctgcaaatgaacagcctcaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 162 - protein, 133 aa, Artificial Sequence
  Description of Artificial Sequence: Synthetic polypeptide
EVQLEESGGGLVQAGDSLRLSCAASGFTFSDYAMSWVRQAPGKGPERVSVIGSVGGTTGVTSYADSVKGRFTITRDNARSTLHLQMNSLKPEDTAVYYCVKGNGLTSTRASDYWGQGTQVTVSSEPKTPKPQT

SEQ ID NO: 163 - DNA, 399 bp, Artificial Sequence
  Description of Artificial Sequence: Synthetic polynucleotide
gaggtgcagctggaggagtctgggggaggattggtgcaggctggggactctctgagactctcctgtgcagcctctggattcaccttcagtgactacgccatgagctgggtccgccaggctccaggaaaggggcccgagcgggtctcagttatcggcagtgtcggaggtaccacaggtgtcacaagttatgcagactccgtgaagggccgattcaccatcaccagagataacgccaggagcacgctgcatctgcaaatgaacagtctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 164 - protein, 133 aa, Artificial Sequence; identical to SEQ ID NO: 134
  Description of Artificial Sequence: Synthetic polypeptide

SEQ ID NO: 165 - DNA, 399 bp, Artificial Sequence; identical to SEQ ID NO: 135
  Description of Artificial Sequence: Synthetic polynucleotide

SEQ ID NO: 166 - protein, 135 aa, Artificial Sequence
  Description of Artificial Sequence: Synthetic polypeptide
EVQLLESGGGLVQAGDSLRLSCTQSGRTFSRYAIGWFRQAPGKEREFVASIRWSGGHTYYADSVKGRFTISKDNAKDTVYLQMNSLKPEDTAVYYCAGGSFDAIADPFSARRYGFWGQGTQVTVSSAHHSEDPIS

SEQ ID NO: 167 - DNA, 405 bp, Artificial Sequence
  Description of Artificial Sequence: Synthetic polynucleotide
gaggtgcagctgctggagtctggggggggattggtgcaggcaggggactctctgagactctcctgtacacaatctggacgcaccttcagcagatatgccataggctggttccgccaggctccagggaaggagcgtgagtttgtagcatccattaggtggagtggcggtcacacatactatgcagactccgtgaagggtcgcttcaccatttccaaggacaacgccaaagacacggtgtatctgcaaatgaacagcctgaaacctgaggacacggccgtttattactgtgcgggtggaagttttgacgctatagccgatcccttctcggcccgccggtatggattctggggccaggggacccaggtcaccgtctcctcggcgcaccacagcgaagaccctattagt

SEQ ID NO: 168 - protein, 133 aa, Artificial Sequence
  Description of Artificial Sequence: Synthetic polypeptide
EVQLEESGGGLVQAGGSLRLSCAASGRTLTYYTGWFRQAPGKEREFVASFAWMGDNTYYADSVKGRFTISGDNAKNTVYLQMNSLKPEDTATYYCAALRFWTTTPQREVLYDNWGQGTQVTVSSAHHSEDPIS

SEQ ID NO: 169 - DNA, 399 bp, Artificial Sequence
  Description of Artificial Sequence: Synthetic polynucleotide
gaggtgcagctggaggagtctgggggaggattggtgcaggcggggggctctctgagactctcctgtgcagcctccggacgcaccctcacttattatactggctggttccgccaggctccagggaaggagcgtgagtttgtagcatcttttgcgtggatgggtgataacacatactacgctgactccgtgaagggccggttcaccatctccggcgacaacgccaagaacacggtgtatctgcaaatgaacagcctgaaacccgaggacacggccacttattactgcgcagcattaagattttggactactacaccgcagagggaggtcctctatgacaactggggccaggggacccaggtcaccgtctcctcagcgcaccacagcgaagaccctattagt

SEQ ID NO: 170 - protein, 135 aa, Artificial Sequence; identical to SEQ ID NO: 108
  Description of Artificial Sequence: Synthetic polypeptide

SEQ ID NO: 171 - DNA, 405 bp, Artificial Sequence; identical to SEQ ID NO: 109
  Description of Artificial Sequence: Synthetic polynucleotide

SEQ ID NO: 172 - protein, 133 aa, Artificial Sequence; identical to SEQ ID NO: 122
  Description of Artificial Sequence: Synthetic polypeptide

SEQ ID NO: 173 - DNA, 399 bp, Artificial Sequence; identical to SEQ ID NO: 123
  Description of Artificial Sequence: Synthetic polynucleotide

SEQ ID NO: 174 - protein, 133 aa, Artificial Sequence; identical to SEQ ID NO: 122
  Description of Artificial Sequence: Synthetic polypeptide

SEQ ID NO: 175 - DNA, 399 bp, Artificial Sequence; identical to SEQ ID NO: 123
  Description of Artificial Sequence: Synthetic polynucleotide

SEQ ID NO: 176 - protein, 133 aa, Artificial Sequence
  Description of Artificial Sequence: Synthetic polypeptide
EVQLLESGGGLVQPGGSLRLSCAASGFTFSDYAMSWVRQAPGKGPERVSVIGSVGGTTGVTSYADSVKGRFTISRDNARSTLHLQMNSLKPEDTAVYYCVKGNGLTSTRASDYWGQGTQVTVSSEPKTPKPQT

SEQ ID NO: 177 - DNA, 399 bp, Artificial Sequence
  Description of Artificial Sequence: Synthetic polynucleotide
gaggtgcagctgctggagtctgggggaggcttggtgcagcctggggggtctctgagactctcctgtgcagcctctggattcaccttcagtgactacgccatgagctgggtccgccaggctccaggaaaggggcccgagcgggtctcagttatcggcagtgtcggaggtaccacaggtgtcacaagttatgcagactccgtgaagggccgattcaccatctccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 178 - protein, 131 aa, Artificial Sequence
  Description of Artificial Sequence: Synthetic polypeptide
EVQLVESGGGLVQAGGSLRLACTASGSDFKRAALGWYRQAPGQERELVAAFNSGGKTYYTDSVKDRFTISRDNAKSTLYLQMNSLKPDDTAMYYCALSRFDYYLPPTQFDYWGQGTQVTVSSEPKTPKPQT

SEQ ID NO: 179 - DNA, 393 bp, Artificial Sequence
  Description of Artificial Sequence: Synthetic polynucleotide
gaggtgcagctggtggagtctgggggaggattggtgcaggctgggggctctctgagactcgcctgtacagcctctggaagcgacttcaagcgcgccgccctgggctggtaccgccaggctccaggacaggagcgcgagttggtcgcagcttttaatagtggaggtaaaacatactacacagattctgtgaaggaccgattcaccatctccagagacaatgccaagagtacgctgtatctccaaatgaacagcctgaaacctgacgacacggccatgtattactgtgcgttatcacggttcgattactatcttccacccacccaatttgactactggggccaggggacccaggtcaccgtctcctcagaacccaagacaccaaaaccacaaact

SEQ ID NO: 180 - protein, 133 aa, Artificial Sequence
  Description of Artificial Sequence: Synthetic polypeptide
EVQLVESGGGLVQAGGSLRLSCAASGRTLTFYTGWFRQAPGKEREFVASIRWSGGNTDYADSVKGRFTISGDNAKNTVYLQMNSLKPEDTAIYYCAALRSWTTTPQREVLYDYWGQGTQVTVSSEPKTPKPQT

SEQ ID NO: 181 - DNA, 399 bp, Artificial Sequence
  Description of Artificial Sequence: Synthetic polynucleotide
gaggtgcagctggtggagtctgggggaggattggtgcaggcggggggctctctgagactctcctgtgcagcctccggacgcaccctcactttttatactggctggttccgccaggctccagggaaggagcgtgagtttgtagcatctattaggtggagtggcggtaacacagactatgcagactccgtgaagggccgattcaccatctccggagacaacgccaagaacacggtgtatctgcaaatgaacagcctgaaacccgaggacacggccatttattactgcgcggcacttagatcttggactactacacctcagagggaggtcctctatgactactggggccaggggacccaggtcaccgtctcctcagaacccaagacaccaaaaccacaaacg

SEQ ID NO: 182 - protein, 133 aa, Artificial Sequence
  Description of Artificial Sequence: Synthetic polypeptide
EVQLVESGGGLVQAGGSLKLSCAASGRTLTYYTAWFRQAPGKEREFVASLGWSGDVTYYADSVKGRFTISGDNAKNTVYLQMNSLKPEDTATYYCAALRSWTTTPQREVLYDNWGHGTQVTVSSAHHSEDPNS

SEQ ID NO: 183 - DNA, 399 bp, Artificial Sequence
  Description of Artificial Sequence: Synthetic polynucleotide
gaggtgcagctggtggagtctgggggaggattggtgcaggcggggggctctctgaaactctcctgtgcagcctccggacgcaccctcacttattatactgcctggttccgccaggctccagggaaggagcgtgagtttgtagcatcgctagggtggagtggcgatgtcacatactatgcagactccgtgaagggccgattcaccatctccggcgacaacgccaagaacacggtatatctgcaaatgaacagcctgaaacccgaggacacggccacttattactgcgcagcacttagatcttggactactacacctcagagggaggtcctctatgacaactggggccacgggacccaggtcaccgtctcctcagcgcaccacagcgaagaccctaatagt

SEQ ID NO: 184 - protein, 133 aa, Artificial Sequence; identical to SEQ ID NO: 122
  Description of Artificial Sequence: Synthetic polypeptide

SEQ ID NO: 185 - DNA, 399 bp, Artificial Sequence; identical to SEQ ID NO: 123
  Description of Artificial Sequence: Synthetic polynucleotide

SEQ ID NO: 186 - protein, 132 aa, Artificial Sequence
  Description of Artificial Sequence: Synthetic polypeptide
EVQLVESGGGLVQAGDSLKLSCVGSGRTFSSYGLGWFRQAPGKEREFLAHITWTAGGTYHADNVKGRFTISRDDAKNTVYLQMNSLKPEDTAVYYCAARSSGDWRVERYYDYWGQGTQVTVSSEPKTPKPQT

SEQ ID NO: 187 - DNA, 396 bp, Artificial Sequence
  Description of Artificial Sequence: Synthetic polynucleotide
gaggtgcagctggtggagtctgggggaggattggtgcaggctggggactctctgaaactctcctgtgtaggctctggacgcaccttcagcagctatgggttgggctggttccgccaggctccagggaaggagcgtgagtttctagcacatattacctggactgctggtggaacataccatgcagacaacgtgaagggccgattcaccatctccagagacgacgccaagaatacggtgtatctacaaatgaacagcctgaaacctgaggacacggccgtttattactgtgcggcacgttcctctggggattggcgtgtcgagagatattatgactactggggccaggggacccaggtcaccgtctcctcagaacccaagacaccaaaaccacaaact

SEQ ID NO: 188 - protein, 141 aa, Artificial Sequence
  Description of Artificial Sequence: Synthetic polypeptide
EVQLEESGGGLVQPGGSLRLSCATSGFTFSNNVMSWVRQAPGKGPERVAVIGSVGGATGATSYADSVKGRFTITRDNARSTLHLQMNGLKPEDTAMYYCAAETSSGLYYSYDDLQTIDFDSWGQGTQVTVSSAHHSEDPNS

SEQ ID NO: 189 - DNA, 423 bp, Artificial Sequence
  Description of Artificial Sequence: Synthetic polynucleotide
gaggtgcagctggaggagtctgggggaggcttggtgcagcctggggggtctctgagactctcctgtgcaacctctggattcaccttcagtaacaacgtcatgagctgggtccgccaggctccaggaaaggggcccgagcgggtcgcagttatcggcagtgtcggaggtgccacaggtgccacaagttatgcagactccgtgaagggccgattcaccatcaccagagataacgccaggagcacgctgcatctgcaaatgaacggcctgaaacccgaggacacggcaatgtattactgtgcggcggagaccagtagcggtctttactacagttacgatgaccttcaaacaattgactttgattcctggggccaggggacccaggtcaccgtctcctcagcgcaccacagcgaagaccctaatagt

SEQ ID NO: 190 - protein, 134 aa, Artificial Sequence
  Description of Artificial Sequence: Synthetic polypeptide
EVQLVESGGGLVQAGGSLRLSCAASERAFKNRALGWFRQAPGKEREFVASIRWSGGNTYYADSVKGRFTISGDNAKNTVYLQMNSLKPEDTAIYYCAALRSWTTTPQREVLYDNWGQGTQVTVSSEPKTPKPQT

SEQ ID NO: 191 - DNA, 402 bp, Artificial Sequence
  Description of Artificial Sequence: Synthetic polynucleotide
gaggtgcagctggtggagtctgggggaggcttggtgcaggctgggggctctctgagactctcctgtgcagcctctgaacgcgccttcaagaaccgtgcacttggctggttccgccaggctccagggaaggagcgtgagtttgtagcatctattaggtggagtggcggtaacacatactatgcagactccgtgaagggccgattcaccatctccggagacaacgccaagaacacggtgtatctgcaaatgaacagcctgaaacccgaggacacggccatttattactgcgcagcacttagatcttggactactacacctcagagggaggtcctctatgacaactggggccaggggacccaggtcaccgtctcctcagaacccaagacaccaaaaccacaaact

SEQ ID NO: 192 - protein, 124 aa, Artificial Sequence
  Description of Artificial Sequence: Synthetic polypeptide
EVQLVESGGGLVQAGGSLRLSCAASEFTFSGYWMHWVRQAPGKGPERVSIIGSVGGTSGVTSYADSVRGRFTVSRDDAKNTVYLHMDSLKAEDTAVYYCNVMQAWGQGTQVTVLSAHHSEDPIS

SEQ ID NO: 193 - DNA, 372 bp, Artificial Sequence
  Description of Artificial Sequence: Synthetic polynucleotide
gaggtgcagctggtggagtctgggggaggattggtgcaggcggggggctctctgagactctcctgtgcagcctctgaattcaccttcagtggctactggatgcactgggtccgccaggctccaggaaaggggcccgagcgggtctcaattatcggcagtgtcggaggtacctcaggtgtcacaagttatgcagactccgtgaggggccgattcactgtctccagagacgacgccaagaacacggtgtatctgcatatggatagtttgaaagctgaggacacggccgtgtattactgtaatgtcatgcaggcttggggccagggcacccaggtcaccgtcttgtcagcgcaccacagcgaagaccctattagt

SEQ ID NO: 194 - protein, 134 aa, Artificial Sequence
  Description of Artificial Sequence: Synthetic polypeptide
EVQLVESGGGLVETGGSLRLSCAGSGRTFSSRHVGWFRQTPGKEREWVGSVAWNTGSEYYADSVKGRFTISKDNAKDTVYLQMNSLKPEDTAIYYCAALRSWTTTPQREVLYDNWGQGTQVTVSSAHHSEDPIS

SEQ ID NO: 195 - DNA, 402 bp, Artificial Sequence
  Description of Artificial Sequence: Synthetic polynucleotide
gaggtgcagctggtggagtctgggggaggattggtcgaaactgggggttctctgagactctcctgtgcaggttctggacgcaccttcagtagccggcacgtgggctggttccgccagactccagggaaggagcgtgagtgggttggaagtgttgcctggaacactggtagtgaatattatgcagactccgtgaagggtcgcttcaccatttccaaggacaacgccaaagacacggtgtatctgcaaatgaacagcctgaaacccgaggacacggccatttattactgcgcggcacttagatcttggactactacacctcagagggaggtcctctatgacaactggggccaggggacccaggtcaccgtctcctcagcgcaccacagcgaagaccctattagt

SEQ ID NO: 196 - protein, 133 aa, Artificial Sequence
  Description of Artificial Sequence: Synthetic polypeptide
EVQLVESGGGLVQPGGSLRLSCAASGFTFSDYAMSWVRQAPGKGPERVSVIGSVGGVGGVTSYADSVKGRFTISRDNARSTLHLQMNSLKPEDTAVYYCVKGNGLTSTRASDYWGQGTQVTVSSEPKTPKPQT

SEQ ID NO: 197 - DNA, 399 bp, Artificial Sequence
  Description of Artificial Sequence: Synthetic polynucleotide
gaggtgcagctggtggagtctgggggaggcttggtgcagcctggggggtctctgagactctcctgtgcagcctctggattcaccttcagtgactacgccatgagctgggtccgccaggctccaggaaaggggcccgagcgggtctcagttattggcagtgtgggaggtgtcggaggtgtcacaagttatgcagactccgtgaagggccgattcaccatctccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 198 - protein, 133 aa, Artificial Sequence
  Description of Artificial Sequence: Synthetic polypeptide
EVQLQESGGGLVQPGGSLRLSCAASGFTFSNQVMSWVRQAPGKGPERVSVIGSVGGATGATSYADSVRGRFTISRDNARSTLHLQMNSLKPEDTAVYYCVKGNGLTSTRASDYWGQGTQVTVLSAHHSEDPNS

SEQ ID NO: 199 - DNA, 399 bp, Artificial Sequence
  Description of Artificial Sequence: Synthetic polynucleotide
gaggtgcagctgcaggagtctgggggaggcttggtgcagcctggggggtctctgagactctcctgtgcagcctctggattcaccttcagtaaccaagtcatgagctgggtccgccaggctccaggaaaggggcccgagcgggtctcagttatcggcagtgtcggaggtgccacaggtgccacaagttatgcagactccgtgaggggccgattcaccatctccagagataacgccaggagcacgctgcatctgcaaatgaacagcctgaaacctgaggacacggccgtgtattactgtgtaaaggggaacggacttacttctactcgcgcgagtgactactggggccaggggacccaggtcaccgtcttgtcagcgcaccacagcgaagaccctaatagt

SEQ ID NO: 200 - protein, 133 aa, Artificial Sequence
  Description of Artificial Sequence: Synthetic polypeptide
EVQLVESGGGLVQAGGSLRLSCVASGEEDFQPYAMGWFRQAPGKEREYVAATTWNGGRIRYGDSVKGRFTISRDHPKNTITLQMTSLKPDDTAVYYCAARYGTVLLTREDYQHWGRGTQVTVSAAHHSEDPIS

SEQ ID NO: 201 - DNA, 399 bp, Artificial Sequence
  Description of Artificial Sequence: Synthetic polynucleotide
gaggtgcagctggtggagtctgggggaggcttggtgcaggctggggggtctctgagactctcctgcgtagcctctggagaggaggattttcagccgtatgccatgggctggttccgccaggctccagggaaggagcgtgaatacgtggccgcgactacatggaatggtggtagaataagatatggagactccgtgaagggccgattcaccatctccagagaccaccccaagaacacgatcactttacaaatgaccagtttgaaacctgacgacacggccgtttattactgtgcagcacggtacggtacagtcctacttacacgcgaagactatcaacactggggccgtgggacccaggtcaccgtttccgcggcgcaccacagcgaagaccctattagt

SEQ ID NO: 202 - protein, 137 aa, Artificial Sequence
  Description of Artificial Sequence: Synthetic polypeptide
EVQLVESGGGLVQAGGSLSLSCAASGRTFSTGAMGWFRQAPGKEREFLARITLIGHGTYYADALKGRFTISRDHAKNTVYLQMNSLKPEDTAVYYCVARDSPCVGNCWYENAGDYEYWGQGTQVTVSSEPKTPKPQT

SEQ ID NO: 203 - DNA, 411 bp, Artificial Sequence
  Description of Artificial Sequence: Synthetic polynucleotide
gaggtgcagctggtggagtctgggggaggattggtgcaggctgggggctctctgagtctctcctgtgcagcctctggacgcaccttcagtaccggtgccatgggctggttccgccaggctccagggaaggagcgtgagtttctggcacgaattactctgattggccacggcacatactatgcagatgccttgaagggccgattcaccatttccagagaccacgccaagaacacggtgtatctgcaaatgaacagcctgaaacctgaggacacggccgtatattactgtgtagcgcgagacagcccgtgcgtgggtaattgttggtacgagaatgcgggcgactatgagtactggggccaggggacccaggtcaccgtctcctcagaacccaagacaccaaaaccacaaact

SEQ ID NO: 204 - protein, 133 aa, Artificial Sequence
  Description of Artificial Sequence: Synthetic polypeptide
EVQLLESGGGLVQAGGSLRLSCAASGFTFSNYAMSWVRQAPGKGPERVSIIGSVGGTSGVTSYADSVKGRFTITRDNARSTLHLQMNSLKPEDTAVYYCVKGNGLTSTRASDYWGQGTQVTVSSAHHSEDPIS

SEQ ID NO: 205 - DNA, 399 bp, Artificial Sequence
  Description of Artificial Sequence: Synthetic polynucleotide
gaggtgcagctgctggagtctgggggaggattggtgcaggctggggggtctctgagactctcctgtgcagcctctggattcaccttcagtaattacgccatgagctgggtccgccaggctccaggaaaggggcccgagcgggtctcaattatcggcagtgtcggaggtacctcaggtgtcacaagttatgcagactccgtgaagggccgattcaccatcaccagagataacgccaggagcacgctgcatcttcaaatgaacagcctgaaacctgaggacacggccgtgtattactgtgtaaaggggaacggacttacttctactcgcgcgagtgactactggggccagggaacccaggtcaccgtctcctcagcgcaccacagcgaagaccctattagt

SEQ ID NO: 206 - protein, 138 aa, Artificial Sequence
  Description of Artificial Sequence: Synthetic polypeptide
EVQLVESGGGLVQAGGSLRLSCAASERTVSVYTMGWFRQAPGKEREFVASIRWSGGPNTYYADSVKGRFTISGDNAKNTVYLQMNSLKPEDTAVYYCVARDSPCVGNCWYENAGDYEYWGQGTQVTVSSEPKTPKPQT

SEQ ID NO: 207 - DNA, 414 bp, Artificial Sequence
  Description of Artificial Sequence: Synthetic polynucleotide
gaggtgcagctggtggagtctgggggaggcctggtgcaggctgggggctctctgagactctcctgtgcagcctctgaacgcaccgtcagtgtctataccatgggctggttccgccaggctccagggaaggagcgtgagtttgtagcgtccattcgctggagtggtggtcccaacacatactatgcagactccgtgaagggccgattcaccatctccggagacaacgccaagaacacggtgtatctgcaaatgaacagcctgaaacctgaggacacggccgtatattactgtgtagcgcgagacagcccgtgcgtgggtaattgttggtacgagaatgcgggcgactatgagtactggggccaggggacccaggtcaccgtctcctcagaacccaagacaccaaaaccacaaact

SEQ ID NO: 208 - protein, 133 aa, Artificial Sequence
  Description of Artificial Sequence: Synthetic polypeptide
EVQLVESGGGLVQPGDSLRLSCAASGFTFSSYAMSWVRQAPGKGPERVSVIGSVGGTTGVTSYADSVRGRFTISRDNARSTLHLQMNSLKPEDTAVYYCVKGNGLTSTRASDYWGQGTQVTVSSEPKTPKPQT

SEQ ID NO: 209 - DNA, 399 bp, Artificial Sequence
  Description of Artificial Sequence: Synthetic polynucleotide
gaggtgcagctggtggagtctgggggaggcttggtgcagcctggggactctctgagactctcctgtgcagcctctggattcaccttcagtagctatgccatgagctgggtccgccaggctccaggaaaggggcccgagcgggtctcagttatcggcagtgtcggaggtaccacaggtgtcacaagttatgcagactccgtgaggggccgattcaccatctccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagaacccaagacaccaaaaccacaaact

SEQ ID NO: 210 - protein, 133 aa, Artificial Sequence
  Description of Artificial Sequence: Synthetic polypeptide
EVQLEESGGGLVQPGGSLRLSCAASGFTFSNSVMSWVRQAPGKGPERVSVIGSVGGATGATSYADSVRGRFTISRDNARSTLHLQMNSLKPEDTAVYYCVKGNGLTSTRASDYWGQGTQVTVSSAHHSEDPIS

SEQ ID NO: 211 - DNA, 399 bp, Artificial Sequence
  Description of Artificial Sequence: Synthetic polynucleotide
gaggtgcagctggaggagtctgggggaggcttggtgcagcctggggggtctctgagactctcctgtgcagcctctggattcaccttcagtaactccgtcatgagctgggtccgccaggctccaggaaaggggcccgagcgggtctcagttatcggcagtgtcggaggtgccacaggtgccacaagttatgcagactccgtgaggggccgattcaccatctccagagataacgccaggagcacgctgcatctgcaaatgaacagcctgaaacctgaggacacggccgtgtattactgtgtaaaggggaacggacttacttctactcgcgcgagtgactactggggccagggaacccaggtcaccgtctcctcagcgcaccacagcgaagaccctattagt

SEQ ID NO: 212 - protein, 133 aa, Artificial Sequence
  Description of Artificial Sequence: Synthetic polypeptide
EVQLLESGGGLVQAGDSLRLSCAASERAYRNRLLGWFRQAPGAERVVVAISWSGGSTYYVDSVKGRFTMSRDNSKNTVYLQMNSLKPEDTATYYCAALRFWTTTPQKEGLYDTWGQGTQVTVSSEPKTPKPQT

SEQ ID NO: 213 - DNA, 399 bp, Artificial Sequence
  Description of Artificial Sequence: Synthetic polynucleotide
gaggtgcagctgctggagtctgggggaggcttggtgcaggctggggactctctgagactctcctgtgcagcctctgaacgcgcctacaggaaccgtcttcttggctggttccgccaggctccaggggcggagcgtgtcgttgtagctattagctggagcggtggtagtacatactatgtagactccgtgaagggccgattcaccatgtccagagacaacagcaagaacacggtgtatctgcaaatgaacagcctgaaacccgaggacacggccacttattactgcgcagcacttagattttggactacaacacctcagaaagagggcctctatgacacctggggccaggggacccaggtcaccgtctcctccgaacccaagacaccaaaaccacaaact

SEQ ID NO: 214 - protein, 133 aa, Artificial Sequence
  Description of Artificial Sequence: Synthetic polypeptide
EVQLQESGGGSLQTGDSLRLACEASEIVVENYVMAWFRQAPGKEREWLARIIWNTGGTHLQEFVKGREGIGYSVKTSTRTVMNSLKPEDTAIYYCAALRSWTTTPQREVLYDNWGHGTQVTVSSAHHSEDPIS

SEQ ID NO: 215 - DNA, 399 bp, Artificial Sequence
  Description of Artificial Sequence: Synthetic polynucleotide
gaggtgcagctgcaggagtctgggggaggatcgctgcagactggggactcactgagactcgcctgtgaagcctctgaaatcgtcgtcgaaaattatgtcatggcctggttccgccaggctccagggaaggagcgtgagtggctagcgcgtattatctggaataccggtggcacacatcttcaagaatttgtgaagggccgagaagggatcggctatagcgtcaaaacttccacccgcacagtaatgaacagcctgaaacccgaggacacggccatttattactgcgcagcacttagatcttggactactacacctcagagggaggtcctctatgacaactggggccacgggacccaggtcaccgtctcctcagcgcaccacagcgaagaccctattagt

SEQ ID NO: 216 - protein, 537 aa, Artificial Sequence
  Description of Artificial Sequence: Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGSGSGKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 217 - DNA, 1614 bp, Artificial Sequence
  Description of Artificial Sequence: Synthetic polynucleotide
atgctgctgctggtgaccagcctgctgctgtgcgagctgccccaccccgccttcctgctgatccccgacatccagatgacccagaccaccagcagcctgagcgccagcctgggcgacagagtgaccatcagctgcagagccagccaggacatcagcaagtacctgaactggtaccagcagaagcccgacggcaccgtgaagctgctgatctaccacaccagcagactgcacagcggcgtgcccagcagattcagcggcagcggcagcggcaccgactacagcctgaccatcagcaacctggagcaggaggacatcgccacctacttctgccagcagggcaacaccctgccctacaccttcggcggcggcaccaagctggagatcaccggcggcggcggcagcggcggcggcggcagcggcggcggcggcagcgaggtgaagctgcaggagagcggccccggcctggtggcccccagccagagcctgagcgtgacctgcaccgtgagcggcgtgagcctgcccgactacggcgtgagctggatcagacagccccccagaaagggcctggagtggctgggcgtgatctggggcagcgagaccacctactacaacagcgccctgaagagcagactgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcagcggcagcggcagcggcaagcccaccaccacccccgcccccagaccccccacccccgcccccaccatcgccagccagcccctgagcctgagacccgaggcctgcagacccgccgccggcggcgccgtgcacaccagaggcctggacttcgcccccagaaagatcgaggtgatgtacccccccccctacctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagataa

SEQ ID NO: 218 - protein, 547 aa, Artificial Sequence
  Description of Artificial Sequence: Synthetic polypeptide
MYRMQLLSCIALSLALVTNSQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGREDPDAVDIWGQGTMVTVSSSGGGGSGGGGSGGGGSGGGGSGGGGSSDIQMTQSPSSLSASVGDRVTITCRASQGIRSWLAWYQQKPEKARKSLIYAASSLQSGVPSRFSGSGSGTDFTLTISSLQPEDFATYYCQQYNSYPLTFGGGTKVEIKSSGSGSPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 219 - DNA, 1641 bp, Artificial Sequence
  Description of Artificial Sequence: Synthetic polynucleotide
atgtacagaatgcagctgctgagctgcatcgccctgagcctggccctggtgaccaacagccaggtgcagctggtgcagagcggcgccgaggtgaagaagcccggcagcagcgtgaaggtgagctgcaaggccttcggcggcaccttcagcagctacgccatcagctgggtgagacaggcccccggccagggcctggagtggatgggcagaatcatcagattcctgggcatcgccaactacgcccagaagttccagggcagagtgaccctgatcgccgacaagagcaccaacaccgcctacatggagctgagcagcctgagaagcgaggacaccgccgtgtactactgcgccggcgagcccggcagagaggaccccgacgccgtggacatctggggccagggcaccatggtgaccgtgagcagcagcggcggcggcggcagcggcggcggcggcagcggcggcggcggcagcggcggcggcggcagcggcggcggcggcagcagcgacatccagatgacccagagccccagcagcctgagcgccagcgtgggcgacagagtgaccatcacctgcagagccagccagggcatcagaagctggctggcctggtaccagcagaagcccgagaaggccagaaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacaacagctaccccctgaccttcggcggcggcaccaaggtggagatcaagagcagcggcagcggcagccccaccaccacccccgcccccagaccccccacccccgcccccaccatcgccagccagcccctgagcctgagacccgaggcctgcagacccgccgccggcggcgccgtgcacaccagaggcctggacttcgcccccagaaagatcgaggtgatgtacccccccccctacctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 220 - protein, 224 aa, Artificial Sequence
  Description of Artificial Sequence: Synthetic polypeptide
MAQVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSGGGGSDEGKRYRVKVIPPNTTNSQSVKIQPYTRQTTPDQEHKFELQFETNGNYDSKIPSTTVAIVVGVIAGFITLIIVFICYICCRKRPRAYNHMVDPLLSFSY

SEQ ID NO: 221 - DNA, 675 bp, Artificial Sequence
  Description of Artificial Sequence: Synthetic polynucleotide
atggcccaggtgcagctggtggagagcggcggcggcctggtgcaccccggcgacagcctgagactgagctgcgccgccagcggcagcgtgttcaacagcgccaccatgggctggtacagacagagccccggcagccagagagagctggtggccaccatcgtggtgggcacccccacctacgccgacagcgtgaagggcagattcaccatcagcagagacaacgccaagaacatcgtgtacctgcagatgaacagcctgaagcccgaggacaccgccgtgtactactgcaactacagagccacctacaccagcggctacagcagagactactggggccagggcacccaggtgaccgtgagcggcggcggcggcagcgatgagggaaaacggtaccgggttaaggttattccgcctaacaccacaaactcccagagtgtcaaaattcagccttacaccaggcagactactcctgaccaggaacacaaattcgaattacagtttgagactaacggtaactatgactccaagattccatctacaacggtcgcgatcgtagtgggcgtgattgcaggcttcatcacattgatcatcgtgttcatctgctatatctgctgtaggaagcgccctcgggcgtacaaccacatggtggaccctctgttgagtttctcatattaa

SEQ ID NO: 222 - protein, 224 aa, Artificial Sequence
  Description of Artificial Sequence: Synthetic polypeptide
MAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVSGGGGSDEGKRYRVKVIPPNTTNSQSVKIQPYTRQTTPDQEHKFELQFETNGNYDSKIPSTTVAIVVGVIAGFITLIIVFICYICCRKRPRAYNHMVDPLLSFSY

SEQ ID NO: 223 - DNA, 675 bp, Artificial Sequence
  Description of Artificial Sequence: Synthetic polynucleotide
atggcccaggtgcagctggtgcagagcggcggcggcctggtgcagcccggcggcagcctgagactgagctgcgccgccagcggcagagccttcaacagcgccgccatgggctggtacagacaggcccccggcagccagagagagctggtggccagcatcagcgccggcaccgccagctacgccgacgccgtgaagggcagattcaccatcagcagagactacgccaagaacatcatctacctgcagatgaacagcctgaagcccgacgacaccgccgtgtacttctgcaactacagaaccacctacaccagcggctacagcgaggactactggggccagggcacccaggtgaccgtgagcggcggcggcggcagcgatgagggaaaacggtaccgggttaaggttattccgcctaacaccacaaactcccagagtgtcaaaattcagccttacaccaggcagactactcctgaccaggaacacaaattcgaattacagtttgagactaacggtaactatgactccaagattccatctacaacggtcgcgatcgtagtgggcgtgattgcaggcttcatcacattgatcatcgtgttcatctgctatatctgctgtaggaagcgccctcgggcgtacaaccacatggtggaccctctgttgagtttctcatattaa

SEQ ID NO: 224 - protein, 391 aa, Artificial Sequence
  Description of Artificial Sequence: Synthetic polypeptide
MYRMQLLSCIALSLALVTNSEVQLQQSGPELVKPGASVRMSCTASGYTFTSYVMHWIKQKPGQGLDWIGYINPYNGGTQYNEKFKGKATLTSDKSSSTAYMELSSLTSEDSAVYYCARRTFPYYFDYWGQGTTLTVSSSGGGGSGGGGSGGGGSGGGGSGGGGSSDVLMTQTPLSLPVSLGDQASISCRSSQSILHSNGNTYLEWYLQKPGQSPKLLIYKVSNRFSGVPDRFSGSGSGTDFTLKISRVEAEDLGVYYCFQGSHAPLTFGAGTKLELKSSGSGSPTTTPAPRPPTPAPTIASQPLSLRPEACRPAAGGAVHTRGLDFAPRKIEVMYPPPYLDNEKSNGTIIHVKGKHLCPSPLFPGPSKPTLIALVTSGALLAVLGITGYFL

SEQ ID NO: 225 - DNA, 1176 bp, Artificial Sequence
  Description of Artificial Sequence: Synthetic polynucleotide
atgtacagaatgcagctgctgagctgcatcgccctgagcctggccctggtgaccaacagcgaggtgcagctgcagcagagcggccccgagctggtgaagcccggcgccagcgtgagaatgagctgcaccgccagcggctacaccttcaccagctacgtgatgcactggatcaagcagaagcccggccagggcctggactggatcggctacatcaacccctacaacggcggcacccagtacaacgagaagttcaagggcaaggccaccctgaccagcgacaagagcagcagcaccgcctacatggagctgagcagcctgaccagcgaggacagcgccgtgtactactgcgccagaagaaccttcccctactacttcgactactggggccagggcaccaccctgaccgtgagcagcagcggcggcggcggcagcggcggcggcggcagcggcggcggcggcagcggcggcggcggcagcggcggcggcggcagcagcgacgtgctgatgacccagacccccctgagcctgcccgtgagcctgggcgaccaggccagcatcagctgcagaagcagccagagcatcctgcacagcaacggcaacacctacctggagtggtacctgcagaagcccggccagagccccaagctgctgatctacaaggtgagcaacagattcagcggcgtgcccgacagattcagcggcagcggcagcggcaccgacttcaccctgaagatcagcagagtggaggccgaggacctgggcgtgtactactgcttccagggcagccacgcccccctgaccttcggcgccggcaccaagctggagctgaagagcagcggcagcggcagccccaccaccacccccgcccccagaccccccacccccgcccccaccatcgccagccagcccctgagcctgagacccgaggcctgcagacccgccgccggcggcgccgtgcacaccagaggcctggacttcgcccccagaaagatcgaggtgatgtacccccccccctacctggacaacgagaagagcaacggcaccatcatccacgtgaagggcaagcacctgtgccccagccccctgttccccggccccagcaagcccaccctgatcgccctggtgaccagcggcgccctgctggccgtgctgggcatcaccggctacttcctgtaa

SEQ ID NO: 226 - protein, 5 aa, Artificial Sequence
  Description of Artificial Sequence: Synthetic peptide
SGGGG

(19) United States
(12) Patent Application Publication
(10) Pub. No.: US 2023/0272035 A1
(43) Pub. Date: Aug. 31, 2023

(54) MODULATORS OF THE IMMUNE ESCAPE MECHANISM FOR UNIVERSAL CELL THERAPY

(71) Applicant: VYCELLIX, INC., Tampa, FL (US)

(72) Inventors: Evren ALICI, Huddinge (SE); Alamdar HUSSAIN, Huddinge (SE)

(73) Assignee: VYCELLIX, INC., Tampa, FL (US)

(21) Appl. No.: 17/782,509

(22) PCT Filed: Dec. 7, 2020
(86) PCT No.: PCT/US2020/063682

§ 371 Date: Jun. 3, 2022

Related U.S. Application Data
(60) Provisional application No. 62/943,807, filed on Dec. 5, 2019.

(51) Int. Cl. C07K 14/725; A61P 37/02

(52) U.S. Cl. CPC C07K 14/7051; A61P 37/02; C07K 2319/03; C07K 2319/33; C07K 2319/02

(57) ABSTRACT

Therapeutic agents capable of detaining bulky proteins such as CD45, CD148, and CD43 in the middle of the cellular interface between a graft cell and CD45 positive host effector cell (such as a T cell, NK cell, B cell, or dendritic cell) are disclosed, as are methods for their use and products made with such therapeutic agents. The therapeutic agents prevent or inhibit the formation of functional immunologic synapses (including physiological SMAC). They also result in continuous dephosphorylation of signal transduction pathways.

### PRIORITY CLAIM AND INCORPORATION BY REFERENCE

This application claims priority to U.S. provisional patent application No. 62/943,807 filed on Dec. 5, 2019, the contents of which are incorporated herein by reference. All references cited herein are expressly incorporated by reference.

### BACKGROUND

Cytotherapy is an auspicious achievement of modem science which is currently being used to replace damaged tissue and/or organs and seems promising for many ailments including diabetes, retinitis pigmentosa, Parkinson's disease, myocardial infarction, blood cancers including lymphomas and leukemia, bone marrow failure syndromes including anaemias and cytopenias, inherited immune disorders including Wiskott-Aldrich Syndrome (WAS) and Severe Combined Immunodeficiency (SCID), hemoglobinopathies including thalassemias, sickle cell anemias and congenital dyserythropoitiec anaemias, inherited metabolic disorders including lysosomal storage disorders, galactosemia, phenylketonuria and glycogen storage diseases, neurological disorders including neuromyelitis optica, cartilage replacements including knee replacements and Crohn's disease, etc. Just like organ transplantation, cytotherapy also faces the challenges of restricted donor availability and immune rejection. This demands for the development of mechanisms that render the cells immune-privileged. Immune-privileged cells will not only allow the generation of “off the shelf” cellular products but may also lead to the generation of “off the shelf” organs.

A universal cell is a cell which can be administered to any patient without triggering an immune response. This has been the holy grail of organ transplant and cellular therapy since these fields were created. The lack of a universal cell limits off the shelf therapies in general and reduces many therapies to close tissue matches between donor and recipient. In almost all cases immunosuppressive drugs are administered with significant side effects.

The obvious side effect of administration of immunuspressive agents is increased general susceptibility to infections and cancer. Commonly used immunosuppressive drugs include cyclosporins, azathioprine, antilymphoblast, antithymocyte globulins, muromonab-CD3, and porcine antilymphocyte globulin (P-ALG). The cyclosporins are known to cause nephrotoxicity, hepatotoxicity, hyperkalemia, hypertension, tremor, gum overgrowth, and hirsutism. Azathioprine supresses the bone marrow suppression, leading to leukopenia. Antilymphoblast and antithymocyte globulins are foreign antibodies that may cause allergic-type reactions such as fever, chill, and hypotension. The initial side effect of monoclonal antibody (muromonab-CD3, OKT3) is similar to that of P-ALG. It includes high fever, shaking chills, headache, rigors, and hypotension. Min, D. I. and Monaco, A. P. (1991), Complications Associated with Immunosuppressive Therapy and Their Management. Pharmacotherapy: The Journal of Human Pharmacology and Drug Therapy, 11: 119S-125S.

The art contains many examples of attempts to make cells compatible with any recipient. The most common approach is disruption of Beta-2 Microglobulin (B2M) which eliminates surface expression of all class I molecules, but leaves the cells vulnerable to lysis by natural killer (NK) cells. Insertion of HLA-E genes at the B2M locus in human pluripotent stem cells (PSCs) confers inducible, regulated, surface expression of HLA-E single-chain dimers (fused to B2M) or trimers (fused to B2M and a peptide antigen), without surface expression of HLA-A, B or C. These HLA-engineered PSCs and their differentiated derivatives are not recognized as allogeneic by CD8⁺ T cells, do not bind anti-HLA antibodies and are resistant to NK-mediated lysis. Gornalusse, Germán G, Hirata, Roli K, Funk, Sarah E, Riolobos, Laura, Lopes, Vanda S, Manske, Gabriel, Prunkard, Donna, Colunga, Aric G, Hanafi, Laila-Aicha, Clegg, Dennis O, Turtle, Cameron, Russell, David W.; HLA-E-expressing pluripotent stem cells escape allogeneic responses and lysis by NK cells, Nature Biotechnology (Vol 35 p 765) 2017/05/15/online; and Glas R, Franksson L, Ohlen C, Hoglund P, Koller B, Ljunggren H G, et al. Major histocompatibility complex class I-specific and -restricted killing of beta 2-microglobulin-deficient cells by CD8+ cytotoxic T lymphocytes. Proc Natl Acad Sci USA. 1992; 89(23):11381-5. While such an approach prevents some cells from being recognized by the immune system, it is a genetic engineering approach that does not provide a true universal cell. Furthermore, potential CIS interactions between HLA-E with NKG2A and NKG2C may affect the graft's function, leading to a suboptimal cellular product. Moreover, the cellular product is generated in multiple gene editing steps consisting of simultaneous knockout of all the HLA class I molecules and knock-in of HLA-E B2M fusion protein.

CRISPR-Cas9 and other gene-editing technologies have started a race to create “off-the-shelf” donor cells that are invisible to the immune system. The common approach for creating such cells involves the manipulation of genes required for immune recognition, in particular HLA class I and II proteins. Other approaches leverage knowledge of immune-cloaking strategies used by certain bacteria, viruses, parasites, the fetus, and cancer cells to induce tolerance to allogeneic cell-based therapies by modifying cells to express immune-suppressive molecules such as PD-L1 and CTL A4-Ig. The same mechanisms that lead to cell and tissue rejection are also implicated in autoimmune disease. There remains a need in the art for a universal cell which is safe and effective.

### SUMMARY OF THE INVENTION

Many pathogenic and non-pathogenic microbes have shaped our immune system and thus have themselves evolved in turn and have mastered immune evasion, especially seen in chronic infections. Epstein Bar Virus (EBV) is one such example of an immune system evader. We have discovered that it is possible to exploit the immune evasion mechanisms evolved by various pathogens which render them immune-privileged. Human cytomegalovirus (HCMV) inhibits T cell activity through engagement of UL11 protein (FIG. 27) with CD45, culminating in disruption of proximal signal transduction required for activation and/or development of T cells. Similarly, E3 protein from Adenovirus (FIG. 21) engages CD45 and inhibits NK and T cells. We have shown that, through genetic modification, we can avoid graft rejection by expressing binding molecules against CD45 on the graft cell surface. (FIG. 7-12). Following the same lines, we have also put together a single-chain of a monoclonal antibody against CD45 (a-CD45-sc) (FIG. 24). Cells modified to express CD45 engagers are hereby reported for their immune evasion properties. For this purpose, cytotoxicity of NK and T cells against target cells expressing UL11, E3.49K or a-CD45-sc or GFP (as control) was tested and compared (FIG. 7-12).

CD45 is a transmembrane protein tyrosine phosphatase (PTPase) expressed on nucleated cells. It has a heavily glycosylated large extracellular domain and tandem intracellular phosphatase domains. CD45 covers approximately 10% of the surface area of B and T cells, where it regulates the development and activation of the cells by governing the membrane proximal signalling. Following cellular synapse formation, CD45 dephosphorylates an inhibitory tyrosine in the tail of SRC family kinases, allowing an “open” un-inhibited conformation. “Open” SRC family kinases achieve an elevated kinase activity through auto-phosphorylation on their own kinase domain activation loops. Active SRC family kinases further phosphorylate protein molecules containing immunoreceptor tyrosine-based activation motifs (ITAMs) and SYK family kinases, thus resulting in signal transduction, propagation and amplification. In successful cellular immune reactions, CD45 is excluded from the immune synapse and is only brought back into the synapse at the cessation of the reaction. CD45 dephosphorylates activation loop phosphorylation and brings down SRC family kinase activity, resulting in the termination of the immune signalling. Moreover, it also dephosphorylates Janus kinases thus dampening cytokine receptor signalling. CD45 may also dephosphorylate other proximal signal transduction molecules including ZAP70 and CD3-Zeta. CD45 is a constitutive active type-I membrane phosphatase consisting of a heavily glycosylated extracellular domain and intracellular tandem phosphatase domains, with intrinsic catalytic activity of membrane proximal domain. Membrane proximal extracellular region consists of Fibronectin type III domains followed by cysteine-rich domain and the distal regions which are heavily glycosylated. The CD45 gene has multiple exons and alternating splicing of 4 (A), 5 (B), 6 (C) exons produces the transcripts of variable length. Human CD45 can be result of alternating exon usage and can produce ABC, AB, BC, B and O isoforms. The shortest product with all three exons (A, B and C) missing is called CD45RO, while the one containing all these exons is the longest called CD45RABC. Different isoforms are used as development and activation markers in various lymphocytes. CD45RO, among all isoforms, is the conserved domain that is targeted.

CD148 is a receptor tyrosine phosphatase with a heavily glycosylated, large fibronectin extracellular domain and an intracellular catalytic domain. Along with hematopoietic lineages, CD148 is also expressed in vascular and duct endothelial cells where it negatively regulates cell proliferation and transformation. Loss of CD148 has been observed in cancer cell lines and re-expression resulted in the suppression of tumor growth both in vitro and in vivo. CD148 dephosphorylates a number of growth factor receptors including VEGFR, EGFR, HGFR and FGFR and other key downstream signaling molecules like p85, PLC γ1, and ERK1/2.

CD43 is a highly glycosylated, mucin type protein with a large extracellular domain and small globular intracellular domain expressed on the hematopoietic cells including stem cells, T cells, monocytes, granulocytes, NK cells, and platelets. CD43 extracellular domain promotes adhesion through interaction with E-selectin, galectin-1 and galectin-3, siglec-1, M-ficolin, integrins, cell surface nucleolin, and ICAM-1 (intercellular adhesion molecule type 1). While the conserved intracellular domain is involved in signal transduction mediating the connection to the cytoskeleton through binding to ezrin, radixin and moesin (ERM) proteins CD43 has a proline-rich sequence resembling SH3 binding consensus and a nuclear localization signal (NLS), which explains the nuclear localization of CD43.

The B Cell Receptor (BCR) is a membrane bound immunoglobulin with a short intracellular domain of three amino acids. BCRs are made up of two identical heavy chains and two light chains. The extracellular domain has the capacity to specifically recognize and bind the antigens. The BCR lacks intracellular signaling which is compensated by two associated ITAMs containing chains Iga (Alpha) and IgP (Beta). Following successful binding to the antigen, the BCR transduces signaling leading to B cells' activation and maturation. Following class switching, BCRs are switched from membrane bound to a released form and are then called antibodies.

The immune synapse is the interface between the target cells and the lymphocytes and is also called Supramolecular Activation Cluster (SMAC) due to the accumulation of activating and regulatory molecules (FIG. 1A-1E, left side). Before the immune synapse formation, molecules are stochastically distributed (FIG. 1A, left side). Upon ligation of TCR with the target MHCp complex (FIG. 1B, left side), LCK is retained while CD45 is mobilized or pushed to the periphery (FIG. 1C, left side). This, as a consequence, results in activation of LCK. Finally, as CD45 is pushed to the periphery, coreceptors ligate resulting in a mature synapse formation (FIG. 1D, left side). This interface, or SMAC, is composed of concentric circles of molecules involved in the immune cell recognition. The inner most central SMAC (cSMAC) consists of TCR/CD3/MHCp, CD28/CD80, SRC family kinase/s, and PKC6. Outside cSMAC is the peripheral SMAC (pSMAC) consisting of an adhesion ring of LFA-1, ICAM-1, and Talins, followed by the outermost circle called distal SMAC (dSMAC) consisting of glycoproteins including CD45, CD43 and CD148.

In our system, we disrupt sequence of formation and structure of the immune synapse by detaining bulky proteins such as CD45 in the middle of the cellular interface between a graft cell and cytotoxic cell such as a T cell or NK cell (FIGS. 1A-E, right side; FIG. 2A-2D). This not only prevents the formation of physiological SMAC (in the case of graft cell-T cell interactions). It also results in continuous dephosphorylation of signal transduction pathways. It also results in the disruption of TCR/CD3/MHCp etc. and even the cells may not reach close enough to engage TCR/CD3/MHCp.

CD148 and CD43 may be used in the same way as CD45, albeit in a less pronounced fashion. In certain embodiments, CD45, CD148, and/or CD43 may be detained alone or in combination with other molecules.

In an embodiment, the invention includes a therapeutic agent comprising one or more molecules or cells configured to modulate the ability of CD45, CD148, or CD43 to form a functional immunological synapse with a cytotoxic cell, thereby preventing cytotoxicity. In an embodiment, the therapeutic agent may comprises a protein, aptamer, peptide nucleic acid (PNA), nanoparticle, or cell which expresses or secretes the one or more molecules. In an embodiment, the therapeutic agent may comprise a protein, preferably a protein comprising an antibody, more preferably comprising a single chain antibody or VHH nanobody. In an embodiment, the therapeutic agent may comprise a nanoparticle, preferably a lipid nanoparticle (LNP), dendrimer, or ribonucleoprotein (RNP). In an embodiment, the therapeutic agent may comprise an extracellular vesicle, preferably an exosome or microvesicle. In an embodiment, the therapeutic agent may comprise a cell, preferably a eukaryotic cell, more preferably an avian cell or mammalian cell, e.g., murine, porcine, bovine, canine, feline, or ovine cell, most preferably a human cell. In an embodiment, the therapeutic agent may comprise a hematopoietic cell, stem cell, lymphoid cell, myeloid cell, erythrocyte, or platelet. In an embodiment, the therapeutic agent may comprise one or more excipients or additives, preferably one or more of fillers, extenders, diluents, wetting agents, solvents, emulsifiers, preservatives, absorption enhancers, sustained-release matrices, salts, buffers, starches, sugars, microcrystalline cellulose, granulating agents, lubricants, binders, disintegrating agents, coloring agents, release agents, coating agents, sweetening agents, flavoring agents, antioxidants, plasticizers, gelling agents, thickeners, hardeners, setting agents, suspending agents, surfactants, carriers, stabilizers, and combinations thereof. In an embodiment, the therapeutic agent may be for oral, dermal, enteral, or parenteral administration. In an embodiment, the therapeutic agent may be delivered via injection (e.g., direct injection into a diseased tissue or system injection), patch or other transdermal delivery device, or lavage. In an embodiment, the therapeutic agent may comprise a component of viral or bacterial origin, preferably ULL or E3/49k, or a fragment thereof. In an embodiment, the therapeutic agent may comprise a component of viral or bacterial origin, e.g., which does not comprise a ULL protein or fragment thereof or which does not comprise an E3/49k protein, or fragment thereof. In an embodiment, the therapeutic agent may comprise SEQ ID NO: 1, 3, 5, 64, 66, 68, 71, 73, 220, 223, or 224, or a protein having at least 80% identity to SEQ ID NO: 1, 3, 5, 64, 66, 68, 71, 73, 220, 223, or 224. In an embodiment, the therapeutic agent may comprise a cell having one or more molecules expressed on the surface of the cell. In an embodiment, the one or more molecules expressed on the surface of the cell comprises a transmembrane protein expressed and the cell comprises a graft cell. In an embodiment, the transmembrane protein may be capable of binding to CD45, CD148, or CD43. In an embodiment, the CD45, CD148, or CD43 of the therapeutic agent may be present on the surface of a cytotoxic cell, preferably a T cell or natural killer (NK) cell. In an embodiment, the transmembrane protein may be capable of retaining CD45, CD148, or CD43 in a developing immunological synapse on the surface of the cytotoxic cell, thereby disrupting functional immunological synapse formation.

In another embodiment, the invention includes a protein complex capable of preventing cytotoxic cell-induced lysis, which protein complex comprises: an engager comprising SEQ ID NO: 1, 3, 5, 64, 66, 68, 71, 73, 220, 223, or 224, or a protein having at least 80% identity to SEQ ID NO: 1, 3, 5, 64, 66, 68, 71, 73, 220, 223, or 224; and a CD45, CD148, or CD43 protein expressed on the surface of a T cell or NK cell.

In another embodiment, the invention includes a method of manufacturing a composition for functional immunological synapse disruption, the method comprising: expressing one or more molecules on the surface of a first cell, the one or more molecules being configured to retain CD45, CD148, or CD43 on the surface of a second cell in an incomplete immunological synapse, thereby disrupting or inhibiting functional immunological synapse formation between the first cell and the second cell.

In another embodiment, the invention includes a method for promoting escape from NK-mediated lysis, comprising administering the therapeutic agent above to a subject in need thereof. In an embodiment, the method may comprise inhibition or disruption of NKG2D binding to MICA, MICB, and/or ULBP. In an embodiment, the method may comprise disruption of activating NK cell receptors selected from: members of the human Killer Immunoglobulin-like Receptor (KIR) family, CD94-NKG2C/E/H heterodimeric receptors, NKG2D, natural cytotoxicity receptors such as NKp30, NKp44, and NKp46, nectin/nectin-like binding receptors DNAM-1/CD226 and CRTAM, receptors expressed by natural killer (NK) cells that regulate their activation, SLAM family receptors (including 2B4/CD244, CRACC/SLAMF7, and NTB-A/SLAMF6), as well as Fc gamma RIIIA/CD16a, CD27, CD100/Semaphorin 4D, and CD160. In an embodiment, the subject may be at risk of having or suffer from one or more of the following conditions: autoimmune disease, blood cancers, including lymphomas and leukemias; bone marrow failure syndromes, including anemias and cytopenias; inherited immune disorders, including WAS and SCID; hemoglobinopathies, including sickle cell disease (SCD) and thalassemia; neurological disorders, including neuromyelitis optica; and graft vs. host disease.

In another embodiment, the invention includes a method for promoting escape from T cell-mediated lysis, comprising administering a therapeutic agent as above to a subject in need thereof. In an embodiment, the method may comprise inhibition or disruption of T cell receptor binding to MHC peptide. In an embodiment, the subject in need thereof may be at risk of having or suffer from one or more of psoriasis and vitiligo.

In another embodiment, the invention includes a method of positionally detaining CD45 on the surface of a cell expressing CD45 to disrupt formation of a functional immunological synapse, comprising: treating the cell expressing CD45 with an agent having affinity for a membrane-proximal region of an extracellular domain of CD45, thereby positionally detaining CD45 with respect to other membrane proteins expressed on the surface of the cell necessary for formation of the functional immunological synapse.

In another embodiment, the invention includes a nonautologous cell comprising an engager on its surface and which is configured to avoid synapse formation with one or more host cytotoxic cells. In an embodiment, the host cytotoxic cell is a natural killer cell, a T cell, or a macrophage. In an embodiment, the cytotoxic cell is a T cell, preferably a gamma-delta T cell, a CD8⁺ T cell, a CD4⁺ T cell, or a mucosal associated invariant T cell. In an embodiment, the nonautologous cell is free of genetic modification. In an embodiment, the nonautologous cell may be treated with an engager.

In another embodiment, the invention includes a method for producing a xenogenic cell for transplantion, the method comprising protecting the xenogenic cell to be transplanted with the therapeutic agent above. In an embodiment, the therapeutic agent may be administered to a host prior to transplantation of the xenogenic cell, or concurrently with the xenogenic cell. In an embodiment, the therapeutic agent may be bound to the surface of the xenogenic cell for transplantation. In an embodiment, the therapeutic agent may be a cell and the cell may be genetically modified to express an engager on its surface or in a extracellular vesicle.

In another embodiment, the invention includes a method of preventing rejection of solid organ or organoid transplant, comprising: transducing or transfecting cells of the solid organ or organoid with a gene to prevent or inhibit binding of cytotoxic cells to cells of the solid organ or organoid transplant. In an embodiment, the gene may code for an engager and the engager may be expressed in an amount or density effective to inhibit functional immunological synapse formation upon exposure of the solid organ or organoid to a cytotoxic cell.

In another embodiment, the invention includes a method of treating cancer comprising: administering a hematopoietic stem cell comprising a membrane-bound engager to a subject in need thereof.

In another embodiment, the invention includes an recombinant protein which may comprise: (i) a signal peptide, (ii) a heavy chain of an antibody, (iii) a first linker, (iv) a light chain of an antibody, (v) optionally, a second linker, (vi) a stalk, (vii) a transmembrane region, and (viii) optionally, an intracellular region. In an embodiment, the recombinant protein may comprise a second linker which links the light chain to the stalk. In an embodiment, the recombinant protein may be a single chain antibody, preferably a single chain antibody which binds specifically to CD45, CD148, or CD43. In an embodiment, each of (i)-(vii) may be present, and may be connected in order from amino terminus to carboxyl terminus of the protein. In an embodiment, the signal peptide may be an IL2 signal peptide; the first linker may comprise an SGGGG motif and/or may vary in length from 5-60, preferably 10-50, more preferably 20-45 amino acids; the second linker, when present, may vary in length from 5 to 60, preferably 5-40, more preferably 7-15 amino acids; the stalk may be at least 8 and no more than 200 amino acids in length, and the transmembrane region may be derived from CD34, CD45, CD28, and/or Cd8a.

In another embodiment, the invention includes a cell comprising an engager and an exogenous suicide gene.

In another embodiment, the invention includes a first cytotoxic cell expressing membrane-bound CD45, CD148, and/or CD43, which cell may be treated to prevent functional immunological synapse formation between a second cytotoxic cell expressing membrane-bound CD45, CD148, and/or CD43. In an embodiment, the cytotoxic cell may be a natural killer cell, a T cell, or a macrophage.

In another embodiment, the invention includes a graft treated to prevent the binding of cytotoxic cells, wherein the treatment comprises exposing the graft to a therapeutic agent as above.

In another embodiment, the invention includes a method of controlling inflammation comprising administering an mRNA or DNA encoding an engager to a subject in need thereof, thereby modulating functional immunological synapse formation to control inflammation. In an embodiment, functional immunological synapse formation may be inhibited, thereby reducing inflammation.

In another embodiment, the invention includes use of an engager for reducing cytotoxic cell response to transplantation. In an embodiment, the use may be performed in the absence of HLA-I and/or HLA-II knockout or knockdown. In an embodiment, the use may be performed in combination with HLA-1 and/or HLA-II knockout or knockdown.

In another embodiment, the invention includes a cell comprising a surface-bound engager and a chimeric antigen receptor (CAR). In an embodiment, the CAR comprises a-CD38CAR (SEQ ID NO: 218) or a variant thereof having at least 80% identity thereto. In an embodiment, the CAR comprises a-CD19CAR (SEQ ID NO: 216) or a variant thereof having at least 80% identity thereto.

In another embodiment, the mention includes an anti-CD45, anti-CD148, or anti-CD43 engager comprising a transmembrane domain configured on the surface of a cell. In an embodiment, the invention includes an engager comprising a membrane bound antibody, nanobody, or single chain to CD 45, CD43 or CD148.

In another embodiment, the invention includes a vector or plasmid for creating an anti-CD45, anti-CD148, or anti-CD43 engager comprising DNA encoding anti-CD45, anti-CD148, or anti-CD43 engager operably linked to a promoter. In an embodiment, the invention includes a vector or plasmid encoding a membrane bound antibody, nanobody, or single chain to CD45, CD148 or CD43.

### DESCRIPTION OF THE FIGURES

FIG. 1A-1E are drawing snapshots showing the Supramolecular Activation Cluster (SMAC) formation stages leading to mature immune synapse.

FIG. 2A is a drawing showing the immune synapse between host T cells and graft cells. The engagement of host TCR with donor MHC-peptide complex leads to the killing of the graft.

FIG. 2B is a drawing showing the interaction between host T cells and graft cells expressing the novel engager keeping CD45 in the middle of the synapse. This leads to no-killing of the graft and lack of a functional immunological synapse formation.

FIG. 2C is a drawing showing the immune synapse between host NK cells and graft cells. The engagement of host activating receptors with recipient ligands leads to the killing of the graft.

FIG. 2D is a drawing showing the interaction between host NK cells and graft cells expressing the novel engager keeping CD45 in the middle of the synapse. This leads to no-killing of the graft and lack of a functional immunological synapse formation.

FIG. 3 is a map of plasmid LeGO-iG2-UL11.

FIG. 4 is a map of plasmid LeGO-iG2-E3.49k.

FIG. 5 is a map of plasmid LeGO-iG2-A-CD45-SC.

FIG. 6 is a drawing showing generation of stable cell lines.

FIG. 7 is a bar graph showing inhibition of cell lysis in cells transformed with a-CD45-sc, E3.49K or UL11 (control is untransformed). The y-axis shows percent specific lysis ⁵¹Cr release in K562 cells incubated with PBMCs. Effector:Target (E:T) ratios shown below bar groupings.

FIG. 8 is a bar graph showing inhibition of cell lysis in cells transformed with a-CD45-sc, E3.49K or UL11 (control is untransformed). The y-axis shows percent specific lysis ⁵¹Cr release in K562 cells incubated with NK92 cells; E:T ratios shown below bar groupings.

FIG. 9 is a line graph showing inhibition of cell lysis in K562 cells transformed with a-CD45-sc, E3.49K or UL11 (control is untransformed) when exposed to PBMC cells. The y-axis shows percent specific lysis ⁵¹Cr release in K562 cells incubated with PBMCs; the x-axis shows E:T ratio.

FIG. 10 is a line graph showing inhibition of cell lysis in K562 cells transformed with a-CD45-sc, E3.49K or UL11 (control is untransformed) when exposed to NK92 cells. The y-axis shows percent specific lysis ⁵¹Cr release in K562 cells incubated with NK92 cells; the x-axis shows E:T ratio.

FIG. 11 is a line graph showing prophetic data regarding inhibition of cell lysis in RPMI88226 cells transformed with a-CD45-sc, E3.49K or UL11 (control is untransformed) when exposed to T cells. The y-axis refers to percent specific lysis ⁵¹Cr release; the x-axis shows E:T ratio.

FIG. 12 is a line graph showing data regarding inhibition of cell lysis in CD34 differentiated T-like cells transformed with a-CD45-sc, E3.49K or UL11 (control is untransformed) when exposed to CD8⁺ T cells. The y-axis refers to percent specific lysis ⁵¹Cr release; the x-axis shows E:T ratio.

FIG. 13 is a map of plasmid LeGO-iG2-a-CD45-(M)-VHH1.

FIG. 14 is a map of plasmid LeGO-iG2-a-CD45-(M)-VHH2.

FIG. 15 is a map of plasmid LeGO-iG2-E3.49K.R1.

FIG. 16 is a map of plasmid LeGO-iG2-E3.49K.R3.

FIG. 17 is a map of plasmid LeGO-iG2-mVHH1-E3TM.

FIG. 18 is a map of plasmid LeGO-iG2-mVHH2-E3TM.

FIG. 19 is a map of plasmid LeGO-iG2-a-CD19CAR.

FIG. 20 is a map of plasmid LeGO-iG2-a-CD38CAR.

FIG. 21 is diagrammatic presentation of E3.49K (SEQ ID NO: 3).

FIG. 22 is diagrammatic presentation of E3.49K.R1 (SEQ ID NO: 66).

FIG. 23 is diagrammatic presentation of E3.49K.R3 (SEQ ID NO: 68).

FIG. 24 is diagrammatic presentation of a-CD45-sc (SEQ ID NO: 5).

FIG. 25 is diagrammatic presentation of m-VHH1-E3-TM (SEQ ID NO: 220).

FIG. 26 is diagrammatic presentation of m-VHH2-E3-TM (SEQ ID NO: 222).

FIG. 27 is diagrammatic presentation of UL11 (SEQ ID NO: 1).

FIG. 28 is diagrammatic presentation of a-CD38CAR (SEQ ID NO: 218).

FIG. 29 is diagrammatic presentation of a-CD19CAR (SEQ ID NO: 216).

FIG. 30 is a line graph showing cell lysis of target cells by NK92 cells expressing a-CD45-sc.

FIG. 31 is a line graph showing cell lysis of target cells by TALL-104 cells expressing a-CD45-sc.

FIG. 32 is the experimental flow chart that was followed for in vivo experiments.

FIG. 33 is a compilation of IVIS images of RPMI-8226 cells transduced with luciferase and CD45 engager, that were treated with PBMCs and Daratumumab. A higher tumor burden compared to those of FIG. 34 is observed although the same dose of RPM18226 cells are administered.

FIG. 34 is a compilation of IVIS images of RPMI-8226 cells transduced with luciferase (but not with CD45 engager), that were treated with PBMCs and Daratumumab. A controlled minimal residual disease is observed.

FIG. 35 is a line graph showing the effects of a-CD45-sc on K562 cells after PBMC exposure. FIG. 35 shows IVIS imaging analysis on K562 tumor bearing mice vs K562 with CD45 Engager. All mice depicted have received PBMCs. Each line represents one mouse.

FIG. 36 is a line graph showing the effects of a-CD45-sc on SKOV3 cells treated with Herceptin and after PBMC exposure. FIG. 36 shows IVIS imaging analysis on SKOV3 tumor bearing mice vs SKOV3 with CD45 Engager. All mice depicted have received PBMCs and Trastuzumab, except the control group that received only PBMCs. Each line represents one mouse.

FIG. 37 is diagrammatic scheme of loading mRNA into EVs.

FIG. 38 is a line graph showing the arthiritis score following therapeutic EVs injections. The higher the score, the more aggressive it is. Each limb was scored using a scale from 0 to 4 based on increasing levels of erythema and swelling.

FIGS. 39A and 39B are bar graphs showing the TNFa (pg/100 μg protein) and IL1b (pg/100 μg protein) secretion in arthritis models following therapeutic EVs injections.

FIG. 40 is schematic flowchart showing the EVs production/isolation and purification of therapeutic EVs.

FIG. 41 is a line graph showing cell lysis of target cells (RPMI8226) by NK92 cells co-expressing a-CD45-sc and a-CD38CAR as assessed by ⁵¹Cr release assay.

FIG. 42 is a line graph showing cell lysis of target cells (CD38KO RPMI8226) by NK92 cells co-expressing a-CD45-sc and a-CD38CAR as assessed by ⁵¹Cr release assay.

FIG. 43 is a bar chart showing degranulation of target cells (Raji and Jurkat) by NK92 cells co-expressing a-CD45-sc and a-CD19CAR.

FIG. 44 is alive cell imaging co-culture of target cell (K562 with NK92 co-expressing a-CD45-sc and a-CD38CAR. Dead cells appear light. Effector cells appear dark. This is a microscopic representation of what is demonstrated in FIG. 42 and FIG. 43.

### INTRODUCTION

The differentiation potential of pluripotent stem cells such as embryonic stem cells (ESC) made it possible to provide unlimited supply of any cell type for transplantations. ESCs were expected to provide “off the shelf” cellular therapies for Parkinson, diabetes, cardiovascular diseases etc, where any damaged tissues needed repair or replacement. Yet the immune rejection drastically limited the use of this opportunity. Induced Pluripotent Stem Cells (iPSCs) provided the solution of generating pluripotent cells from the patient and then differentiating them to the required cell type. IPSC generation, genetic-repair and differentiation and therapeutic and safety validation for individual patients is not affordable in terms of expediency and cost. Despite immune-rejection, which remains the Achilles' heel of the cell therapy field, notable progress has been made in the form of mesenchymal stem cells, CAR-T cells, and adult stem cells. A number of strategies have been developed to prevent immune rejection for in vivo persistence of allografts.

Immunosuppression with continuous cyclosporine and cyclophosphamide was the only option in organ transplants and in treating autoimmunity. Furthermore, use of cyclophosphamide and fludarabine treatment regimens have been utilized for transient host lymphodepletion in order to create a milieu where donor cells can be retained for a period of approximately two weeks in the context of donor lymphocyte infusions, Chimeric Antigen Receptor modified T cell therapies, as well as other genetically modified cell infusions. It is also used in alternative medicine for extended in vivo expression of gene therapy vectors. In cellular transplants, to prevent host T cell mediated rejection of allografts, donor cells HLA have been knocked out as a possible host CD8+ T cell (HLA Class I knockout) and CD4+ T cell (HLA Class II knockout) mediated immune evasion strategy. At the same time, non-classical HLA expression was forced on these cells to prevent NK cell mediated cytotoxicity. Similarly, CTLA4Ig was used to prevent T cells CD28 coreceptor ligation and thus immune reaction against the donor cells and CD40 mAb was employed to dampen APC and B cell functions. Some studies exploited viral proteins redirected to intracellular signaling and processing of antigens to prevent immune reaction. For example, ICP4, a cytosolic protein from HSV inhibits TAP mediated transport of peptide to endoplasmic reticulum (ER while HCMV proteins US11/2 lead to degradation of MHC-I, US3 retains MHC-I in ER, and US6 blocks TAP.

We aim to exploit immune evasion methods employed by viruses that target the direct extracellular, intercellular trans interactions with the regulatory proteins of T cells and NK cells. UL11 is a member of RL11 protein family and is expressed on the surface of CMV infected cells and binds CD45 on leukocytes (FIG. 3, 27). CD45, a protein tyrosine phosphatase is a key regulator in T Cell antigen receptor (TCR) signal transduction. CD45 activates SRC family kinases by removing their C-terminal inhibitory phosphorylation. Activated SRC family kinases phosphorylate the ITAMs in CD3-TCR complex and propagate the signal; thereby activating the T cells. CD45 inhibition blocks TCR mediated signal transduction and results in severe combined immunodeficiency (SCIDs) in humans. UL11 binds CD45 and blocks downstream signal transduction, thus blocking both the activation and the development of T cells.

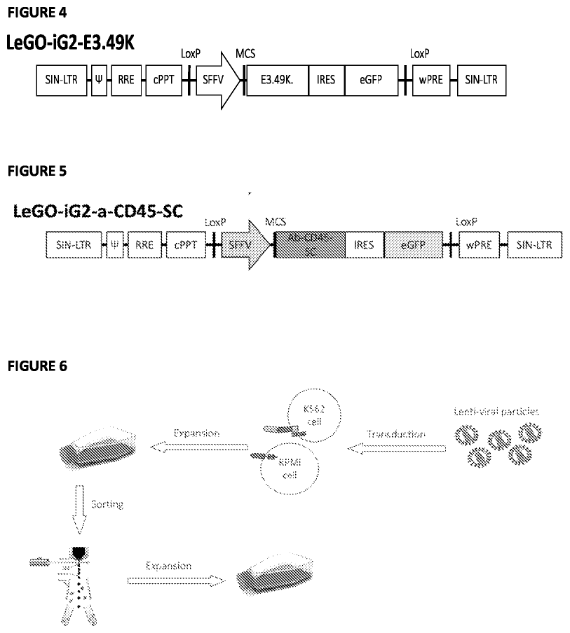

The E3 transcription unit of human Adenoviruses, which comprises proteins with immunomodulatory functions that enable persistent, subclinical infections in immunocompetent individuals (FIG. 4, 21). E3.49K from adenovirus (Ad) species-D is unique as it acts on the un-infected cells, unlike E3s from other adenovirus species which affect only infected cells. E3.49K is a highly glycosylated type-I protein which following cleavage, releases the extracellular 49 kDa molecule. E3.49K has been shown to inhibit both NK cell mediated lysis of target cells lacking MHC-I and TCR complex mediated activation/development of T cells. Our design includes the individual proteins and a chimeric protein with UL11 protein linked to extracellular 49K of the E3.49K. Also, a third single-chain antibody targeting the CD45 has been added for the same purpose. We are also testing single domain antibodies. We have expressed these proteins on the target cells and test them for NK and T cell mediated lysis.

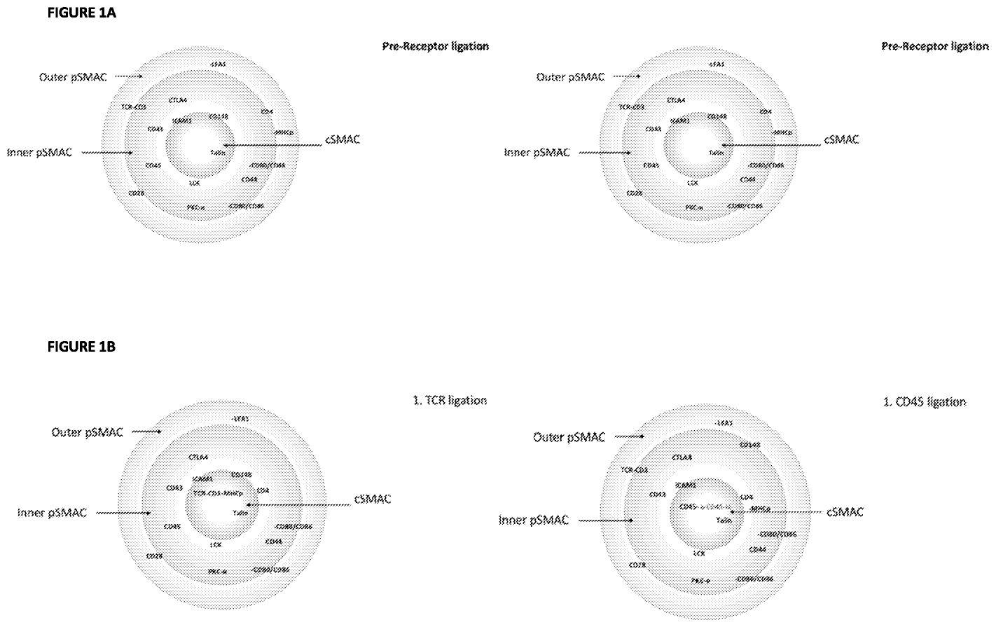

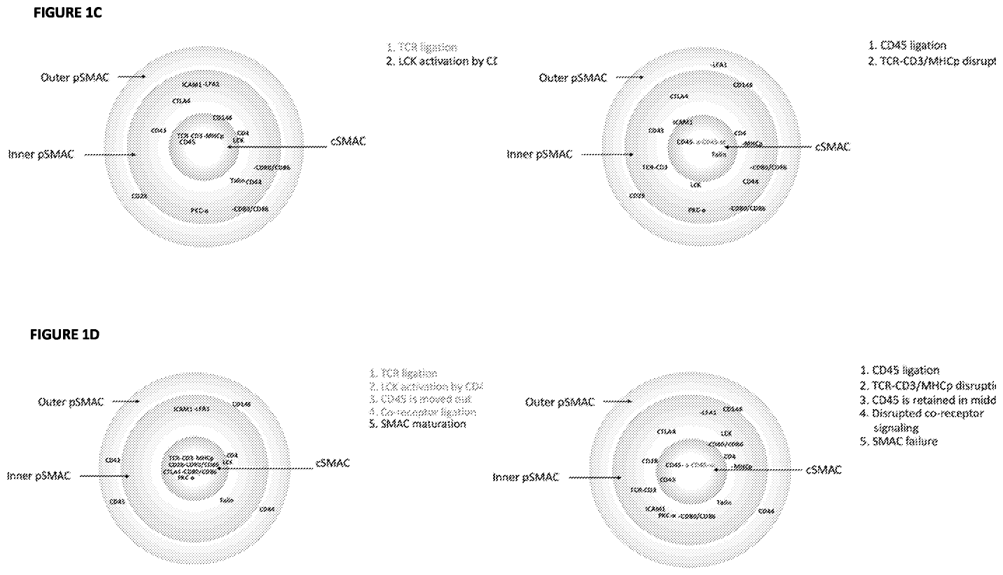

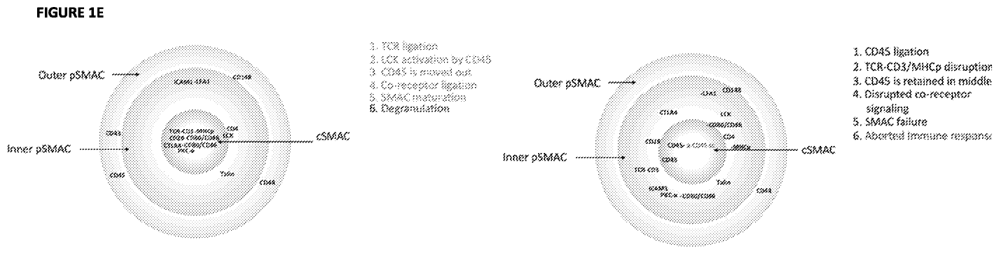

Referring to FIG. 1A (left side), in the very initial TCR receptor activation process, CD45, along with other bulky molecules, is excluded from the immune synapse. The immune synapse consists of rings including different gradients of molecules involved in the immune recognition/reaction. The inner most central Supramolecular Activation Cluster (cSMAC) are TCR/CD3/MHCp, CD28/CD80, SRC family kinase/s, PKC6. Outside cSMAC is pSMAC that comprises an adhesion ring of LFA-1, ICAM-1, and Talins. Glycoproteins including CD45, CD148 and CD43 are moved outside these rings.

CD148 is a receptor tyrosine phosphatase with a heavily glycosylated, large fibronectin extracellular domain and an intracellular catalytic domain. Along with hematopoietic lineages, CD148 is also expressed in vascular and duct endothelial cells where it negatively regulates cell proliferation and transformation. Loss of CD148 has been observed in cancer cell lines and re-expression resulted in the suppression of tumor growth both in vitro and in vivo. CD148 dephosphorylates a number of growth factor receptors including VEGFR, EGFR, HGFR and FGFR and other key downstream signaling molecules like p85, PLC yl, and ERK1/2.

CD43 is a highly glycosylated, mucin type protein with a large extracellular domain and small globular intracellular domain expressed on the hematopoietic cells including stem cells, T lymphocytes, monocytes, granulocytes, NK cell and platelets. CD43 extracellular domain promotes adhesion through interaction with E-selectin, galectin-1 and galectin-3, siglec-1, M-ficolin, integrins, cell surface nucleolin, and ICAM-1 (intercellular adhesion molecule type 1). While the conserved intracellular domain is involved in signal transduction mediating the connection to the cytoskeleton through binding to ezrin, radixin and moesin (ERM) proteins. CD43 has a proline-rich sequence resembling SH3 binding consensus and a nuclear localization signal (NLS), which explains the nuclear localization of CD43.

The B Cell Receptor (BCR) is a membrane bound immunoglobulin with a short intracellular domain of three amino acids. BCRs are made up of two identical heavy chains and two light chains. Extracellular domains have the capacity to specifically recognize and bind antigens. BCR lacks intracellular signaling which is compensated by two associated ITAMs containing chains Igα and Igβ. Following successful binding to the antigen, BCR transduces signaling leading to B cells activation and maturation. Following class switching, BCRs are switched from membrane bound to released form and are then called antibodies.

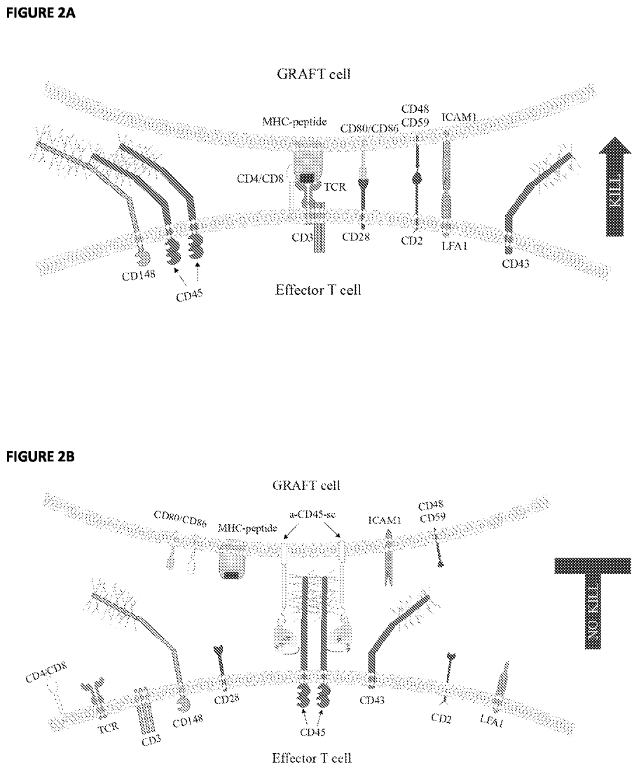

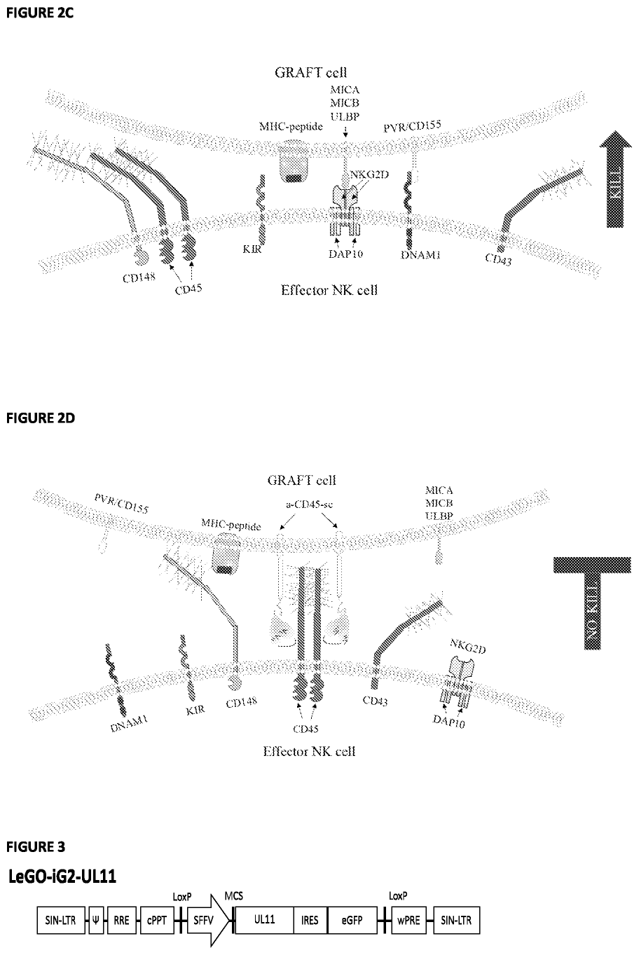

Referring to FIG. 2A, the drawings show the formation of a synapse between a T cell and a target cell in the absence of the present invention leading to target cell lysis. In FIG. 2B, using an engager of the present invention, the physiological synapse is prevented and no lysis occurs.

Referring to FIG. 2C the drawings show the formation of a synapse between a NK cell and a target cell in the absence of the present invention leading to target cell lysis. In FIG. 2D, using an engager of the present invention the physiological synapse is prevented and no lysis occurs.

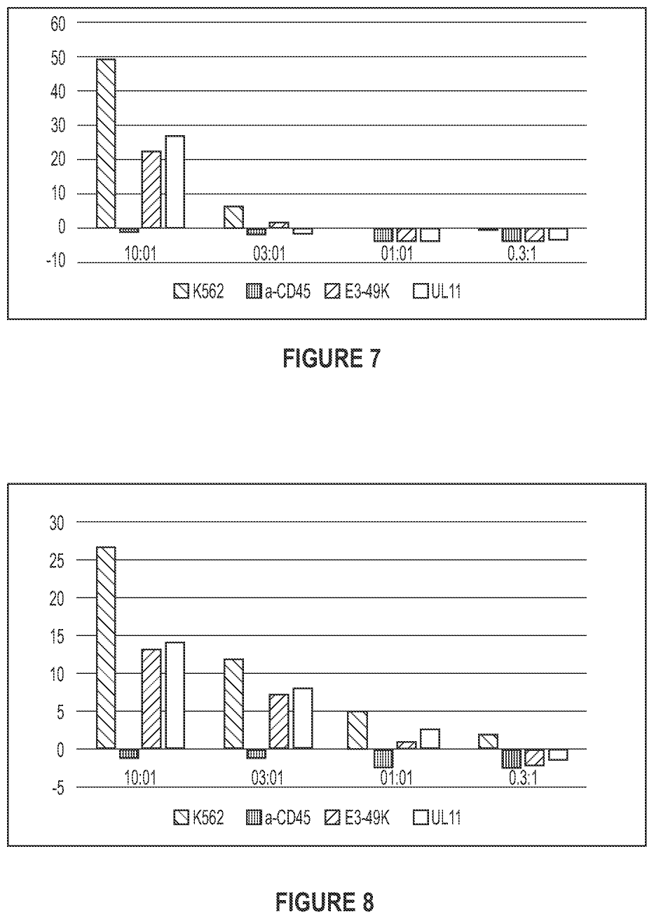

While our present data show that forced retention of CD45 in the immune synapse via an engager prevents cell lysis by cytotoxic cells (FIGS. 7-12), inhibition of cytotoxic cell through CD43 and CD148 immune synapse retention may also prevent lysis. Engagers may be molecules that are used to interfere with CD45, CD43 and CD148 binding. An “engager” is a molecule or group of molecules that can bind to CD45, CD43, or CD148 and thereby inhibit or prevent functional immunological synapse formation. A “functional immunological synapse” is an immune synapse that may form between a CD45, CD148, or CD43 positive cell and a graft cell, including a non-autologous cell. We have used single chain, single domain, and antibodies as effectors. Using the teachings disclosed herein, one of skill in the art will be able to identify other engagers.

Engagers should be present in a sufficient amount to bind CD45, CD43 or CD148. As shown in more detail below, we have shown that we can modulate or shut down the NK or T cell response to a foreign cell. In certain embodiments, the compositions and methods disclosed herein are non-agonistic.

In an embodiment, variants of the amino acid sequences disclosed herein are also contemplated. For example, the amino acid sequence may have at least 75%, 80%, 85%, 90%, 91%, 92%, 93%, 94%, 95%, 96%, 97%, 98%, or 99% identity to one or more of the amino acid sequences disclosed. In certain preferred embodiments, an exemplary amino acid sequence may be an amino acid sequence which has at least 90%, 95%, 96%, 97%, 98%, or 99% identity to one or more of the disclosed amino acid sequences. In an embodiment, the variant amino acid sequence retains the function ascribed to it herein (for example, the ability to bind CD45, CD43, or CD148 and/or prevent or inhibit functional immunological synapse formation and/or to confer immune escape and/or prevent cytotoxicity).

In an embodiment, variants of the nucleic acid sequences disclosed herein are also contemplated. In an embodiment, the nucleic acid sequence may have at least 75%, 80%, 85%, 90%, 91%, 92%, 93%, 94%, 95%, 96%, 97%, 98%, or 99% identity to one or more of the nucleic acid sequences disclosed. In certain preferred embodiments, an exemplary amino acid sequence may be a nucleic acid sequence which has at least 90%, 95%, 96%, 97%, 98%, or 99% identity to one or more of the disclosed nucleic acid sequences. In an embodiment, the variant nucleic acid sequence retains the function ascribed to it herein and/or encodes the (variant) amino acid as disclosed herein.

In an embodiment, an engager may comprise an amino acid sequence in which 1 to 50 amino acids are deleted, substituted, inserted, and/or added in the amino acid sequence of, for example, SEQ ID NO: 1 (UL11), 3 (E3.49K), 5 (a-CD45-sc), 64 (a-CD148-sc), 66 (E3.49K.R1), 68 (E3.49K.R3), 71 (a-CD45(M)-VHH-1), 73 (a-CD45(M)-VHH-2), 220 (m-VHH1-E3-TM), 223 (m-VHH2-E3-TM), or 224 (a-CD43-sc), and have an activity of binding to CD45 and/or inhibiting or preventing functional immunological synapse formation. In preferred embodiments, engagers as disclosed herein include protein sequences consisting of an amino acid sequence in which, for example, 1 to 49, 1 to 48, 1 to 47, 1 to 46, 1 to 45, 1 to 44, 1 to 43, 1 to 42, 1 to 41, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, or 1 amino acid residue is deleted, substituted, inserted, and/or added in the amino acid sequence of SEQ ID NOs: 1, 3, 5, 64, 66, 68, 71, 73, 220, 223, or 224 and having an activity of inhibiting or preventing functional immunological synapse formation.

We will conduct assays for antibodies' competition and for deletion mutants to determine engagers' binding sites. Immunoprecipitation will be used to identify interacting motifs. The top candidates are collected for further experiments.

All three CD45 engagers (E3.49K, UL11 and a-CD45-sc) bind to all isoforms of CD45 suggesting an interaction with the membrane proximal region including fibronectin-III and cysteine-rich domains. Immunoprecipitation studies reveal a physical interaction between CD45 and E3.49K, UL11 or anti-CD45-sc while Ab competition experiments and deletion mutations further supporting the idea that E3.49K, UL11 and a-CD45-sc mainly interact with membrane proximal region of CD45 common to all isoforms.

The present invention also contemplates the use of aptamers directed to CD45, CD43 and CD148. Aptamers are short strands of nucleic acid or proteins or other nature that can specifically bind the target molecules with high affinity, similar to antibodies. These aptamers have the capacity to target small ions, molecules, cells, tissues, or organs. This application covers the aptamers, whether made up of nucleic acid, proteins or other molecules that can specifically bind to the target molecules CD45 and/or CD148 and/or CD43. These aptamers may be naturally existing, or de novo synthesized Colas P, Cohen B, Jessen T, Grishina I, McCoy J, Brent R. Genetic selection of peptide aptamers that recognize and inhibit cyclin-dependent kinase 2. Nature. 1996; 380(6574):548-50; and Zhang Y, Lai B S, Juhas M. Recent Advances in Aptamer Discovery and Applications. Molecules. 2019; 24(5).

The strategy described here could be utilised in the absence of HLA-I and HLA-II knock out or knock down strategies. However, it can also be envisioned that combination of HLA class-I (for example B2M) and/or HLA class II (for example CIITA) together with a CD45/CD148/CD43 engager could lead to a synergistic abrogation of host cellular cytotoxicity.

Suitable stem cells include without limit embryonic stem cells, ES-like stem cells, fetal stem cells, adult stem cells pluripotent stem cells, induced pluripotent stem cells, multipotent stem cells, oligopotent stem cells, unipotent stem cells and others.

### Example 1. Modulation with UL11 and E3.49K

We first set out to show that we could modulate CD45 inclusion in the immune synapse using UL11 and E3.49K

Materials and Methods

Vectors were created incorporating the HCMV-M (Merlin strain; HHV5) protein UL11 sequence which was downloaded from the uniprot. The UL11 sequence is shown below as SEQ ID NO: 1.

UL11 https://www.uniprot.org/uniprot/Q6SWB9
>sp|Q6SWB9 | UL11P HCMVM Protein UL11
OS = Human cytomegalovirus (strain
Merlin) OX = 295027 GN = UL11 PE = 1 SV = 1
(SEQ ID NO: 1)
MLFRYITFHREKVLYLTAACIFGVYISLHDACIPVV
GKIGTNVTLNAVDVLPPRDQVRWSYGPGGQGYMLCI
FTGTSTTTFNNTRFNFSCLSNYSLLLINVTTQYSTT
YRTMTSLDHWLHQRHNHGSRWTLDTCYNLTVNENGT
FPTTTTKKPTTTTRTTTTTTQRTTTTRTTTTAKKTT
ISTTHHKHPSPKKSTTPNSHVEHHVGFEATAAETPL
QPSPQHQHLATHALWVLAVVIVIIIIIIFYFRIPQK
LWLLWQHDKHGIVLIPQTDL

Codon optimization for human cells expression was carried out using CLC Workbench 8. Genes were synthesized by from GeneArt Thermofischer Scientific. Genes were cloned in LeGO-iG2-IRES-GFP plasmid and lentiviral particles were generated. K562 and RPMI82261 cells were transduced with the viral particles and grown in RPMI 1640 medium supplemented with 10% FBS. Transduced cells were expanded and sorted for GFP expression. Sorted cells were expanded and killing assay and degranulation assays were performed. This was performed for UL11 and E3.49K generating plasmids LeGO-iG2-UL11 (FIG. 3), LeGO-iG2-E3.49k (FIG. 4) and LeGO-iG2-a-CD45-sc (FIG. 5). The sequences for the genes inserted into these plasmids are shown below.

UL11-Codon Optimized for Human Cells Expression.

SEQ ID NO: 2 below is the optimized UL11 codon for human cells.

(SEQ ID NO: 2)
ATGTTGTTCAGGTACATCACTTTCCATAGAGAGAAG
GTGCTATACCTGACCGCCGCCTGCATATTCGGGGTG
TATATCTCCCTGCACGACGCGTGTATCCCCGTGGTA
GGCAAAATTGGTACGAACGTTACCCTGAATGCGGTG
GACGTGCTCCCCCCTCGTGACCAAGTGCGGTGGAGC
TATGGGCCGGGCGGGCAGGGATATATGCTCTGCATC
TTTACTGGCACATCAACCACTACTTTCAATAATACC
CGCTTCAATTTCAGCTGCCTGAGCAATTATTCTCTC
CTGTTGATTAATGTGACCACCCAATACTCAACAACT
TATAGAACAATGACCTCTCTGGACCACTGGCTGCAT
CAGAGGCATAACCACGGGAGTCGCTGGACACTGGAC
ACTTGTTACAATCTAACCGTTAACGAAAATGGCACT
TTCCCTACAACCACCACAAAGAAACCCACTACTACA
ACACGAACTACCACAACTACTACGCAGCGAACTACC
ACTACCCGGACCACCACCACAGCTAAGAAGACAACA
ATAAGCACTACTCACCACAAGCACCCTAGCCCAAAG
AAAAGCACTACTCCTAACTCACATGTTGAGCATCAT
GTGGGTTTTGAAGCTACGGCCGCAGAGACACCCCTG
CAACCCTCTCCGCAGCATCAGCACCTCGCTACCCAC
GCCCTTTGGGTTCTTGCAGTTGTGATCGTCATCATT
ATCATAATCATTTTTTATTTTAGGATTCCTCAGAAG
CTGTGGTTGCTTTGGCAGCACGACAAGCATGGCATT
GTGCTTATTCCTCAAACGGACCTGGTAA

Human adenovirus D serotype 17 protein E3.49K was downloaded from uniprot. https://www.uniprot.org/uniprot/Q77N38 The E3.49K sequence is shown below as SEQ ID NO: 3.

E3.49K
>tr|Q77N38|Q77N38 9ADEN 48.9 kDa
OS = Human adenovirus D37
OX = 52275 GN = E3 PE = 4 SV = 1
(SEQ ID NO: 3)
MNTVIRIVLLSLLVAFSQAGFHTINATWWANITLVG
PPDTPVTWYDTQGLWFCNGSRVKNPQIRHTCNDQNL
TLIHVNKTYERTYMGYNRQGTKKEDYKVVVIPPPPA
TVKPQPEPEYVFVYMGENKTLEGPPGTPVTWFNQDG
KKFCEGEKVLHPEFNHTCDKQNLILLFVNFTHDGAY
LGYNHQGTQRTHYEVTVLDLFPDSGQMKIENHSEET
EQKNDEHHNWQKQGGQKQGGQKTNQTKVNDRRKTAQ
KRPSKLKPATIEAMLVTVTAGSNLTLVGPKAEGKVT
WFDGDLKRPCEPNYRLRHECNNQNLTLINVTKDYEG
TYYGTNDKDEGKRYRVKVNTTNSQSVKIQPYTROTT
PDQEHKFELQFETNGNYDSKIPSTTVAIVVGVIAGF
ITLIIVFICYICCRKRPRAYNHMVDPLLSFSY

E3.49K Codon Optimized for Human Cells Expression

SEQ. ID NO:4

(SEQ ID NO: 4)
ATGAACACGGTGATCCGCATAGTCCTTCTGTCTCTGCTGGTGGCTTTCTCC
CAGGCCGGCTTCCACACAATTAATGCCACCTGGTGGGCTAACATTACTCTC
GTAGGCCCCCCGGATACCCCCGTGACTTGGTACGACACTCAGGGTCTGTGG
TTCTGTAACGGGAGTCGAGTGAAAAATCCTCAAATTCGCCATACCTGTAAC
GACCAAAATCTGACCTTGATCCACGTGAACAAGACATACGAGCGTACATAT
ATGGGCTACAATAGGCAGGGTACAAAGAAAGAGGACTATAAAGTGGTAGTG
ATTCCGCCTCCCCCCGCAACAGTCAAGCCCCAACCAGAGCCTGAGTATGTC
TTCGTGTATATGGGCGAGAACAAGACCCTGGAAGGACCTCCAGGAACACCC
GTTACCTGGTTTAACCAGGATGGAAAGAAGTTTTGCGAAGGGGAGAAAGTG
CTTCACCCCGAGTTCAATCATACCTGCGACAAGCAGAACCTGATCCTGCTT
TTTGTGAATTTCACCCATGACGGTGCGTACCTCGGTTATAACCATCAAGGC
ACCCAGCGGACCCATTATGAGGTTACTGTCCTCGATCTCTTCCCCGACAGT
GGTCAGATGAAAATCGAAAACCATAGTGAGGAAACTGAGCAGAAAAATGAC
GAGCATCACAACTGGCAGAAACAAGGCGGACAAAAGCAGGGCGGCCAGAAG
ACAAATCAGACAAAAGTCAATGATCGACGCAAAACCGCCCAGAAACGTCCT
AGCAAACTAAAGCCAGCAACTATTGAGGCAATGCTGGTGACAGTAACTGCT
GGAAGTAACCTGACCCTCGTGGGGCCCAAGGCGGAGGGGAAAGTAACCTGG
TTCGACGGCGATCTAAAACGCCCCTGTGAACCAAACTACAGACTTAGACAC
GAATGCAACAACCAGAACCTGACTCTGATTAACGTGACCAAGGACTACGAA
GGAACATACTACGGGACGAATGATAAGGATGAGGGAAAACGGTACCGGGTT
AAGGTTAACACCACAAACTCCCAGAGTGTCAAAATTCAGCCTTACACCAGG
CAGACTACTCCTGACCAGGAACACAAATTCGAATTACAGTTTGAGACTAAC
GGTAACTATGACTCCAAGATTCCATCTACAACGGTCGCGATCGTAGTGGGC
GTGATTGCAGGCTTCATCACATTGATCATCGTGTTCATCTGCTATATCTGC
TGTAGGAAGCGCCCTCGGGCGTACAACCACATGGTGGACCCTCTGTTGAGT
TTCTCATATTAA

### Example 2: Generation of a Single Chain Recognizing CD45 (a-CD45-Sc)

Using methods shown in Example 1, single chains recognizing CD45 were designed as set out below resulting in plasmid LeGO-iG2-a-CD45-sc shown in FIGS. 5 and 24. Lin Y, Pagel J M, Axworthy D, Pantelias A, Hedin N, Press OW. A genetically engineered anti-CD45 single-chain antibody-streptavidin fusion protein for pretargeted radioimmunotherapy of hematologic malignancies. Cancer Res. 2006; 66(7):3884-92. In the preferred embodiment, the engager is actually present on the target cell surface as shown in FIGS. 7-12.

a-CD45-sc (SEQ ID NO: 5) is the protein for the anti-CD45 antibody along with stalk and transmembrane region joined through linker regions. SEQ ID NO: 6 is DNA sequence of the same molecule. In the sequence below, the underlined lowercase region is the IL2 signal peptide, the lowercase is the heavy chain, underlined capitalized regions are linkers, the capitalized regions without underlining are light chains, the bold capitalized regions are the stalk and the bold underlined regions are the CD34 transmembrane region.

(SEQ ID NO: 5)
myrmqllscialslalvtnsqvqlvesggglvqpggslklscaasgfdfsr
ywmswvrqapgkglewigeinptsstinftpslkdkvfisrdnakntlylq
mskvrsedtalyycargnyyrygdamdywgqgtsvtvskiSGGGGSGGGGS
GGGGSGGGGSGGGGSSDIVLTQSPASLAVSLGQRATISCRASKSVSTSGYS
YLHWYQQKPGQPPKLLIYLASNLESGVPARFSGSGSGTDFTLNIHPVEEED
AATYYCQHSRELPFTFGSGTKLEIKSSGSGSPTTTPAPRPPTPAPTIASQP
LSLRPEACRPAAGGAVHTRGLDFAPRKIEVMYPPPYLDNEKSNGTIIHVKG
KHLCPSPLFPGPSKPTLIALVTSGALLAVLGITGYFL

First a cDNA was generated using an IL-2 signal peptide, VH, Linker, VL, and linker together with a single chain (SC) stalk and a CD34 transmembrane region. SEQ ID NO: 6 below codes for plasmid LeGO-iG2-a-CD45-sc.

In the sequence below, the underlined lowercase region is the IL2 signal peptide, the lowercase is the heavy chain, underlined capitalized regions are linkers, the capitalized regions without underlining are light chains, the bold capitalized regions are the stalk and the bold underlined regions are the CD34 transmembrane region.

(SEQ ID NO: 6)
atgtacaggatgcaactcctgtcttgcattgcactaagtcttgcacttgtc
acaaacagtcaggttcagctggtggaatcaggaggtggcctggtgcagcct
ggaggatccctgaaactctcctgtgcagcctcaggattcgatttcagtaga
tactggatgagttgggtccggcaggctccagggaaagggctagaatggatt
ggagagattaatccaactagcagtacgataaactttacgccatctctaaag
gataaagtcttcatctccagagacaacgccaaaaatacgctgtacctgcaa
atgagcaaagtgagatccgaggacacagccctttattactgtgcaagaggg
aactactataggtacggagatgctatggactactggggtcaaggaacctca
gtcaccgtgagcaagatcTCTGGTGGCGGTGGCTCGGGCGGTGGTGGGTCG
GGTGGCGGCGGCTCGGGTGGTGGTGGGTCGGGCGGCGGCGGCTCGAGCGAC
ATCGTGCTGACCCAGTCTCCTGCTTCCTTAGCTGTATCTCTGGGACAGAGG
GCCACCATCTCATGCAGGGCCAGCAAAAGTGTCAGTACATCTGGCTATAGT
TATCTGCACTGGTACCAACAGAAACCAGGACAGCCACCCAAACTCCTCATC
TATCTTGCATCCAACCTAGAATCTGGGGTCCCTGCCAGGTTCAGTGGCAGT
GGGTCTGGGACAGACTTCACCCTCAACATCCATCCTGTGGAGGAGGAGGAT
GCTGCAACCTATTACTGTCAGCACAGTAGGGAGCTTCCATTCACGTTCGGC
TCGGGGACAAAGTTGGAAATAAAGAGCTCTGGCTCTGGTTCGCCCACCACG
ACGCCAGCGCCGCGACCACCAACACCGGCGCCCACCATCGCGTCGCAGCCC
CTGTCCCTGCGCCCAGAGGCGTGCCGGCCAGCGGCGGGGGGCGCAGTGCAC
ACGAGGGGGCTGGACTTCGCCCCTAGGAAAATTGAAGTTATGTATCCTCCT
CCTTACCTAGACAATGAGAAGAGCAATGGAACCATTATCCATGTGAAAGGG
AAACACCTTTGTCCAAGTCCCCTATTTCCCGGACCTTCTAAGCCCACCCTG
ATTGCACTGGTCACCTCGGGAGCCCTGCTGGCTGTCTTGGGCATCACTGGC
TATTTCCTGTAA

Generation of CD45 Single Chain

A single chain antibody is a fusion protein of the light and heavy chains joined by a linker. The CD45 single chain protein translation is shown below in SEQ ID NO: 7 The heavy chain is shown in lowercase letters and the light chain is shown in capital letters. Linkers are underlined capital letters.

In the sequence below, the lowercase is the heavy chain, underlined capitalized regions are linkers, the capitalized regions without underlining are light chains.

(SEQ ID NO: 7)
qvqlvesggglvqpggslklscaasgfxfsrywmszvrqapgkglewigei
nptsstinxtpslkdkvfisrdnakntlylqmskvrsedtazyycargnyy
rygdamdywgqgtsvtvskiSGGGGSGGGGSGGGGSGGGGSGGGGSSDIVL
TQSPASLAVSLGQPATISCRASKSVSTSGYSYLHWYQQKPGQPPKLLIYLA
SNLESGVPARFSGSGSGTDFTLNIHPVEEEDAATYYCQHSRELPFTXGSGT
KLEIKSSGSGS

The heavy chain is encoded by SEQ ID NO: 8

(SEQ ID NO: 8)
QVQLVESGGGLVQPGGSLKLSCAASGFXFSRYWMSXVRQAPGKGLEWIGEI
NPTSSTINXTPSLKDKVFISRDNAKNTLYLQMSKVRSEDTAXYYCARGNYY
RYGDAMDYWGQGTSVTVSKI

The light chain is encoded by SEQ ID NO: 9.

(SEQ ID NO. 9)
DIVLTQSPASLAVSLGQRATISCRASKSVSTSGYSYLHWYQQKPGQPPKLL
IYLASNLESGVPARFSGSGSGTDFTLNIHPVEEEDAATYYCQHSRELPFTX
GSGTKLEIK

The Stalk

The stalk is a structural domain between the single chain and the cell's outer membrane. This, or portions thereof, may sometimes be referred to as a hinge or a spacer. The stalk serves to position the antibody region at a desired location outside the cell membrane. The stalk is preferably between 8 and 200 amino acids in total length. The stalk needs to project from the cell membrane surface but should not be so long that it folds. This stalk is fused to the single chain antibody and binds it to a transmembrane domain. In this instance we utilized a CD8a/CD28 extracellular domain fusion construct for the stalk.

The CD8a/CD28 extracellular domain fusion construct comprising the stalk region is encoded by the 5′3′ Frame 1 SEQ ID NO: 10.

(SEQ ID NO: 10)
cccaccacgacgccagcgccgcgaccaccaacaccggcgcccaccatcgcg
tcgcagcccctgtccctgcgcccagaggcgtgccggccagcggcggggggc
gcagtgcacacgagggggctggacttcgcccctaggaaaattgaagttatg
tatcctcctccttacctagacaatgagaagagcaatggaaccattatccat
gtgaaagggaaacacctttgtccaagtcccctatttcccggaccttctaag
ccc

SEQ ID NO: 10 encodes the protein in SEQ ID NO: 11 below.

(SEQ ID NO: 11)
PTTTPAPRPPTPAPTIASQPLSLRPEACRPAAGGAVHTRGLDFAPRKIEVM
YPPPYLDNEKSNGTIIHVKGKHLCPSPLFPGPSKP

The stalk comprised the following underlined Homo sapiens CD8a sequences underlined below in SEQ ID NO: 12 as part the CD8a region thereof.

>sp|P01732|CD8A_HUMAN T-cell surface glycoprotein
CD8 alpha chain OS = Homo sapiens OX = 9606 GN =
CD8A PE = 1 SV = 1
(SEQ ID NO: 12)
MALPVTALLLPLALLLHAARPSQFRVSPLDRTWNLGETVELKCQVLLSNPT
SGCSWLFQPRGAAASPTFLLYLSQNKPKAAEGLDTQRFSGKRLGDTFVLTL
SDFRRENEGYYFCSALSNSIMYFSHFVPVFLPAKPTTTPAPRPPTPAPTIA
SQPLSLRPEACRPAAGGAVHTRGLDFACDIYIWAPLAGTCGVLLLSLVITL
YCNHRNRRRVCKCPRPVVKSGDKPSLSARYV

The stalk region underlined above is shown below as SEQ ID NO: 13:

(SEQ ID NO: 13)
PTTTPAPRPPTPAPTIASQPLSLRPEACRPAAGGAVHTRGLDFA

CD8a

The CD8 a nucleotide sequence is shown below as SEQ ID NO: 14. The underlined region encodes the stalk.

Nucleotide Sequence (708 nt):
(SEQ ID NO: 14)
ATGGCCTTACCAGTGACCGCCTTGCTCCTGCCGCTGGCCTTGCTGCTCCAC
GCCGCCAGGCCGAGCCAGTTCCGGGTGTCGCCGCTGGATCGGACCTGGAAC
CTGGGCGAGACAGTGGAGCTGAAGTGCCAGGTGCTGCTGTCCAACCCGACG
TCGGGCTGCTCGTGGCTCTTCCAGCCGCGCGGCGCCGCCGCCAGTCCCACC
TTCCTCCTATACCTCTCCCAAAACAAGCCCAAGGCGGCCGAGGGGCTGGAC
ACCCAGCGGTTCTCGGGCAAGAGGTTGGGGGACACCTTCGTCCTCACCCTG
AGCGACTTCCGCCGAGAGAACGAGGGCTACTATTTCTGCTCGGCCCTGAGC
AACTCCATCATGTACTTCAGCCACTTCGTGCCGGTCTTCCTGCCAGCGAAG
CCCACCACGACGCCAGCGCCGCGACCACCAACACCGGCGCCCACCATCGCG
TCGCAGCCCCTGTCCCTGCGCCCAGAGGCGTGCCGGCCAGCGGCGGGGGGC
GCAGTGCACACGAGGGGGCTGGACTTCGCCTGTGATATCTACATCTGGGCG
CCCTTGGCCGGGACTTGTGGGGTCCTTCTCCTGTCACTGGTTATCACCCTT
TACTGCAACCACAGGAACCGAAGACGTGTTTGCAAATGTCCCCGGCCTGTG
GTCAAATCGGGAGACAAGCCCAGCCTTTCGGCGAGATACGTCTAA

The CD8a stalk is encoded by the polynucleotide SEQ ID NO: 15 shown below:

(SEQ ID NO: 15)
CCCACCACGACGCCAGCGCCGCGACCACCAACACCGGCGCCCACCATCGCG
TCGCAGCCCCTGTCCCTGCGCCCAGAGGCGTGCCGGCCAGCGGCGGGGGGC
GCAGTGCACACGAGGGGGCTGGACTTCGCC

CD8a Translation (235 Aa):

(SEQ ID NO: 16)
MALPVTALLLPLALLLHAARPSQFRVSPLDRTWNLGETVELKCQVLLSNPT
SGCSWLFQPRGAAASPTFLLYLSQNKPKAAEGLDTQRFSGKRLGDTFVLTL
SDFRRENEGYYFCSALSNSIMYFSHFVPVFLPAKPTTTPAPRPPTPAPTIA
SQPLSLRPEACRPAAGGAVHTRGLDFACDIYIWAPLAGTCGVLLLSLVITL
YCNHRNRRRVCKCPRPVVKSGDKPSLSARYV

We used the underlined part of SEQ ID NO: 16 as the stalk and transmembrane as shown in SEQ ID NO: 17.

(SEQ ID NO: 17)
PTTTPAPRPPTPAPTIASQPLSLRPEACRPAAGGAVHTRGLDFACDIYIWA
PLAGTCGVLLLSLVITLYC

We used the underlined sequences from CD28 to further complete the stalk/hinge. The CD28 protein is encoded by SEQ ID NO: 18 below:

>sp|P10747|CD28_HUMAN T-cell-specific surface
glycoprotein CD28 OS = Homo sapiens OX = 9606 GN =
CD28 PE = 1 SV = 1
(SEQ ID NO: 18)
MLRLLLALNLFPSIQVTGNKILVKQSPMLVAYDNAVNLSCKYSYNLFSREF
RASLHKGLDSAVEVCVVYGNYSQQLQVYSKTGFNCDGKLGNESVTFYLQNL
YVNQTDIYFCKIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRS

The cDNA for CD28 Nucleotide Sequence is set out below as SEQ ID NO: 19:

(SEQ ID NO: 19)
ATGCTCAGGCTGCTCTTGGCTCTCAACTTATTCCCTTCAATTCAAGTAAC
AGGAAACAAGATTTTGGTGAAGCAGTCGCCCATGCTTGTAGCGTACGACA
ATGCGGTCAACCTTAGCTGCAAGTATTCCTACAATCTCTTCTCAAGGGAG
TTCCGGGCATCCCTTCACAAAGGACTGGATAGTGCTGTGGAAGTCTGTGT
TGTATATGGGAATTACTCCCAGCAGCTTCAGGTTTACTCAAAAACGGGGT
TCAACTGTGATGGGAAATTGGGCAATGAATCAGTGACATTCTACCTCCAG
AATTTGTATGTTAACCAAACAGATATTTACTTCTGCAAAATTGAAGTTAT
GTATCCTCCTCCTTACCTAGACAATGAGAAGAGCAATGGAACCATTATCC
ATGTGAAAGGGAAACACCTTTGTCCAAGTCCCCTATTTCCCGGACCTTCT
AAGCCCTTTTGGGTGCTGGTGGTGGTTGGTGGAGTCCTGGCTTGCTATAG
CTTGCTAGTAACAGTGGCCTTTATTATTTTCTGGGTGAGGAGTAAGAGGA
GCAGGCTCCTGCACAGTGACTACATGAACATGACTCCCCGCCGCCCCGGG
CCCACCCGCAAGCATTACCAGCCCTATGCCCCACCACGCGACTTCGCAGC
CTATCGCTCCTGA

In the final construct the underlined portion of SEQ ID NO: 19 is set out below as SEQ ID NO: 20 and serves as the art of the stalk.

(SEQ ID NO: 20)
KIEVMYPPPYLDNEKSNGTIIHVKGKHLCPSPLFPGPSKP

Transmembrane Region

The transmembrane region serves to anchor the stalk/protein to the cell. The transmembrane region was taken from CD34 FASTA, the protein sequence of which is set out below as SEQ ID NO: 21.

>sp|P28906|CD34_HUMAN Hematopoietic progenitor
cell antigen CD34 OS = Homo sapiens OX = 9606
GN = CD34 PE = 1 SV = 2 385 AA
(SEQ ID NO: 21)
MLVRRGARAGPRMPRGWTALCLLSLLPSGFMSLDNNGTATPELPTQGTFS
NVSTNVSYQETTTPSTLGSTSLHPVSQHGNEATTNITETTVKFTSTSVIT
SVYGNTNSSVQSQTSVISTVFTTPANVSTPETTLKPSLSPGNVSDLSTTS
TSLATSPTKPYTSSSPILSDIKAEIKCSGIREVKLTQGICLEQNKTSSCA
EFKKDRGEGLARVLCGEEQADADAGAQVCSLLLAQSEVRPQCLLLVLANR
TEISSKLQLMKKHQSDLKKLGILDFTEQDVASHQSYSQKTLIALVTSGAL
LAVLGITGYFLMNRRSWSPTGERLGEDPYYTENGGGQGYSSGPGTSPEAQ
GKASVNRGAQENGTGQATSRNGHSARQHVVADTEL

We used the following sequence taken from the underlined portion of SEQ ID NO: 20 as the transmembrane LIALVTSGALLAVLGITGYFL (SEQ ID NO: 22).

The protein of SEQ ID NO: 21 is coded by cDNA SEQ ID NO: 23 below.

(SEQ ID NO: 23)
ATGCTGGTCCGCAGGGGCGCGCGCGCAGGGCCCAGGATGCCGCGGGGCTG
GACCGCGCTTTGCTTGCTGAGTTTGCTGCCTTCTGGGTTCATGAGTCTTG
ACAACAACGGTACTGCTACCCCAGAGTTACCTACCCAGGGAACATTTTCA
AATGTTTCTACAAATGTATCCTACCAAGAAACTACAACACCTAGTACCCT
TGGAAGTACCAGCCTGCACCCTGTGTCTCAACATGGCAATGAGGCCACAA
CAAACATCACAGAAACGACAGTCAAATTCACATCTACCTCTGTGATAACC
TCAGTTTATGGAAACACAAACTCTTCTGTCCAGTCACAGACCTCTGTAAT
CAGCACAGTGTTCACCACCCCAGCCAACGTTTCAACTCCAGAGACAACCT
TGAAGCCTAGCCTGTCACCTGGAAATGTTTCAGACCTTTCAACCACTAGC
ACTAGCCTTGCAACATCTCCCACTAAACCCTATACATCATCTTCTCCTAT
CCTAAGTGACATCAAGGCAGAAATCAAATGTTCAGGCATCAGAGAAGTGA
AATTGACTCAGGGCATCTGCCTGGAGCAAAATAAGACCTCCAGCTGTGCG
GAGTTTAAGAAGGACAGGGGAGAGGGCCTGGCCCGAGTGCTGTGTGGGGA
GGAGCAGGCTGATGCTGATGCTGGGGCCCAGGTATGCTCCCTGCTCCTTG
CCCAGTCTGAGGTGAGGCCTCAGTGTCTACTGCTGGTCTTGGCCAACAGA
ACAGAAATTTCCAGCAAACTCCAACTTATGAAAAAGCACCAATCTGACCT
GAAAAAGCTGGGGATCCTAGATTTCACTGAGCAAGATGTTGCAAGCCACC
AGAGCTATTCCCAAAAGACCCTGATTGCACTGGTCACCTCGGGAGCCCTG
CTGGCTGTCTTGGGCATCACTGGCTATTTCCTGATGAATCGCCGCAGCTG
GAGCCCCACAGGAGAAAGGCTGGGCGAAGACCCTTATTACACGGAAAACG
GTGGAGGCCAGGGCTATAGCTCAGGACCTGGGACCTCCCCTGAGGCTCAG
GGAAAGGCCAGTGTGAACCGAGGGGCTCAGGAAAACGGGACCGGCCAGGC
CACCTCCAGAAACGGCCATTCAGCAAGACAACACGTGGTGGCTGATACCG
AATTGTGA

The cDNA for the transmembrane protein of SEQ ID NO: 22 was taken from the underlined region of SEQ ID NO: 23 and is set out below as SEQ ID NO: 24.

SEQ ID NO: 24)
ACCCTGATTGCACTGGTCACCTCGGGAGCCCTGCTGGCTGTCTTGGGCAT
CACTGGCTATTTCCTG

Like the transmembrane domain of CD34, transmembrane regions from other proteins, (membrane bound), can also be utilized. There is probably no limitation on which transmembrane domains are used. Commonly used examples of other proteins with transmembrane domain include but are not limited to CD45, CD28 and CD8a are given below.

CD45

The transmembrane region of CD45 is underlined in protein SEQ ID NO: 25 below.

>sp|P08575|PTPRC_HUMAN Receptor-type
tyrosine-protein phosphatase C OS = Homo sapiens
OX = 9606 GN = PTPRC PE = 1 SV = 3
(SEQ ID NO: 25)
MTMYLWLKLLAFGFAFLDTEVFVTGQSPTPSPTGLTTAKMPSVPLSSDPL
PTHTTAFSPASTFERENDFSETTTSLSPDNTSTQVSPDSLDNASAFNTTG
VSSVQTPHLPTHADSQTPSAGTDTQTFSGSAANAKLNPTPGSNAISDVPG
ERSTASTFPTDPVSPLTTTLSLAHHSSAALPARTSNTTITANTSDAYLNA
SETTTLSPSGSAVISTTTIATTPSKPTCDEKYANITVDYLYNKETKLFTA
KLNVNENVECGNNTCTNNEVHNLTECKNASVSISHNSCTAPDKTLILDVP
PGVEKFQLHDCTQVEKADTTICLKWKNIETFTCDTQNITYRFQCGNMIFD
NKEIKLENLEPEHEYKCDSEILYNNHKFTNASKIIKTDFGSPGEPQIIFC
RSEAAHQGVITWNPPQRSFHKSAPPSQVWNMTVSMTSDNSMHVKCRPPRD
RNGPHERYHLEVEAGNTLVRNESHKNCDFRVKDLQYSTDYTFKAYFHNGD
YPGEPFILHHSTSYNSKALIAFLAFLIIVTSIALLVVLYKIYDLHKKRSC
NLDEQQELVERDDEKQLMNVEPIHADILLETYKRKIADEGRLFLAEFQSI
PRVFSKFPIKEARKPFNQNKNRYVDILPYDYNRVELSEINGDAGSNYINA
SYIDGFKEPRKYIAAQGPRDETVDDFWRMIWEQKATVIVMVTRCEEGNRN
KCAEYWPSMEEGTRAFGDVVVKINQHKRCPDYIIQKLNIVNKKEKATGRE
VTHIQFTSWPDHGVPEDPHLLLKLRRRVNAFSNFFSGPIVVHCSAGVGRT
GTYIGIDAMLEGLEAENKVDVYGYVVKLRRQRCLMVQVEAQYILIHQALV
EYNQFGETEVNLSELHPYLHNMKKRDPPSEPSPLEAEFQRLPSYRSWRTQ
HIGNQEENKSKNRNSNVIPYDYNRVPLKHELEMSKESEHDSDESSDDDSD
SEEPSKYINASFIMSYWKPEVMIAAQGPLKETIGDFWQMIFQRKVKVIVM
LTELKHGDQEICAQYWGEGKQTYGDIEVDLKDTDKSSTYTLRVFELRHSK
RKDSRTVYQYQYTNWSVEQLPAEPKELISMIQVVKQKLPQKNSSEGNKHH
KSTPLLIHCRDGSQQTGIFCALLNLLESAETEEVVDIFQVVKALRKARPG
MVSTFEQYQFLYDVIASTYPAQNGQVKKNNHQEDKIEFDNEVDKVKQDAN
CVNPLGAPEKLPEAKEQAEGSEPTSGTEGPEHSVNGPASPALNQGS
CD45 transmembrane Domain
>sp|P08575|578-598
(SEQ ID NO: 26)
ALIAFLAFLIIVTSIALLVVL

CD45 DNA Sequence

(SEQ ID NO: 27)
ATGACCATGTATTTGTGGCTTAAACTCTTGGCATTTGGCTTTGCCTTTCTGGACACAGAAGT
ATTTGTGACAGGGCAAAGCCCAACACCTTCCCCCACTGGATTGACTACAGCAAAGATGCCCA
GTGTTCCACTTTCAAGTGACCCCTTACCTACTCACACCACTGCATTCTCACCCGCAAGCACC
TTTGAAAGAGAAAATGACTTCTCAGAGACCACAACTTCTCTTAGTCCAGACAATACTTCCAC
CCAAGTATCCCCGGACTCTTTGGATAATGCTAGTGCTTTTAATACCACAGGTGTTTCATCAG
TACAGACGCCTCACCTTCCCACGCACGCAGACTCGCAGACGCCCTCTGCTGGAACTGACACG
CAGACATTCAGCGGCTCCGCCGCCAATGCAAAACTCAACCCTACCCCAGGCAGCAATGCTAT
CTCAGATGTCCCAGGAGAGAGGAGTACAGCCAGCACCTTTCCTACAGACCCAGTTTCCCCAT
TGACAACCACCCTCAGCCTTGCACACCACAGCTCTGCTGCCTTACCTGCACGCACCTCCAAC
ACCACCATCACAGCGAACACCTCAGATGCCTACCTTAATGCCTCTGAAACAACCACTCTGAG
CCCTTCTGGAAGCGCTGTCATTTCAACCACAACAATAGCTACTACTCCATCTAAGCCAACAT
GTGATGAAAAATATGCAAACATCACTGTGGATTACTTATATAACAAGGAAACTAAATTATTT
ACAGCAAAGCTAAATGTTAATGAGAATGTGGAATGTGGAAACAATACTTGCACAAACAATGA
GGTGCATAACCTTACAGAATGTAAAAATGCGTCTGTTTCCATATCTCATAATTCATGTACTG
CTCCTGATAAGACATTAATATTAGATGTGCCACCAGGGGTTGAAAAGTTTCAGTTACATGAT
TGTACACAAGTTGAAAAAGCAGATACTACTATTTGTTTAAAATGGAAAAATATTGAAACCTT
TACTTGTGATACACAGAATATTACCTACAGATTTCAGTGTGGTAATATGATATTTGATAATA
AAGAAATTAAATTAGAAAACCTTGAACCCGAACATGAGTATAAGTGTGACTCAGAAATACTC
TATAATAACCACAAGTTTACTAACGCAAGTAAAATTATTAAAACAGATTTTGGGAGTCCAGG
AGAGCCTCAGATTATTTTTTGTAGAAGTGAAGCTGCACATCAAGGAGTAATTACCTGGAATC
CCCCTCAAAGATCATTTCATAATTTTACCCTCTGTTATATAAAAGAGACAGAAAAAGATTGC
CTCAATCTGGATAAAAACCTGATCAAATATGATTTGCAAAATTTAAAACCTTATACGAAATA
TGTTTTATCATTACATGCCTACATCATTGCAAAAGTGCAACGTAATGGAAGTGCTGCAATGT
GTCATTTCACAACTAAAAGTGCTCCTCCAAGCCAGGTCTGGAACATGACTGTCTCCATGACA
TCAGATAATAGTATGCATGTCAAGTGTAGGCCTCCCAGGGACCGTAATGGCCCCCATGAACG
TTACCATTTGGAAGTTGAAGCTGGAAATACTCTGGTTAGAAATGAGTCGCATAAGAATTGCG
ATTTCCGTGTAAAAGATCTTCAATATTCAACAGACTACACTTTTAAGGCCTATTTTCACAAT
GGAGACTATCCTGGAGAACCCTTTATTTTACATCATTCAACATCTTATAATTCTAAGGCACT
GATAGCATTTCTGGCATTTCTGATTATTGTGACATCAATAGCCCTGCTTGTTGTTCTCTACA
AAATCTATGATCTACATAAGAAAAGATCCTGCAATTTAGATGAACAGCAGGAGCTTGTTGAA
AGGGATGATGAAAAACAACTGATGAATGTGGAGCCAATCCATGCAGATATTTTGTTGGAAAC
TTATAAGAGGAAGATTGCTGATGAAGGAAGACTTTTTCTGGCTGAATTTCAGAGCATCCCGC
GGGTGTTCAGCAAGTTTCCTATAAAGGAAGCTCGAAAGCCCTTTAACCAGAATAAAAACCGT
TATGTTGACATTCTTCCTTATGATTATAACCGTGTTGAACTCTCTGAGATAAACGGAGATGC
AGGGTCAAACTACATAAATGCCAGCTATATTGATGGTTTCAAAGAACCCAGGAAATACATTG
CTGCACAAGGTCCCAGGGATGAAACTGTTGATGATTTCTGGAGGATGATTTGGGAACAGAAA
GCCACAGTTATTGTCATGGTCACTCGATGTGAAGAAGGAAACAGGAACAAGTGTGCAGAATA
CTGGCCGTCAATGGAAGAGGGCACTCGGGCTTTTGGAGATGTTGTTGTAAAGATCAACCAGC
ACAAAAGATGTCCAGATTACATCATTCAGAAATTGAACATTGTAAATAAAAAAGAAAAAGCA
ACTGGAAGAGAGGTGACTCACATTCAGTTCACCAGCTGGCCAGACCACGGGGTGCCTGAGGA
TCCTCACTTGCTCCTCAAACTGAGAAGGAGAGTGAATGCCTTCAGCAATTTCTTCAGTGGTC
CCATTGTGGTGCACTGCAGTGCTGGTGTTGGGCGCACAGGAACCTATATCGGAATTGATGCC
ATGCTAGAAGGCCTGGAAGCCGAGAACAAAGTGGATGTTTATGGTTATGTTGTCAAGCTAAG
GCGACAGAGATGCCTGATGGTTCAAGTAGAGGCCCAGTACATCTTGATCCATCAGGCTTTGG
TGGAATACAATCAGTTTGGAGAAACAGAAGTGAATTTGTCTGAATTACATCCATATCTACAT
AACATGAAGAAAAGGGATCCACCCAGTGAGCCGTCTCCACTAGAGGCTGAATTCCAGAGACT
TCCTTCATATAGGAGCTGGAGGACACAGCACATTGGAAATCAAGAAGAAAATAAAAGTAAAA
ACAGGAATTCTAATGTCATCCCATATGACTATAACAGAGTGCCACTTAAACATGAGCTGGAA
ATGAGTAAAGAGAGTGAGCATGATTCAGATGAATCCTCTGATGATGACAGTGATTCAGAGGA
ACCAAGCAAATACATCAATGCATCTTTTATAATGAGCTACTGGAAACCTGAAGTGATGATTG
CTGCTCAGGGACCACTGAAGGAGACCATTGGTGACTTTTGGCAGATGATCTTCCAAAGAAAA
GTCAAAGTTATTGTTATGCTGACAGAACTGAAACATGGAGACCAGGAAATCTGTGCTCAGTA
CTGGGGAGAAGGAAAGCAAACATATGGAGATATTGAAGTTGACCTGAAAGACACAGACAAAT
CTTCAACTTATACCCTTCGTGTCTTTGAACTGAGACATTCCAAGAGGAAAGACTCTCGAACT
GTGTACCAGTACCAATATACAAACTGGAGTGTGGAGCAGCTTCCTGCAGAACCCAAGGAATT
AATCTCTATGATTCAGGTCGTCAAACAAAAACTTCCCCAGAAGAATTCCTCTGAAGGGAACA
AGCATCACAAGAGTACACCTCTACTCATTCACTGCAGGGATGGATCTCAGCAAACGGGAATA
TTTTGTGCTTTGTTAAATCTCTTAGAAAGTGCGGAAACAGAAGAGGTAGTGGATATTTTTCA
AGTGGTAAAAGCTCTACGCAAAGCTAGGCCAGGCATGGTTTCCACATTCGAGCAATATCAAT
TCCTATATGACGTCATTGCCAGCACCTACCCTGCTCAGAATGGACAAGTAAAGAAAAACAAC
CATCAAGAAGATAAAATTGAATTTGATAATGAAGTGGACAAAGTAAAGCAGGATGCTAATTG
TGTTAATCCACTTGGTGCCCCAGAAAAGCTCCCTGAAGCAAAGGAACAGGCTGAAGGTTCTG
AACCCACGAGTGGCACTGAGGGGCCAGAACATTCTGTCAATGGTCCTGCAAGTCCAGCTTTA
AATCAAGGTTCATAG

CD28

The transmembrane region of CD28 is underlined in protein SEQ ID NO: 28 below.

>sp|P10747|CD28_HUMAN T-cell-specific
surface glycoprotein CD28 OS = Homo sapiens
OX = 9606 GN = CD28 PE = 1 SV = 1
(SEQ ID NO: 28)
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS
CD28 Transmembrane Domain
>sp|P10747|153-179
(SEQ ID NO: 29)
FWVLVVVGGVLACYSLLVTVAFIIFWV
CD28 DNA sequence
(SEQ ID NO: 30)
ATGCTCAGGCTGCTCTTGGCTCTCAACTTATTCCCTTCAATTCAAGTAAC
AGGAAACAAGATTTTGGTGAAGCAGTCGCCCATGCTTGTAGCGTACGACA
ATGCGGTCAACCTTAGCTGCAAGTATTCCTACAATCTCTTCTCAAGGGAG
TTCCGGGCATCCCTTCACAAAGGACTGGATAGTGCTGTGGAAGTCTGTGT
TGTATATGGGAATTACTCCCAGCAGCTTCAGGTTTACTCAAAAACGGGGT
TCAACTGTGATGGGAAATTGGGCAATGAATCAGTGACATTCTACCTCCAG
AATTTGTATGTTAACCAAACAGATATTTACTTCTGCAAAATTGAAGTTAT
GTATCCTCCTCCTTACCTAGACAATGAGAAGAGCAATGGAACCATTATCC
ATGTGAAAGGGAAACACCTTTGTCCAAGTCCCCTATTTCCCGGACCTTCT
AAGCCCTTTTGGGTGCTGGTGGTGGTTGGTGGAGTCCTGGCTTGCTATAG
CTTGCTAGTAACAGTGGCCTTTATTATTTTCTGGGTGAGGAGTAAGAGGA
GCAGGCTCCTGCACAGTGACTACATGAACATGACTCCCCGCCGCCCCGGG
CCCACCCGCAAGCATTACCAGCCCTATGCCCCACCACGCGACTTCGCAGC
CTATCGCTCCTGA

The underlined region of SEQ ID NO: 30 is the transmembrane domain encoding SEQ ID NO: 29.

CD8a

The protein sequence for CD8a is set out in SEQ ID NO: 31. The transmembrane region of CD8a is underlined.

>sp|P01732|CD8A_HUMAN T-cell surface
glycoprotein CD8 alpha chain OS = Homo sapiens
OX = 9606 GN = CD8A PE = 1 SV = 1
(SEQ ID NO: 31)
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

CD8a Transmembrane Domain

>sp|P01732|183-203
(SEQ ID NO: 32)
IYIWAPLAGTCGVLLLSLVIT
CD8a DNA sequence
(SEQ ID NO: 33)
ATGGCCTTACCAGTGACCGCCTTGCTCCTGCCGCTGGCCTTGCTGCTCCA
CGCCGCCAGGCCGAGCCAGTTCCGGGTGTCGCCGCTGGATCGGACCTGGA
ACCTGGGCGAGACAGTGGAGCTGAAGTGCCAGGTGCTGCTGTCCAACCCG
ACGTCGGGCTGCTCGTGGCTCTTCCAGCCGCGCGGCGCCGCCGCCAGTCC
CACCTTCCTCCTATACCTCTCCCAAAACAAGCCCAAGGCGGCCGAGGGGC
TGGACACCCAGCGGTTCTCGGGCAAGAGGTTGGGGGACACCTTCGTCCTC
ACCCTGAGCGACTTCCGCCGAGAGAACGAGGGCTACTATTTCTGCTCGGC
CCTGAGCAACTCCATCATGTACTTCAGCCACTTCGTGCCGGTCTTCCTGC
CAGCGAAGCCCACCACGACGCCAGCGCCGCGACCACCAACACCGGCGCCC
ACCATCGCGTCGCAGCCCCTGTCCCTGCGCCCAGAGGCGTGCCGGCCAGC
GGCGGGGGGCGCAGTGCACACGAGGGGGCTGGACTTCGCCTGTGATATCT
ACATCTGGGCGCCCTTGGCCGGGACTTGTGGGGTCCTTCTCCTGTCACTG
GTTATCACCCTTTACTGCAACCACAGGAACCGAAGACGTGTTTGCAAATG
TCCCCGGCCTGTGGTCAAATCGGGAGACAAGCCCAGCCTTTCGGCGAGAT
ACGTCTAA

The underlined region of SEQ ID NO: 33 encodes the transmembrane region of the protein.

Signal Peptide

We used the underlined signal peptide encoding sequences of IL-2 human, SEQ ID NO: 34.

Nucleotide Sequence (462 nt):
(SEQ ID NO: 34)
ATGTACAGGATGCAACTCCTGTCTTGCATTGCACTAAGTCTTGCACTTGT
CACAAACAGTGCACCTACTTCAAGTTCTACAAAGAAAACACAGCTACAAC
TGGAGCATTTACTGCTGGATTTACAGATGATTTTGAATGGAATTAATAAT
TACAAGAATCCCAAACTCACCAGGATGCTCACATTTAAGTTTTACATGCC
CAAGAAGGCCACAGAACTGAAACATCTTCAGTGTCTAGAAGAAGAACTCA
AACCTCTGGAGGAAGTGCTAAATTTAGCTCAAAGCAAAAACTTTCACTTA
AGACCCAGGGACTTAATCAGCAATATCAACGTAATAGTTCTGGAACTAAA
GGGATCTGAAACAACATTCATGTGTGAATATGCTGATGAGACAGCAACCA
TTGTAGAATTTCTGAACAGATGGATTACCTTTTGTCAAAGCATCATCTCA
ACACTGACTTGA

The protein sequence for IL-2 is set out in SEQ ID NO: 35. The signal peptide is underlined. https://www.uniprot.org/uniprot/P60568

(SEQ ID NO: 35)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

The IL-2 signal peptide is: MYRMQLLSCIALSLALVTNS (SEQ ID NO: 36)

Similar to IL-2 signal peptide, signal peptides of other proteins (secreted or membrane bound) can also be utilized. Examples of such proteins with signal peptides include, but are not limited to IFNg, and IL2Ra/CD25, given below.

The protein sequence for IFNg is set out in SEQ ID NO: 37. The signal peptide is underlined.

### IFNg

>sp|P01579|IFNG_HUMAN Interferon
gamma OS = Homo sapiens OX = 9606
GN = IFNG PE = 1 SV = 1
(SEQ ID NO: 37)
MKYTSYILAFQLCIVLGSLGCYCQDPYVKEAENLKKYFNAGHSDVADNG
TLFLGILKNWKEESDRKIMQSQIVSFYFKLFKNFKDDQSIQKSVETIKED
MNVKFFNSNKKKRDDFEKLTNYSVTDLNVQRKAIHELIQVMAELSPAAKT
GKRKRSQMLFRGRRASQ

IFN Gamma Signal Peptide

>sp|P01579|1-23
(SEQ ID NO: 38)
MKYTSYILAFQLCIVLGSLGCYC

IFNg DNA sequence. Signal peptide nucleotide sequence is underlined in SEQ ID NO: 39.

(SEQ ID NO: 39)
ATGAAATATACAAGTTATATCTTGGCTTTTCAGCTCTGCATCGTTTTGGG
TTCTCTTGGCTGTTACTGCCAGGACCCATATGTAAAAGAAGCAGAAAACC
TTAAGAAATATTTTAATGCAGGTCATTCAGATGTAGCGGATAATGGAACT
CTTTTCTTAGGCATTTTGAAGAATTGGAAAGAGGAGAGTGACAGAAAAAT
AATGCAGAGCCAAATTGTCTCCTTTTACTTCAAACTTTTTAAAAACTTTA
AAGATGACCAGAGCATCCAAAAGAGTGTGGAGACCATCAAGGAAGACATG
AATGTCAAGTTTTTCAATAGCAACAAAAAGAAACGAGATGACTTCGAAAA
GCTGACTAATTATTCGGTAACTGACTTGAATGTCCAACGCAAAGCAATAC
ATGAACTCATCCAAGTGATGGCTGAACTGTCGCCAGCAGCTAAAACAGGG
AAGCGAAAAAGGAGTCAGATGCTGTTTCGAGGTCGAAGAGCATCCCAGTA
A

The protein sequence for IL2Ra/CD25 is set out in SEQ ID NO: 40 The signal peptide is underlined

>sp|P01589|IL2RA_HUMAN Interleukin-2 receptor sub-
unit alpha OS = Homo sapiens OX = 9606 GN = IL2RA
PE = 1 SV = 1
(SEQ ID NO: 40)
MDSYLLMWGLLTFIMVPGCQAELCDDDPPEIPHATFKAMAYKEGTMLNCE
CKRGFRRIKSGSLYMLCTGNSSHSSWDNQCQCTSSATRNTTKQVTPQPEE
QKERKTTEMQSPMQPVDQASLPGHCREPPPWENEATERIYHFVVGQMVYY
QCVQGYRALHRGPAESVCKMTHGKTRWTQPQLICTGEMETSQFPGEEKPQ
ASPEGRPESETSCLVTTTDFQIQTEMAATMETSIFTTEYQVAVAGCVFLL
ISVLLLSGLTWQRRORKSRRTI

CD25 Signal Peptide

>sp|P01589|1-21
(SEQ ID NO: 41)
MDSYLLMWGLLTFIMVPGCQA

IL2Ra DNA Sequence. DNA encoding the IL2Ra signal peptide is underlined in SEQ ID NO:42.

(SEQ ID NO: 42)
ATGGATTCATACCTGCTGATGTGGGGACTGCTCACGTTCATCATGGTGCC
TGGCTGCCAGGCAGAGCTCTGTGACGATGACCCGCCAGAGATCCCACACG
CCACATTCAAAGCCATGGCCTACAAGGAAGGAACCATGTTGAACTGTGAA
TGCAAGAGAGGTTTCCGCAGAATAAAAAGCGGGTCACTCTATATGCTCTG
TACAGGAAACTCTAGCCACTCGTCCTGGGACAACCAATGTCAATGCACAA
GCTCTGCCACTCGGAACACAACGAAACAAGTGACACCTCAACCTGAAGAA
CAGAAAGAAAGGAAAACCACAGAAATGCAAAGTCCAATGCAGCCAGTGGA
CCAAGCGAGCCTTCCAGGTCACTGCAGGGAACCTCCACCATGGGAAAATG
AAGCCACAGAGAGAATTTATCATTTCGTGGTGGGGCAGATGGTTTATTAT
CAGTGCGTCCAGGGATACAGGGCTCTACACAGAGGTCCTGCTGAGAGCGT
CTGCAAAATGACCCACGGGAAGACAAGGTGGACCCAGCCCCAGCTCATAT
GCACAGGTGAAATGGAGACCAGTCAGTTTCCAGGTGAAGAGAAGCCTCAG
GCAAGCCCCGAAGGCCGTCCTGAGAGTGAGACTTCCTGCCTCGTCACAAC
AACAGATTTTCAAATACAGACAGAAATGGCTGCAACCATGGAGACGTCCA
TATTTACAACAGAGTACCAGGTAGCAGTGGCCGGCTGTGTTTTCCTGCTG
ATCAGCGTCCTCCTCCTGAGTGGGCTCACCTGGCAGCGGAGACAGAGGAA
GAGTAGAAGAACAATCTAG

a-CD45-sc translation is set out as SEQ ID NO: 43.

In the sequence below, the bold lowercase is the heavy chain, underlined capitalized regions are linkers, the bold capitalized regions without underlining are light chains.

CD45 VH VL

(SEQ ID NO: 43)
qvqlvesggglvqpggslklscaasgfxfsrywmsxvrqapgkglewige
inptsstinxtpslkdkvfisrdnakntlylqmskvrsedtaxyycargn
yyrygdamdywgqgtsvtvskiSGGGGSGGGGSGEGGSGGGGSGGGGSSD
IVLTQSPASLAVSIGQRATISCRASKSVSTSGYSYLHWYQQKPGQPPKLL
IYLASNLESGVPARFSGSGSGTDFTLNIHPVEEEDAATYYCQHSRELPFT
XGSGTKLEIKSSGSGS

Homo sapiens CD8a molecule (CD8A), transcript variant 4, non-coding RNA Sequence ID: NR_027353.1Length: 2621Number of Matches: 1

### Related Information

Gene-associated gene details
UniGene-clustered expressed sequence tags
GEO Profiles-microarray expression data
PubChem BioAssay-bioactivity screening
Genome Data Viewer-aligned genomic context

### Range 1: 885 to 1015GenBankGraphics

Alignment statistics for match #1

Score Expect Identities Gaps Strand
243 bits(131) 3e−60 131/131(100%) 0/131(0%) Plus/Plus

| | | | |
|---|---|---|---|
| Query | 1 | CCACCACGACGCCAGCGCCGCGACCACCAACACCGGCGCCCACCATCGCGTCGCAGCCCC | 60 (SEQ ID NO: 44) |
| Sbjct | 885 | CCACCACGACGCCAGCGCCGCGACCACCAACACCGGCGCCCACCATCGCGTCGCAGCCCC | 944 (SEQ ID NO: 45) |
| Query | 61 | TGTCCCTGCGCCCAGAGGCGTGCCGGCCAGCGGCGGGGGGCGCAGTGCACACGAGGGGGC | 120 (SEQ ID NO: 46) |
| Sbjct | 945 | TGTCCCTGCGCCCAGAGGCGTGCCGGCCAGCGGCGGGGGGCGCAGTGCACACGAGGGGGC | 1004 (SEQ ID NO: 47) |
| Query | 121 | TGGACTTCGCC | 131 (SEQ ID NO: 48) |
| Sbjct | 1005 | TGGACTTCGCC | 1015 (SEQ ID NO: 49) |

Homo sapiens CD28 molecule (CD28), transcript variant 1, mRNA

### Sequence ID: NM_006139.4Length: 4721Number of Matches: 1

### Related Information

Gene-associated gene details
PubChem BioAssay-bioactivity screening
Genome Data Viewer-aligned genomic context

### Range 1: 395 to 514GenBankGraphics

Alignment statistics for match #1

Score Expect Identities Gaps Strand
222 bits(120) 4e−54 120/120(100%) 0/120(0%) Plus/Plus

| | | | |
|---|---|---|---|
| Query | 138 | AAAATTGAAGTTATGTATCCTCCTCCTTACCTAGACAATGAGAAGAGCAATGGAACCATT | 197 (SEQ ID NO: 50) |
| Sbjct | 395 | AAAATTGAAGTTATGTATCCTCCTCCTTACCTAGACAATGAGAAGAGCAATGGAACCATT | 454 (SEQ ID NO: 51) |
| Query | 198 | ATCCATGTGAAAGGGAAACACCTTIGTCCAAGTCCCCTATTTCCCGGACCTTCTAAGCCC | 257 (SEQ ID NO: 52) |
| Sbjct | 455 | ATCCATGTGAAAGGGAAACACCTTTGTCCAAGTCCCCTATTTCCCGGACCTTCTAAGCCC | 514 (SEQ ID NO: 53) |

a-CD45-Sc; Additional Engagers

We created additional engagers. anti-CD45 (9.4)single chain. This comes from human HIB-10508=9.4=IgG2a=Mouse anti-Human-CD45.

The protein sequence is shown is SEQ ID NO: 54 below. The underlined lowercase region is the IL2 signal peptide, the lowercase is the heavy chain, underlined capitalized regions are linkers, the capitalized regions without underlining are light chains, the bold capitalized regions are the stalk and the bold underlined regions are the CD34.

(SEQ ID NO: 54)
myrmqllscialslalvtnsqvqlqqlgaelarpgasvkmsckasgytft
sysiqwvkqrpgqglewigyinpssgyikynqhfrdratltadrssstay
mqlssltsedsavyycargnsgsfdywgqgttltvssaSGGGGSGGGGSG
GGGSGGGGSGGGGSSDIVLTQAAPSVPVTPGESLSISCRSSKSLLHSSGI
TYLYWFLQRPGQSPQLLIYRMSNLASGVPDRFSGSGSGTAFTLRISRVEA
EDVGVYYCMQHLEYPFTFGGGTKLEIKSSGSGSTGPTTTPAPRPPTPAPT
IASQPLSLRPEACRPAAGGAVHTRGLDFAPRKIEVMYPPPYLDNEKSNGT
IIHVKGKHLCPSPLFPGPSKPTLIALVTSGALLAVLGITGYFL

This yields the anti-CD45 (9.4) single chain codon optimized cDNA shown as SEQ ID NO: 55 below.

(SEQ ID NO: 55)
ATGTACAGAATGCAGCTGCTGAGCTGCATCGCCCTGAGCCTGGCCCTGGT
GACCAACAGCCAGGTGCAGCTGCAGCAGCTGGGCGCCGAGCTGGCCAGAC
CCGGCGCCAGCGTGAAGATGAGCTGCAAGGCCAGCGGCTACACCTTCACC
AGCTACAGCATCCAGTGGGTGAAGCAGAGACCCGGCCAGGGCCTGGAGTG
GATCGGCTACATCAACCCCAGCAGCGGCTACATCAAGTACAACCAGCACT
TCAGAGACAGAGCCACCCTGACCGCCGACAGAAGCAGCAGCACCGCCTAC
ATGCAGCTGAGCAGCCTGACCAGCGAGGACAGCGCCGTGTACTACTGCGC
CAGAGGCAACAGCGGCAGCTTCGACTACTGGGGCCAGGGCACCACCCTGA
CCGTGAGCAGCGCCAGCGGCGGCGGCGGCAGCGGCGGCGGCGGCAGCGGC
GGCGGCGGCAGCGGCGGCGGCGGCAGCGGCGGCGGCGGCAGCAGCGACAT
CGTGCTGACCCAGGCCGCCCCCAGCGTGCCCGTGACCCCCGGCGAGAGCC
TGAGCATCAGCTGCAGAAGCAGCAAGAGCCTGCTGCACAGCAGCGGCATC
ACCTACCTGTACTGGTTCCTGCAGAGACCCGGCCAGAGCCCCCAGCTGCT
GATCTACAGAATGAGCAACCTGGCCAGCGGCGTGCCCGACAGATTCAGCG
GCAGCGGCAGCGGCACCGCCTTCACCCTGAGAATCAGCAGAGTGGAGGCC
GAGGACGTGGGCGTGTACTACTGCATGCAGCACCTGGAGTACCCCTTCAC
CTTCGGCGGCGGCACCAAGCTGGAGATCAAGAGCAGCGGCAGCGGCAGCA
CCGGTCCCACCACCACCCCCGCCCCCAGACCCCCCACCCCCGCCCCCACC
ATCGCCAGCCAGCCCCTGAGCCTGAGACCCGAGGCCTGCAGACCCGCCGC
CGGCGGCGCCGTGCACACCAGAGGCCTGGACTTCGCCCCCAGAAAGATCG
AGGTGATGTACCCCCCCCCCTACCTGGACAACGAGAAGAGCAACGGCACC
ATCATCCACGTGAAGGGCAAGCACCTGTGCCCCAGCCCCCTGTTCCCCGG
CCCCAGCAAGCCCACCCTGATCGCCCTGGTGACCAGCGGCGCCCTGCTGG
CCGTGCTGGGCATCACCGGCTACTTCCTGTAA

We created an anti-CD45 (GAP8.3) single chain from light chain and heavy chain. The light chain and heavy chain sequences were obtained from GAP 8.3 hybridoma (ATCC® HB-12™)=IgG2a, kappa.=immunoglobulin; monoclonal antibody; against human leukocyte (monocytes, lymphocytes, granulocytes); against CD45.

In the sequence below (SEQ ID NO: 56), the underlined lowercase area is the IL2 signal peptide, the lowercase is the heavy chain, underlined capitalized regions are linkers, the capitalized regions without underlining are light chains, the bold capitalized regions are the stalk and the bold underlined regions are the CD34 transmembrane region.

(SEQ ID NO: 56)
myrmqllscialslalvtnsevqlqlqqsgpelvktgasvkisckasgys
ftgyfihwvkqshgkslewigyiscyngatsynqkfkgkatftvdtssst
aymqfnsvtsedsavyycvrnyygnldamdywgqgtsvtvssaSGGGGSG
GGGSGGGGSGGGGSGGGGSSDIVMTQSHKFMSTSVGDRVSITCKASQDVS
TAVAWYQQKPGQSPKILIYSASYRYTGVPDRFTGSGSGTDFTFTISSVQA
EDLAVYYCQQHYSTPRTFGGGTKLEIKRADAAQTCISSGSGSTGPTTTPA
PRPPTPAPTIASQPLSLRPEACRPAAGGAVHTRGLDFAPRKIEVMYPPPY
LDNEKSNGTIIHVKGKHLCPSPLFPGPSKPTLIALVTSGALLAVLGITGY
FL

anti-CD45 (GAP8.3) single chain codon optimized cDNA to protein SEQ ID NO: 56 is shown as SEQ ID NO: 57 below.

(SEQ ID NO: 57)
ATGTACAGAATGCAGCTGCTGAGCTGCATCGCCCTGAGCCTGGCCCTGGT
GACCAACAGCGAGGTGCAGCTGCAGCTGCAGCAGAGCGGCCCCGAGCTGG
TGAAGACCGGCGCCAGCGTGAAGATCAGCTGCAAGGCCAGCGGCTACAGC
TTCACCGGCTACTTCATCCACTGGGTGAAGCAGAGCCACGGCAAGAGCCT
GGAGTGGATCGGCTACATCAGCTGCTACAACGGCGCCACCAGCTACAACC
AGAAGTTCAAGGGCAAGGCCACCTTCACCGTGGACACCAGCAGCAGCACC
GCCTACATGCAGTTCAACAGCGTGACCAGCGAGGACAGCGCCGTGTACTA
CTGCGTGAGAAACTACTACGGCAACCTGGACGCCATGGACTACTGGGGCC
AGGGCACCAGCGTGACCGTGAGCAGCGCCAGCGGCGGCGGCGGCAGCGGC
GGCGGCGGCAGCGGCGGCGGCGGCAGCGGCGGCGGCGGCAGCGGCGGCGG
CGGCAGCAGCGACATCGTGATGACCCAGAGCCACAAGTTCATGAGCACCA
GCGTGGGCGACAGAGTGAGCATCACCTGCAAGGCCAGCCAGGACGTGAGC
ACCGCCGTGGCCTGGTACCAGCAGAAGCCCGGCCAGAGCCCCAAGATCCT
GATCTACAGCGCCAGCTACAGATACACCGGCGTGCCCGACAGATTCACCG
GCAGCGGCAGCGGCACCGACTTCACCTTCACCATCAGCAGCGTGCAGGCC
GAGGACCTGGCCGTGTACTACTGCCAGCAGCACTACAGCACCCCCAGAAC
CTTCGGCGGCGGCACCAAGCTGGAGATCAAGAGAGCCGACGCCGCCCAGA
CCTGCATCAGCAGCGGCAGCGGCAGCACCGGTCCCACCACCACCCCCGCC
CCCAGACCCCCCACCCCCGCCCCCACCATCGCCAGCCAGCCCCTGAGCCT
GAGACCCGAGGCCTGCAGACCCGCCGCCGGCGGCGCCGTGCACACCAGAG
GCCTGGACTTCGCCCCCAGAAAGATCGAGGTGATGTACCCCCCCCCCTAC
CTGGACAACGAGAAGAGCAACGGCACCATCATCCACGTGAAGGGCAAGCA
CCTGTGCCCCAGCCCCCTGTTCCCCGGCCCCAGCAAGCCCACCCTGATCG
CCCTGGTGACCAGCGGCGCCCTGCTGGCCGTGCTGGGCATCACCGGCTAC
TTCCTGTAA

Anti-CD45m(M1) single chain was created from mouse M1/89.18.7.HK (ATCC@R TIB-124™)=IgG2b.=Rat anti-Mouse-CD45

In the sequence below (SEQ ID NO: 58), the underlined lowercase area is the IL2 signal peptide, the lowercase is the heavy chain, underlined capitalized regions are linkers, the capitalized regions without underlining are light chains, the bold capitalized regions are the stalk and the bold underlined regions are the CD34.

(SEQ ID NO: 58)
myrmqllscialslalvtnsqvqlkesgpglvkpsltlsltctvsgfsln
sygviwvrqppgkglewlgvkwgygntnynsalksrlninrdtsksqvfl
kmdnvqtedtamyfcarsrfnyggpldywgqgvmvtvssaSGGGGSGGGG
SGGGGSGGGGSGGGGSSDIVLTQSPKSMSMSVGERVTLTCKASENVVTYV
SWYQQKPEQSPKLLIYGASNRYTGVPDRFTGSGSATDFTLTISSVQAEDL
ADYHCGQGYSYPYTFGGGTKLEIKRADAAPTVSSSGSGSTGPTTTPAPRP
PTPAPTIASQPLSLRPEACRPAAGGAVHTRGLDFAPRKIEVMYPPPYLDN
EKSNGTIIHVKGKHLCPSPLFPGPSKPTLIALVTSGALLAVLGITGYFL

Anti-CD45(M1) codon optimized for human cell expression.

(SEQ ID NO: 59)
ATGTACAGAATGCAGCTGCTGAGCTGCATCGCCCTGAGCCTGGCCCTGGT
GACCAACAGCCAGGTGCAGCTGAAGGAGAGCGGCCCCGGCCTGGTGAAGC
CCAGCCTGACCCTGAGCCTGACCTGCACCGTGAGCGGCTTCAGCCTGAAC
AGCTACGGCGTGATCTGGGTGAGACAGCCCCCCGGCAAGGGCCTGGAGTG
GCTGGGCGTGAAGTGGGGCTACGGCAACACCAACTACAACAGCGCCCTGA
AGAGCAGACTGAACATCAACAGAGACACCAGCAAGAGCCAGGTGTTCCTG
AAGATGGACAACGTGCAGACCGAGGACACCGCCATGTACTTCTGCGCCAG
AAGCAGATTCAACTACGGCGGCCCCCTGGACTACTGGGGCCAGGGCGTGA
TGGTGACCGTGAGCAGCGCCAGCGGCGGCGGCGGCAGCGGCGGCGGCGGC
AGCGGCGGCGGCGGCAGCGGCGGCGGCGGCAGCGGCGGCGGCGGCAGCAG
CGACATCGTGCTGACCCAGAGCCCCAAGAGCATGAGCATGAGCGTGGGCG
AGAGAGTGACCCTGACCTGCAAGGCCAGCGAGAACGTGGTGACCTACGTG
AGCTGGTACCAGCAGAAGCCCGAGCAGAGCCCCAAGCTGCTGATCTACGG
CGCCAGCAACAGATACACCGGCGTGCCCGACAGATTCACCGGCAGCGGCA
GCGCCACCGACTTCACCCTGACCATCAGCAGCGTGCAGGCCGAGGACCTG
GCCGACTACCACTGCGGCCAGGGCTACAGCTACCCCTACACCTTCGGCGG
CGGCACCAAGCTGGAGATCAAGAGAGCCGACGCCGCCCCCACCGTGAGCA
GCAGCGGCAGCGGCAGCACCGGTCCCACCACCACCCCCGCCCCCAGACCC
CCCACCCCCGCCCCCACCATCGCCAGCCAGCCCCTGAGCCTGAGACCCGA
GGCCTGCAGACCCGCCGCCGGCGGCGCCGTGCACACCAGAGGCCTGGACT
TCGCCCCCAGAAAGATCGAGGTGATGTACCCCCCCCCCTACCTGGACAAC
GAGAAGAGCAACGGCACCATCATCCACGTGAAGGGCAAGCACCTGTGCCC
CAGCCCCCTGTTCCCCGGCCCCAGCAAGCCCACCCTGATCGCCCTGGTGA
CCAGCGGCGCCCTGCTGGCCGTGCTGGGCATCACCGGCTACTTCCTGTAA

anti-CD45(4B2) single chain 4B2 (ATCC®R HB-196™)=Mouse anti-human CD45.

In the sequence below (SEQ ID NO: 60), the underlined lowercase area is the IL2 signal peptide, the lowercase is the heavy chain, underlined capitalized regions are linkers, the capitalized regions without underlining are light chains, the bold capitalized regions are the stalk and the bold underlined regions are the CD34.

(SEQ ID NO: 60)
myrmqllscialslalvtnsqvqlkesgaelarpgasvkmsckasgytft
sytmqwvkqrpgqglewigyinpssgyikynqkfkdkvtltadkssttay
mqlsrltsedsavyycarrgsyffdfwgqgtsvtvssaSGGGGSGGGGSG
GGGSGGGGSGGGGSSDIVLTQDELSNPVTSGESVSISCRSSKSLLYKDGK
TYLNWFLQRPGQSPQLLIYLMSTRASGVSDRFSGSGSGTDFTLEISRVKA
EDVGVYYCQQLVEYPFTFGGGTKLEVKRADAAPTVSSSGSGSTGPTTTPA
PRPPTPAPTIASQPLSLRPEACRPAAGGAVHTRGLDFAPRKIEVMYPPPY
SLDNEKSNGTIIHVKGKHLCPSPLFPGPSKPTLIALVTGALLAVLGITGY
FL

anti-CD45(4B2) codon optimized for human cell expression

(SEQ ID NO: 61)
atgtacagaatgcagctgctgagctgcatcgccctgagcctggccctggt
gaccaacagccaggtgcagctgaaggagagcggcgccgagctggccagac
ccggcgccagcgtgaagatgagctgcaaggccagcggctacaccttcacc
agctacaccatgcagtgggtgaagcagagacccggccagggcctggagtg
gatcggctacatcaaccccagcagcggctacatcaagtacaaccagaagt
tcaaggacaaggtgaccctgaccgccgacaagagcagcaccaccgcctac
atgcagctgagcagactgaccagcgaggacagcgccgtgtactactgcgc
cagaagaggcagctacttcttcgacttctggggccagggcaccagcgtga
ccgtgagcagcgccagcggcggcggcggcagcggcggcggcggcagcggc
ggcggcggcagcggcggcggcggcagcggcggcggcggcagcagcgacat
cgtgatcacccaggacgagctgagcaaccccgtgaccagcggcgagagcg
tgagcatcagctgcagaagcagcaagagcctgctgtacaaggacggcaag
acctacctgaactggttcctgcagagacccggccagagcccccagctgct
gatctacctgatgagcaccagagccagcggcgtgagcgacagattcagcg
gcagcggcagcggcaccgacttcaccctggagatcagcagagtgaaggcc
gaggacgtgggcgtgtactactgccagcagctggtggagtaccccttcac
cttcggcggcggcaccaagctggaggtgaagagagccgacgccgccccca
ccgtgagcagcagcggcagcggcagcACCGGTCCCACCACCACCCCCGCC
CCCAGACCCCCCACCCCCGCCCCCACCATCGCCAGCCAGCCCCTGAGCCT
GAGACCCGAGGCCTGCAGACCCGCCGCCGGCGGCGCCGTGCACACCAGAG
GCCTGGACTTCGCCCCCAGAAAGATCGAGGTGATGTACCCCCCCCCCTAC
CTGGACAACGAGAAGAGCAACGGCACCATCATCCACGTGAAGGGCAAGCA
CCTGTGCCCCAGCCCCCTGTTCCCCGGCCCCAGCAAGCCCACCCTGATCG
CCCTGGTGACCAGCGGCGCCCTGCTGGCCGTGCTGGGCATCACCGGCTAC
TTCCTGTAA

Anti-CD3 (OKT3) single chain taken from Arakawa F, Kuroki M, Kuwahara M, Senba T, Ozaki H, Matsuoka Y, Misumi Y, Kanda H, Watanabe T. Cloning and sequencing of the VH and V kappa genes of an anti-CD3 monoclonal antibody, and construction of a mouse/human chimeric antibody. J Biochem. 1996 September; 120(3):657-62. doi: 10.1093/oxfordjournals.jbchem.a021462. PMID: 8902633. In the sequence below (SEQ ID NO: 62), the underlined lowercase area is the IL2 signal peptide, the lowercase is the heavy chain, underlined capitalized regions are linkers, the capitalized regions without underlining are light chains, the bold capitalized regions are the stalk and the bold underlined regions are the CD34.

(SEQ ID NO: 62)
myrmqllscialslalvtnsqvqlqqsgaelarpgasvkmsckasgytft
rytmhwvkqrpgqglewigyinpsrgytnynqkfkdkatlttdkssstay
mqlssltsedsavyycaryyddhycldywgqgttltvssakSGGGGSGGG
GSGGGGSGGGGSGGGGSSQIVLTQSPAIMSASPGEKVTMTCSASSSVSYM
NWYQQKSGTSPKRWIYDTSKLASGVPAHFRGSGSGTSYSLTISGMEAEDA
ATYYCQQWSSNPFTFGSGTKLEINRSSGSGSTGPTTTPAPRPPTPAPTIA
SQPLSLRPEACRPAAGGAVHTRGLDFAPRKIEVMYPPPYLDNEKSNGTII
HVKGKHLCPSPLFPGPSKPTLIALVTSGALLAVLGITGYFL

Anti-CD3 (OKT3) single chain codon optimized for human cell expression shown as SEQ ID NO: 63 below.

(SEQ ID NO: 63)
ATGTACAGAATGCAGCTGCTGAGCTGCATCGCCCTGAGCCTGGCCCTGGT
GACCAACAGCCAGGTGCAGCTGCAGCAGAGCGGCGCCGAGCTGGCCAGAC
CCGGCGCCAGCGTGAAGATGAGCTGCAAGGCCAGCGGCTACACCTTCACC
AGATACACCATGCACTGGGTGAAGCAGAGACCCGGCCAGGGCCTGGAGTG
GATCGGCTACATCAACCCCAGCAGAGGCTACACCAACTACAACCAGAAGT
TCAAGGACAAGGCCACCCTGACCACCGACAAGAGCAGCAGCACCGCCTAC
ATGCAGCTGAGCAGCCTGACCAGCGAGGACAGCGCCGTGTACTACTGCGC
CAGATACTACGACGACCACTACTGCCTGGACTACTGGGGCCAGGGCACCA
CCCTGACCGTGAGCAGCGCCAAGAGCGGCGGCGGCGGCAGCGGCGGCGGC
GGCAGCGGCGGCGGCGGCAGCGGCGGCGGCGGCAGCGGCGGCGGCGGCAG
CAGCCAGATCGTGCTGACCCAGAGCCCCGCCATCATGAGCGCCAGCCCCG
GCGAGAAGGTGACCATGACCTGCAGCGCCAGCAGCAGCGTGAGCTACATG
AACTGGTACCAGCAGAAGAGCGGCACCAGCCCCAAGAGATGGATCTACGA
CACCAGCAAGCTGGCCAGCGGCGTGCCCGCCCACTTCAGAGGCAGCGGCA
GCGGCACCAGCTACAGCCTGACCATCAGCGGCATGGAGGCCGAGGACGCC
GCCACCTACTACTGCCAGCAGTGGAGCAGCAACCCCTTCACCTTCGGCAG
CGGCACCAAGCTGGAGATCAACAGAAGCAGCGGCAGCGGCAGCACCGGTC
CCACCACCACCCCCGCCCCCAGACCCCCCACCCCCGCCCCCACCATCGCC
AGCCAGCCCCTGAGCCTGAGACCCGAGGCCTGCAGACCCGCCGCCGGCGG
CGCCGTGCACACCAGAGGCCTGGACTTCGCCCCCAGAAAGATCGAGGTGA
TGTACCCCCCCCCCTACCTGGACAACGAGAAGAGCAACGGCACCATCATC
CACGTGAAGGGCAAGCACCTGTGCCCCAGCCCCCTGTTCCCCGGCCCCAG
CAAGCCCACCCTGATCGCCCTGGTGACCAGCGGCGCCCTGCTGGCCGTGC
TGGGCATCACCGGCTACTTCCTGTAA

Anti-CD148 single chain was taken from published patent application US 2005/0287,140 A1 from sequence AB1. In the sequence below (SEQ ID NO: 64), the underlined lowercase area is the IL2 signal peptide, the lowercase is the heavy chain, underlined capitalized regions are linkers, the capitalized regions without underlining are light chains, the bold capitalized regions are the stalk and the bold underlined regions are the transmembrane region of CD34.

(SEQ ID NO: 64)
myrmqllscialslalvtnsevqllesggglvqpggslrlscaasgftfs
syamswvrqapgkglewvsaisgsggstyyadsvkgrftisrdnskntly
lqmnslraedtavyycargrtevatpgaywgqgtmvtvssaSGGGGSGGG
GSGGGGSGGGGSGGGGSSQAVLTQPSSVSGAPGQRVTISCTGSSSNIGAG
YDVHWYQQLPGTAPKLLIYGNSNRPSGVPDRFSGSKSGTSASLAVTGLQA
EDEADYYCQSYDSSLSDVVFGGGTKLTVLSSGSGSTGPTTTPAPRPPTPA
PTIASQPLSLRPEACRPAAGGAVHTRGLDFAPRKIEVMYPPPYLDNEKSN
GTIIHVKGKHLCPSPLFPGPSKPTLIALVTSGALLAVLGITGYFL

Anti-CD148 single chain codon optimized for human cell expression shown as cDNA in SEQ ID NO: 65 below.

(SEQ ID NO: 65)
ATGTACAGAATGCAGCTGCTGAGCTGCATCGCCCTGAGCCTGGCCCTGGT
GACCAACAGCGAGGTGCAGCTGCTGGAGAGCGGCGGCGGCCTGGTGCAGC
CCGGCGGCAGCCTGAGACTGAGCTGCGCCGCCAGCGGCTTCACCTTCAGC
AGCTACGCCATGAGCTGGGTGAGACAGGCCCCCGGCAAGGGCCTGGAGTG
GGTGAGCGCCATCAGCGGCAGCGGCGGCAGCACCTACTACGCCGACAGCG
TGAAGGGCAGATTCACCATCAGCAGAGACAACAGCAAGAACACCCTGTAC
CTGCAGATGAACAGCCTGAGAGCCGAGGACACCGCCGTGTACTACTGCGC
CAGAGGCAGAACCGAGGTGGCCACCCCCGGCGCCTACTGGGGCCAGGGCA
CCATGGTGACCGTGAGCAGCGCCAGCGGCGGCGGCGGCAGCGGCGGCGGC
GGCAGCGGCGGCGGCGGCAGCGGCGGCGGCGGCAGCGGCGGCGGCGGCAG
CAGCCAGGCCGTGCTGACCCAGCCCAGCAGCGTGAGCGGCGCCCCCGGCC
AGAGAGTGACCATCAGCTGCACCGGCAGCAGCAGCAACATCGGCGCCGGC
TACGACGTGCACTGGTACCAGCAGCTGCCCGGCACCGCCCCCAAGCTGCT
GATCTACGGCAACAGCAACAGACCCAGCGGCGTGCCCGACAGATTCAGCG
GCAGCAAGAGCGGCACCAGCGCCAGCCTGGCCGTGACCGGCCTGCAGGCC
GAGGACGAGGCCGACTACTACTGCCAGAGCTACGACAGCAGCCTGAGCGA
CGTGGTGTTCGGCGGCGGCACCAAGCTGACCGTGCTGAGCAGCGGCAGCG
GCAGCACCGGTCCCACCACCACCCCCGCCCCCAGACCCCCCACCCCCGCC
CCCACCATCGCCAGCCAGCCCCTGAGCCTGAGACCCGAGGCCTGCAGACC
CGCCGCCGGCGGCGCCGTGCACACCAGAGGCCTGGACTTCGCCCCCAGAA
AGATCGAGGTGATGTACCCCCCCCCCTACCTGGACAACGAGAAGAGCAAC
GGCACCATCATCCACGTGAAGGGCAAGCACCTGTGCCCCAGCCCCCTGTT
CCCCGGCCCCAGCAAGCCCACCCTGATCGCCCTGGTGACCAGCGGCGCCC
TGCTGGCCGTGCTGGGCATCACCGGCTACTTCCTGTAA

E3.49K R1 Mutant

The E3.49K R1 mutant was created through deletion of some extracellular region of E3.49k taken from Uniprot (FIGS. 15 and 22). In the sequence below (SEQ ID NO: 66), the underlined lowercase area is the E3.49K signal peptide, the lowercase is R1 domain, underlined capitalized regions are linkers, the capitalized regions without underlining are E3.49K extracellular membrane proximal region, and the bold underlined regions are the transmembrane region of E3.49K followed by bold capitalized intracellular regions of E3.49K.

(SEQ ID NO: 66)
mntvirivllsllvafsqagfhtinatwwanitlvgppdtpvtwydtqgl
wfcngsrvknpqirhtcndqnltlihvnktyertymgynrqgtkkedykv
vviGGGGSDEGKRYRVKVIPPNTTNSQSVKIQPYTRQTTPDQEHKFELQF
ETNGNYDSKIPSTTVAIVVGVIAGFITLIIVFICYICCRKRPRAYNHMVD
PLLSFSY

E3.49K R1

An E3.49K R1 codon optimized for human cells expression shown in cDNA in SEQ ID NO: 67 below.

In the sequence below (SEQ ID NO: 67), the underlined lowercase area is the E3.49K signal peptide, the lowercase is R1 domain, underlined capitalized regions are linkers, the capitalized regions without underlining are E3.49K extracellular membrane proximal region, and the bold underlined regions are the transmembrane region of E3.49K followed by capitalized bold capitalized intracellular regions of E3.49K

(SEQ ID NO: 67)
atgaacacggtgatccgcatagtccttctgtctctgctggtggctttctc
ccaggccggcttccacacaattaatgccacctggtgggctaacattactc
tcgtaggccccccggatacccccgtgacttggtacgacactcagggtctg
tggttctgtaacgggagtcgagtgaaaaatcctcaaattcgccatacctg
taacgaccaaaatctgaccttgatccacgtgaacaagacatacgagcgta
catatatgggctacaataggcagggtacaaagaaagaggactataaagtg
gtagtgattGGCGGCGGCGGCAGCGATGAGGGAAAACGGTACCGGGTTAA
GGTTATTCCGCCTAACACCACAAACTCCCAGAGTGTCAAAATTCAGCCTT
ACACCAGGCAGACTACTCCTGACCAGGAACACAAATTCGAATTACAGTTT
GAGACTAACGGTAACTATGACTCCAAGATTCCATCTACAACGGTCGCGAT
CGTAGTGGGCGTGATTGCAGGCTTCATCACATTGATCATCGTGTTCATCT
GCTATATCTGCTGTAGGAAGCGCCCTCGGGCGTACAACCACATGGTGGAC
CCTCTGTTGAGTTTCTCATATTAA

E3.49K-Ig-R3 mutant from E3.49k taken from Uniprot (FIGS. 16 and 23).

In the sequence below (SEQ ID NO: 68), the underlined lowercase area is the E3.49K signal peptide, underlined capitalized regions are linkers, the lowercase is R3 domain, the capitalized regions without underlining are E3.49K extracellular membrane proximal region, and the bold underlined regions are the transmembrane region of E3.49K followed by bold capitalized intracellular regions of E3.49K.

(SEQ ID NO: 68)
mntvirivllsllvafsqagfhtinatwwanitlvGGGGSvtvtagsnlt
lvgpkaegkvtwfdgdlkrpcepnyrlrhecnnqnltlinvtkdyegtyy
gtndkdegkryrvkvNTTNSQSVKIQPYTRQTTPDQEHKFELQFETNGNY
DSKIPSTTVAIVVGVIAGFITLIIVFICYICCRKRPRAYNHMVDPLLSFS
Y

E3.49K-Ig-R3 codon optimized for human cell expression cDNA in SEQ ID NO 69 below.

(SEQ ID NO: 69)
atgaacacggtgatccgcatagtccttctgtctctgctggtggctttctc
ccaggccggcttccacacaattaatgccacctggtgggctaacattactc
tcgtaGGCGGCGGCGGCAGCgtgacagtaactgctggaagtaacctgacc
ctcgtggggcccaaggcggaggggaaagtaacctggttcgacggcgatct
aaaacgcccctgtgaaccaaactacagacttagacacgaatgcaacaacc
agaacctgactctgattaacgtgaccaaggactacgaaggaacatactac
gggacgaatgataaggatgagggaaaacggtaccgggttaaggttAACAC
CACAAACTCCCAGAGTGTCAAAATTCAGCCTTACACCAGGCAGACTACTC
CTGACCAGGAACACAAATTCGAATTACAGTTTGAGACTAACGGTAACTAT
GACTCCAAGATTCCATCTACAACGGTCGCGATCGTAGTGGGCGTGATTGC
AGGCTTCATCACATTGATCATCGTGTTCATCTGCTATATCTGCTGTAGGA
AGCGCCCTCGGGCGTACAACCACATGGTGGACCCTCTGTTGAGTTTCTCA
TATTAA

All three of the CD45 engagers E3.49K, UL11 and anti-CD45-single chain (a-CD45-sc) created above bind to all isoforms of CD45. This suggests an interaction with the membrane proximal region of CD45 including fibronectin-III and cysteine-rich domains. Immunoprecipitation studies reveal a physical interaction between CD45 and E3.49K, UL11 or a-CD45-sc while antibody competition experiments and deletion mutations further support the idea that E3.49K, UL11, and a-CD45-sc mainly interact with membrane proximal regions of CD45 common to all isoforms. We will generate additional antibodies specific to different isoforms and epitopes of CD45. This will also be evaluated for CD43 and CD148.

### Example 3: Creation of a VHH-Nanobody

A nanobody is a single monomeric variable antibody domain that selectively binds the specific antigen, like antibodies. Nanobodies are much smaller (12-15 kDa) compared to common antibodies (150-160 kDa). Nanobodies are generally engineered from heavy-chain antibodies found in camelids which are also called VHH fragments, or single domains. VHH-fragments given below are specifically against murine CD45. Codon optimization was carried out with CLC Main Workbench, as mentioned above.

The VHH Generation Method.

Female camelids are given intramuscular and/or intradermal injections of purified (human) antigen every three weeks. Purified protein antigens in phosphate-buffered saline (PBS)/HEPES-buffered saline (HBS) are prepared and concentrated to ≥1 mg/mL. Approximately 3 mg of protein is used for the complete protocol, including the immunization, panning, and confirmation of clones. Three to four days after the 3rd and 5th injections, a small test bleed is performed from each animal to obtain sera for testing. The presence of antigen-specific antibodies are confirmed by ELISA using the sera obtained from test bleeds at pre-immune, three-week, and five-week time points. The final bleed is taken while the antibody titer is still increasing.

Following immunization, peripheral blood lymphocytes are isolated by centrifugation on a Ficoll discontinuous gradient. Total RNA is extracted from the peripheral blood lymphocytes and first strand cDNA is synthesized from total or polyA+RNA, using cDNA synthesis kit. Bacteriophage libraries are generated from this cDNA. Single domain antibodies are panned by adding the phage solution to antigen coated plate wells. Specific phages (elute) are added to TGT phage display competent cells and grown at 37° C. for 30 min. Serial dilution of the bacteria is plated and grown overnight at 37° C. Colonies from the plate are inoculated to a 96-well plate and incubated overnight at 37° C., without shaking. The next day, the plate is shaken at 170 rpm at 37° C. for 1 hr. 2 uL medium is used to PCR amplify and screen positive clones. Positive clones are grown in 10 ml Luria Bertani medium (LB) and grown overnight with shaking at 37° C. A miniprep is performed and clones are sequenced. Repeatedly identified sequences are likely the high affinity binding sequences. These sequences can be used to generate the engagers and their affinity and avidity can be confirmed using a pull-down assay, and ELISA.

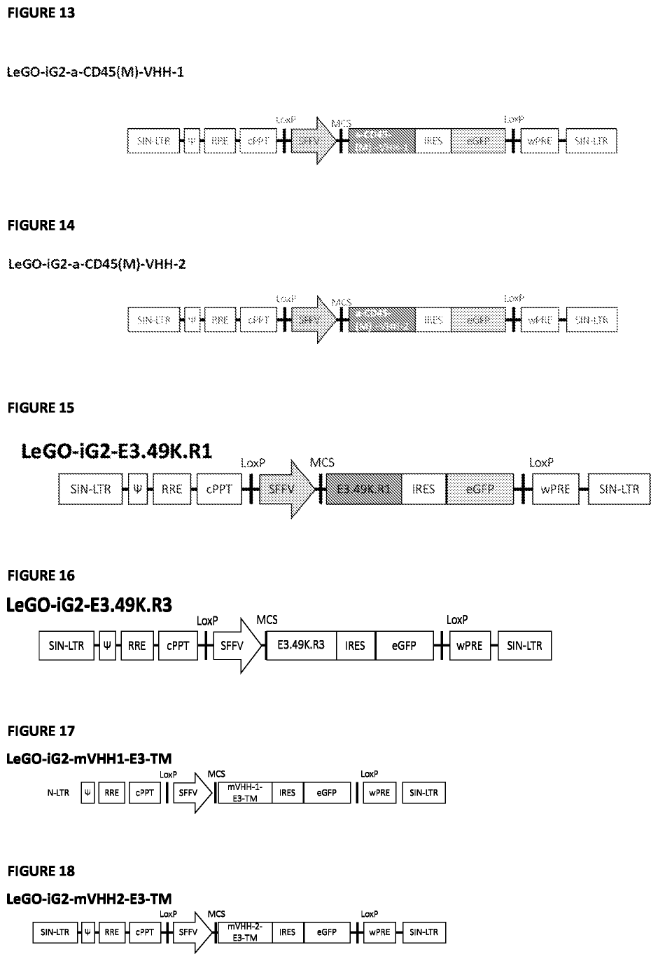

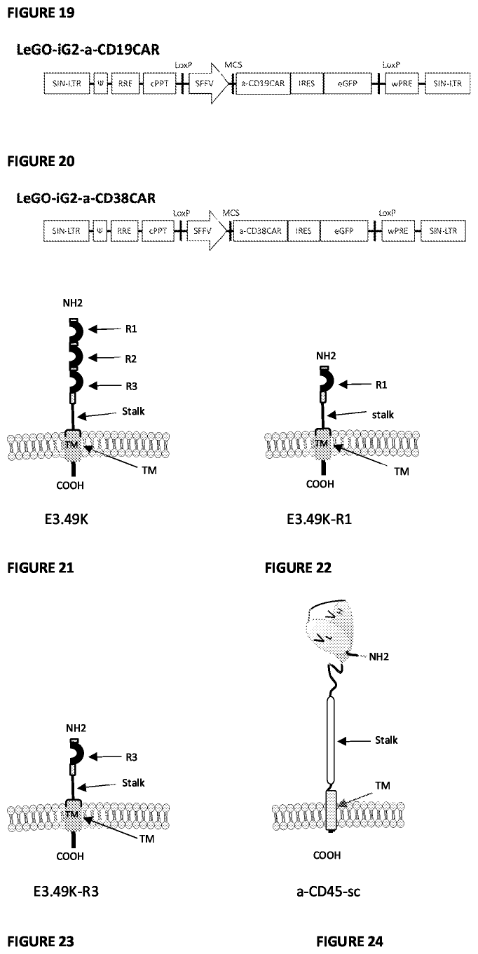

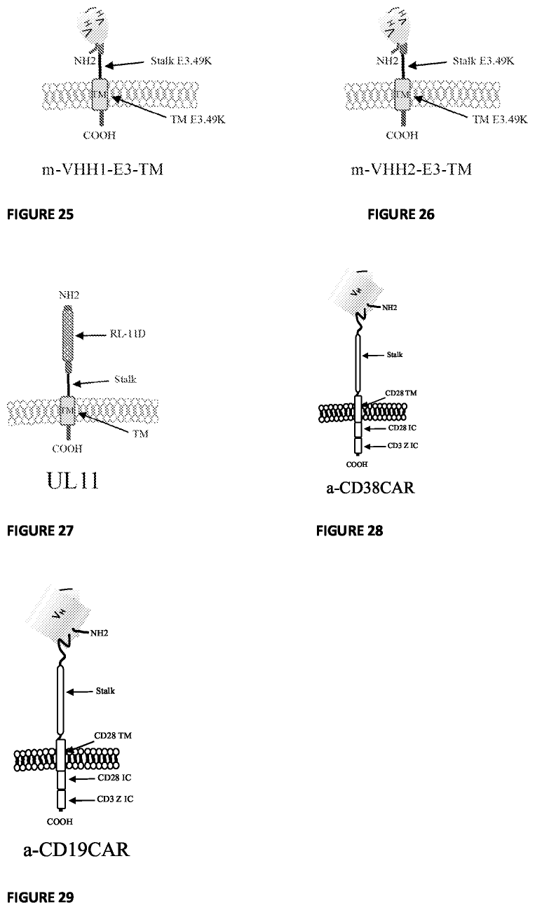

A cDNA was created for the VHH-Nanobody a-CD45-1(Murine) Codon optimized for human cells expression (xenografting to mouse, transduce human cells with human cells against murine CD45) Rossotti M, Tabares S, Alfaya L, Leizagoyen C, Moron G, Gonzalez-Sapienza G. Streamlined method for parallel identification of single domain antibodies to membrane receptors on whole cells. Biochim Biophys Acta. 2015; 1850(7):1397-404. The cDNA is shown in FIG. 13 as part of LeGO-iG2-a-CD45(M)-VHH-1 and shown in SEQ ID NO: 70 below.

(SEQ ID NO: 70)
ATGGCCCAGGTGCAGCTGGTGGAGAGCGGCGGCGGCCTGGTGCACCCCGG
CGACAGCCTGAGACTGAGCTGCGCCGCCAGCGGCAGCGTGTTCAACAGCG
CCACCATGGGCTGGTACAGACAGAGCCCCGGCAGCCAGAGAGAGCTGGTG
GCCACCATCGTGGTGGGCACCCCCACCTACGCCGACAGCGTGAAGGGCAG
ATTCACCATCAGCAGAGACAACGCCAAGAACATCGTGTACCTGCAGATGA
ACAGCCTGAAGCCCGAGGACACCGCCGTGTACTACTGCAACTACAGAGCC
ACCTACACCAGCGGCTACAGCAGAGACTACTGGGGCCAGGGCACCCAGGT
GACCGTGAGC

VHH-Nanobody a-CD45-1(Murine) Protein Sequence

(SEQ ID NO: 71)
MAQVQLVESGGGLVHPGDSLRLSCAASGSVFNSATMGWYRQSPGSQRELV
ATIVVGTPTYADSVKGRFTISRDNAKNIVYLQMNSLKPEDTAVYYCNYRA
TYTSGYSRDYWGQGTQVTVS

Currently we have two different VHH Engagers against murine CD45 which bind different epitopes and have to be tested(5).

VHH-Nanobody a-CD45-2(Murine) Codon optimized for human cells expression (DNA sequence) This is shown in FIG. 14 as LeGO-iG2-a-CD45(M)-VHH-2.

(SEQ ID NO: 72)
ATGGCCCAGGTGCAGCTGGTGCAGAGCGGCGGCGGCCTGGTGCAGCCCGG
CGGCAGCCTGAGACTGAGCTGCGCCGCCAGCGGCAGAGCCTTCAACAGCG
CCGCCATGGGCTGGTACAGACAGGCCCCCGGCAGCCAGAGAGAGCTGGTG
GCCAGCATCAGCGCCGGCACCGCCAGCTACGCCGACGCCGTGAAGGGCAG
ATTCACCATCAGCAGAGACTACGCCAAGAACATCATCTACCTGCAGATGA
ACAGCCTGAAGCCCGACGACACCGCCGTGTACTTCTGCAACTACAGAACC
ACCTACACCAGCGGCTACAGCGAGGACTACTGGGGCCAGGGCACCCAGGT
GACCGTGAGC

VHH-Nanobody a-CD45-2(Murine) (Amino Acid Sequence)

(SEQ ID NO: 73)
MAQVQLVQSGGGLVQPGGSLRLSCAASGRAFNSAAMGWYRQAPGSQRELV
ASISAGTASYADAVKGRFTISRDYAKNIIYLQMNSLKPDDTAVYFCNYRT
TYTSGYSEDYWGQGTQVTVS

### Example 4: Single Domain Human Nanobody Sequences

We have generated engagers comprising human single domain/nanobody sequences using the methods disclosed above for Example 3. The generated protein and cDNA sequences are set forth in SEQ ID NO: 74 to 215. These were created for the inventors by Nanotag.

a-CD45-h-VHH-01

(SEQ ID NO: 74)
EVQLVESGGGLVQPGGSLRLSCAASERAYRNRLLGWFRQVPGKEREFVAW
IRPIDSSTNYADSVRGRFTISRDNARSTLHLQMNSLKPEDTAVYYCVKGN
GLTSTRASDYWGQGTQVTVLSAHHSEDPIS

This protein sequence is encoded by the cDNA shown in SEQ ID NO: 75 below.

(SEQ ID NO: 75)
GAGGTGCAGCTGGTGGAGTCTGGCGGAGGCTTGGTGCAGCCTGGGGGGTC
TCTGAGACTCTCCTGTGCAGCCTCTGAACGCGCCTACAGGAACCGTCTTC
TTGGCTGGTTCCGCCAGGTTCCAGGGAAGGAGCGTGAATTTGTGGCATGG
ATCAGACCCATTGATAGCAGCACAAATTATGCAGACTCCGTGAGGGGCCG
ATTCACCATCTCCAGAGATAACGCCAGGAGCACGCTGCATCTGCAAATGA
ACAGCCTGAAACCTGAGGACACGGCCGTGTATTACTGTGTAAAGGGGAAC
GGACTTACTTCTACTCGCGCGAGTGACTACTGGGGCCAGGGGACCCAGGT
CACCGTCTTGTCAGCGCACCACAGCGAAGACCCTATTAGT

a-CD45-h-VHH-02

(SEQ ID NO: 76)
EVQLLESGGGLVQAGDSLRLSCAASGLTNPERRLAWFRQAPGKEREFVAS
IRWSGGPNTHYGDSVKGRFTISRDNGKNTVALQMNNLKPEDTAVYFCAAA
VRLTAPLNFDTSYDYWGQGTQVTISSEPKTPKPQT

This protein sequence is encoded by the cDNA shown in SEQ ID NO: 77 below.

(SEQ ID NO: 77)
GAGGTGCAGCTGCTGGAGTCTGGGGGAGGATTGGTGCAGGCTGGGGACTC
TCTGAGACTCTCCTGTGCAGCTTCTGGACTGACTAACCCTGAAAGACGCT
TGGCCTGGTTCCGCCAGGCTCCAGGGAAGGAGCGTGAGTTTGTAGCGTCC
ATTCGCTGGAGTGGTGGTCCCAACACACACTATGGCGACTCCGTGAAGGG
CCGATTCACCATCTCCAGAGACAACGGCAAGAACACGGTGGCTCTACAAA
TGAACAACCTGAAACCTGAGGACACGGCCGTTTATTTCTGTGCAGCGGCT
GTGCGTCTAACTGCGCCTCTCAATTTTGACACCTCGTATGACTACTGGGG
CCAGGGGACCCAGGTCACCATCTCCTCAGAACCCAAGACACCAAAACCAC
AAACT

a-CD45-h-VHH-03

(SEQ ID NO: 78)
EVQLEESGGGLVQPGGSLRLSCAASGFTFSNQVMSWVRQAPGKGPERVAV
IGSVGGATGATSYADSVRGRFTISRDNARSTLHLQMNSLKPEDTAVYYCA
ARVRGSTGDFGSWGQGTQVTVSSEPKTPKPQT

This protein sequence is encoded by the cDNA shown in SEQ ID NO: 79 below.

(SEQ ID NO: 79)
GAGGTGCAGCTGGAGGAGTCTGGGGGAGGCTTGGTGCAGCCTGGGGGGTC
TCTGAGACTCTCCTGTGCAGCCTCTGGATTCACCTTCAGTAACCAAGTCA
TGAGCTGGGTCCGCCAGGCTCCAGGAAAGGGGCCCGAGCGGGTCGCAGTT
ATCGGCAGTGTCGGAGGTGCCACAGGTGCCACAAGTTATGCAGACTCCGT
GAGGGGCCGATTCACCATCTCCAGAGATAACGCCAGGAGCACGCTGCATC
TGCAAATGAACAGCCTGAAACCTGAGGACACGGCCGTGTATTACTGTGCA
GCGAGGGTACGCGGCAGCACAGGGGACTTTGGTTCCTGGGGCCAGGGGAC
CCAGGTCACCGTCTCCTCGGAACCCAAGACACCAAAACCACAAACT.

a-CD45-h-VHH-04

(SEQ ID NO: 80)
EVQLVESGGGLVETGGSLRLSCAGSGRTFSSRHVGWFRQTPGKEREFVAS
IRWSGGHTYYADSVKGRFTISRDNGKNTVALQMNNLKPEDTAVYFCAAAV
RLTAPLNFDTSYDYWGQGTQVTISSEPKTPKPQT

This protein sequence is encoded by the cDNA shown in SEQ ID NO: 81 below.

(SEQ ID NO: 81)
GAGGTGCAGCTGGTGGAGTCTGGGGGAGGATTGGTCGAAACTGGGGGTTC
TCTGAGACTCTCCTGTGCAGGTTCTGGACGCACCTTCAGTAGCCGGCACG
TGGGCTGGTTCCGCCAGACTCCAGGGAAGGAGCGTGAGTTTGTAGCATCC
ATTAGGTGGAGTGGCGGTCACACATACTATGCAGACTCCGTGAAGGGCCG
ATTCACCATCTCCAGAGACAACGGCAAGAACACGGTGGCTCTACAAATGA
ACAACCTGAAACCTGAGGACACGGCCGTTTATTTCTGTGCAGCGGCTGTG
CGTCTAACTGCGCCTCTCAATTTTGACACCTCGTATGACTACTGGGGCCA
GGGGACCCAGGTCACCATCTCCTCAGAACCCAAGACACCAAAACCACAAA
CT

a-CD45-h-VHH-05

(SEQ ID NO: 82)
EVQLVESGGGLVQPGGSLRLSCAASGFTFSNQVMSWVRQAPGKGPERVAV
IGSVGGATGATSYADSVRGRFTISRDNARSTLHLQMNSLKPEDTAVYYCV
KGNGLTSTRASDYWGQGTQVTVSSEPKTPKPQT

This protein sequence is encoded by the cDNA shown in SEQ ID NO: 83 below.

(SEQ ID NO: 83)
GAGGTGCAGCTGGTGGAGTCTGGGGGAGGCTTGGTGCAGCCTGGGGGGTC
TCTGAGACTCTCCTGTGCAGCCTCTGGATTCACCTTCAGTAACCAAGTCA
TGAGCTGGGTCCGCCAGGCTCCAGGAAAGGGGCCCGAGCGGGTCGCAGTT
ATCGGCAGTGTCGGAGGTGCCACAGGTGCCACAAGTTATGCAGACTCCGT
GAGGGGCCGATTCACCATCTCCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-06

(SEQ ID NO: 84)
EVQLQESGGGLVQPGGSLRLSCVASGFTFSIYAMSWVRQAPGKGPERVAV
IGSVGGATGVTSYADSVKDRFTISRDNARSTLHLQMNSLKPEDTAVYYCV
KGNGLTSTRASDYWGQGTQVTVSSEPKTPKPQT

This protein sequence is encoded by the cDNA shown in SEQ ID NO: 85 below.

(SEQ ID NO: 85)
GAGGTGCAGCTGCAGGAGTCTGGGGGAGGCTTGGTGCAGCCTGGGGGGTC
TCTGAGACTCTCCTGTGTAGCCTCTGGATTCACCTTCAGTATCTACGCCA
TGAGCTGGGTCCGCCAGGCTCCAGGAAAGGGGCCCGAGCGGGTCGCAGTT
ATCGGCAGTGTCGGAGGTGCCACAGGTGTCACAAGTTATGCAGACTCCGT
GAAGGACCGATTCACCATCTCCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-07

(SEQ ID NO: 86)
EVQLVESGGGLVQAGGSLKLSCAASGRTLTYYTAWFRQAPGKEREFVASL
GWSGDVTYYADSVKGRFTISGDNAKNTVYLQMNSLKPEDTATYYCNVMQA
WGQGTQVTVSSEPKTPKPQT

This protein sequence is encoded by the cDNA shown in SEQ ID NO: 87 below.

(SEQ ID NO: 87)
GAGGTGCAGCTGGTGGAGTCTGGGGGAGGATTGGTGCAGGCGGGGGGCTC
TCTGAAACTCTCCTGTGCAGCCTCCGGACGCACCCTCACTTATTATACTG
CCTGGTTCCGCCAGGCTCCAGGGAAGGAGCGTGAGTTTGTAGCATCGCTA
GGGTGGAGTGGCGATGTCACATACTATGCAGACTCCGTGAAGGGCCGATT
CACCATCTCCGGCGACAACGCCAAGAACACGGTATATCTGCAAATGAACA
GCCTGAAACCCGAGGACACGGCCACTTATTACTGTAATGTCATGCAGGCT
TGGGGTCAGGGGACCCAGGTCACCGTCTCCTCAGAACCCAAGACACCAAA
ACCACAAACT

a-CD45-h-VHH-08

(SEQ ID NO: 88)
EVQLLESGGGLVQAGDSLRLSCAASGLTNPERRLAWFRQAPGKEREFVAS
IRWSGGPNTHYGDSVKGRFTISRDNGKNTVALQMNNLKPEDTAVYFCAAA
VRLTAPLNFDTSYDYWGQGTQVTISSEPKTPKPQT

This protein sequence is encoded by the cDNA shown in SEQ ID NO: 89 below.

(SEQ ID NO: 89)
GAGGTGCAGCTGCTGGAGTCTGGGGGAGGATTGGTGCAGGCTGGGGACTC
TCTGAGACTCTCCTGTGCAGCTTCTGGACTGACCAACCCTGAAAGACGCT
TGGCCTGGTTCCGCCAGGCTCCAGGGAAGGAGCGTGAGTTTGTAGCGTCC
ATTCGCTGGAGTGGTGGTCCCAACACACACTATGGGGACTCCGTGAAGGG
CCGATTCACCATCTCCAGAGACAACGGCAAGAACACGGTGGCTCTACAAA
TGAACAACCTGAAACCTGAGGACACGGCCGTTTATTTCTGTGCAGCGGCT
GTGCGTCTAACTGCGCCTCTCAATTTTGACACCTCGTATGACTACTGGGG
CCAGGGGACCCAGGTCACCATCTCCTCAGAACCCAAGACACCAAAACCAC
AAACT

a-CD45-h-VHH-09

(SEQ ID NO: 90)
EVQLLESGGGLVQAGGSLRLSCAASGRTLTFYTGWFRQAPGKEREFVASI
RWSGGHTYYADSVKGRFTISGDNAKNTVYLQMNSLKPEDTAIYYCAALRS
WTTTPQREDLYDVWGQGTQVTVSSEPKTPKPQT

This protein sequence is encoded by the cDNA shown in SEQ ID NO: 91 below.

(SEQ ID NO: 91)
GAGGTGCAGCTGCTGGAGTCTGGGGGAGGATTGGTGCAGGCGGGGGGCTC
TCTGAGACTCTCCTGTGCAGCCTCCGGACGCACCCTCACTTTTTATACTG
GCTGGTTCCGCCAGGCTCCAGGGAAGGAGCGTGAGTTTGTAGCATCCATT
AGGTGGAGTGGCGGTCACACATACTATGCAGACTCCGTGAAGGGCCGATT
CACCATCTCCGGAGACAACGCCAAGAACACGGTGTATCTACAAATGAACA
GCCTGAAACCCGAGGACACGGCCATTTATTACTGCGCAGCACTTAGATCT
TGGACTACTACACCTCAGAGGGAGGACCTCTATGATGTCTGGGGCCAGGG
GACCCAGGTCACCGTCTCCTCAGAACCCAAGACACCAAAACCACAAACT

a-CD45-h-VHH-10

(SEQ ID NO: 92)
EVQLQESGGGLVQAGGSLRLSCAASGRTLTFYTGWFRQAPGKEREFVASI
RWSGGNTYYADSVKGRFTITGDNAKNTVYLQMNSLKPEDTAIYYCAALRS
WTTTPQREVLYDNWGHGTQVTVSSAHHSEDPIS

This protein sequence is encoded by the cDNA shown in SEQ ID NO: 93 below.

(SEQ ID NO: 93)
GAGGTGCAGCTGCAGGAGTCTGGGGGAGGATTGGTG
CAGGCGGGGGGCTCTCTGAGACTCTCCTGTGCAGCC
TCCGGACGCACCCTCACTTTTTATACTGGCTGGTTC
CGCCAGGCTCCAGGGAAGGAGCGTGAGTTTGTAGCA
TCTATTAGGTGGAGTGGCGGTAACACATACTATGCA
GACTCCGTGAAGGGCCGATTCACCATCACCGGAGAC
AACGCCAAGAACACGGTGTATCTGCAAATGAACAGC
CTGAAACCCGAGGACACGGCCATTTATTACTGCGCA
GCACTTAGATCTTGGACTACTACACCTCAGAGGGAG
GTCCTCTATGACAACTGGGGCCACGGGACCCAGGTC
ACCGTCTCCTCAGCGCACCACAGCGAAGACCCTATT
AGT

a-CD45-h-VHH-11

(SEQ ID NO: 94)
EVQLEESGGGLVQAGDSLRLSCACSERAYRNRLLGW
FRQAPGKEREFVANIRPIDSASDYAGSVKGRFTISR
DIAKRTVYLQMNSLKPEDTAVYYCASTYMFDSVRED
EYDYWGQGTQVTVSSEPKTPKPQT

This protein sequence is encoded by the cDNA shown in SEQ ID NO: 95 below.

(SEQ ID NO: 95)
GAGGTGCAGCTGGAGGAGTCTGGGGGAGGATTGGTG
CAGGCTGGGGACTCTCTGAGACTCTCCTGTGCTTGC
TCTGAACGCGCCTATAGGAACCGTCTTCTTGGCTGG
TTCCGCCAGGCTCCAGGAAAGGAGCGTGAATTTGTA
GCAAATATCAGACCCATTGATAGCGCCTCCGATTAT
GCAGGCTCCGTGAAGGGCCGATTCACCATCTCTAGA
GACATCGCCAAGAGAACGGTGTATCTGCAAATGAAC
AGCCTGAAACCTGAGGACACGGCCGTTTATTATTGT
GCGTCCACATACATGTTCGATAGTGTCCGGGAGGAT
GAATATGACTACTGGGGCCAGGGAACCCAGGTCACC
GTCTCCTCAGAACCCAAGACACCAAAACCACAAACT

a-CD45-h-VHH-12

(SEQ ID NO: 96)
EVQLVESGGGLVQAGGSLRLSCVVSGRTLTFYTGWF
RQAPGKEREFVASIRWSGGNTYYADSVKGRFTITRD
NARSTLHLQMNSLKPEDTAVYYCVKGNGLTSTRASD
YWGQGTQVTISSEPKTPKPQT

This protein sequence is encoded by the cDNA shown in SEQ ID NO: 97 below.

(SEQ ID NO: 97)
GAGGTGCAGCTGGTGGAGTCTGGGGGAGGATTGGTG
CAGGCTGGGGGCTCTCTGAGACTCTCGTGTGTAGTC
TCTGGACGCACCCTCACTTTTTATACTGGCTGGTTC
CGCCAGGCTCCAGGGAAGGAGCGTGAGTTTGTAGCA
TCTATTAGGTGGAGTGGCGGTAACACATACTATGCA
GACTCCGTGAAGGGCCGATTCACCATCACCAGAGAT
AACGCCAGGAGCACGCTGCATCTTCAAATGAACAGC
CTGAAACCTGAGGACACGGCCGTGTATTACTGTGTA
AAGGGGAACGGACTTACTTCTACTCGCGCGAGTGAC
TACTGGGGCCAGGGGACCCAGGTCACCATCTCCTCA
GAACCCAAGACACCAAAACCACAAACT

a-CD45-h-VHH-13

(SEQ ID NO: 98)
EVQLLESGGGLVQAGGSLRLSCVASGRGFSRYDMGW
FRQASGKEREFVAAISWSNSTTAYADSVKGRFAISR
DNNKNMVYLQMNSLKPEDTAVYYCAARVRGSTGDFG
SWGQGTQVTVSSEPKTPKPQT

This protein sequence is encoded by the cDNA shown in SEQ ID NO: 99 below.

(SEQ ID NO: 99)
GAGGTGCAGCTGCTGGAGTCTGGGGGAGGCTTGGTG
CAGGCTGGGGGCTCTCTGAGACTCTCCTGTGTAGCC
TCTGGACGGGGCTTCAGTAGGTATGACATGGGCTGG
TTCCGCCAGGCTTCAGGGAAGGAGCGTGAGTTTGTA
GCAGCAATTAGCTGGAGTAATAGTACCACGGCCTAT
GCAGACTCCGTGAAGGGCCGATTCGCCATCTCAAGA
GACAACAACAAGAATATGGTGTATCTGCAAATGAAC
AGCCTGAAACCGGAGGACACGGCCGTGTATTACTGT
GCAGCGAGGGTACGCGGCAGCACAGGGGACTTTGGT
TCCTGGGGCCAGGGGACCCAGGTCACCGTCTCCTCG
GAACCCAAGACACCAAAACCACAAACT

a-CD45-h-VHH-14

(SEQ ID NO: 100)
EVQLVESGGGLVQAGGSLSLSCAASGRTFSTGAMGW
FRQAPGKEREFLARITLIGHGTYYADALKGRFTISR
DHAKNTVYLQMNSLKPEDTAVYYCVARDSPCVGNCW
YENAGDYNYWGQGTQVTVSSEPKTPKPQT

This protein sequence is encoded by the cDNA shown in SEQ ID NO: 101 below.

(SEQ ID NO: 101)
GAGGTGCAGCTGGTGGAGTCTGGGGGAGGATTGGTG
CAGGCTGGGGGCTCTCTGAGTCTCTCCTGTGCAGCC
TCTGGACGCACCTTCAGTACCGGTGCCATGGGCTGG
TTCCGCCAGGCTCCAGGGAAGGAGCGTGAGTTTCTG
GCACGAATTACTCTGATTGGCCACGGCACATACTAT
GCAGATGCCTTGAAGGGCCGATTCACCATTTCCAGA
GACCACGCTAAGAACACGGTGTATCTGCAAATGAAC
AGCCTGAAACCTGAGGACACGGCCGTATATTACTGT
GTAGCGCGAGACAGCCCGTGCGTGGGTAATTGTTGG
TACGAGAATGCGGGCGACTATAATTACTGGGGCCAG
GGGACCCAGGTCACCGTCTCCTCAGAACCCAAGACA
CCAAAACCACAAACT

a-CD45-h-VHH-15

(SEQ ID NO: 102)
EVQLLESGGGLVQAGGSLRLSCVSSGDSISGVVVRW
YRQVPGKQREWIGGIGTSDNPEYADSVWGRFVLSRD
NAGSRVNLQMNNLKLEDTATYYCNAVHKWGPGTQVT
VSSEPKTPKPQ

This protein sequence is encoded by the cDNA shown in SEQ ID NO: 103 below.

(SEQ ID NO: 103)
GAGGTGCAGCTGCTGGAGTCTGGGGGAGGCCTGGTG
CAGGCTGGGGGGTCTCTGAGACTCTCCTGTGTAAGT
TCTGGAGACAGTATCAGTGGAGTGGTCGTCCGTTGG
TACCGCCAGGTTCCAGGGAAGCAGCGCGAGTGGATC
GGAGGTATTGGTACTAGTGATAACCCAGAATATGCG
GACTCCGTCTGGGGCCGATTCGTCCTCTCCAGAGAC
AATGCCGGGAGCCGCGTAAATCTGCAAATGAACAAC
CTGAAACTTGAGGACACGGCCACCTATTACTGCAAT
GCAGTGCACAAATGGGGCCCGGGTACCCAGGTCACC
GTCTCTTCTGAACCCAAGACACCAAAACCACAAAC

a-CD45-h-VHH-16

(SEQ ID NO: 104)
EVQLLESGGGLVQPGGSLRLSCAASGFTFSNAVMSW
VRQAPGKEREFVASIRWSGGNTYYADSVKGRFTITG
DNAKNTVYLQMNSLKPEDTAIYYCAALRSWTTTPQR
EVLYDNWGHGTQVTVSSEPKTPKPQT

This protein sequence is encoded by the cDNA shown in SEQ ID NO: 105 below.

(SEQ ID NO: 105)
GAGGTGCAGCTGCTGGAGTCTGGGGGAGGCTTGGTG
CAGCCTGGGGGGTCTCTGAGACTCTCCTGTGCAGCC
TCTGGATTCACCTTCAGTAACGCCGTCATGAGCTGG
GTCCGCCAGGCTCCAGGGAAGGAGCGTGAGTTTGTA
GCATCTATTAGGTGGAGTGGCGGTAACACATACTAT
GCAGACTCCGTGAAGGGCCGATTCACCATCACCGGA
GACAACGCCAAGAACACGGTGTATCTGCAAATGAAC
AGCCTGAAACCCGAGGACACGGCCATTTATTACTGC
GCAGCACTTAGATCTTGGACTACTACACCTCAGAGG
GAGGTCCTCTATGACAACTGGGGCCACGGGACCCAG
GTCACCGTCTCCTCAGAACCCAAGACACCAAAACCA
CAAACT

a-CD45-h-VHH-17

(SEQ ID NO: 106)
EVQLEESGGGLVQAGGSLRLSCAASGRTFSSYRLGW
FRQAPGKEREFVAGWSGGSTYYADSVKGRFTISRDN
AKNTVYLQMNSLKPEDTAVYYCVKGNGLTSTRASDY
WGQGTQVTVSSEPKTPKPQT

This protein sequence is encoded by the cDNA shown in SEQ ID NO: 107 below.

(SEQ ID NO: 107)
GAGGTGCAGCTGGAGGAGTCTGGGGGAGGATTGGTG
CAGGCTGGGGGCTCTCTGAGACTCTCCTGTGCAGCC
TCTGGACGCACCTTCAGTAGCTATCGACTGGGCTGG
TTCCGCCAGGCTCCAGGGAAGGAGCGTGAGTTTGTA
GCAGGCTGGAGTGGTGGTAGCACATACTATGCAGAC
TCCGTGAAGGGCCGATTCACCATCTCCAGAGACAAC
GCCAAGAACACGGTGTATCTGCAAATGAACAGCCTC
AAACCTGAGGACACGGCCGTGTATTACTGTGTAAAG
GGGAACGGACTTACTTCTACTCGCGCGAGTGACTAC
TGGGGCCAGGGAACCCAGGTCACCGTCTCCTCAGAA
CCCAAGACACCAAAACCACAAACT

a-CD45-h-VHH-18

(SEQ ID NO: 108)
EVQLVESGGGLVQAGDSLRLSCAASGLTNPERRLAW
FRQAPGKEREFVASIRWSGGPNTHYGDSVKGRFTIS
RDNGKNTVALQMNNLKPEDTAVYFCAAAVRLTAPLN
FDTSYDYWGQGTQVTISSEPKTPKPQT

This protein sequence is encoded by the cDNA shown in SEQ ID NO: 109 below.

(SEQ ID NO: 109)
GAGGTGCAGCTGGTGGAGTCTGGGGGAGGATTGGTG
CAGGCTGGGGACTCTCTGAGACTCTCCTGTGCAGCT
TCTGGACTGACCAACCCTGAAAGACGCTTGGCCTGG
TTCCGCCAGGCTCCAGGGAAGGAGCGTGAGTTTGTA
GCGTCCATTCGCTGGAGTGGTGGTCCCAACACACAC
TATGGAGACTCCGTGAAGGGCCGATTCACCATCTCC
AGAGACAACGGCAAGAACACGGTGGCTCTACAAATG
AACAACCTGAAACCTGAGGACACGGCCGTTTATTTC
TGTGCAGCGGCTGTGCGTCTAACTGCGCCTCTCAAT
TTTGACACCTCGTATGACTACTGGGGCCAGGGGACC
CAGGTCACCATCTCCTCAGAACCCAAGACACCAAAA
CCACAAACT

a-CD45-h-VHH-19

(SEQ ID NO: 110)
EVQLLESGGGLVQPGGSLRLSCAASGFTFSNSVMSW
VRQAPGKGPERVSIIGSVGGTSGVTSYADSVKGRFT
ITRDNARSTLHLQMNSLKPEDTAVYYCVKGNGLTST
RASDYWGQGTQVTVSSEPKTPKPQT

This protein sequence is encoded by the cDNA shown in SEQ ID NO: 111 below.

(SEQ ID NO: 111)
GAGGTGCAGCTGCTGGAGTCTGGGGGAGGCTTGGTG
CAGCCTGGGGGGTCTCTGAGACTCTCCTGTGCAGCC
TCTGGATTCACCTTCAGTAACAGCGTCATGAGCTGG
GTCCGCCAGGCTCCAGGAAAGGGGCCCGAGCGGGTC
TCAATTATCGGCAGTGTCGGAGGTACCTCAGGTGTC
ACAAGTTATGCAGACTCCGTGAAGGGCCGATTCACC
ATCACCAGAGATAACGCCAGGAGCACGCTGCATCTG
CAAATGAACAGCCTGAAACCTGAGGACACGGCCGTG
TATTACTGTGTAAAGGGGAACGGACTTACTTCTACT
CGCGCGAGTGACTACTGGGGCCAGGGAACCCAGGTC
ACCGTCTCCTCAGAACCCAAGACACCAAAACCACAA
ACT

a-CD45-h-VHH-20

(SEQ ID NO: 112)
EVQLEESGGGLVQAGDSLRLSCVVSGSISSIYAMGW
VREDPGKERVVVAGINSGAIRWYADSVKGRFTISGD
NAKNTVYLQMNSLKPEDTAVYFCAAAVRLTAPLNFD
TSYDYWGQGTQVTVSSEPKTPKPQT

This protein sequence is encoded by the cDNA shown in SEQ ID NO: 113 below.

(SEQ ID NO: 113)
GAGGTGCAGCTGGAGGAGTCTGGGGGAGGATTGGTGC
AGGCTGGGGACTCTCTGAGACTCTCCTGTGTAGTCT
CTGGAAGCATCTCCAGTATCTATGCCATGGGATGGG
TCCGCGAGGATCCAGGGAAGGAGCGCGTAGTGGTTG
CAGGTATTAATAGCGGAGCTATCAGATGGTACGCAG
ACTCTGTGAAGGGCCGATTCACCATCTCCGGAGACA
ACGCCAAGAACACGGTGTATCTGCAAATGAACAGCC
TGAAACCTGAGGACACGGCCGTTTATTTCTGTGCAG
CGGCTGTGCGTCTAACTGCGCCTCTCAATTTTGACA
CCTCGTATGACTACTGGGGCCAGGGGACCCAGGTCA
CCGTCTCCTCAGAACCCAAGACACCAAAACCACAAA
CT

a-CD45-h-VHH-21

(SEQ ID NO: 114)
EVQLLESGGGLVQPGGSLRLSCAASGFTFSNYAMSW
VRQAPGKGPERVSIIGSVGGTSGVTSYADSVKGRFT
ITRDNARSTLHLQMNSLKPEDTAVYYCVKGNGLTST
RASDYWGQGTQVTVSAEPKTPKPQT

This protein sequence is encoded by the cDNA shown in SEQ ID NO: 115 below.

(SEQ ID NO: 115)
GAGGTGCAGCTGCTGGAGTCTGGGGGAGGCTTGGTG
CAGCCTGGGGGGTCTCTGAGACTCTCCTGTGCAGCC
TCTGGATTCACCTTCAGTAACTACGCCATGAGCTGG
GTCCGCCAGGCTCCAGGAAAGGGGCCCGAGCGGGTC
TCAATTATCGGCAGTGTCGGAGGTACCTCAGGTGTC
ACAAGTTATGCAGACTCCGTGAAGGGCCGATTCACC
ATCACCAGAGATAACGCCAGGAGCACGCTGCATCTG
CAAATGAACAGCCTGAAACCTGAGGACACGGCCGTG
TATTACTGTGTAAAGGGGAACGGACTTACTTCTACT
CGCGCGAGTGACTACTGGGGCCAGGGAACCCAGGTC
ACCGTCTCCGCAGAACCCAAGACACCAAAACCACAA
ACT

a-CD45-h-VHH-22

(SEQ ID NO: 116)
EVQLEESGGGLVQPGGSLRLSCAASGFTFSNAVMSW
VRQAPGKGPERVSIIGSVGGTSGVTSYADSVKGRFT
ITRDNARSTLHLQMNSLKPEDTAVYYCVKGNGLTST
RASDYWGQGTQVTVSSEPKTPKPQT

This protein sequence is encoded by the cDNA shown in SEQ ID NO: 117 below.

(SEQ ID NO: 117)
GAGGTGCAGCTGGAGGAGTCTGGGGGAGGCTTGGTG
CAGCCTGGGGGGTCTCTGAGACTCTCCTGTGCAGCC
TCTGGATTCACCTTCAGTAACGCCGTCATGAGCTGG
GTCCGCCAGGCTCCAGGAAAGGGGCCCGAGCGGGTC
TCAATTATCGGCAGTGTCGGAGGTACCTCAGGTGTC
ACAAGTTATGCAGACTCCGTGAAGGGCCGATTCACC
ATCACCAGAGATAACGCCAGGAGCACGCTGCATCTG
CAAATGAACAGCCTGAAACCTGAGGACACGGCCGTG
TATTACTGTGTAAAGGGGAACGGACTTACTTCTACT
CGCGCGAGTGACTACTGGGGCCAGGGAACCCAGGTC
ACCGTCTCCTCAGAACCCAAGACACCAAAACCACAA
ACT

a-CD45-h-VHH-23

(SEQ ID NO: 118)
EVQLEESGGGLVQPGGSLRLSCAASGFTFSNQVMSW
VRQAPGKGPERVSVIGSVGGATGATSYADSVRGRFT
ISRDNARSTLHLQMNSLKPEDTAVYYCVKGNGLTST
RASDYWGQGTQVTVSSEPKTPKPQT

This protein sequence is encoded by the cDNA shown in SEQ ID NO: 119 below.

(SEQ ID NO: 119)
GAGGTGCAGCTGGAGGAGTCTGGGGGAGGCTTGGTG
CAGCCTGGGGGGTCTCTGAGACTCTCCTGTGCAGCC
TCTGGATTCACCTTCAGTAACCAAGTCATGAGCTGG
GTCCGCCAGGCTCCAGGAAAGGGGCCCGAGCGGGTC
TCAGTTATCGGCAGTGTCGGAGGTGCCACAGGTGCC
ACAAGTTATGCAGACTCCGTGAGGGGCCGATTCACC
ATCTCCAGAGATAACGCCAGGAGCACGCTGCATCTG
CAAATGAACAGCCTCAAACCTGAGGACACGGCCGTG
TATTACTGTGTAAAGGGGAACGGACTTACTTCTACT
CGCGCGAGTGACTACTGGGGCCAGGGAACCCAGGTC
ACCGTCTCCTCAGAACCCAAGACACCAAAACCACAA
ACT

a-CD45-h-VHH-24

(SEQ ID NO: 120)
EVQLEESGGGLVETGDSLRLSCSASGGGFSFNAIGW
YRQGPGKGRELVAAGTSGSTTYYAPSVKGRFIFSRD
SAKNTVYLQMNNLNPEDTAIYYCATPALGQMEYDVV
SGDGLAHWGKGTLVIVSSAHHSEDPNS

This protein sequence is encoded by the cDNA shown in SEQ ID NO: 121 below.

(SEQ ID NO: 121)
GAGGTGCAGCTGGAGGAGTCTGGGGGAGGCCTGGTG
GAGACTGGGGATTCTCTGAGACTCTCCTGCTCTGCC
TCTGGGGGCGGTTTTAGTTTCAATGCCATAGGCTGG
TACCGGCAGGGGCCGGGAAAGGGGCGCGAATTGGTC
GCAGCAGGTACTAGTGGAAGTACCACATATTACGCG
CCCTCTGTGAAGGGCCGATTCATCTTCTCCAGAGAC
AGTGCCAAAAACACCGTCTATCTGCAAATGAACAAC
CTGAACCCTGAAGACACGGCCATCTATTACTGTGCC
ACACCGGCACTTGGACAAATGGAGTATGACGTAGTG
AGCGGCGACGGCTTGGCCCACTGGGGCAAAGGGACC
CTGGTCATCGTCTCTTCAGCGCACCACAGCGAAGAC
CCTAATAGT

a-CD45-h-VHH-25

(SEQ ID NO: 122)
EVQLVESGGGLVQPGGSLRLSCAASGFTFSNQVMSW
VRQAPGKGPERVSVIGSVGGATGATSYADSVRGRFT
ISRDNARSTLHLQMNSLKPEDTAVYYCVKGNGLTST
RASDYWGQGTQVTVSSEPKTPKPQT

This protein sequence is encoded by the cDNA shown in SEQ ID NO: 123 below.

(SEQ ID NO: 123)
GAGGTGCAGCTGGTGGAGTCTGGGGGAGGCTTGGTGCAGCCTGGGGGGTC
TCTGAGACTCTCCTGTGCAGCCTCTGGATTCACCTTCAGTAACCAAGTCA
TGAGCTGGGTCCGCCAGGCTCCAGGAAAGGGGCCCGAGCGGGTCTCAGTT
ATCGGCAGTGTCGGAGGTGCCACAGGTGCCACAAGTTATGCAGACTCCGT
GAGGGGCCGATTCACCATCTCCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-26

(SEQ ID NO: 124)
EVQLVESGGGLVQPGGSLRLSCAASGFTFSNHVMSWVRQAPGKGPERVSI
IGSVGGTSGVTSYADSVKGRFTITRDNARSTLHLQMNSLKPEDTAVYYCV
KGNGLTSTRASDYWGQGTQVTVSSEPKTPKPQT

This protein sequence is encoded by the cDNA shown in SEQ ID NO: 125 below.

(SEQ ID NO: 125)
GAGGTGCAGCTGGTGGAGTCTGGGGGAGGCTTGGTGCAGCCTGGGGGGTC
TCTGAGACTCTCCTGTGCAGCCTCTGGATTCACCTTCAGTAACCACGTCA
TGAGCTGGGTCCGCCAGGCTCCAGGAAAGGGGCCCGAGCGGGTCTCAATT
ATCGGCAGTGTCGGAGGTACCTCAGGTGTCACAAGTTATGCAGACTCCGT
GAAGGGCCGATTCACCATCACCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-27

(SEQ ID NO: 126)
EVQLVESGGGLVQPGGSLRLSCATSGLTNPERRLAWFRQEPGKEREFVAS
IRWSGGPNTHYGDSVKGRFTISRDNGKNTVALQMNNLKPEDTAVYYCAAR
DSPCVGNCWYENAGDYEYWGQGTQVTVSSEPKTPKPQT

This protein sequence is encoded by the cDNA shown in SEQ ID NO: 127 below.

(SEQ ID NO: 127)
GAGGTGCAGCTGGTGGAGTCTGGGGGAGGCTTGGTGCAGCCTGGGGGGTC
TCTGAGACTCTCCTGTGCAACTTCTGGACTGACCAACCCTGAAAGACGCT
TGGCCTGGTTCCGCCAGGAACCAGGGAAGGAGCGTGAGTTTGTAGCGTCC
ATTCGCTGGAGTGGTGGTCCCAACACACACTATGGGGACTCCGTGAAGGG
CCGATTCACCATCTCCAGAGACAACGGCAAGAACACGGTGGCTCTACAAA
TGAACAACCTGAAACCTGAGGACACGGCCGTTTATTACTGTGCAGCGCGA
GACAGCCCGTGCGTGGGTAATTGTTGGTACGAGAATGCGGGCGACTATGA
GTACTGGGGCCAGGGGACCCAGGTCACCGTCTCCTCAGAACCCAAGACAC
CAAAACCACAAACT

a-CD45-h-VHH-28

(SEQ ID NO: 128)
EVQLVESGGGLVQAGGSLSLSCAASGRTFSTGAMGWFRQAPGKEREFLAR
ITLIGHGTYYADALKGRFTISRDHAKNTVYLQMNSLKPEDTAVYYCVARD
SPCVGNCWYENAGDYNYWGQGTQVTVSSEPKTPKPQT

This protein sequence is encoded by the cDNA shown in SEQ ID NO: 129 below.

(SEQ ID NO: 129)
GAGGTGCAGCTGGTGGAGTCTGGGGGAGGATTGGTGCAGGCTGGGGGCTC
TCTGAGTCTCTCCTGTGCAGCCTCTGGACGCACCTTCAGTACCGGTGCCA
TGGGCTGGTTCCGCCAGGCTCCAGGGAAGGAGCGTGAGTTTCTGGCACGA
ATTACTCTGATTGGCCACGGCACATACTATGCAGATGCCTTGAAGGGCCG
ATTCACCATTTCCAGAGACCACGCTAAGAACACGGTGTATCTGCAAATGA
ACAGCCTGAAACCTGAGGACACGGCCGTATATTACTGTGTAGCGCGAGAC
AGCCCGTGCGTGGGTAATTGTTGGTACGAGAATGCGGGCGACTATAATTA
CTGGGGCCAGGGGACCCAGGTCACCGTCTCCTCAGAACCCAAGACACCAA
AACCACAAACT

a-CD45-h-VHH-29

(SEQ ID NO: 130)
EVQLVESGGGLVQAGDSLTLSCAASERAYRNRLLGWFRQVPGKEREFVAW
IRPIDSSTNYADSVKGRFTITRDNARSTLHLQMNSLKPEDTAVYYCVKGN
GLTSTRASDYWGQGTQVTVSSEPKTPKPQT

This protein sequence is encoded by the cDNA shown in SEQ ID NO: 131 below.

(SEQ ID NO: 131)
GAGGTGCAGCTGGTGGAGTCTGGGGGAGGATTGGTGCAGGCTGGGGACTC
GCTGACACTCTCCTGTGCAGCCTCTGAACGCGCCTACAGGAACCGTCTTC
TTGGCTGGTTCCGCCAGGTTCCAGGGAAGGAGCGTGAATTTGTGGCATGG
ATCAGACCCATTGATAGCAGCACAAATTATGCAGACTCCGTGAAGGGCCG
ATTCACCATCACCAGAGATAACGCCAGGAGCACGCTGCATCTGCAAATGA
ACAGCCTGAAACCTGAGGACACGGCCGTGTATTACTGTGTAAAGGGGAAC
GGACTTACTTCTACTCGCGCGAGTGACTACTGGGGCCAGGGAACCCAGGT
CACCGTCTCCTCAGAACCCAAGACACCAAAACCACAAACT

a-CD45-h-VHH-30

(SEQ ID NO: 132)
EVQLEESGGGSVQAGGSLRLSCAASGFTFSNSVMSWVRQAPGKGPERVSI
IGSVGGTSGVTSYADSVKGRFTITRDNARSTLHLQMNSLKPEDTAVYYCV
KGNGLTSTRASDYWGQGTQVTVSSEPKTPKPQT

This protein sequence is encoded by the cDNA shown in SEQ ID NO: 133 below.

(SEQ ID NO: 133)
GAGGTGCAGCTGGAGGAGTCTGGGGGAGGATCGGTGCAGGCTGGGGGGTC
TCTGAGACTCTCCTGTGCAGCCTCTGGATTCACCTTCAGTAACTCCGTCA
TGAGCTGGGTCCGCCAGGCTCCAGGAAAGGGGCCCGAGCGGGTCTCAATT
ATCGGCAGTGTCGGAGGTACCTCAGGTGTCACAAGTTATGCAGACTCCGT
GAAGGGCCGATTCACCATCACCAGAGATAACGCCAGGAGCACGCTGCATC
TACAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-31

(SEQ ID NO: 134)
EVQLEESGGGLVQPGGSLRLSCAASGFTFSNSVMSWVRQAPGKGPERVSI
IGSVGGTSGVTSYADSVKGRFTITRDNARSTLHLQMNSLKPEDTAVYYCV
KGNGLTSTRASDYWGQGTQVTVSSEPKTPKPQT

This protein sequence is encoded by the cDNA shown in SEQ ID NO: 135 below.

(SEQ ID NO: 135)
GAGGTGCAGCTGGAGGAGTCTGGGGGAGGCTTGGTGCAGCCTGGGGGGTC
TCTGAGACTCTCCTGTGCAGCCTCTGGATTCACCTTCAGTAACAGCGTCA
TGAGCTGGGTCCGCCAGGCTCCAGGAAAGGGGCCCGAGCGGGTCTCAATT
ATCGGCAGTGTCGGAGGTACCTCAGGTGTCACAAGTTATGCAGACTCCGT
GAAGGGCCGATTCACCATCACCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-32

(SEQ ID NO: 136)
EVQLLESGGGLVQAGGSLRLSCAASGRTLTFYTGWFRQAPGKEREFVASI
RWSGGNTYYADSVKGRFTISGDNAKNTVYLQMNSLKPEDTAIYYCAALRS
WTTTPQREVLYDNWGQGTQVTVSSAHHSEDPIS

This protein sequence is encoded by the cDNA shown in SEQ ID NO: 137 below.

(SEQ ID NO: 137)
GAGGTGCAGCTGCTGGAGTCTGGGGGAGGATTGGTGCAGGCGGGGGGCTC
TCTGAGACTCTCCTGTGCAGCCTCCGGACGCACCCTCACTTTTTATACTG
GCTGGTTCCGCCAGGCTCCAGGGAAGGAGCGTGAGTTTGTAGCTTCTATT
AGGTGGAGTGGCGGTAACACATACTATGCAGACTCCGTGAAGGGCCGATT
CACCATCTCCGGAGACAACGCCAAGAACACGGTGTATCTGCAAATGAACA
GCCTGAAACCCGAGGACACGGCCATTTATTACTGCGCAGCACTTAGATCT
TGGACTACTACACCTCAGAGGGAGGTCCTCTATGACAACTGGGGCCAGGG
GACCCAGGTCACCGTCTCCTCAGCGCACCACAGCGAAGACCCTATTAGT

a-CD45-h-VHH-33

(SEQ ID NO: 138)
EVOLVESGGGLVQAGDSLRLSCAASGLTNPERRLAWFRQAPGKEREFVA
SIRWSGGPNTHYGDSVKGRFTISRDNAKNMVYLQMDNIKPEDTARYFCA
SSYTFSSVREDDYDYWGQGTQVTVLSAHHSEDPIS

This protein sequence is encoded by the cDNA shown in SEQ ID NO: 139 below.

(SEQ ID NO: 139)
GAGGTGCAGCTGGTGGAGTCTGGGGGAGGATTGGTGCAGGCTGGGGACT
CTCTGAGACTCTCCTGTGCAGCTTCTGGACTGACCAACCCTGAAAGACG
CTTGGCCTGGTTCCGCCAGGCTCCAGGGAAGGAGCGTGAGTTTGTAGCG
TCCATTCGCTGGAGTGGTGGTCCCAACACACACTATGGAGACTCCGTGA
AGGGCCGATTTACCATCTCTCGAGATAACGCCAAGAACATGGTGTACCT
GCAAATGGACAACATAAAACCTGAAGACACGGCCCGTTATTTCTGTGCG
TCCTCATACACCTTCAGCAGTGTCCGGGAGGATGACTATGACTACTGGG
GCCAGGGGACCCAGGTCACCGTCTTGTCAGCGCACCACAGCGAAGACCC
TATTAGT

a-CD45-h-VHH-34

(SEQ ID NO: 140)
EVQLVESGGGLVQAGGSLRLSCAASGRTVSRYDMGWFRQAPGAERVVVA
ISWSGGSTYYVDSVKGRFTMSRDNSKNTVYLQMNSLKPEDTAVYYCAVR
TERSSLDFHSWGQGTQVTVSSEPKTPKPQT

This protein sequence is encoded by the cDNA shown in SEQ ID NO: 141 below.

(SEQ ID NO: 141)
GAGGTGCAGCTGGTGGAGTCTGGGGGAGGATTGGTGCAGGCTGGGGGCT
CTCTGAGACTCTCCTGTGCAGCCTCTGGACGCACCGTCAGTAGATATGA
CATGGGCTGGTTCCGCCAGGCTCCAGGGGCGGAGCGTGTCGTTGTAGCT
ATTAGCTGGAGCGGTGGTAGTACATACTATGTAGACTCCGTGAAGGGCC
GATTCACCATGTCCAGAGACAACAGCAAGAACACGGTATATCTGCAAAT
GAACAGCCTGAAACCTGAGGACACGGCCGTTTATTACTGTGCAGTCAGA
ACCGAACGCTCCAGTCTTGACTTTCATTCCTGGGGCCAGGGGACCCAGG
TCACCGTCTCCTCGGAACCCAAGACACCAAAACCACAAACT

a-CD45-h-VHH-35

(SEQ ID NO: 142)
EVQLEESGGGLVQAGDSLRLSCAASERAYRNRLLGWFRQVPGKEREFVA
WIRPIDSSTNYADSVKGRFTISRDNDKNTVYLQMDNMKPEDTALYYCAS
TYYYSSIREDDYDYWGQGTQVTVSSEPKTPKPQT

This protein sequence is encoded by the cDNA shown in SEQ ID NO: 143 below.

(SEQ ID NO: 143)
GAGGTGCAGCTGGAGGAGTCTGGGGGAGGATTGGTGCAGGCTGGGGACT
CTCTGAGACTCTCCTGTGCAGCCTCTGAACGCGCCTACAGGAACCGTCT
TCTTGGCTGGTTCCGCCAGGTTCCAGGGAAGGAGCGTGAATTTGTGGCA
TGGATCAGACCCATTGATAGCAGCACAAATTATGCAGACTCCGTGAAGG
GCCGATTCACCATCTCTAGAGATAACGACAAGAACACGGTGTATTTGCA
AATGGACAATATGAAACCTGAGGACACGGCCCTCTATTATTGTGCGTCC
ACATACTACTACAGTAGTATCCGGGAGGATGACTATGACTACTGGGGCC
AGGGGACCCAGGTCACCGTCTCCTCAGAACCCAAGACACCAAAACCACA
AACT

a-CD45-h-VHH-36

(SEQ ID NO: 144)
EVOLVESGGGLVQAGGSLRLSCAASGRAFSNRALGWFRQAPGKEREFVA
WIRGIGSSTNYAGSVQGRFTISRDNAKNTLYLQMDKLKPEDTAVYYCAS
TYMFDSVREDEYDYWGQGTQVTVSSEPKTPKPQT

This protein sequence is encoded by the cDNA shown in SEQ ID NO: 145 below.

(SEQ ID NO: 145)
GAGGTGCAGCTGGTGGAGTCTGGGGGAGGATTGGTGCAGGCTGGGGGCT
CTCTGAGACTCTCCTGTGCAGCCTCTGGACGCGCCTTCAGTAACCGTGC
ACTTGGCTGGTTCCGCCAGGCTCCAGGGAAGGAGCGTGAGTTTGTAGCG
TGGATTAGAGGCATCGGTAGCAGCACAAATTATGCAGGCTCCGTACAGG
GCCGATTCACCATCTCCAGAGACAACGCCAAGAACACGCTGTATCTGCA
GATGGACAAGCTGAAACCTGAGGACACGGCCGTTTATTATTGTGCGTCC
ACATACATGTTCGATAGTGTGCGGGAGGATGAATATGACTACTGGGGCC
AGGGGACCCAGGTCACCGTCTCCTCAGAACCCAAGACACCAAAACCACA
AACT

a-CD45-h-VHH-37

(SEQ ID NO: 146)
EVQLQESGGGLLQTGDSLRLACEASEIVVENYVMAWFRQAPGKEREWLA
RIIWNTGGTHLQEFVKGRLTISRDIAKKTVYLQMNSLKPEDTAVYYCAG
GSFDAIADPFSARRYGFWGQGTQVTVSSEPKTPKPQT

This protein sequence is encoded by the cDNA shown in SEQ ID NO: 147 below.

(SEQ ID NO: 147)
GAGGTGCAGCTGCAGGAGTCTGGGGGAGGATTGCTGCAGACTGGGGACT
CACTGAGACTCGCCTGTGAAGCCTCTGAAATCGTCGTCGAAAATTATGT
CATGGCCTGGTTCCGCCAGGCTCCAGGGAAGGAGCGTGAGTGGCTAGCG
CGTATTATTTGGAATACCGGTGGCACACATCTTCAAGAATTTGTGAAGG
GCCGACTCACCATCTCTAGAGACATCGCCAAGAAAACGGTGTATCTGCA
AATGAACAGCCTGAAACCTGAGGACACGGCCGTTTATTACTGTGCCGGT
GGAAGTTTTGACGCTATAGCCGATCCCTTCTCGGCCCGCCGGTATGGGT
TCTGGGGCCAGGGGACCCAGGTCACCGTCTCCTCAGAACCCAAGACACC
AAAACCACAAACT

a-CD45-h-VHH-38

(SEQ ID NO: 148)
EVQLQESGGGLVQAGGSLRLSCVSSGDSISGVVVRWYRQVPGKQREWIG
GIGTSDNPEYADSVWGRFVLSRDNAGSRVNLQMNNLKLEDTATYYCNAV
HKWGPGTQVTVSSEPKTPKPQT

This protein sequence is encoded by the cDNA shown in SEQ ID NO: 149 below.

(SEQ ID NO: 149)
GAGGTGCAGCTGCAGGAGTCTGGGGGAGGCCTGGTGCAGGCTGGGGGGT
CTCTGAGACTCTCCTGTGTAAGTTCTGGAGACAGTATCAGTGGAGTGGT
CGTCCGTTGGTACCGCCAGGTTCCAGGGAAGCAGCGCGAGTGGATCGGA
GGTATTGGTACTAGTGATAACCCAGAATATGCGGACTCCGTCTGGGGCC
GATTCGTCCTCTCCAGAGACAATGCCGGGAGCCGCGTAAATCTGCAAAT
GAACAACCTGAAACTTGAGGACACGGCCACCTATTACTGCAATGCAGTG
CACAAATGGGGCCCGGGTACCCAGGTCACCGTCTCTTCTGAACCCAAGA
CACCAAAACCACAAACT

a-CD45-h-VHH-39

(SEQ ID NO: 150)
EVQLVESGGGLVQPGGSLRLSCAASGFTFSNQVMSWVRQAPGKEREFVA
WIRGIGGSTHYAGSVEGRFTISRDSAKNTLYLQMDNVKPEDTAVYYCAS
TYMFDSVREDEYDYWGQGTEVTVSSAHHSEDPNS

This protein sequence is encoded by the cDNA shown in SEQ ID NO: 151 below.

(SEQ ID NO: 151)
GAGGTGCAGCTGGTGGAGTCTGGGGGAGGCTTGGTGCAGCCTGGGGGGT
CTCTGAGACTCTCCTGTGCAGCCTCTGGATTCACCTTCAGTAACCAAGT
CATGAGCTGGGTCCGCCAGGCTCCAGGGAAGGAGCGTGAGTTTGTAGCG
TGGATTAGAGGCATCGGTGGCAGCACACATTATGCAGGCTCCGTGGAGG
GCCGATTCACCATCTCCAGAGACAGCGCCAAGAACACGTTGTATCTACA
GATGGACAACGTGAAACCCGAGGACACGGCCGTTTATTATTGTGCGTCC
ACATACATGTTCGATAGTGTCCGGGAGGATGAATATGACTACTGGGGCC
AGGGGACCGAGGTCACCGTCTCCTCAGCGCACCACAGCGAAGACCCTAA
TAGT

a-CD45-h-VHH-40

(SEQ ID NO: 152)
EVQLLESGGGLVQPGGSLRLSCAASGFTFSNHVMSWVRQAPGKGPERVS
VIGSVGGATGATSYADSVRGRFTISRDSAKNTLYLQMDNVKPEDTAVYY
CASTYMFDSVREDEYDYWGQGTEVTVSSEPKTPKPQT

This protein sequence is encoded by the cDNA shown in SEQ ID NO: 153 below.

(SEQ ID NO: 153)
GAGGTGCAGCTGCTGGAGTCTGGGGGAGGCTTGGTGCAGCCTGGGGGGTC
TCTGAGACTCTCCTGTGCAGCCTCTGGATTCACCTTCAGTAACCACGTCA
TGAGTTGGGTCCGCCAGGCTCCAGGAAAGGGGCCCGAGCGGGTCTCAGTT
ATCGGCAGTGTCGGAGGTGCCACAGGTGCCACAAGTTATGCAGACTCCGT
GAGGGGCCGATTCACCATCTCCAGAGACAGCGCCAAGAACACGTTGTATC
TACAGATGGACAACGTGAAACCCGAGGACACGGCCGTTTATTATTGTGCG
TCCACATACATGTTCGATAGTGTCCGGGAGGATGAATATGACTACTGGGG
CCAGGGGACCGAGGTCACCGTCTCCTCAGAACCCAAGACACCAAAACCAC
AAACT

a-CD45-h-VHH-41

(SEQ ID NO: 154)
EVQLEESGGGLVQTGGSLRLSCAASGGTFSSYVMGWFRQAPGKEREFVAW
IRPIDSSTNYADSVKGRFTISRDDAKNSLYLQMDNMKPEDTALYYCASTY
YYSSIREDDYDYWGRGTQVTVLSAHHSEDPNS

This protein sequence is encoded by the cDNA shown in SEQ ID NO: 155 below.

(SEQ ID NO: 155)
GAGGTGCAGCTGGAGGAGTCTGGGGGAGGATTGGTACAGACCGGGGGATC
TTTGAGACTCTCCTGTGCAGCCTCTGGCGGCACCTTCAGTAGCTATGTCA
TGGGCTGGTTCCGCCAGGCTCCAGGGAAGGAGCGTGAATTTGTGGCATGG
ATCAGACCCATTGATAGCAGCACAAATTATGCAGACTCCGTGAAGGGCCG
ATTCACCATCTCTAGGGATGACGCCAAGAACTCGCTGTATCTGCAAATGG
ACAATATGAAACCTGAGGACACGGCCCTCTATTATTGTGCGTCCACATAC
TACTACAGTAGTATCCGGGAGGATGACTATGACTACTGGGGCCGGGGGAC
CCAGGTCACCGTCTTGTCAGCGCACCACAGCGAAGACCCTAATAGT

a-CD45-h-VHH-42

(SEQ ID NO: 156)
EVQLVESGGGLVQPGGSLRLSCATSGFTFSNNVMSWVRQAPGKGPERVAV
IGSVGGTTGATSYADSVKGRFTITRDNARSTLHLQMNSLKPEDTAVYYCV
KGNGLTSTRASDYWGQGTQVTVSSEPKTPKPQT

This protein sequence is encoded by the cDNA shown in SEQ ID NO: 157 below.

(SEQ ID NO: 157)
GAGGTGCAGCTGGTGGAGTCTGGGGGAGGCTTGGTGCAGCCTGGGGGGTC
TCTGAGACTCTCCTGTGCAACCTCTGGATTCACCTTCAGTAACAACGTCA
TGAGCTGGGTCCGCCAGGCTCCAGGAAAGGGGCCCGAGCGGGTCGCAGTT
ATCGGCAGTGTCGGAGGTACCACGGGTGCCACAAGTTATGCAGACTCCGT
GAAGGGCCGATTCACCATCACCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-43

(SEQ ID NO: 158)
EVQLVESGGGLVQARGSLRLSCVASGRTLTYYTGWFRQAPGKEREFVASF
AWSGGNTYYADSVKGRFTISRDNARSTLHLQMNSLKPEDTAVYYCVKGNG
LTSTRASDYWGQGTQVTVSSEPKTPKPQT

This protein sequence is encoded by the cDNA shown in SEQ ID NO: 159 below.

(SEQ ID NO: 159)
GAGGTGCAGCTGGTGGAGTCTGGGGGAGGATTGGTGCAGGCGAGGGGCTC
TCTGAGACTCTCCTGTGTAGCCTCCGGCCGCACCCTCACTTACTATACTG
GCTGGTTCCGCCAGGCTCCAGGAAAGGAGCGTGAGTTTGTAGCATCTTTT
GCGTGGAGTGGCGGTAACACATACTATGCAGACTCCGTGAAGGGCCGATT
CACCATCTCCAGAGATAACGCCAGGAGCACGCTGCATCTGCAAATGAACA
GCCTGAAACCTGAGGACACGGCCGTGTATTACTGTGTAAAGGGGAACGGA
CTTACTTCTACTCGCGCGAGTGACTACTGGGGCCAGGGAACCCAGGTCAC
CGTCTCCTCAGAACCCAAGACACCAAAACCACAAACT

a-CD45-h-VHH-44

(SEQ ID NO: 160)
EVQLVESGGGLVQPGGSLRLSCAASGFTFSNQVMSWVRQAPGKGPERVSI
IGSVGGTSGVTSYADSVKGRFTITRDNARSTLHLQMNSLKPEDTAVYYCV
KGNGLTSTRASDYWGQGTQVTVSSEPKTPKPQT

This protein sequence is encoded by the cDNA shown in SEQ ID NO: 161 below.

(SEQ ID NO: 161)
GAGGTGCAGCTGGTGGAGTCTGGGGGAGGCTTGGTGCAGCCTGGGGGGTC
TCTGAGACTCTCCTGTGCAGCCTCTGGATTCACCTTCAGTAACCAAGTCA
TGAGCTGGGTCCGCCAGGCTCCAGGAAAGGGGCCCGAGCGGGTCTCAATT
ATCGGCAGTGTCGGAGGTACCTCAGGTGTCACAAGTTATGCAGACTCCGT
GAAGGGCCGATTCACCATCACCAGAGATAACGCCAGGAGCACGCTGCATC
TGCAAATGAACAGCCTCAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-45

(SEQ ID NO: 162)
EVQLEESGGGLVQAGDSLRLSCAASGFTFSDYAMSWVRQAPGKGPERVSV
IGSVGGTTGVTSYADSVKGRFTITRDNARSTLHLQMNSLKPEDTAVYYCV
KGNGLTSTRASDYWGQGTQVTVSSEPKTPKPQT

This protein sequence is encoded by the cDNA shown in SEQ ID NO: 163 below.

(SEQ ID NO: 163)
GAGGTGCAGCTGGAGGAGTCTGGGGGAGGATTGGTGCAGGCTGGGGACTC
TCTGAGACTCTCCTGTGCAGCCTCTGGATTCACCTTCAGTGACTACGCCA
TGAGCTGGGTCCGCCAGGCTCCAGGAAAGGGGCCCGAGCGGGTCTCAGTT
ATCGGCAGTGTCGGAGGTACCACAGGTGTCACAAGTTATGCAGACTCCGT
GAAGGGCCGATTCACCATCACCAGAGATAACGCCAGGAGCACGCTGCATC
TGCAAATGAACAGTCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-46

(SEQ ID NO: 164)
EVQLEESGGGLVQPGGSLRLSCAASGFTFSNSVMSWVRQAPGKGPERVSI
IGSVGGTSGVTSYADSVKGRFTITRDNARSTLHLQMNSLKPEDTAVYYCV
KGNGLTSTRASDYWGQGTQVTVSSEPKTPKPQT

This protein sequence is encoded by the cDNA shown in SEQ ID NO: 165 below.

(SEQ ID NO: 165)
GAGGTGCAGCTGGAGGAGTCTGGGGGAGGCTTGGTGCAGCCTGGGGGGTC
TCTGAGACTCTCCTGTGCAGCCTCTGGATTCACCTTCAGTAACAGCGTCA
TGAGCTGGGTCCGCCAGGCTCCAGGAAAGGGGCCCGAGCGGGTCTCAATT
ATCGGCAGTGTCGGAGGTACCTCAGGTGTCACAAGTTATGCAGACTCCGT
GAAGGGCCGATTCACCATCACCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-47

(SEQ ID NO: 166)
EVQLLESGGGLVQAGDSLRLSCTQSGRTFSRYAIGWFRQAPGKEREFVAS
IRWSGGHTYYADSVKGRFTISKDNAKDTVYLQMNSLKPEDTAVYYCAGGS
FDAIADPFSARRYGFWGQGTQVTVSSAHHSEDPIS

This protein sequence is encoded by the cDNA shown in SEQ ID NO: 167 below.

(SEQ ID NO: 167)
GAGGTGCAGCTGCTGGAGTCTGGGGGGGGATTGGTGCAGGCAGGGGACTC
TCTGAGACTCTCCTGTACACAATCTGGACGCACCTTCAGCAGATATGCCA
TAGGCTGGTTCCGCCAGGCTCCAGGGAAGGAGCGTGAGTTTGTAGCATCC
ATTAGGTGGAGTGGCGGTCACACATACTATGCAGACTCCGTGAAGGGTCG
CTTCACCATTTCCAAGGACAACGCCAAAGACACGGTGTATCTGCAAATGA
ACAGCCTGAAACCTGAGGACACGGCCGTTTATTACTGTGCGGGTGGAAGT
TTTGACGCTATAGCCGATCCCTTCTCGGCCCGCCGGTATGGATTCTGGGG
CCAGGGGACCCAGGTCACCGTCTCCTCGGCGCACCACAGCGAAGACCCTA
TTAGT

a-CD45-h-VHH-48

(SEQ ID NO: 168)
EVQLEESGGGLVQAGGSLRLSCAASGRTLTYYTGWF
RQAPGKEREFVASFAWMGDNTYYADSVKGRFTISGD
NAKNTVYLQMNSLKPEDTATYYCAALRFWTTTPQRE
VLYDNWGQGTQVTVSSAHHSEDPIS

This protein sequence is encoded by the cDNA shown in SEQ ID NO: 169 below.

(SEQ ID NO: 169)
GAGGTGCAGCTGGAGGAGTCTGGGGGAGGATTGGTG
CAGGCGGGGGGCTCTCTGAGACTCTCCTGTGCAGCC
TCCGGACGCACCCTCACTTATTATACTGGCTGGTTC
CGCCAGGCTCCAGGGAAGGAGCGTGAGTTTGTAGCA
TCTTTTGCGTGGATGGGTGATAACACATACTACGCT
GACTCCGTGAAGGGCCGGTTCACCATCTCCGGCGAC
AACGCCAAGAACACGGTGTATCTGCAAATGAACAGC
CTGAAACCCGAGGACACGGCCACTTATTACTGCGCA
GCATTAAGATTTTGGACTACTACACCGCAGAGGGAG
GTCCTCTATGACAACTGGGGCCAGGGGACCCAGGTC
ACCGTCTCCTCAGCGCACCACAGCGAAGACCCTATT
AGT

a-CD45-h-VHH-49

(SEQ ID NO: 170)
EVQLVESGGGLVQAGDSLRLSCAASGLTNPERRLAW
FRQAPGKEREFVASIRWSGGPNTHYGDSVKGRFTIS
RDNGKNTVALQMNNLKPEDTAVYFCAAAVRLTAPLN
FDTSYDYWGQGTQVTISSEPKTPKPQT

This protein sequence is encoded by the cDNA shown in SEQ ID NO: 171 below.

(SEQ ID NO: 171)
GAGGTGCAGCTGGTGGAGTCTGGGGGAGGATTGGTG
CAGGCTGGGGACTCTCTGAGACTCTCCTGTGCAGCT
TCTGGACTGACCAACCCTGAAAGACGCTTGGCCTGG
TTCCGCCAGGCTCCAGGGAAGGAGCGTGAGTTTGTA
GCGTCCATTCGCTGGAGTGGTGGTCCCAACACACAC
TATGGAGACTCCGTGAAGGGCCGATTCACCATCTCC
AGAGACAACGGCAAGAACACGGTGGCTCTACAAATG
AACAACCTGAAACCTGAGGACACGGCCGTTTATTTC
TGTGCAGCGGCTGTGCGTCTAACTGCGCCTCTCAAT
TTTGACACCTCGTATGACTACTGGGGCCAGGGGACC
CAGGTCACCATCTCCTCAGAACCCAAGACACCAAAA
CCACAAACT

a-CD45-h-VHH-50

(SEQ ID NO: 172)
EVQLVESGGGLVQPGGSLRLSCAASGFTFSNQVMSW
VRQAPGKGPERVSVIGSVGGATGATSYADSVRGRFT
ISRDNARSTLHLQMNSLKPEDTAVYYCVKGNGLTST
RASDYWGQGTQVTVSSEPKTPKPQT

This protein sequence is encoded by the cDNA shown in SEQ ID NO: 173 below.

(SEQ ID NO: 173)
GAGGTGCAGCTGGTGGAGTCTGGGGGAGGCTTGGTG
CAGCCTGGGGGGTCTCTGAGACTCTCCTGTGCAGCC
TCTGGATTCACCTTCAGTAACCAAGTCATGAGCTGG
GTCCGCCAGGCTCCAGGAAAGGGGCCCGAGCGGGTC
TCAGTTATCGGCAGTGTCGGAGGTGCCACAGGTGCC
ACAAGTTATGCAGACTCCGTGAGGGGCCGATTCACC
ATCTCCAGAGATAACGCCAGGAGCACGCTGCATCTG
CAAATGAACAGCCTGAAACCTGAGGACACGGCCGTG
TATTACTGTGTAAAGGGGAACGGACTTACTTCTACT
CGCGCGAGTGACTACTGGGGCCAGGGAACCCAGGTC
ACCGTCTCCTCAGAACCCAAGACACCAAAACCACAA
ACT

a-CD45-h-VHH-51

(SEQ ID NO: 174)
EVQLVESGGGLVQPGGSLRLSCAASGFTFSNQVMSW
VRQAPGKGPERVSVIGSVGGATGATSYADSVRGRFT
ISRDNARSTLHLQMNSLKPEDTAVYYCVKGNGLTST
RASDYWGQGTQVTVSSEPKTPKPQT

This protein sequence is encoded by the cDNA shown in SEQ ID NO: 175 below.

(SEQ ID NO: 175)
GAGGTGCAGCTGGTGGAGTCTGGGGGAGGCTTGGTG
CAGCCTGGGGGGTCTCTGAGACTCTCCTGTGCAGCC
TCTGGATTCACCTTCAGTAACCAAGTCATGAGCTGG
GTCCGCCAGGCTCCAGGAAAGGGGCCCGAGCGGGTC
TCAGTTATCGGCAGTGTCGGAGGTGCCACAGGTGCC
ACAAGTTATGCAGACTCCGTGAGGGGCCGATTCACC
ATCTCCAGAGATAACGCCAGGAGCACGCTGCATCTG
CAAATGAACAGCCTGAAACCTGAGGACACGGCCGTG
TATTACTGTGTAAAGGGGAACGGACTTACTTCTACT
CGCGCGAGTGACTACTGGGGCCAGGGAACCCAGGTC
ACCGTCTCCTCAGAACCCAAGACACCAAAACCACAA
ACT

a-CD45-h-VHH-52

(SEQ ID NO: 176)
EVQLLESGGGLVQPGGSLRLSCAASGFTFSDYAMSW
VRQAPGKGPERVSVIGSVGGTTGVTSYADSVKGRFT
ISRDNARSTLHLQMNSLKPEDTAVYYCVKGNGLTST
RASDYWGQGTQVTVSSEPKTPKPQT

The protein sequence is encoded by the cDNA shown in SEQ ID NO: 177 below.

(SEQ ID NO: 177)
GAGGTGCAGCTGCTGGAGTCTGGGGGAGGCTTGGTG
CAGCCTGGGGGGTCTCTGAGACTCTCCTGTGCAGCC
TCTGGATTCACCTTCAGTGACTACGCCATGAGCTGG
GTCCGCCAGGCTCCAGGAAAGGGGCCCGAGCGGGTC
TCAGTTATCGGCAGTGTCGGAGGTACCACAGGTGTC
ACAAGTTATGCAGACTCCGTGAAGGGCCGATTCACC
ATCTCCAGAGATAACGCCAGGAGCACGCTGCATCTG
CAAATGAACAGCCTGAAACCTGAGGACACGGCCGTG
TATTACTGTGTAAAGGGGAACGGACTTACTTCTACT
CGCGCGAGTGACTACTGGGGCCAGGGAACCCAGGTC
ACCGTCTCCTCAGAACCCAAGACACCAAAACCACAA
ACT

a-CD45-h-VHH-53

(SEQ ID NO: 178)
EVQLVESGGGLVQAGGSLRLACTASGSDFKRAALGW
YRQAPGQERELVAAFNSGGKTYYTDSVKDRFTISRD
NAKSTLYLQMNSLKPDDTAMYYCALSRFDYYLPPTQ
FDYWGQGTQVTVSSEPKTPKPQT

This protein sequence is encoded by the cDNA shown in SEQ ID NO: 179 below.

(SEQ ID NO: 179)
GAGGTGCAGCTGGTGGAGTCTGGGGGAGGATTGGTG
CAGGCTGGGGGCTCTCTGAGACTCGCCTGTACAGCC
TCTGGAAGCGACTTCAAGCGCGCCGCCCTGGGCTGG
TACCGCCAGGCTCCAGGACAGGAGCGCGAGTTGGTC
GCAGCTTTTAATAGTGGAGGTAAAACATACTACACA
GATTCTGTGAAGGACCGATTCACCATCTCCAGAGAC
AATGCCAAGAGTACGCTGTATCTCCAAATGAACAGC
CTGAAACCTGACGACACGGCCATGTATTACTGTGCG
TTATCACGGTTCGATTACTATCTTCCACCCACCCAA
TTTGACTACTGGGGCCAGGGGACCCAGGTCACCGTC
TCCTCAGAACCCAAGACACCAAAACCACAAACT

a-CD45-h-VHH-54

(SEQ ID NO: 180)
EVQLVESGGGLVQAGGSLRLSCAASGRTLTFYTGWF
RQAPGKEREFVASIRWSGGNTDYADSVKGRFTISGD
NAKNTVYLQMNSLKPEDTAIYYCAALRSWTTTPQRE
VLYDYWGQGTQVTVSSEPKTPKPQT

This protein sequence is encoded by the cDNA shown in SEQ ID NO: 181 below.

(SEQ ID NO: 181)
GAGGTGCAGCTGGTGGAGTCTGGGGGAGGATTGGTG
CAGGCGGGGGGCTCTCTGAGACTCTCCTGTGCAGCC
TCCGGACGCACCCTCACTTTTTATACTGGCTGGTTC
CGCCAGGCTCCAGGGAAGGAGCGTGAGTTTGTAGCA
TCTATTAGGTGGAGTGGCGGTAACACAGACTATGCA
GACTCCGTGAAGGGCCGATTCACCATCTCCGGAGAC
AACGCCAAGAACACGGTGTATCTGCAAATGAACAGC
CTGAAACCCGAGGACACGGCCATTTATTACTGCGCG
GCACTTAGATCTTGGACTACTACACCTCAGAGGGAG
GTCCTCTATGACTACTGGGGCCAGGGGACCCAGGTC
ACCGTCTCCTCAGAACCCAAGACACCAAAACCACAA
ACG

a-CD45-h-VHH-55

(SEQ ID NO: 182)
EVQLVESGGGLVQAGGSLKLSCAASGRTLTYYTAWF
RQAPGKEREFVASLGWSGDVTYYADSVKGRFTISGD
NAKNTVYLQMNSLKPEDTATYYCAALRSWTTTPQRE
VLYDNWGHGTQVTVSSAHHSEDPNS

This protein sequence is encoded by the cDNA shown in SEQ ID NO: 183 below.

(SEQ ID NO: 183)
GAGGTGCAGCTGGTGGAGTCTGGGGGAGGATTGGTG
CAGGCGGGGGGCTCTCTGAAACTCTCCTGTGCAGCC
TCCGGACGCACCCTCACTTATTATACTGCCTGGTTC
CGCCAGGCTCCAGGGAAGGAGCGTGAGTTTGTAGCA
TCGCTAGGGTGGAGTGGCGATGTCACATACTATGCA
GACTCCGTGAAGGGCCGATTCACCATCTCCGGCGAC
AACGCCAAGAACACGGTATATCTGCAAATGAACAGC
CTGAAACCCGAGGACACGGCCACTTATTACTGCGCA
GCACTTAGATCTTGGACTACTACACCTCAGAGGGAG
GTCCTCTATGACAACTGGGGCCACGGGACCCAGGTC
ACCGTCTCCTCAGCGCACCACAGCGAAGACCCTAAT
AGT

a-CD45-h-VHH-56

(SEQ ID NO: 184)
EVQLVESGGGLVQPGGSLRLSCAASGFTFSNQVMSW
VRQAPGKGPERVSVIGSVGGATGATSYADSVRGRFT
ISRDNARSTLHLQMNSLKPEDTAVYYCVKGNGLTST
RASDYWGQGTQVTVSSEPKTPKPQT

This protein sequence is encoded by the cDNA shown in SEQ ID NO: 185 below.

(SEQ ID NO: 185)
GAGGTGCAGCTGGTGGAGTCTGGGGGAGGCTTGGTG
CAGCCTGGGGGGTCTCTGAGACTCTCCTGTGCAGCC
TCTGGATTCACCTTCAGTAACCAAGTCATGAGCTGG
GTCCGCCAGGCTCCAGGAAAGGGGCCCGAGCGGGTC
TCAGTTATCGGCAGTGTCGGAGGTGCCACAGGTGCC
ACAAGTTATGCAGACTCCGTGAGGGGCCGATTCACC
ATCTCCAGAGATAACGCCAGGAGCACGCTGCATCTG
CAAATGAACAGCCTGAAACCTGAGGACACGGCCGTG
TATTACTGTGTAAAGGGGAACGGACTTACTTCTACT
CGCGCGAGTGACTACTGGGGCCAGGGAACCCAGGTC
ACCGTCTCCTCAGAACCCAAGACACCAAAACCACAA
ACT

a-CD45-h-VHH-57

(SEQ ID NO: 186)
EVQLVESGGGLVQAGDSLKLSCVGSGRTFSSYGLGW
FRQAPGKEREFLAHITWTAGGTYHADNVKGRFTISR
DDAKNTVYLQMNSLKPEDTAVYYCAARSSGDWRVER
YYDYWGQGTQVTVSSEPKTPKPQT

This protein sequence is encoded by the cDNA shown in SEQ ID NO: 187 below.

(SEQ ID NO: 187)
GAGGTGCAGCTGGTGGAGTCTGGGGGAGGATTGGTG
CAGGCTGGGGACTCTCTGAAACTCTCCTGTGTAGGC
TCTGGACGCACCTTCAGCAGCTATGGGTTGGGCTGG
TTCCGCCAGGCTCCAGGGAAGGAGCGTGAGTTTCTA
GCACATATTACCTGGACTGCTGGTGGAACATACCAT
GCAGACAACGTGAAGGGCCGATTCACCATCTCCAGA
GACGACGCCAAGAATACGGTGTATCTACAAATGAAC
AGCCTGAAACCTGAGGACACGGCCGTTTATTACTGT
GCGGCACGTTCCTCTGGGGATTGGCGTGTCGAGAGA
TATTATGACTACTGGGGCCAGGGGACCCAGGTCACC
GTCTCCTCAGAACCCAAGACACCAAAACCACAAACT

a-CD45-h-VHH-58

(SEQ ID NO: 188)
EVQLEESGGGLVQPGGSLRLSCATSGFTFSNNVMSW
VRQAPGKGPERVAVIGSVGGATGATSYADSVKGRFT
ITRDNARSTLHLQMNGLKPEDTAMYYCAAETSSGLY
YSYDDLQTIDFDSWGQGTQVTVSSAHHSEDPNS

This protein sequence is encoded by the cDNA shown in SEQ ID NO: 189 below.

(SEQ ID NO: 189)
GAGGTGCAGCTGGAGGAGTCTGGGGGAGGCTTGGTG
CAGCCTGGGGGGTCTCTGAGACTCTCCTGTGCAACC
TCTGGATTCACCTTCAGTAACAACGTCATGAGCTGG
GTCCGCCAGGCTCCAGGAAAGGGGCCCGAGCGGGTC
GCAGTTATCGGCAGTGTCGGAGGTGCCACAGGTGCC
ACAAGTTATGCAGACTCCGTGAAGGGCCGATTCACC
ATCACCAGAGATAACGCCAGGAGCACGCTGCATCTG
CAAATGAACGGCCTGAAACCCGAGGACACGGCAATG
TATTACTGTGCGGCGGAGACCAGTAGCGGTCTTTAC
TACAGTTACGATGACCTTCAAACAATTGACTTTGAT
TCCTGGGGCCAGGGGACCCAGGTCACCGTCTCCTCA
GCGCACCACAGCGAAGACCCTAATAGT

a-CD45-h-VHH-59

(SEQ ID NO: 190)
EVQLVESGGGLVQAGGSLRLSCAASERAFKNRALGW
FRQAPGKEREFVASIRWSGGNTYYADSVKGRFTISG
DNAKNTVYLQMNSLKPEDTAIYYCAALRSWTTTPQR
EVLYDNWGQGTQVTVSSEPKTPKPQT

This protein sequence is encoded by the cDNA shown in SEQ ID NO: 191 below.

(SEQ ID NO: 191)
GAGGTGCAGCTGGTGGAGTCTGGGGGAGGCTTGGTG
CAGGCTGGGGGCTCTCTGAGACTCTCCTGTGCAGCC
TCTGAACGCGCCTTCAAGAACCGTGCACTTGGCTGG
TTCCGCCAGGCTCCAGGGAAGGAGCGTGAGTTTGTA
GCATCTATTAGGTGGAGTGGCGGTAACACATACTAT
GCAGACTCCGTGAAGGGCCGATTCACCATCTCCGGA
GACAACGCCAAGAACACGGTGTATCTGCAAATGAAC
AGCCTGAAACCCGAGGACACGGCCATTTATTACTGC
GCAGCACTTAGATCTTGGACTACTACACCTCAGAGG
GAGGTCCTCTATGACAACTGGGGCCAGGGGACCCAG
GTCACCGTCTCCTCAGAACCCAAGACACCAAAACCA
CAAACT

a-CD45-h-VHH-60

(SEQ ID NO: 192)
EVQLVESGGGLVQAGGSLRLSCAASEFTFSGYWMHW
VRQAPGKGPERVSIIGSVGGTSGVTSYADSVRGRFT
VSRDDAKNTVYLHMDSLKAEDTAVYYCNVMQAWGQG
TQVTVLSAHHSEDPIS

This protein sequence is encoded by the cDNA shown in SEQ ID NO: 193 below.

(SEQ ID NO: 193)
GAGGTGCAGCTGGTGGAGTCTGGGGGAGGATTGGTG
CAGGCGGGGGGCTCTCTGAGACTCTCCTGTGCAGCC
TCTGAATTCACCTTCAGTGGCTACTGGATGCACTGG
GTCCGCCAGGCTCCAGGAAAGGGGCCCGAGCGGGTC
TCAATTATCGGCAGTGTCGGAGGTACCTCAGGTGTC
ACAAGTTATGCAGACTCCGTGAGGGGCCGATTCACT
GTCTCCAGAGACGACGCCAAGAACACGGTGTATCTG
CATATGGATAGTTTGAAAGCTGAGGACACGGCCGTG
TATTACTGTAATGTCATGCAGGCTTGGGGCCAGGGC
ACCCAGGTCACCGTCTTGTCAGCGCACCACAGCGAA
GACCCTATTAGT

a-CD45-h-VHH-61

(SEQ ID NO: 194)
EVQLVESGGGLVETGGSLRLSCAGSGRTFSSRHVGW
FRQTPGKEREWVGSVAWNTGSEYYADSVKGRFTISK
DNAKDTVYLQMNSLKPEDTAIYYCAALRSWTTTPQR
EVLYDNWGQGTQVTVSSAHHSEDPIS

This protein sequence is encoded by the cDNA shown in SEQ ID NO: 195 below.

(SEQ ID NO: 195)
GAGGTGCAGCTGGTGGAGTCTGGGGGAGGATTGGTCGAAACTGGGGGTTC
TCTGAGACTCTCCTGTGCAGGTTCTGGACGCACCTTCAGTAGCCGGCACG
TGGGCTGGTTCCGCCAGACTCCAGGGAAGGAGCGTGAGTGGGTTGGAAGT
GTTGCCTGGAACACTGGTAGTGAATATTATGCAGACTCCGTGAAGGGTCG
CTTCACCATTTCCAAGGACAACGCCAAAGACACGGTGTATCTGCAAATGA
ACAGCCTGAAACCCGAGGACACGGCCATTTATTACTGCGCGGCACTTAGA
TCTTGGACTACTACACCTCAGAGGGAGGTCCTCTATGACAACTGGGGCCA
GGGGACCCAGGTCACCGTCTCCTCAGCGCACCACAGCGAAGACCCTATTA
GT

a-CD45-h-VHH-62

(SEQ ID NO: 196)
EVQLVESGGGLVQPGGSLRLSCAASGFTFSDYAMSWVRQAPGKGPERVSV
IGSVGGVGGVTSYADSVKGRFTISRDNARSTLHLQMNSLKPEDTAVYYCV
KGNGLTSTRASDYWGQGTQVTVSSEPKTPKPQT

This protein sequence is encoded by the cDNA shown in SEQ ID NO: 197 below.

(SEQ ID NO: 197)
GAGGTGCAGCTGGTGGAGTCTGGGGGAGGCTTGGTGCAGCCTGGGGGGTC
TCTGAGACTCTCCTGTGCAGCCTCTGGATTCACCTTCAGTGACTACGCCA
TGAGCTGGGTCCGCCAGGCTCCAGGAAAGGGGCCCGAGCGGGTCTCAGTT
ATTGGCAGTGTGGGAGGTGTCGGAGGTGTCACAAGTTATGCAGACTCCGT
GAAGGGCCGATTCACCATCTCCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-63

(SEQ ID NO: 198)
EVQLQESGGGLVQPGGSLRLSCAASGFTFSNQVMSWVRQAPGKGPERVSV
IGSVGGATGATSYADSVRGRFTISRDNARSTLHLQMNSLKPEDTAVYYCV
KGNGLTSTRASDYWGQGTQVTVLSAHHSEDPNS

This protein sequence is encoded by the cDNA shown in SEQ ID NO: 199 below.

(SEQ ID NO: 199)
GAGGTGCAGCTGCAGGAGTCTGGGGGAGGCTTGGTGCAGCCTGGGGGGTC
TCTGAGACTCTCCTGTGCAGCCTCTGGATTCACCTTCAGTAACCAAGTCA
TGAGCTGGGTCCGCCAGGCTCCAGGAAAGGGGCCCGAGCGGGTCTCAGTT
ATCGGCAGTGTCGGAGGTGCCACAGGTGCCACAAGTTATGCAGACTCCGT
GAGGGGCCGATTCACCATCTCCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
GACCCAGGTCACCGTCTTGTCAGCGCACCACAGCGAAGACCCTAATAGT

a-CD45-h-VHH-64

(SEQ ID NO: 200)
EVQLVESGGGLVQAGGSLRLSCVASGEEDFQPYAMGWFRQAPGKEREYVA
ATTWNGGRIRYGDSVKGRFTISRDHPKNTITLQMTSLKPDDTAVYYCAAR
YGTVLLTREDYQHWGRGTQVTVSAAHHSEDPIS

This protein sequence is encoded by the cDNA shown in SEQ ID NO: 201 below.

(SEQ ID NO: 201)
GAGGTGCAGCTGGTGGAGTCTGGGGGAGGCTTGGTGCAGGCTGGGGGGTC
TCTGAGACTCTCCTGCGTAGCCTCTGGAGAGGAGGATTTTCAGCCGTATG
CCATGGGCTGGTTCCGCCAGGCTCCAGGGAAGGAGCGTGAATACGTGGCC
GCGACTACATGGAATGGTGGTAGAATAAGATATGGAGACTCCGTGAAGGG
CCGATTCACCATCTCCAGAGACCACCCCAAGAACACGATCACTTTACAAA
TGACCAGTTTGAAACCTGACGACACGGCCGTTTATTACTGTGCAGCACGG
TACGGTACAGTCCTACTTACACGCGAAGACTATCAACACTGGGGCCGTGG
GACCCAGGTCACCGTTTCCGCGGCGCACCACAGCGAAGACCCTATTAGT

a-CD45-h-VHH-65

(SEQ ID NO: 202)
EVQLVESGGGLVQAGGSLSLSCAASGRTFSTGAMGWFRQAPGKEREFLAR
ITLIGHGTYYADALKGRFTISRDHAKNTVYLQMNSLKPEDTAVYYCVARD
SPCVGNCWYENAGDYEYWGQGTQVTVSSEPKTPKPQT

This protein sequence is encoded by the cDNA shown in SEQ ID NO: 203 below.

(SEQ ID NO: 203)
GAGGTGCAGCTGGTGGAGTCTGGGGGAGGATTGGTGCAGGCTGGGGGCTC
TCTGAGTCTCTCCTGTGCAGCCTCTGGACGCACCTTCAGTACCGGTGCCA
TGGGCTGGTTCCGCCAGGCTCCAGGGAAGGAGCGTGAGTTTCTGGCACGA
ATTACTCTGATTGGCCACGGCACATACTATGCAGATGCCTTGAAGGGCCG
ATTCACCATTTCCAGAGACCACGCCAAGAACACGGTGTATCTGCAAATGA
ACAGCCTGAAACCTGAGGACACGGCCGTATATTACTGTGTAGCGCGAGAC
AGCCCGTGCGTGGGTAATTGTTGGTACGAGAATGCGGGCGACTATGAGTA
CTGGGGCCAGGGGACCCAGGTCACCGTCTCCTCAGAACCCAAGACACCAA
AACCACAAACT

a-CD45-h-VHH-66

(SEQ ID NO: 204)
EVQLLESGGGLVQAGGSLRLSCAASGFTFSNYAMSWVRQAPGKGPERVSI
IGSVGGTSGVTSYADSVKGRFTITRDNARSTLHLQMNSLKPEDTAVYYCV
KGNGLTSTRASDYWGQGTQVTVSSAHHSEDPIS

This protein sequence is encoded by the cDNA shown in SEQ ID NO: 205 below.

(SEQ ID NO: 205)
GAGGTGCAGCTGCTGGAGTCTGGGGGAGGATTGGTGCAGGCTGGGGGGTC
TCTGAGACTCTCCTGTGCAGCCTCTGGATTCACCTTCAGTAATTACGCCA
TGAGCTGGGTCCGCCAGGCTCCAGGAAAGGGGCCCGAGCGGGTCTCAATT
ATCGGCAGTGTCGGAGGTACCTCAGGTGTCACAAGTTATGCAGACTCCGT
GAAGGGCCGATTCACCATCACCAGAGATAACGCCAGGAGCACGCTGCATC
TTCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGCGCACCACAGCGAAGACCCTATTAGT

a-CD45-h-VHH-67

(SEQ ID NO: 206)
EVQLVESGGGLVQAGGSLRLSCAASERTVSVYTMGWFRQAPGKEREFVAS
IRWSGGPNTYYADSVKGRFTISGDNAKNTVYLQMNSLKPEDTAVYYCVAR
DSPCVGNCWYENAGDYEYWGQGTQVTVSSEPKTPKPQT

This protein sequence is encoded by the cDNA shown in SEQ ID NO: 207 below.

(SEQ ID NO: 207)
GAGGTGCAGCTGGTGGAGTCTGGGGGAGGCCTGGTGCAGGCTGGGGGCTC
TCTGAGACTCTCCTGTGCAGCCTCTGAACGCACCGTCAGTGTCTATACCA
TGGGCTGGTTCCGCCAGGCTCCAGGGAAGGAGCGTGAGTTTGTAGCGTCC
ATTCGCTGGAGTGGTGGTCCCAACACATACTATGCAGACTCCGTGAAGGG
CCGATTCACCATCTCCGGAGACAACGCCAAGAACACGGTGTATCTGCAAA
TGAACAGCCTGAAACCTGAGGACACGGCCGTATATTACTGTGTAGCGCGA
GACAGCCCGTGCGTGGGTAATTGTTGGTACGAGAATGCGGGCGACTATGA
GTACTGGGGCCAGGGGACCCAGGTCACCGTCTCCTCAGAACCCAAGACAC
CAAAACCACAAACT

a-CD45-h-VHH-68

(SEQ ID NO: 208)
EVQLVESGGGLVQPGDSLRLSCAASGFTFSSYAMSWVRQAPGKGPERVSV
IGSVGGTTGVTSYADSVRGRFTISRDNARSTLHLQMNSLKPEDTAVYYCV
KGNGLTSTRASDYWGQGTQVTVSSEPKTPKPQT

This protein sequence is encoded by the cDNA shown in SEQ ID NO: 209 below.

(SEQ ID NO: 209)
GAGGTGCAGCTGGTGGAGTCTGGGGGAGGCTTGGTGCAGCCTGGGGACTC
TCTGAGACTCTCCTGTGCAGCCTCTGGATTCACCTTCAGTAGCTATGCCA
TGAGCTGGGTCCGCCAGGCTCCAGGAAAGGGGCCCGAGCGGGTCTCAGTT
ATCGGCAGTGTCGGAGGTACCACAGGTGTCACAAGTTATGCAGACTCCGT
GAGGGGCCGATTCACCATCTCCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGAACCCAAGACACCAAAACCACAAACT

a-CD45-h-VHH-69

(SEQ ID NO: 210)
EVQLEESGGGLVQPGGSLRLSCAASGFTFSNSVMSWVRQAPGKGPERVSV
IGSVGGATGATSYADSVRGRFTISRDNARSTLHLQMNSLKPEDTAVYYCV
KGNGLTSTRASDYWGQGTQVTVSSAHHSEDPIS

This protein sequence is encoded by the cDNA shown in SEQ ID NO: 211 below.

(SEQ ID NO: 211)
GAGGTGCAGCTGGAGGAGTCTGGGGGAGGCTTGGTGCAGCCTGGGGGGTC
TCTGAGACTCTCCTGTGCAGCCTCTGGATTCACCTTCAGTAACTCCGTCA
TGAGCTGGGTCCGCCAGGCTCCAGGAAAGGGGCCCGAGCGGGTCTCAGTT
ATCGGCAGTGTCGGAGGTGCCACAGGTGCCACAAGTTATGCAGACTCCGT
GAGGGGCCGATTCACCATCTCCAGAGATAACGCCAGGAGCACGCTGCATC
TGCAAATGAACAGCCTGAAACCTGAGGACACGGCCGTGTATTACTGTGTA
AAGGGGAACGGACTTACTTCTACTCGCGCGAGTGACTACTGGGGCCAGGG
AACCCAGGTCACCGTCTCCTCAGCGCACCACAGCGAAGACCCTATTAGT

a-CD45-h-VHH-70

(SEQ ID NO: 212)
EVQLLESGGGLVQAGDSLRLSCAASERAYRNRLLGWFRQAPGAERVVVAI
SWSGGSTYYVDSVKGRFTMSRDNSKNTVYLQMNSLKPEDTATYYCAALRF
WTTTPQKEGLYDTWGQGTQVTVSSEPKTPKPQT

This protein sequence is encoded by the cDNA shown in SEQ ID NO: 213 below.

(SEQ ID NO: 213)
GAGGTGCAGCTGCTGGAGTCTGGGGGAGGCTTGGTGCAGGCTGGGGACTC
TCTGAGACTCTCCTGTGCAGCCTCTGAACGCGCCTACAGGAACCGTCTTC
TTGGCTGGTTCCGCCAGGCTCCAGGGGCGGAGCGTGTCGTTGTAGCTATT
AGCTGGAGCGGTGGTAGTACATACTATGTAGACTCCGTGAAGGGCCGATT
CACCATGTCCAGAGACAACAGCAAGAACACGGTGTATCTGCAAATGAACA
GCCTGAAACCCGAGGACACGGCCACTTATTACTGCGCAGCACTTAGATTT
TGGACTACAACACCTCAGAAAGAGGGCCTCTATGACACCTGGGGCCAGGG
GACCCAGGTCACCGTCTCCTCCGAACCCAAGACACCAAAACCACAAACT

a-CD45-h-VHH-71

(SEQ ID NO: 214)
EVQLQESGGGSLQTGDSLRLACEASEIVVENYVMAWFRQAPGKEREWLAR
IIWNTGGTHLQEFVKGREGIGYSVKTSTRTVMNSLKPEDTAIYYCAALRS
WTTTPQREVLYDNWGHGTQVTVSSAHHSEDPIS

This protein sequence is encoded by the cDNA shown in SEQ ID NO: 215 below.

(SEQ ID NO: 215)
GAGGTGCAGCTGCAGGAGTCTGGGGGAGGATCGCTGCAGACTGGGGACTC
ACTGAGACTCGCCTGTGAAGCCTCTGAAATCGTCGTCGAAAATTATGTCA
TGGCCTGGTTCCGCCAGGCTCCAGGGAAGGAGCGTGAGTGGCTAGCGCGT
ATTATCTGGAATACCGGTGGCACACATCTTCAAGAATTTGTGAAGGGCCG
AGAAGGGATCGGCTATAGCGTCAAAACTTCCACCCGCACAGTAATGAACA
GCCTGAAACCCGAGGACACGGCCATTTATTACTGCGCAGCACTTAGATCT
TGGACTACTACACCTCAGAGGGAGGTCCTCTATGACAACTGGGGCCACGG
GACCCAGGTCACCGTCTCCTCAGCGCACCACAGCGAAGACCCTATTAGT

### Example 5: Generation of Chimeric Antigen Receptors (CARs)

In the sequence below, the underlined lowercase region is the IL2 signal peptide, the lowercase is the heavy chain, underlined capitalized regions are linkers, the capitalized regions without underlining are light chains, the bold capitalized regions are the stalk and the bold underlined regions are the CD28 transmembrane region, capitalized italic is CD28 intracellular region, underlined capitalized italic bold is CD3Z intracellular region.

a-CD19CAR

(SEQ ID NO: 216)
mlllvtslllcelphpafllipdiqmtqttsslsaslgdrvtiscrasqd
iskylnwyqqkpdgtvklliyhtsrlhsgvpsrfsgsgsgtdysltisnl
eqediatyfcqqgntlpytfgggtkleitGGGGSGGGGSGGGGSEVKLQE
SGPGLVAPSQSLSVTCTVSGVSLPDYGVSWIRQPPRKGLEWLGVIWGSET
TYYNSALKSRLTIIKDNSKSQVFLKMNSLQTDDTAIYYCAKHYYYGGSYA
MDYWGQGTSVTVSSSGSGSGKPTTTPAPRPPTPAPTIASQPLSLRPEAC
RPAAGGAVHTRGLDFAPRKIEVMYPPPYLDNEKSNGTIIHVKGKHLCPSP
LFPGPSKPFWVLVVVGGVLACYSLLVTVAFIIFWVRSKRSRLLHSDYMNM
TPRRPGPTRKHYQPYAPPRDFAAYRS

[symbol]

[symbol]

[symbol]

[symbol]

[symbol]

[symbol]

This protein sequence is encoded by the cDNA shown in SEQ ID NO: 217 below.

CD19CAR codon optimized

(SEQ ID NO: 217)
ATGCTGCTGCTGGTGACCAGCCTGCTGCTGTGCGAGCTGCCCCACCCCGC
CTTCCTGCTGATCCCCGACATCCAGATGACCCAGACCACCAGCAGCCTGA
GCGCCAGCCTGGGCGACAGAGTGACCATCAGCTGCAGAGCCAGCCAGGAC
ATCAGCAAGTACCTGAACTGGTACCAGCAGAAGCCCGACGGCACCGTGAA
GCTGCTGATCTACCACACCAGCAGACTGCACAGCGGCGTGCCCAGCAGAT
TCAGCGGCAGCGGCAGCGGCACCGACTACAGCCTGACCATCAGCAACCTG
GAGCAGGAGGACATCGCCACCTACTTCTGCCAGCAGGGCAACACCCTGCC
CTACACCTTCGGCGGCGGCACCAAGCTGGAGATCACCGGCGGCGGCGGCA
GCGGCGGCGGCGGCAGCGGCGGCGGCGGCAGCGAGGTGAAGCTGCAGGAG
AGCGGCCCCGGCCTGGTGGCCCCCAGCCAGAGCCTGAGCGTGACCTGCAC
CGTGAGCGGCGTGAGCCTGCCCGACTACGGCGTGAGCTGGATCAGACAGC
CCCCCAGAAAGGGCCTGGAGTGGCTGGGCGTGATCTGGGGCAGCGAGACC
ACCTACTACAACAGCGCCCTGAAGAGCAGACTGACCATCATCAAGGACAA
CAGCAAGAGCCAGGTGTTCCTGAAGATGAACAGCCTGCAGACCGACGACA
CCGCCATCTACTACTGCGCCAAGCACTACTACTACGGCGGCAGCTACGCC
ATGGACTACTGGGGCCAGGGCACCAGCGTGACCGTGAGCAGCAGCGGCAG
CGGCAGCGGCAAGCCCACCACCACCCCCGCCCCCAGACCCCCCACCCCCG
CCCCCACCATCGCCAGCCAGCCCCTGAGCCTGAGACCCGAGGCCTGCAGA
CCCGCCGCCGGCGGCGCCGTGCACACCAGAGGCCTGGACTTCGCCCCCAG
AAAGATCGAGGTGATGTACCCCCCCCCCTACCTGGACAACGAGAAGAGCA
ACGGCACCATCATCCACGTGAAGGGCAAGCACCTGTGCCCCAGCCCCCTG
TTCCCCGGCCCCAGCAAGCCCTTCTGGGTGCTGGTGGTGGTGGGCGGCGT
GCTGGCCTGCTACAGCCTGCTGGTGACCGTGGCCTTCATCATCTTCTGGG
TGAGAAGCAAGAGAAGCAGACTGCTGCACAGCGACTACATGAACATGACC
CCCAGAAGACCCGGCCCCACCAGAAAGCACTACCAGCCCTACGCCCCCCC
CAGAGACTTCGCCGCCTACAGAAGCAGAGTGAAGTTCAGCAGAAGCGCCG
ACGCCCCCGCCTACCAGCAGGGCCAGAACCAGCTGTACAACGAGCTGAAC
CTGGGCAGAAGAGAGGAGTACGACGTGCTGGACAAGAGAAGAGGCAGAGA
CCCCGAGATGGGCGGCAAGCCCAGAAGAAAGAACCCCCAGGAGGGCCTGT
ACAACGAGCTGCAGAAGGACAAGATGGCCGAGGCCTACAGCGAGATCGGC
ATGAAGGGCGAGAGAAGAAGAGGCAAGGGCCACGACGGCCTGTACCAGGG
CCTGAGCACCGCCACCAAGGACACCTACGACGCCCTGCACATGCAGGCCC
TGCCCCCCAGATAA

a-CD38CAR

In the sequence below, the underlined lowercase region is the IL2 signal peptide, the lowercase is the heavy chain, underlined capitalized regions are linkers, the capitalized regions without underlining are light chains, the bold capitalized regions are the stalk and the bold underlined regions are the CD28 transmembrane region, capitalized italic is CD28 intracellular region, capitalized italic bold is CD3Z intracellular region.

(SEQ ID NO: 218)
myrmqllscialslalvtnsqvqlvqsgaevkkpgssvkvsckafggtfs
syaiswvrqapgqglewmgriirflgianyaqkfqgrvtliadkstntay
melsslrsedtavyycagepgredpdavdiwgqgtmvtvssSGGGGSGGG
GSGGGGSGGGGSGGGGSSDIQMTQSPSSLSASVGDRVTITCRASQGIRSW
LAWYQQKPEKARKSLIYAASSLQSGVPSRFSGSGSGTDFTLTISSLQPED
FATYYCQQYNSYPLTFGGGTKVEIKSSGSGSPTTTPAPRPPTPAPTIASQ
PLSLRPEACRPAAGGAVHTRGLDFAPRKIEVMYPPPYLDNEKSNGTIIHV
KGKHLCPSPLFPGPSKPFWVLVVVGGVLACYSLLVTVAFIIFWVRSKRSR
LLHSDYMNMTPRRPGPTRKHYQPYAPPRDFAAYRS

[symbol]

[symbol]

[symbol]

[symbol]

[symbol]

[symbol]

[symbol]

This protein sequence is encoded by the cDNA shown in SEQ ID NO: 219 below.

Codon optimized a-CD38CAR

(SEQ ID NO: 219)
ATGTACAGAATGCAGCTGCTGAGCTGCATCGCCCTGAGCCTGGCCCTGGT
GACCAACAGCCAGGTGCAGCTGGTGCAGAGCGGCGCCGAGGTGAAGAAGC
CCGGCAGCAGCGTGAAGGTGAGCTGCAAGGCCTTCGGCGGCACCTTCAGC
AGCTACGCCATCAGCTGGGTGAGACAGGCCCCCGGCCAGGGCCTGGAGTG
GATGGGCAGAATCATCAGATTCCTGGGCATCGCCAACTACGCCCAGAAGT
TCCAGGGCAGAGTGACCCTGATCGCCGACAAGAGCACCAACACCGCCTAC
ATGGAGCTGAGCAGCCTGAGAAGCGAGGACACCGCCGTGTACTACTGCGC
CGGCGAGCCCGGCAGAGAGGACCCCGACGCCGTGGACATCTGGGGCCAGG
GCACCATGGTGACCGTGAGCAGCAGCGGCGGCGGCGGCAGCGGCGGCGGC
GGCAGCGGCGGCGGCGGCAGCGGCGGCGGCGGCAGCGGCGGCGGCGGCAG
CAGCGACATCCAGATGACCCAGAGCCCCAGCAGCCTGAGCGCCAGCGTGG
GCGACAGAGTGACCATCACCTGCAGAGCCAGCCAGGGCATCAGAAGCTGG
CTGGCCTGGTACCAGCAGAAGCCCGAGAAGGCCAGAAAGAGCCTGATCTA
CGCCGCCAGCAGCCTGCAGAGCGGCGTGCCCAGCAGATTCAGCGGCAGCG
GCAGCGGCACCGACTTCACCCTGACCATCAGCAGCCTGCAGCCCGAGGAC
TTCGCCACCTACTACTGCCAGCAGTACAACAGCTACCCCCTGACCTTCGG
CGGCGGCACCAAGGTGGAGATCAAGAGCAGCGGCAGCGGCAGCCCCACCA
CCACCCCCGCCCCCAGACCCCCCACCCCCGCCCCCACCATCGCCAGCCAG
CCCCTGAGCCTGAGACCCGAGGCCTGCAGACCCGCCGCCGGCGGCGCCGT
GCACACCAGAGGCCTGGACTTCGCCCCCAGAAAGATCGAGGTGATGTACC
CCCCCCCCTACCTGGACAACGAGAAGAGCAACGGCACCATCATCCACGTG
AAGGGCAAGCACCTGTGCCCCAGCCCCCTGTTCCCCGGCCCCAGCAAGCC
CTTCTGGGTGCTGGTGGTGGTGGGCGGCGTGCTGGCCTGCTACAGCCTGC
TGGTGACCGTGGCCTTCATCATCTTCTGGGTGAGAAGCAAGAGAAGCAGA
CTGCTGCACAGCGACTACATGAACATGACCCCCAGAAGACCCGGCCCCAC
CAGAAAGCACTACCAGCCCTACGCCCCCCCCAGAGACTTCGCCGCCTACA
GAAGCAGAGTGAAGTTCAGCAGAAGCGCCGACGCCCCCGCCTACCAGCAG
GGCCAGAACCAGCTGTACAACGAGCTGAACCTGGGCAGAAGAGAGGAGTA
CGACGTGCTGGACAAGAGAAGAGGCAGAGACCCCGAGATGGGCGGCAAGC
CCAGAAGAAAGAACCCCCAGGAGGGCCTGTACAACGAGCTGCAGAAGGAC
AAGATGGCCGAGGCCTACAGCGAGATCGGCATGAAGGGCGAGAGAAGAAG
AGGCAAGGGCCACGACGGCCTGTACCAGGGCCTGAGCACCGCCACCAAGG
ACACCTACGACGCCCTGCACATGCAGGCCCTGCCCCCCAGA

m-VHH1-E3-TM

In the protein sequence below (SEQ ID NO: 220), the lowercase region is anti murine CD45 VHH, the underlined capitalized regions are linkers, the capitalized regions without underlining is the extracellular membrane proximal region of E3.49K, the bold capitalized underlined regions are E3.49K transmembrane region, and bold capitalized region is intracellular region of E3.49K.

(SEQ ID NO: 220)
maqvqlvesggglvhpgdslrlscaasgsvfnsatmgwyrqspgsqrelv
ativvgtptyadsvkgrftisrdnaknivylqmnslkpedtavyycnyra
tytsgysrdywgqgtqvtvsGGGGSDEGKRYRVKVIPPNTTNSQSVKIQP
YTRQTTPDQEHKFELQFETNGNYDSKIPSTTVAIVVGVIAGFITLIIVFI
CYICCRKRPRAYNHMVDPLLSFSY

In the sequence DNA below (SEQ ID NO: 221), the lowercase region is anti murine CD45 VHH, the underlined capitalized regions are linkers, the capitalized regions without underlining is the extracellular membrane proximal region of E3.49K, the bold capitalized underlined regions are E3.49K transmembrane region, and bold capitalized region is intracellular region of E3.49K. This sequence encodes for protein SEQ ID NO: 220.

(SEQ ID NO: 221)
atggcccaggtgcagctggtggagagcggcggcggcctggtgcaccccgg
cgacagcctgagactgagctgcgccgccagcggcagcgtgttcaacagcg
ccaccatgggctggtacagacagagccccggcagccagagagagctggtg
gccaccatcgtggtgggcacccccacctacgccgacagcgtgaagggcag
attcaccatcagcagagacaacgccaagaacatcgtgtacctgcagatga
acagcctgaagcccgaggacaccgccgtgtactactgcaactacagagcc
acctacaccagcggctacagcagagactactggggccagggcacccaggt
gaccgtgagcGGCGGCGGCGGCAGCGATGAGGGAAAACGGTACCGGGTTA
AGGTTATTCCGCCTAACACCACAAACTCCCAGAGTGTCAAAATTCAGCCT
TACACCAGGCAGACTACTCCTGACCAGGAACACAAATTCGAATTACAGTT
TGAGACTAACGGTAACTATGACTCCAAGATTCCATCTACAACGGTCGCGA
TCGTAGTGGGCGTGATTGCAGGCTTCATCACATTGATCATCGTGTTCATC
TGCTATATCTGCTGTAGGAAGCGCCCTCGGGCGTACAACCACATGGTGGA
CCCTCTGTTGAGTTTCTCATATTAA

mVHH2-E3-TM

In the protein sequence below (SEQ ID NO: 222), the lowercase region is anti murine CD45 VHH, the underlined capitalized regions are linkers, the capitalized regions without underlining is the extracellular membrane proximal region of E3.49K, the bold capitalized underlined regions are E3.49K transmembrane region, and bold capitalized region is intracellular region of E3.49K.

(SEQ ID NO: 222)
maqvqlvqsggglvqpggslrlscaasgrafnsaamgwyrqapgsqrelv
asisagtasyadavkgrftisrdyakniiylqmnslkpddtavyfcnyrt
tytsgysedywgqgtqvtvsGGGGSDEGKRYRVKVIPPNTTNSQSVKIQP
YTRQTTPDQEHKFELQFETNGNYDSKIPSTTVAIVVGVIAGFITLIIVFI
CYICCRKRPRAYNHMVDPLLSFSY

In the sequence DNA below (SEQ ID NO: 223), the lowercase region is anti murine CD45 VHH, the underlined capitalized regions are linkers, the capitalized regions without underlining is the extracellular membrane proximal region of E3.49K, the bold capitalized underlined regions are E3.49K transmembrane region, and bold capitalized region is intracellular region of E3.49K. This sequence encodes for protein SEQ ID NO: 222.

(SEQ ID NO: 223)
atggcccaggtgcagctggtgcagagcggcggcggcctggtgcagcccgg
cggcagcctgagactgagctgcgccgccagcggcagagccttcaacagcg
ccgccatgggctggtacagacaggcccccggcagccagagagagctggtg
gccagcatcagcgccggcaccgccagctacgccgacgccgtgaagggcag
attcaccatcagcagagactacgccaagaacatcatctacctgcagatga
acagcctgaagcccgacgacaccgccgtgtacttctgcaactacagaacc
acctacaccagcggctacagcgaggactactggggccagggcacccaggt
gaccgtgagcGGCGGCGGCGGCAGCGATGAGGGAAAACGGTACCGGGTTA
AGGTTATTCCGCCTAACACCACAAACTCCCAGAGTGTCAAAATTCAGCCT
TACACCAGGCAGACTACTCCTGACCAGGAACACAAATTCGAATTACAGTT
TGAGACTAACGGTAACTATGACTCCAAGATTCCATCTACAACGGTCGCGA
TCGTAGTGGGCGTGATTGCAGGCTTCATCACATTGATCATCGTGTTCATC
TGCTATATCTGCTGTAGGAAGCGCCCTCGGGCGTACAACCACATGGTGGA
CCCTCTGTTGAGTTTCTCATATTAA

a-CD43-sc

a-CD43-sc (SEQ ID NO: 224) is the protein for the anti-CD43 antibody along with stalk and transmembrane region joined through linker regions. SEQ ID NO: 225 is DNA sequence of the same molecule. In the sequence below, the underlined lowercase region is the IL2 signal peptide, the lowercase is the heavy chain, underlined capitalized regions are linkers, the capitalized regions without underlining are light chains, the bold capitalized regions are the stalk and the bold underlined regions are the CD34 transmembrane region.

(SEQ ID NO: 224)
myrmgllscialslalvtnsevqlqqsgpelvkpgasvrmsctasgytfts
yvmhwikqkpgqgldwigyinpynggtqynekfkgkatltsdkssstayme
lssltsedsavyycarrtfpyyfdywgqgttltvssSGGGGSGGGGSGGGG
SGGGGSGGGGSSDVLMTQTPLSLPVSLGDQASISCRSSQSILHSNGNTYLE
WYLQKPGQSPKLLIYKVSNRFSGVPDRFSGSGSGTDFTLKISRVEAEDLGV
YYCFQGSHAPLTFGAGTKLELKSSGSGSPTTTPAPRPPTPAPTIASQPLSL
RPEACRPAAGGAVHTRGLDFAPRKIEVMYPPPYLDNEKSNGTIIHVKGKHL
CPSPLFPGPSKPTLIALVTSGALLAVLGITGYFL

In the sequence DNA below (SEQ ID NO: 225), the underlined lowercase region is the IL2 signal peptide, the lowercase is the heavy chain, underlined capitalized regions are linkers, the capitalized regions without underlining are light chains, the bold capitalized regions are the stalk and the bold underlined regions are the CD34 transmembrane region.

(SEQ ID NO: 225)
atgtacagaatgcagctgctgagctgcatcgccctgagcctggccctggt
gaccaacagcgaggtgcagctgcagcagagcggccccgagctggtgaagc
ccggcgccagcgtgagaatgagctgcaccgccagcggctacaccttcacc
agctacgtgatgcactggatcaagcagaagcccggccagggcctggactg
gatcggctacatcaacccctacaacggcggcacccagtacaacgagaagt
tcaagggcaaggccaccctgaccagcgacaagagcagcagcaccgcctac
atggagctgagcagcctgaccagcgaggacagcgccgtgtactactgcgc
cagaagaaccttcccctactacttcgactactggggccagggcaccaccc
tgaccgtgagcagcAGCGGCGGCGGCGGCAGCGGCGGCGGCGGCAGCGGC
GGCGGCGGCAGCGGCGGCGGCGGCAGCGGCGGCGGCGGCAGCAGCGACGT
GCTGATGACCCAGACCCCCCTGAGCCTGCCCGTGAGCCTGGGCGACCAGG
CCAGCATCAGCTGCAGAAGCAGCCAGAGCATCCTGCACAGCAACGGCAAC
ACCTACCTGGAGTGGTACCTGCAGAAGCCCGGCCAGAGCCCCAAGCTGCT
GATCTACAAGGTGAGCAACAGATTCAGCGGCGTGCCCGACAGATTCAGCG
GCAGCGGCAGCGGCACCGACTTCACCCTGAAGATCAGCAGAGTGGAGGCC
GAGGACCTGGGCGTGTACTACTGCTTCCAGGGCAGCCACGCCCCCCTGAC
CTTCGGCGCCGGCACCAAGCTGGAGCTGAAGAGCAGCGGCAGCGGCAGCC
CCACCACCACCCCCGCCCCCAGACCCCCCACCCCCGCCCCCACCATCGCC
AGCCAGCCCCTGAGCCTGAGACCCGAGGCCTGCAGACCCGCCGCCGGCGG
CGCCGTGCACACCAGAGGCCTGGACTTCGCCCCCAGAAAGATCGAGGTGA
TGTACCCCCCCCCCTACCTGGACAACGAGAAGAGCAACGGCACCATCATC
CACGTGAAGGGCAAGCACCTGTGCCCCAGCCCCCTGTTCCCCGGCCCCAG
CAAGCCCACCCTGATCGCCCTGGTGACCAGCGGCGCCCTGCTGGCCGTGC
TGGGCATCACCGGCTACTTCCTGTAA

### Example 6. Creation of Lentiviral vectors

The transfer vector containing the gene of interest is transfected into 293T cells along with packaging vectors (pMDLg/pRRE and pRSV-Rev), and an envelope vector (phCMV-VSV-G) and the viral supernatant is harvested.

Small-Scale Production of VSV-G Pseudotyped Lentiviral Vectors

This method describes the production of VSV-G pseudotyped lentiviral vectors in 6-well plates using a calcium phosphate transfection kit. The produced amount of virus supernatant per well were 4 ml, and the virus concentration is dependent on the vector used.

Materials Table 1.

| Material | Company | Catalog no |
|---|---|---|
| 293FT cells | Invitrogen | R700-07 |
| DMEM (high glucose) | Invitrogen | 11965-092 |
| FBS | Invitrogen | 16000-044 |
| PBS | Invitrogen | 10010-023 |
| MEM non-essential amino | Sigma-Aldrich | M7145 |
| acid solution (100×) | | |
| L-glutamine (200 mM) | Sigma-Aldrich | G7513 |
| Sodium pyruvate (100 mM) | Sigma-Aldrich | S8636 |
| HEPES solution (1M) | Sigma-Aldrich | H3537 |
| Poly-D-Lysine coated | BD Biocoat | 354550 |
| 150 mm Dish | | |
| TrypLE Express stable | Invitrogen | 12604-013 |
| trypsin replacement | | |
| Calcium phosphate | Sigma-Aldrich | CAPHOS-1KT |
| transfection kit | | |
| Chloroquine | Sigma-Aldrich | C6628 |
| 0.45 μm Millex HV filters | Milipore | SLHV 033RS |

General Materials:

T75 flasks, cell culture pipettes, micropipettes and tips, 1.5 ml microcentrifuge tubes, 5 ml syringes, Trypan Blue, Virkon, M-ytdes.

Plasmids

Vector: depending on choice
Gag-pol plasmid: pMDLg/pRRE
Rev plasmid: pRSV-Rev
Envelope plasmid: phCMV-VSV-G

Procedure

Maintaining 293FT Cells

Maintain cells by splitting every other day 1:5-1:4 and keep them in a T75 or T150 flask. After thawing the cells, culture them for at least 3 passages before using them for virus production so that they recover and start exponential growth. Use of cells with high passage numbers are not recommended as this negatively affects the virus production.

Day 1: Plating & Transfection

Plate 500.000 cells/well in complete growth medium.
Pipette off the medium from the flask
Wash the cells with 10 ml of room temperature PBS
Add 1 ml (T75) or 2 ml (T150) of TrypLE Express and incubate at 37° C. for 5 minutes
After incubation, add 10-20 ml of complete medium into the flask, resuspend thoroughly and dissociate all the clumps by pipetting up and down several times
Count the cells using Trypan Blue and make a cell suspension of 250.000 cells/ml
Put 2 ml of this suspension into a well (Poly-L-Lysine coated 6-well plate). Prepare one well for each vector.
Put the plate in the incubator for at least 7-8 hours

After incubation, transfect the cells. Confirm that the cells have attached and you have a confluency of optimally 80%. If the cells are viable and at an appropriate density, put the plate back in the incubator and go on with preparation of the transfection mix. Abort the transfection if confluency is below 60%.
Prepare 1 ml of full growth medium with the addition of Chloroquine at a final concentration of 25 μM. Put it into the incubator for pre-warming. It is important that the components of the calcium phosphate precipitation kit are brought to room temperature before starting the transfection.
Prepare the plasmid mixture into a microcentrifuge tube as follows (4 μg DNA in total):
2 μg of LeGO-iG2 vector (including transgene)
1 μg of pMDLg/pRRE (Gag/Pol)
0.75 μg of pRSV-REV (Rev)
0.25 μg of phCMV-VSV-G (Envelope)
Mix the plasmids, and adjust the volume up to 54 μl with ddH₂O
Add 6 μl of 2.5 M CaCl₂) solution to the DNA mixture
Into a separate microtube, put 60 μl of 2×HeBS buffer. Add the CaCl₂/DNA mixture and vortex
Let this mixture sit at room temperature for 15 minutes. Do not allow it to sit more than 30 minutes as this might decrease the transfection efficiency.
During these 15 minutes, take your dish out, discard the medium and add 1 ml prewarmed full growth medium containing of 25 μM Chloroquine.
After the 15 minute incubation, add the 120p mixture into the well in a dropwise manner while gently swirling the dish in circular motion.
Close the lid of the dish and put it back into the incubator for 10-12 hours of incubation.

Day 2: Medium Change

10-12 hours post-transfection:
Aspirate the medium containing the transfection mix and chloroquine from the wells and discard.
Add 2 ml of full growth medium per well. Make sure the medium is prewarmed at least to room temperature, preferably 37° C. Put the cells into the incubator.

Day 3: Collection of Supernatant 1

24 hours post medium change:
Check the cells under UV microscope for GFP expression. The transfection efficiency should be above 90%.
Prepare a 0.45 μm filter, a 5 ml syringe, 5 ml microtube and a 1.5 tube per well.
Harvest the medium from the dish using the 5 ml syringe. Apply the filter and filter the supernatant into the 5 ml microtube. Be gentle while filtering, do not create air bubbles, do not apply extreme force. When you are done, drop the syringe and filter into Virkon solution.
Take a 100 μl aliquot from the filtered supernatant into the 1.5 ml microcentrifuge tube (virus titration). Aliquot the rest of the supernatant as you wish and freeze at −80° C. for long-term storage.
Add 2 ml of full growth medium per dish. Make sure the medium is prewarmed at least to room temperature, preferably 37° C. Put the cells into the incubator.

Day 4: Collection of supernatant 2

48 hours post medium change:
Collect virus supernatants in the same manner as the previous day.
Discard the plates.

### Example 7: Generation of Cell Lines

Using the lentiviral particles harvested in the previous example, K562 and RPMI8226 (FIG. 6) cells were transduced, sorted and expanded. The resulting cells were tested in the following fashion.

Preparation of Target Cells
1. Take a sample from the target cells for cell counting.
2. Take 1-2×10⁶ cells in a tube
3. Centrifuge the cells
4. Discard the supernatant
5. Wash with PBS
6. Discard supernatant
7. Centrifuge again and pipette off the liquid to get a “dry” pellet
8. Add 0.1 ml ⁵¹Cr to the cell pellet, mix well
9. Incubate for 1 hour, shake the vial every 15 mins.

Preparation of Effector cells
1. Take a sample from the cultured cells for cell counting.
2. Spin down the cells and resuspend pellet in warm RPMI+10% FCS, for a concentration of 0.3×10⁶ cells/ml.
3. Add 150 μl/well in triplicate of the diluted sample in the first row.
4. Add 100 μl/well of RPMI+10% FCS in triplicate in rows 2-4 and in the 3 wells of MIN release.
5. Add 100 μl/well of dH₂O+1% Triton X100 in the 3 wells of MAX release.
6. Prepare serial dilutions at one to three ratios throughout the wells.
7. Put the plate in the incubator.

Target Cells
1. Wash target cells in PBS twice
2. Resuspend the cells in 1 ml RPMI+10% FBS
3. Take a sample for cell counting
4. Add 100 μl/well of the target cells, mix
5. Incubate for 4 hours
6. Spin plate at 300 g for 3 mins.
7. Pipette 20 ul of cell suspension to each of the respective wells of LumaPlate 96-well.
8. Let the plates in the chromium hood overnight so that they are dry.

Calculate Percent Specific Lysis:[(Experimental Release−Spontaneous Release)/(Maximum Release−Spontaneous Release)]*100

Differentiation of Functionally Mature NK Cells from Induced Pluripotent Stem Cells (iPSCs)

Induced Pluripotent Stem Cells (iPSCs) were differentiated into functionally mature NK cells, using feeder-independent differentiation protocol. These NK cells display both functional maturation and phenotypic signatures representative of blood-derived NK cells and possess potent anti-tumour effector functions.

Human iPSC Generation Culture and Differentiation into Hematopoietic Cells

Healthy male dermal fibroblasts were reprogrammed using the StemRNA 3rd Generation Reprogramming kit.

Thawed iPSC lines are cultured in mTesR™ 1 (StemCell Technologies, 85850) feeder-free maintenance medium for 5 days on hESC-Qualified Matrigel (Corning, 354277) coated 6-well plates to reach 80% confluency.

Passaged iPSC lines using 0.5 mM EDTA in PBS, 01-862-1B).

Prior to passaging, prepared hematopoietic differentiation medium (HPDM), consisting of StemDiff™ APEL™2 (StemCell Technologies, 05270), 40 ng/mL SCF (PeproTech, 300-07), 20 ng/mL BMP4 (PeproTech, 120-05), and 20 ng/mL VEGF (PeproTech, 100-20B), and supplement with 10 μM Rock inhibitor (Y-27632, Tocris, 1254) for the first 3 days.

Passaged iPSCs and seeded in 100 uL of HPDM, supplemented with Rock Inhibitor in each well of an ultra-low attachment, round-bottom 96-well plate (Corning, CLS3474) at a density of 3,000 cells/well.

Centrifuged cells for 5 min at 220 g to facilitate formation of embryoid body (EB) structures and incubated, undisturbed, for 3 days at 37° C. and 5% CO₂.

Performed media changes on day 3, 6, and 9 by removing 70 uL of medium from each well and adding 100 uL of freshly prepared HPDM, without Rock inhibitor.

Collected hematopoietic progenitor cells on day 11 for flow cytometric analysis or transfer to NK cell differentiation cultures using a wide-bore p200 pipette (Fisher Scientific, 14-222-730).

Hematopoietic Cell Differentiation into NK Cells

Seeded hematopoietic progenitor cells in the second phase of NK cell differentiation from iPSCs, in a standard cell-culture treated 6-well plate at a concentration of 32 EBs per well in 4 mLs NK cell differentiation media (NKDM), consisting of StemDiff APEL 2 (StemCell Technologies, 05270), 20 ng/mL SCF (PeproTech, 300-07), 20 ng/mL IL-7 (PeproTech, 200-07), 10 ng/mL IL-15 (PeproTech, 1110-15) and 10 ng/mL Flt3L (PeproTech, 300-19), and supplemented with 5 ng/mL IL-3 (PeproTech, 200-03).

Performed half media changes twice per week for four weeks with freshly prepared NKDM containing IL-3 for the first week only, and without IL-3 for the following three weeks

After 4 weeks of NK cell differentiation culture, collected cells and either analyze phenotypically via flow cytometry or expanded for three to four weeks in CTS OpTmizer™ T Cell Expansion medium (ThermoFisher, A1048501) supplemented with 5% hAB serum (Corning, 35-060-CI), 1% penicillin/streptomycin (Gibco, 15140122), 0.2 mM L-glutamine (Gibco, 25030081), 10 ng/mL rhIL-15 (Gold Biotechnology, 1110-15), 500 IU/mL rhIL-2 (Akron Biotech, AK8223), and 25 ng/mL rhIL-21 (Gold Biotechnology, 1110-21), prior to cytotoxicity and functionality assays.

### Example 8: Test Assays

Embryonic stem cells (ESCs) or Induced Pluripotent Stem cells (iPSCs) were cultured according to procedure described in Khan F A, Almohazey D, Alomari M, Almofty S A. Isolation, Culture, and Functional Characterization of Human Embryonic Stem Cells: Current Trends and Challenges. Stem Cells Int. 2018; 2018:1429351. All cell lines are tested for mycoplasma contamination and only mycoplasma free cells are used in studies. Karyotyping of cell lines are carried out in our lab's cytogenetics facility using standard protocols.

Embryoid bodies are generated by dispase dissociation of ESC/iPSC cultures, plating 5×10⁶ cells/per well of an ultra-low attachment 6-well plate containing X-VIVO medium along with supplements. Medium is changed every 3rd day and cultures is maintained for 15-20 days.

Hematopoietic differentiation and gene modification of ESCs/iPSCs is achieved with electroporation of standard mammalian expression vector/or an excisable lentiviral vector NK cells were differentiated by co-culture with OP9 and OP9-DLLI cells as described by Zeng J, Tang S Y, Toh L L, Wang S. Generation of “Off-the-Shelf” Natural Killer Cells from Peripheral Blood Cell-Derived Induced Pluripotent Stem Cells. Stem Cell Reports. 2017; 9(6):1796-812.

ESCs/iPSCs Differentiation to RPE (Retinal Pigment Epithelium)

The ESCs/iPSCs colonies are passaged by using EDTA and differentiated to RPE by using the protocol developed by Buchholz (Buchholz D E, Pennington B O, Croze R H, Hinman C R, Coffey P J, Clegg D O. Rapid and efficient directed differentiation of human pluripotent stem cells into retinal pigmented epithelium. Stem Cells Transl Med. 2013; 2(5):384-93) w % ere used in treating macular degeneration, Briefly, hESC line HS980 was established and cultured under xeno-free and defined conditions on rhLN-521, and passaged using standard protocols. For differentiation, cells were plated at a density of 2.4.3 104 cells/cm2 on 20 mg/mL hrLN-111-coated dishes using NutriStem hESC XF medium and Rho-kinase inhibitor during the first 24 h. NutriStem hESC XF without basic fibroblast growth factor and transforming growth factor b was then replaced and from day 6 after plating, 100 ng/mL of activin A was added to the medium for a total of 5 weeks.

Normal human CD56+NK cells and CD8+ T cells are isolated by positive/negative enrichment (Miltenvi CliniMACS system) of blood cells collected from healthy human donors. NK-92 cells (CRL-2407) were obtained from the American Type Culture Collection (Manassas, Va.) and cultured as described in the product sheet.

Cytotoxicity Testing by Chromium Release Assays

Target cells were evaluated for their susceptibility to NK-cell-mediated lysis by 4 h ⁵¹Cr release assay. 48 h before the assay, NK cells are cultured in NK medium containing IL2. Target cells are labeled with 50 μCi of ⁵¹Cr for 2 h at 37° C. ⁵¹Cr-labeled cells are plated per well of a 96-well-i-bottom plate. NK cells are added at different ratios to target cells and incubated for 4 h at 37° C. Controls include labeled cells without NK cells (spontaneous release) and labeled cells lysed with 1% Triton X-100 (total lysis). 20 μl of each reaction supernatant are added to Luma scintillation plate and dried overnight in the hood. Following radioactivity reading, the percent specific lysis are calculated. A more detailed protocol is set out below,
a. Take a sample from K-562 for cell counting.
b. Take 1-2×106 cells in a tube
c. Centrifuge the cells
d. Discard the supernatant
e. Add 0.1 ml 51Cr to the cell pellet
f. Incubate for 1 hour
g. Take a sample from the cultured cells for cell counting.
h. Dilute the sample to 0.3×106 cells/ml in RPMI+10% FCS. Total volume 1 ml.
i. Mark a 96-well plate.
j. Add 150 μl/well in triplicate of the diluted sample in the first row.
k. Add 100 μl/well of RPMI+10% FCS in 3 steps down and in the 3 wells of MIN release.
l. Add 100 μl/well of dH₂O+2M HCL in the 3 wells of MAX release.
m. Take 50 μl from the first row and add to the Second row, mix and take 50 μl from the second row to the third. Continue like this and throw away 50 μl from the last row.
n. Put the plate in the incubator.
o. Wash K-562 in PBS twice
p. Resuspend the cells in 1 ml 10% RPMI
q. Take a sample for cell counting
r. Calculate how much cells you need.
s. Add 100 μl/well of the target cells.
t. Incubate for at least 4 hours.
u. Mark the tubes for the gamma counter.
v. Take 70 μl sample/tube. Be careful and avoid the cell.
w. Analyse in the gamma counter.
x. Incucyte-based Cytotoxicity Measurement.

### Incucyte-based Cytotoxicity Measurement

Incucyte was used for the measurement of immune cell mediated cytotoxicity and infiltration of single tumor spheroids. Spheroids mimic in vivo conditions more accurate than cell monolayers exhibiting several characteristics that determine solid tumor killing and infiltration like cell-to-cell adhesion within the tumor, increased cell survival as well as diffusion gradients for oxygen, nutrients and waste products from the outer cell ring to the inner core. IncuCyte-based measurement of immune cell cytotoxicity allows real-time observations.

Procedure:
1. Cytolight Green vial is resuspended by adding 21.5 ul DMSO to a new vial, to prepare a 5 mM stock solution
2. 2.8 ul of stock solution is added to 360 ul of PBS to create the 100× dilution.
3. Effector cells are taken in a 15 ml tube and spun at 400×g for 5 mins.
4. Pellets are washed with 5 ml PBS and washing solution is removed carefully.
5. Pellets are resuspended in 6 ml PBS and 60 ul of 100×Cytolight Green solution is added to each tube
6. Cells are incubated for 20 mins at 37° C. and mixed every 5 minutes.
7. A clean plate along with the lid is placed in incubator to pre-warm the lid.
8. 3.6 ml of 100% FBS is added to bind excess Cytolight reagent. Cells are mixed and centrifuged at 400×g for 5 mins. Supernatant is aspirated and cells are resuspended in 500 ul medium.
9. Cells are counted, and concentration is adjusted through adding medium.
10. CytotoxRed stock solution is prepared by bringing 1 vial of CytotoxRed (5 μL) to RT and briefly centrifuging, and adding 45 μL of PBS to CytotoxRed
11. CytotoxRed working concentration is prepared by adding 32.5 ul of CytotoxRed in 6.5 ml in total volume SCGM containing 10% FBS.
12. Plate is assembled and 100 ul of CytotoxRed is added. 50 ul of Target cells, 50 ul of effector cells or media is added.
13. Plate is placed in the Incucyte and red cells are counted for 4 hours.

Cytotoxicity Testing with CD8+ T Cells

Normal human CD8+ T cells were primed by coculturing with IFN-γ treated target cells (2×10⁶ CD8+ cells and 5×10⁵ target cells/2 ml) in culture medium supplemented with 50 U/mI IL-2, 25 nn/ml IFN-γ. On day 7, cocultures are replenished with 1.75×10⁶ fresh IFN-γ treated target cells. On day 14, these primed CD8+ T-cells are collected by centrifugation and used in chromium release assays as described above for NK cells.

Generation of Cell Lines

Using the lentiviral particles harvested in Example 10, K562 and RPMI cells were transduced and expanded. The resulting cells were tested for the expression of the target genes either using GFP as marker or by labelling cells with the corresponding antibodies or florescent labelled proteins.
1. Prepare medium DMEM/RPMI 10% FBS=400 ul/well
2. Remove 50 ul of supernatant from each well.
3. Add lentiviral vector and tx medium.
4. Mark a 24-well plate with date, name, cell type and what virus you use for transduction.
5. Set the temperature of the centrifuge to 32° C.
6. Detach the cells with a cell scraper and resuspend all cells with a serological pipette by pipetting up and down several times.
7. Count the cells using trypan blue and make a cell suspension with 10⁶ cells/ml in medium+10% FBS.
8. Distribute 250 ul of cells to 24 well plate.
9. Take required amount of protamine sulfate stock for a final concentration of 8 ug/ml.
10 Avoid repeated freeze/thaw of the stock.
11 Add the medium according to the calculations.
12. Keep the virus on the dry ice until usage. Thaw the required amount of virus quickly.
13. Mix the virus carefully so that the contact with air is minimal.
14. Add the calculated amount of virus to each well.
15. Pipette protamine sulfate (8 ug/ml) and IL-2 (1000 IU/ml) to each well in case of NK cells.
16. Mix the cells carefully by pipetting up and down.
17. Centrifuge the plate with 1000×g for 1 hr at 32° C. without break.
18. Take out the plate and incubate between 4 hr and overnight (depending on your construct and virus titer; should be tested) in the incubator.
19. At the end of the incubation, centrifuge the plate again with 1000×g for 1 hr at 32° C.
20. Remove 80% of the medium carefully from all the wells and fill with 500 ul fresh pre-warmed medium with serum.
21. Put the plate back into the incubator.
   Day 1 & 2: Check the cells under the microscope and search for colonies.
   Day 3: Analyse the cells with flow cytometry.

In Vivo Reactivity with Allogeneic CD8+ T Cells and NK Cells

All mouse housing, breeding, and surgical procedures were approved by the animal ethics committee in Stockholm, Sweden. The mice were purchased from the Charles River Laboratories. NSG mice have been previously described (Shultz L. D., Lyons B. L., Burzenski L. M., Gott B., Chen X., Chaleff S., Kotb M., Gillies S. D., King M., Mangada J., Greiner D. L., Handgretinger R. (2005) Human lymphoid and myeloid cell development in NOD/LtSz-scid IL2Rγnull mice engrafted with mobilized human hemopoietic stem cells. J. Immunol. 174, 6477-6489 [PubMed: 15879151]), and bred and maintained in the AKM5 animal facility at the Karolinska Institute, Huddinge, Sweden.

The mice were acquired from Jackson laboratories (NOD.Cg-Prkdcscid Il2rgtmlWjI/SzJ—JAX stock number 005557. Originated at The Jackson Laboratory, bred on license by Charles River in Europe.).

Male mice (8-10 weeks old) were subcutaneously injected with either UC or K562 cell lines (1×106). All cells were tested and found free from mycoplasma before injection. The mice were intravenously injected with human PBMCs (10×106). Measurements of subcutaneous tumor size were started when mice had measurable tumors. The tumor size was measured at least twice a week for four weeks with slide calipers and tumor volume was calculated. When tumor volume reached 1 cm3 the mice were euthanized and the tumor and organs were removed.

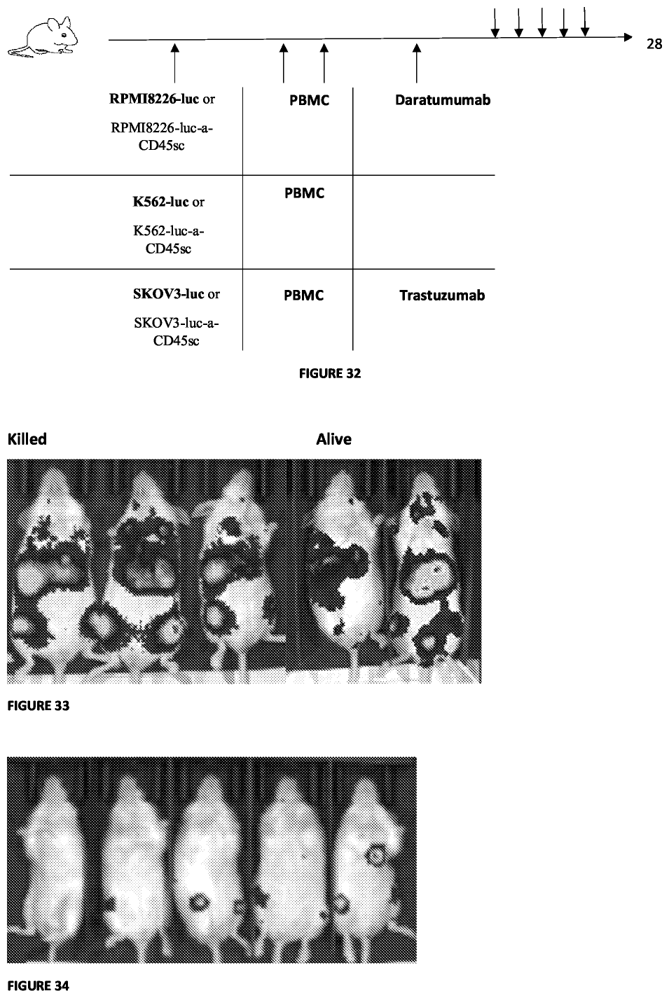

In a parallel study, male NSG mice (6-8 weeks old) were subcutaneously injected with either CD45 engager and luciferase reporter gene modified or only luciferase reporter gene modified (without CD45 engager modification) K562, RPM18226, and SKOV3 cell lines (1×106). All cells were tested and found free from mycoplasma before injection. The mice were intravenously injected with human PBMCs (10×106) divided into two consecutive days (5×106 PBMCs per day), one day after the tumor administration. Mice were then injected 1—In RPM18226 injected group, Daratumumab (ADCC competent AntiCD38 antibody) and 2—In SKOV3 injected group, Trastuzumab (AntiHer2 antibody) subcutenously 3 days after tumor cell injection, at 8 mg/kg for both antibodies. Mice under isoflurane were fluorescently imaged by using the In Vivo Imaging System (IVIS) Spectrum (Perkin Elmer, Santa Clara, Calif., USA) and analyzed using IVIS imaging software (Perkin Elmer). Imaging was performed on all animals on day 0, and twice weekly until the mice were euthanized and the tumor and organs were removed (FIG. 32).

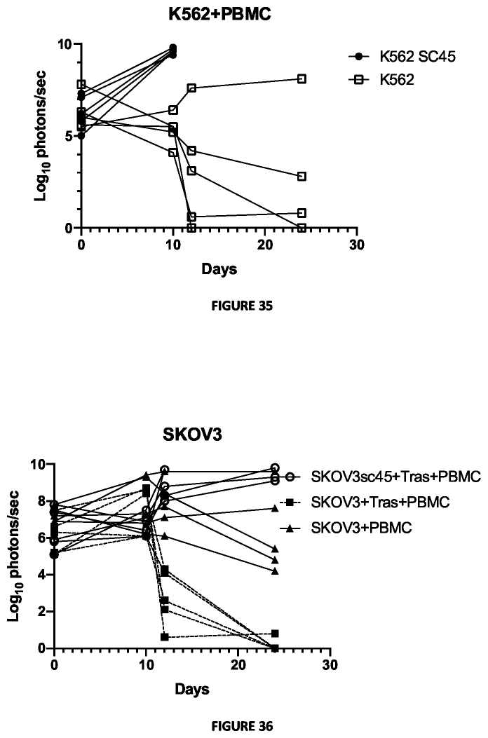

IVIS imaging demonstrated that, control mice in RPMI-8226 group that received PBMCs and Daratumumab controlled the tumor development (FIG. 34). However injection of RPMI-8226 cells expressing CD45 engagers together with PBMCs and Daratumumab lead to tumor development (FIG. 33 Photos from IVIS imaging depicting RPMI-8226 expressing Luciferase and CD45 Engagers. Mice are treated with PBMCs and Daratumumab.). In a similar fashion, CD45 engager modified K562 cells, even with PBMC administration, led to higher immune evasion, compared to administration of K562 cells with consecutive PBMC administration (FIG. 35). Finally, IVIS imaging of control mice in SKOV3 group that received PBMCs and Trastuzumab controlled the tumor development while injection of SKOV3 cells expressing CD45 engagers together with PBMCs and Trastuzumab lead to tumor development (FIG. 36).

Flow Cytometry

The staining and washes are performed in flow cytometry acquisition buffer. A single cell suspension of the cells is incubated with blocking reagent for 10 min on ice and then stained with antibodies and viability staining for 30-60 min on ice. Samples are analyzed on a Fortessa/Symphony flow cytometer (BD Biosciences) and the data are analyzed using FlowJo software (TreeStar, Ashland, Oreg.). For sorting AriaFusion (BD Biosciences) machine is used. Sorted cell are cultured in medium with antibiotics for two weeks. Onwards, cells are cultured without antibiotics.

Extracellular Vesicles (EVs) Mediated a-CD45-Sc mRNA Delivery Ameliorates Collagen-Induced Arthritis

Extracellular vesicles (EVs) from the target cells are isolated/purified either using ultracentrifugation, tangential flow filtration, or through size exclusion chromatography. Number and size of EVs are analyzed through Nanosight tracking analysis system (NTA). EVs are used for the mRNA delivery of the transgene used to generate antibody or nanobody in vivo. We also tested the engagers expressed on extracellular vesicles at different densities.

Isolation and Purification of EVs

Conditioned medium (CM) was harvested and pre-cleared by low speed centrifugation at 700×g for 5 min. To remove large cell debris and apoptotic bodies, the CM was centrifugated at 2,000×g for 10 min. Finally, to eliminate any remaining unwanted larger vesicles, CM were then filtered by using bottle top filters (Corning, low protein binding) with 0.22 μm pore sized cellulose acetate membrane. Then the CM medium was diafiltrated by ultra-filtration using tangential flow filtration (TFF, MicroKross, 20 cm2, SpectrumLabs) with a cut-off of 300 kDa. Finally, the CM was concentrated by using Amicon Ultra-15 10 kDa weight cut-off spin filters (Millipore) with spin filter at 4000×g for a certain time based on the sample concentration. Then, the EVs quality and concentration were analyzed using ZetaView (FIG. 40).

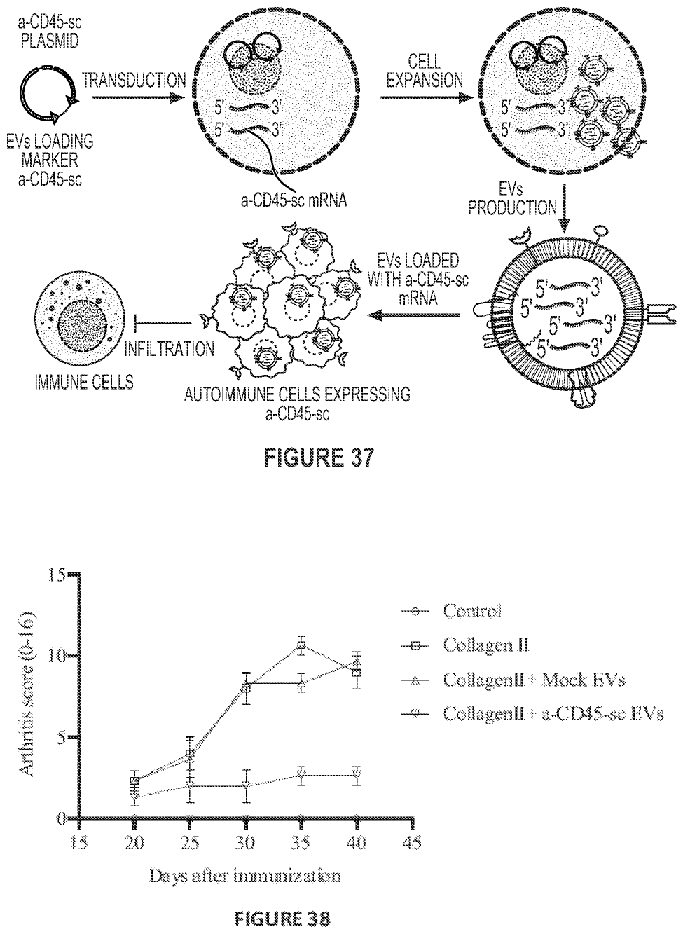

Endogenous passive loading of a-CD45-sc mRNA into EVs. EV-producer cells are modified to overexpress the a-CD45-sc mRNA, which is then overloaded into vesicles during EV biogenesis, along with their original cargo and the protein translated from the overexpressed mRNA transcripts. EV-mediated cargo delivery upon a-CD45-sc mRNA loading. Bioengineered EVs are taken up by autoimmune cells. Endosomal degradation leads to the delivery of a-CD45-sc mRNA into the cytoplasm. Translation of delivered a-CD45-sc mRNA to protein resulting in the inhibition of autoimmune incidences (FIG. 37).

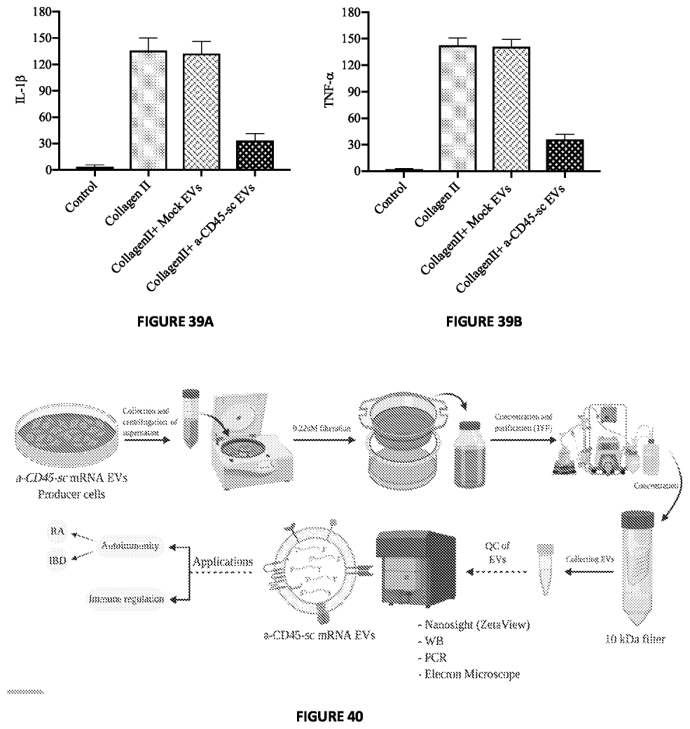

The Collagen-induced arthritis (CIA) mouse model is a well-established and frequently used model mimicking the clinical symptoms and immunopathogenesis of human RA. Mice immunized with Collagen II (CII) increased arthritis scores. The control group displayed no gross changes. Interestingly, MSC EVs loaded with a-CD45-sc mRNA exhibited inhibitory effects on arthritis severity (FIG. 38). In contrast, the mRNA mock MSC EVs had no effect. Additionally, the pathogenesis of RA involves activated immune cells promoting macrophages to release pro-inflammatory cytokines. Therefore, the levels of TNF-α, IL-1β in serum were measured by sandwich ELISA. Remarkably, MSC EVs loaded with a-CD45-sc mRNA reduced the levels of TNF-α and IL-1β in serum of CIA mice (FIGS. 39A and B). These results indicated that a-CD45-sc effectively attenuates inflammation in CIA mice (FIG. 38). a-CD45-sc EVs ameliorates Collagen-induced arthritis (CIA) severity. CIA was induced by active immunization with chicken Collagen II (CII) in DBA/1J mice. a-CD45-sc mRNA or mock mRNA loaded MSC EVs were injected at day 0, day 7, day 14 and day 21 after induction of arthritis. 2.5E11 EVs were tail i.v injected. Arthritis score was examined every 5 days. Data are expressed as mean±SD (n=5).

Referring to FIGS. 39A and 39B a-CD45-sc EVs inhibits pro-inflammatory cytokines production in CIA mice. CIA was induced by active immunization with chicken Collagen II (CII) in DBA/1J mice. a-CD45-sc mRNA or mock mRNA loaded MSC EVs were injected at day 0 and Day 10 after induction of arthritis. After i.v injections of 5E11 EVs from a-sc-CD45 or mock mRNA, the levels of cytokines (TNF-α and IL-1β) were measured on day 40. Data are expressed as mean±SD.

Transgene Expression Systems

For the transgene expression in the target and/or effector cells, lentiviral and retroviral system are used. For transient expression, either electroporation or chemical based methods are used. The transgenes are delivered either vector-based or as mRNA with or without nanoparticles through chemical or electrochemical delivery system. For gene delivery, system of biocompatible materials such as lipid, naked DNA, chromosomes, plasmid, cationic polymers, and conjugate complexes can be utilized.

Suicide Genes

Depending on the clinical application and the cells, suicide genes may be incorporated into the cells. This will enable destruction of cells using normally nontoxic agents such as ganciclovir. Representative suicide genes are shown in Table 3 below.

TABLE 3
Suicide Genes

| | | | | | |
|---|---|---|---|---|---|
| | | Mechanism of | | | Bystander |
| Transgene | Origin | action | Prodrug | Immunogenicity | effect |

Group 1: cell cycle independent

| | | | | | |
|---|---|---|---|---|---|
| Escherichia | Bacterial | Converts CB1954 to | CB1954 (5- | + | + |
| coli | | 4-hydroxylamino | (aziridin-1-yl)-2,4- | | |
| Ntr | | derivatives that react | dinitrobenzamide | | |
| | | with cellular | | | |
| | | thioesters, | | | |
| | | generating | | | |
| | | hydroxylamine | | | |
| | | alkylating agents | | | |
| | | that cross-link DNA | | | |
| CYP 2B1 | Rat | Converts | Cyclophosphamide | + | + |
| (cytochrome | | cyclophosphamide to | | | |
| p450) | | its active | | | |
| | | compounds: | | | |
| | | phosphoramide | | | |
| | | mustard and acrolein | | | |
| CYP 4B1 | Rabbit | Converts 2- | 2-aminoanthracene | + | + |
| (cytochrome | | aminoanthracene to | | | |
| p450) | | DNA-alkylating | | | |
| | | agents | | | |
| iCasp9 | Human | Aggregation and | Chemical inducer | − | |
| | | activation of iCasp9 | of dimerization | | |
| | | by CID | AP20187 | | |
| | | administration, | | | |
| | | downstream | | | |
| | | activation of caspase | | | |
| | | cascadeand | | | |
| | | apoptosis | | | |
| CD20 | Human | ADCC | Anti-CD20 | − | |
| | | | monoclonal | | |
| | | | antibody | | |
| | | | (rituximab) | | |
| tmpk | Human | Phosphorylation of | AZT | − | NR |
| | | AZT to AZT-TP | | | |
| DED-FADD | Human | Fas-crosslinking | CID AP1903 | − | |
| | | recruits death- | | | |
| | | inducing signaling | | | |
| | | complex, activates | | | |
| | | proteolytic caspase | | | |
| | | cascade and | | | |
| | | apoptosis | | | |

Group 2: cell cycle dependent

| | | | | | |
|---|---|---|---|---|---|
| HSV-tk | Viral | HSV-tk | GCV | + | + |
| | | phosphorylation of | | | |
| | | GCV to GCV-MP, | | | |
| | | rate limiting step of | | | |
| | | the conversion into | | | |
| | | cytotoxic products | | | |
| CD | Bacterial | Hydrolytic | 5-fluorocytosine | + | + |
| | fungal | deamination of | | | |
| | | cytosine to uracil | | | |
| | | block of DNA | | | |
| | | synthesis | | | |
| E. coli | Bacterial | XGPRT | 6-thioxanthine | + | + |
| xanthine- | | phosphorylates 6- | | | |
| GPT gene | | thioxanthine to | | | |
| | | thioanthine MP that | | | |
| | | is converted to 6- | | | |
| | | thioguanine | | | |
| | | monophosphate | | | |
| E. coli PNP | Bacterial | deoD (PNP) | 6-MePdR | + | + |
| (deoD) | | converts MePdR to | | | |
| | | MeP | | | |
| VZV-tk | Viral | VZV-tk | 6-methoxypurine | + | + |
| | | phosphotylation of | arabinonucleoside | | |
| | | 6-methoxypurine | (ara-M) | | |
| | | arabinonucleoside, | | | |
| | | rate-limiting step of | | | |
| | | the conversion into | | | |
| | | cytotoxic products | | | |
| Linamarase | Plant | Linamarase encodes | Linamarin | + | + |
| (b- | | a cyanogenic b- | | | |
| glucosidase) | | glucosidase that | | | |
| | | hydrolyses linamarin | | | |
| | | to acetone, glucose | | | |
| | | and cyanide. | | | |
| | | Cyanide inhibits the | | | |
| | | cytochrome c | | | |
| | | oxidase of the | | | |
| | | mitochondrial | | | |
| | | respiratory chain, | | | |
| | | blocking the | | | |
| | | oxidative | | | |
| | | phosphorylation and | | | |
| | | causing cell death | | | |
| b-lactamase | Bacterial | Converts | Vinca cephaloid | + | |
| | | vincacephalosporin | | | |
| | | to vinca alkaloid | | | |
| E. coli | Bacterial | Generation of | Anthracyclins | + | |
| b- | | cytotoxic | (Daun02) | | |
| galactosidase | | daunomycin | | | |

### Example 9: Results

Following the chromium cytotoxicity assay of above and referring to FIG. 7, the percent specific lysis of K562 cells with peripheral blood mononuclear cell (PBMC) using the ⁵¹Chromium assay described above. K562 control cells, and K562 expressing E3.49K, UL11 ai-CD45-sc were incubated for 4 hrs with the PBMCs at Effector:Target (E:T) ratios of 10:01, 3:01, 1:01 and 0.3:1. Cells were centrifuged and 20 uL supernatant was added to Luma plates. The plates were dried overnight and read on gamma-counter the following day. FIG. 7 shows a reduction of cell lysis for cells expressing ULll and E3.49K and complete inhibition of lysis for cells expressing a-CD45-sc.

Referring to FIG. 8, the experiment was repeated using NK92 cells instead of PBMCs. K562 control cells, K562 expressing E3.49K, K562 expressing engagers UL11 and K562 and a-CD45-sc were incubated for 4 hrs with the PBMCs with E:T ratios of 10:01, 3:01, 1:01 and 0.3:1. Cells were centrifuged and 20 uL supernatant was added to Luma plates. Plates were dried overnight and read on gamma-counter. As in FIG. 7, the results of FIG. 8 clearly show a reduction of cell lysis for cells expressing UL11 and E3.49K and complete inhibition of lysis for cells expressing a-CD45-sc.

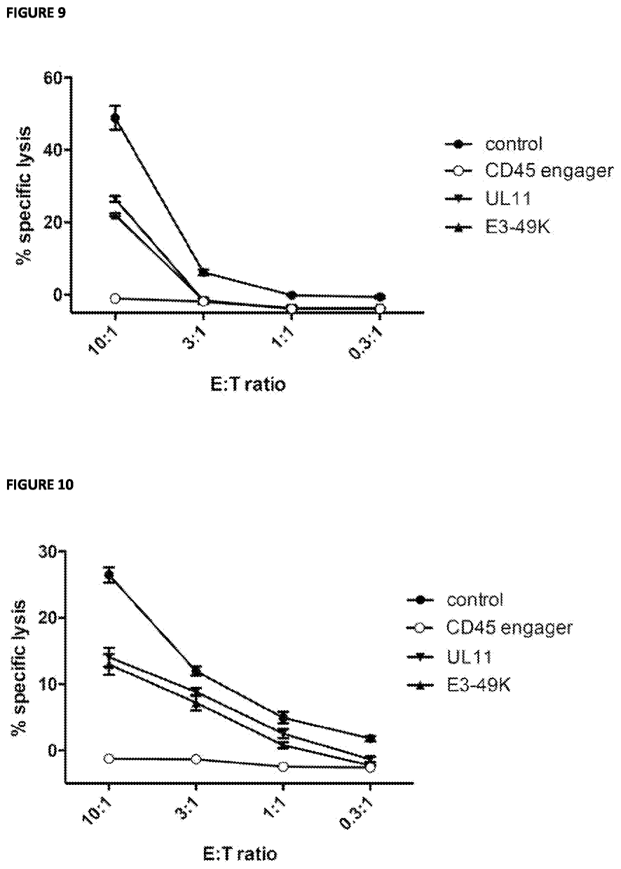

FIG. 9 shows the percent specific lysis in K562 cells using a ⁵¹Cr release assay. K562 control cells, K562 expressing E3.49K, UL11 a-CD45-sc were incubated for 4 hrs with the NK92 with E:T as shown in FIG. 9. Cells were centrifuged and 20 uL supernatant was added to Luma plates. Plates were dried overnight and read on gamma-counter. As in FIGS. 7 and 8, the results of FIG. 9 clearly show a reduction of cell lysis for cells expressing UL11 and E3.49K and complete inhibition of lysis for cells expressing a-CD45-sc.

FIG. 10 shows the percent specific lysis of K562 cells using a ⁵¹Cr release assay. K562 control cells, K562 expressing E3. E3.49K, UL11 a-CD45-sc were incubated for 4 hrs with the PBMCs with E:T as described. Cells were centrifuged and 20 μL supernatant was added to Luma plates. Plates were dried overnight and read on gamma-counter. As in FIGS. 7, 8, and 9 the results of FIG. 10 clearly show a reduction of cell lysis for cells expressing UL11 and E3.49K and complete inhibition of lysis for cells expressing a-CD45-sc

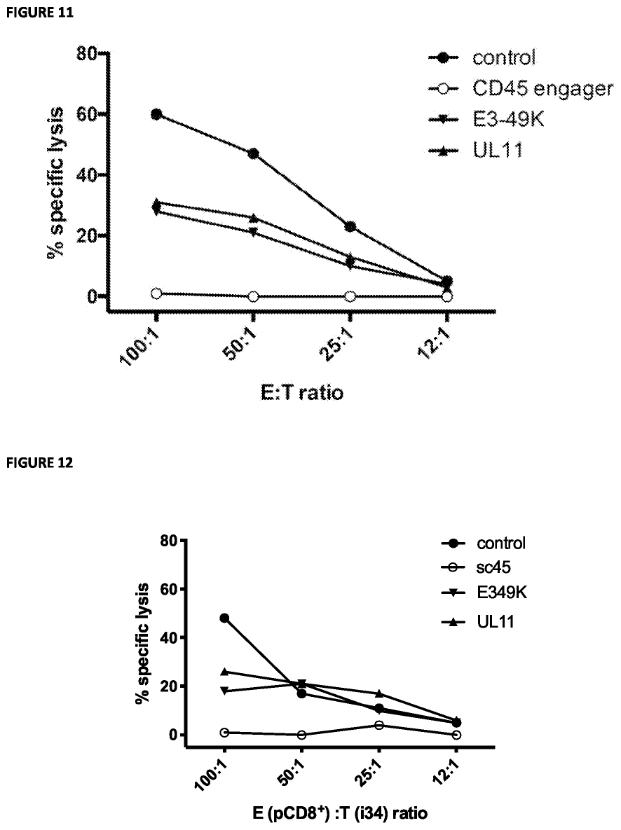

FIG. 11 shows the percent specific lysis of RPMI88226 using a ⁵¹Cr release assay. RPMI88226 control cells, RPMI88226 expressing E3.49K, UL11 or a-CD45-sc were incubated for 4 hrs with T cells with E:T as described. Cells were centrifuged and 20 uL supernatant was added to Luma plates. Plates were dried overnight and read on gamma-counter. As in FIGS. 7-12, results clearly show a reduction of cell lysis for cells expressing UL11 and E3.49K and complete inhibition of lysis for cells expressing a-CD45-sc.

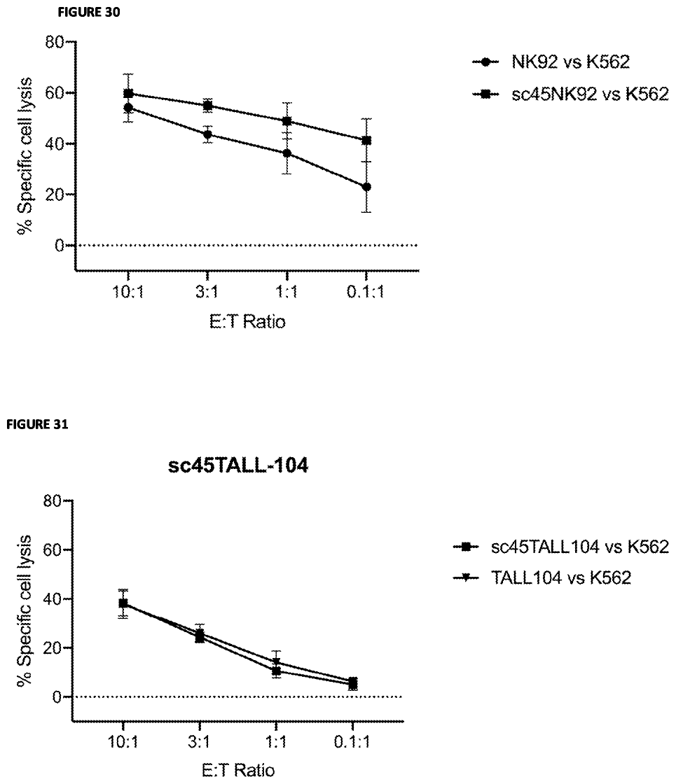

In an effort to assess if expression of CD45 engager affects the function of the graft, in the case where graft cell is an effector cell (NK cell or T cell), NK-92 and TALL-104 cell lines were transduced with a-CD45-sc. NK92 cells were maintained as mentioned above. TALL-104 cells were maintained at 37° C. in 10% CO2 in IMDM (Gibco) supplemented with 10% heat-inactivated fetal bovine serum (Atlanta Biologicals, Norcross, Ga.) and 100 units/ml of recombinant human IL-2. The cell line repeatedly tested negative for mycoplasma contamination using a commercial polymerase chain reaction kit. Both NK-92 cells and TALL-104 cells were tested at 4-6 concentrations in 4-h ⁵¹Cr-release assays against a fixed number (10⁴/well) of ⁵¹Cr-labeled K562 cells in suspension. The unmodified NK-92 and TALL-104 were used as control effector cells. The percentage of specific ⁵¹Cr release was calculated from the mean of three replicates. FIG. 30 depicts the comparative assessment of NK-92 cells with a-CD45-sc gene modification. FIG. 31 depicts the comparative assessment of TALL-104 cells with a-CD45-sc gene modification.

### Example 10: Chimeric Antigen Receptor (CAR) Modified Cells

We evaluated our invention in a-CD38CAR and a-CD19CAR cells through the following experiments.

A. Assessment of the Chimeric Antigen Receptor Against CD38 (a-CD38CAR) Mediated Cytotoxic Capacity of the NK/T Cells Against the (CD38⁺) Target Cells is Affected by a-CD45-sc.

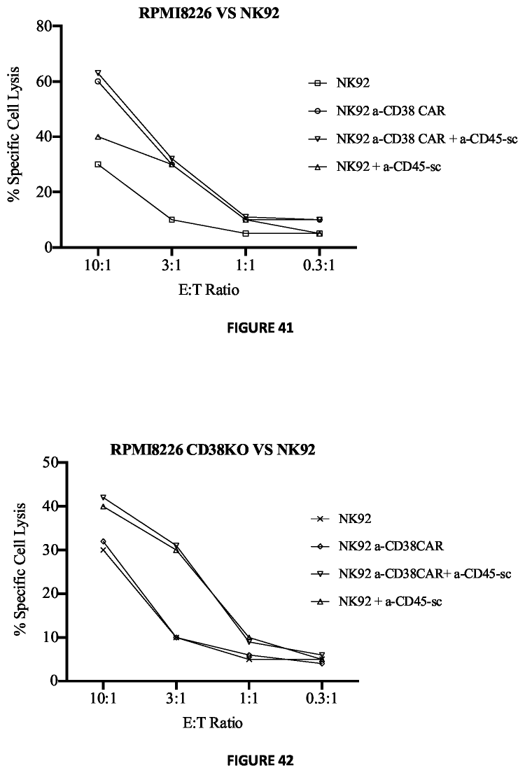

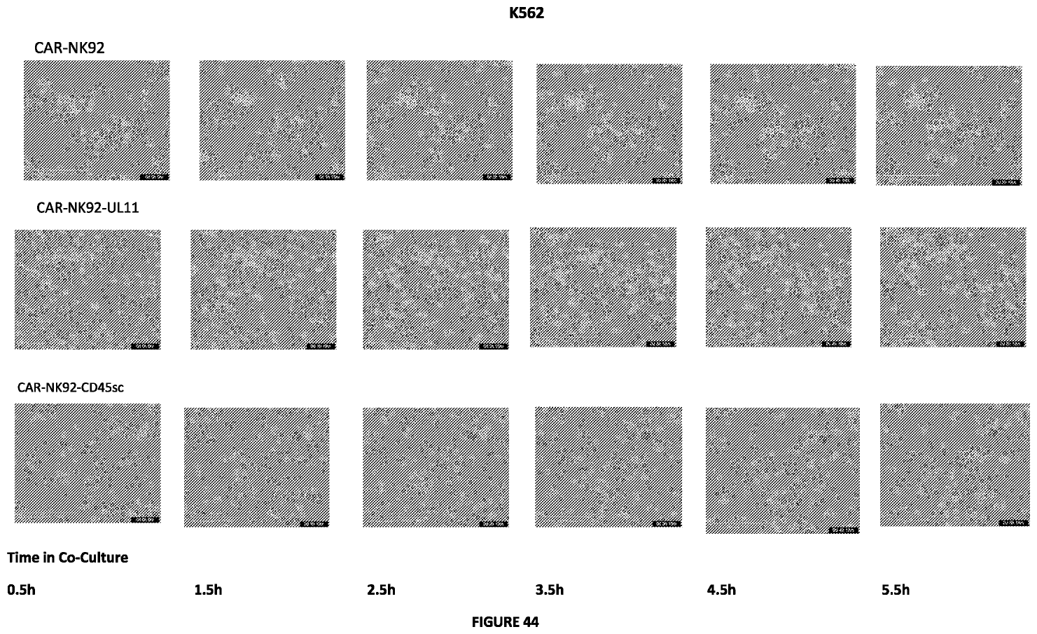

To analyze the impact of a-CD45-sc on the function of a-CD38CAR (SEQ ID NO: 218/219), we expressed the a-CD45-sc (SEQ ID NO: 5), on cells expressing CAR and evaluated their cytotoxic capacity against the target cells (FIGS. 24, 41). This experiment can be readily utilized with single chains and single domains against CD45 with the expectation of similar results. The following Seq. ID. Numbers can be used. NK92 cells were transduced with the viral particles carrying the a-CD38CAR and were sorted. Sorted and expanded NK92 cells were either transduced again with a-CD45-sc, UL11, E3.49K or with the control. RPM18226 knockout for CD38 or wild type cells were used as target. Effector cells were labeled with the CytoLight Green while target cells were labeled with Cytotox Red. Both effector and target cells were incubated at 1:1 in 96-well flat-bottom plate in the Incucyte. Referring to FIG. 44, Red cell indicated the target cell death and were counted for 4 hours. Data were analyzed on GraphPad Prism.

Referring to FIG. 41 RPM18226 cells were incubated with NK92 control cells, NK92 cells expressing a-CD38CAR, a-CD38CAR+a-CD45-sc, or a-CD45-sc. Following 4 hrs incubation cells were centrifuged and 20 uL supernatant was added to Luma plates.

B. Assessment of Whether Chimeric Antigen Receptor Against CD19 (CARCD19) Mediated Cytotoxic Capacity of the NK/T Cells Against the (CD19+) Target Cells is Affected by a-CD45-sc.

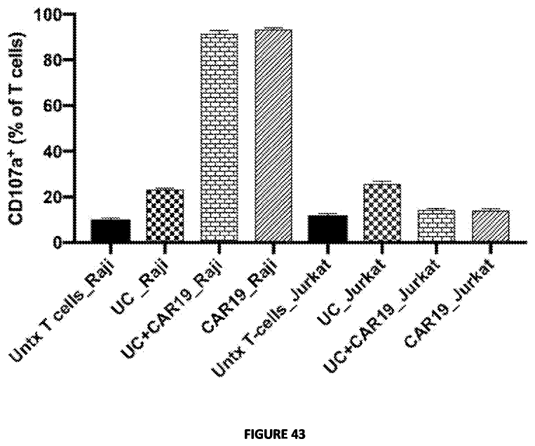

To analyze the impact of a-CD45-sc on the function of a-CD19CAR (SEQ ID NO: 216/217), we expressed the a-CD45-sc (SEQ ID NO: 5) on cells expressing CAR and evaluated their cytotoxic capacity against the target cells. PBMCs were transduced with the viral particles carrying the a-CD19CAR (FIG. 29). Expanded PBMCs expressing a-CD19CAR were either transduced again with a-CD45-sc, or with the control. Jurkat and Raji cells were used as target. Degranulation assay was carried out for 4 hours and cells were labelled with CD107a, along with the CD3, CD56, Live/Dead-APC-H7 and CD19h-Biotin. Following degranulation, cells were run on flowcytometer. Data were analyzed on Flowjo (FIG. 43).

The RPMI8226 CD38.KO cell line was produced using the CRISPR-Cas9 technology. More specifically, cells were transduced with lentiviral vectors encoding for the Cas9 gene, a gRNA targeting the exon 1 of the CD38 gene and a puromycin selection gene. Following assessment of the transduction efficacy by flow cytometry, cells were treated for two weeks with puromycin to allow for the selective survival of the transduced cells. Further flow cytometric analyses confirmed the knock-out of CD38 in the selected population.

Referring to FIG. 42, RPMI8226 CD38 KO cells were incubated with NK92 control cells, NK92 cells expressing a-CD38 CAR, a-CD38 CAR+a-CD45-sc, or a-CD45-sc. Following 4 hrs incubation cells were centrifuged and 20 uL supernatant was added to Luma plates.

### Example 11: Clinical Applications

The present invention can be used to treat any cells or tissue prior to it being introduced into the body. It can also be used to treat autoimmune disease. blood cancers, including lymphomas and leukemias; bone marrow failure syndromes, including anemias and cytopenias; inherited immune disorders, including WAS and SCID; hemoglobinopathies, including sickle cell disease (SCD) and thalassemia; neurological disorders, including neuromyelitis optica; cartilage replacements, for example joint replacements such as knee and hip replacements; prophylactically managing cytotoxicity.

Currently tissue transplants require immunosuppression with drugs. Immunosupression may be required for cell transplants. Immunosuppressants leave patients severely immunocompromised and at high risk of opportunistic infections. Using the constructs and methods taught in Examples 1-13 above, we can design tissue and cell therapies that will not need additional immunosuppression, or may only need low doses of immunosuppressive drugs.

Cells and tissues treated can be any mammalian cell or hybrids between humans and other mammals. Cyranoski D. Japan approves first human-animal embryo experiments. Nature. 2019.

One of skill in the art will appreciate that the scope of this invention is not limited to these examples and will understand that the present invention is potentially applicable to any cell or tissue therapy involving the introduction of any non autologous cell or tissue or modified autologous cell or tissue to a living mammalian organism where there is potential for the body to recognize and reject the introduced cell or tissue.

A. Transplants

1. Solid Organ Transplants

It can be envisioned that this strategy can be utilized by transient or permanent genetic modification of solid organs with vectors coding for a CD43, CD45 and or CD148 engager thus rendering the graft safe from T and NK cell based immune responses due to lack of synapse formation. This can hypothetically be utilized in any organ or part of an organ or organoid transplants including but not limited to: the muscular system (including joints, ligaments, muscle, tendons), the digestive system, (including mouth, teeth, tongue, salivary glands, parotid glands, submandibular glands, sublingual glands, pharynx, esophagus, stomach, small intestine, duodenum, jejunum, ileum, large intestine, liver, gallbladder, mesentery, pancreas, anal canal), respiratory system (including nasal cavity, pharynx, larynx, trachea, bronchi, lungs, diaphragm), urinary system (including kidneys, ureter, bladder, urethra); female reproductive system (ovaries, fallopian tubes, uterus, vagina, vulva, clitoris, placenta); male reproductive system, (including testes, epididymis, vas deferens, seminal vesicles, prostate, bulbourethral glands, penis, scrotum); endocrine system (including pituitary gland, pineal gland, thyroid gland, parathyroid glands, adrenal glands, pancreas); circulatory system (including heart, patent foramen ovale, arteries, veins, capillaries), lymphatic system (including lymphatic vessel, lymph node, bone marrow, thymus, spleen, gut-associated lymphoid tissue, tonsils, interstitium); nervous system (including brain, cerebrum, cerebral hemispheres, diencephalon, the brainstem, midbrain, pons, medulla oblongata, cerebellum, the spinal cord, the ventricular system, choroid plexus, peripheral nervous system, cranial nerves, spinal nerves, ganglia, enteric nervous system, sensory organs, eye, cornea, iris, ciliary body, lens, retina, ear, outer ear, earlobe, eardrum, middle ear, ossicles, inner ear, cochlea, vestibule of the ear, semicircular canals, olfactory epithelium, tongue, taste buds, integumentary system, main article: integumentary system, mammary glands, skin and subcutaneous tissue. The organs of part thereof can be genetically modified using genetic modification strategies that were previously defined. Aravalli R N, Belcher J D, Steer C J. Liver-targeted gene therapy: Approaches and challenges. Liver Transpl. 2015; 21(6):718-37. In essence, a batch of vectors can be utilized for in vivo or ex vivo gene delivery by hydrodynamic delivery or similar strategies. This potentially allows the use of tissues between species.

2. Tissue Transplantation

Similar to solid organ transplants, utilization of these engagers can enable use of tissues or part of the organs to be transplanted. Composite transplantation (hand, extremity, face) can be enabled by utilizing CD45 engagers. The first face transplantation was done in 2005. Ethical questions about face transplantation are even more prominent than those about extremity transplantation because the surgical procedure is extremely demanding and the immunosuppression required puts the recipient at considerable risk of opportunistic infections.

Immunosuppression usually consists of induction therapy (antithymocyte globulin [ATG] and/or IL-2 receptor blocker), followed by triple maintenance immunosuppression with a corticosteroid, an antiproliferative drug (eg, basiliximab), and a calcineurin inhibitor (see table). Sometimes topical creams containing calcineurin inhibitors or corticosteroids are used. Utilization of engagers for these tissues through genetic modification would decrease or even abrogate the need for lifelong immunosuppression.

Skin allografts use donor skin (typically from cadavers). Skin allografts are used for patients with extensive burns or other conditions causing such massive skin loss that the patient does not have enough undamaged skin to provide the graft. Allografts can be used to cover broad denuded areas and thus reduce fluid and protein losses and discourage invasive infection. Unlike solid organ transplants, skin allografts are ultimately rejected, due to immune rejection. Utilization of engagers for these tissues through genetic modification would prolong engraftment without the need of immunosuppression and risk for infections. When valves are damaged or diseased and do not work the way they should they may need to be repaired or replaced. Conditions that may cause heart valve dysfunction are valve stenosis (stiffness) and valve regurgitation (leaky valve). The diseased valve may be repaired using a ring to support the damaged valve, or the entire valve may be removed and replaced by an artificial valve. Artificial valves may be made of carbon coated plastic or tissue (made from animal valves or human valves taken from donors). Allogeneic and xenogeneic valves have the challenge of immune rejection. Thus the patients may need to receive life-long immunosuppression. Modification of the valve grafts with CD45 engagers may abrogate this need for immunosuppression and prolong time to rejection.

Nerve transplant and nerve transfer surgeries are offering new hope for patients who have had extremities paralyzed or severely damaged by accidents. In most cases, the replacement nerves come from cadavers or, occasionally, living donors. Either way, patients must receive immunosuppressant drugs until their nerves regenerate, which can take up to 2 years. Modification of the nerve grafts with CD45 engagers, which are contemplated herein, may abrogate this need for immunosuppression and prolong time to rejection.

Cartilage transplantation is used for children with congenital nasal or ear defects and adults with severe injuries or joint destruction (eg, severe osteoarthritis). Chondrocytes are more resistant to rejection, possibly because the sparse population of cells in hyaline cartilage is protected from cellular attack by the cartilaginous matrix around them. However, the graft still has the risk of rejection, especially in the elderly population. Includion of CD45 engagers for these tissues through genetic modification would increase engraftment.

Bone transplantation is used for reconstruction of large bony defects (eg, after massive resection of bone cancer). No viable donor bone cells survive in the recipient, but dead matrix from allografts can stimulate recipient osteoblasts to recolonize the matrix and lay down new bone. This matrix acts as scaffolding for bridging and stabilizing defects until new bone is formed. Cadaveric allografts are preserved by freezing to decrease immunogenicity of the bone and by glycerolization to maintain chondrocyte viability. Utilization of CD45 engagers for soft bone tissue through genetic modification would decrease the need for this process, thus decrease perioperative processes and decrease postoperative morbidity by faster engraftment of the bone tissue.

Similar strategies can be utilized for adrenal tissue allografting for Parkinson's disease or fetal thymus implanted patients with DiGeorge syndrome.

In the U.S., the most commonly transplanted tissues are bones, tendons, ligaments, skin, and heart valves. Of about 2 million tissue grafts distributed each year, it is thought that only about 1 million grafts are transplanted.

3. Cell Transplants

We can envision that the modified cells with the engagers can be co-modified essentially any transgene, including but not limited to, suicide genes, chemokine receptors, activating or inhibitory receptors, chimeric antigen receptors. We can also envision that CD43, CD45 and or CD148 engager modified cells can be gene edited using endonucleases or CRISPR/Cas9 or other technologies for removal of immunological checkpoint receptors, chemokine receptors, hypoxia responsive receptors, central differentiation regulators among other genes. Use of non-human cells and tissues are contemplated for use in humans.

a. Stem Cell Transplantation for Cancer Treatment and Gene Corrected Stem Cells for Single Gene Disorders or Complex Genetic Disorders.

Allogeneic stem cell transplantation (from cord blood, peripheral blood, bone marrow or other sources) is an accepted therapeutic approach for various diseases such as malignancies including but not limited to acute myeloid leukemia, myelodysplasias, multiple myeloma, and it is being tested in various solid organ tumors/cancers such as liver cancer, breast cancer and kidney cancer, including metastases. Similarly, this approach has shown success in genetic disorders affecting the hematopoietic system such as severe combined immune deficiencies such as SCID-X, Wiskott Aldrich syndrome as well as anemias. There are multiple shortcomings of this approach but one of the challenges remain to be engraftment failures and the need of high chimerism levels. It can be envisioned that donor derived hematopoietic stem cells can be gene modified ex vivo with vectors coding for CD43, CD45 and or CD148 engagers construct mentioned in this application or derivatives thereof and can be utilized to achieve partial or full chimerism, potentially in the absence of a lymphodepleting regimen. This way, host immune cells would be rendered ineffective against the graft and engraftment could be facilitated.

b. Platelet Transfusion

It can also be envisioned to utilize the engagers in platelets by either direct modification of platelets or platelet generating cells in order to avoid rapid platelet rejection potentially mediated by NK cells and other effector cells. This could be utilized before manifestation of platelet refractoriness for patients that receive multiple platelet infusions during their life span.

c. Erythrocyte/Red Blood Cell Transfusion

This strategy can also potentially be utilized to modify erythroid progenitors and RBCs to avoid cellular rejection of an RBC infusion product if the patient has not developed antibodies to the RBC antigens before the time of administration for patients that require multiple RBC transfusions.

d. Multipotent and Pluripotent Cell Therapy or Cell Derivative Therapy Thereof

The above methodology can also be used to produce or generate iPSC or hES cell lines and cells derived there from. Thus, in one embodiment of the invention, compositions and methods are provided to make a target cell that has a CD43, CD45 and/or CD148 engager, and thereby creating a hypoimmunogenic cell. Such a hypoimmunogenic cell is expected to be less prone to immune rejection by a subject into whom such cells are transplanted. When transplanted, this hypoimmunogenic cell should engraft (not be rejected). In one embodiment, such a target cell is capable of engrafting and surviving with little to no immune suppression required of the recipient.

This methodology can be utilized to generate various tissues/cells differentiated from pluripotent/multipotent cells for patients receiving cell replacement therapy such as cartilage degeneration, age related macular degeneration (ESC/iPSC derived RPE administration), stargardt disease, osteogenesis imperfecta (MSC administration intrafetal or after partum) and other diseases.

e. Donor Leukocyte Infusions

Donor leukocyte infusions (DLI) including NK cell, T cell, macrophage infusions with or without additional gene modifications coding for transgenes such as the T Cell Receptor, chimeric antigen receptors, dimeric antigen receptors, or any other genes can be envisioned to utilize a co-transduction of CD43, CD45 and/or CD148 engagers to be able to render the graft safe to infuse by avoiding a functional immunological synapse formation that, without the engager, can lead to recipient cell mediated rejection of the graft cells. T cells can include any T cells, including but not limited to suppressor T cells, regulatory T cells, gamma delta T cells, mucosal associated invariant T cells (MAIT), as well as innate lymphoid cells of all subtypes.

f. Gene Modified T Cell Therapies

This strategy can also be utilized in T cells from allogeneic sources such as bone marrow CD34, donor derived T cells, iPSC derived T cell, hESC derived T cells with or without further gene modification using chimeric antigen receptors, chemokine receptors, T-Cell receptors, activating receptors, cell adhesion receptors. This could be done either by an additional transduction or a co-transduction of CD43, CD45 and/or CD148 engagers to be able to render the graft safe to infuse by avoiding a functional immunological synapse formation that, without the engager can lead to recipient cell mediated rejection of the graft cells, can avoid cell mediated graft rejection. Currently CAR modified T cells are commonly used to treat cancer.

Representative CAR T cells include CARs for hematological cancers currently being investigated include but are not limited to, the following targets and genes: BCMA (TNFRSF17), CD123 (IL3RA), CD138 (SDCl), CD19 (CD19) marketed CD19CARs inclulde axicabtagene ciloleucel (Yescarta™) and tisagenlecleucel (Kymriah™), CD20 (MS4A1), CD22 (CD22), CD38 (CD38), CDS (CDS), lg K chain (IgK), LeY (FUT3), NKG2D ligand (NKG2D), RORl (RORI) and WTl (WTl).

CARs for solid tumors include, but are not limited to, the following targets and genes: Target (Gene), C-Met (MET), CAIX (CA9), com (PROMl), CD171 (LlCAM), CD70 (CD70), CEA (CEACAMS), EGFR (EGFR), EGFR viii (EGFRVIII), Ep-CAM (EPCAM), EphA2 (EPHA2), FAP (FAP), GD2), GPC3 (GPC3), HER2 (ERBB2), HPV16-E6 (HPVE6), IL13Ra2 (IL13RA2), LeY (FUT3), MAGEA3 (MAGEA3), MAGEA4 (MAGEA4), MARTI (MLANA), Mesothlin (MSLN), MUCI (MUCl), MUC16 (MUC16), NY-ESO-1 (CTAGIB), PD-Ll (CD274), PSCA (PSCA), PSMA (FOLHl), RORI (RORI) and VEGFR2 (KOR).

g. Gene Modified NK Cell Therapies

This strategy can also be utilized in NK cells from allogeneic sources such as bone marrow CD34, donor derived T cells, IPSC derived T cell, hESC derived NK cells with or without further gene modification using chimeric antigen receptors, chemokine receptors, T-Cell receptors, activating receptors, cell adhesion receptors. This could be done either by an additional transduction or a co-transduction of CD43, CD45 and/or CD148 engagers to be able to render the graft safe to infuse by avoiding a functional immunological synapse formation that, without the engager can lead to recipient cell mediated rejection of the graft cells, can avoid cell mediated graft rejection.

h. Gene Modified Macrophage Therapies

This strategy can also be utilized in Macrophages from allogeneic sources such as bone marrow CD34, donor derived T cells, IPSC derived T cell, hESC derived Macrophages with or without further gene modification using chimeric antigen receptors, chemokine receptors, T-Cell receptors, activating receptors, cell adhesion receptors. This could be done either by an additional transduction or a co-transduction of CD43, CD45 and/or CD148 engagers to be able to render the graft safe to infuse by avoiding a functional immunological synapse formation that, without the engager can lead to recipient cell mediated rejection of the graft cells, can avoid cell mediated graft rejection.

i. Gene Corrected Cells for Other Genetic Disorders

It can be envisioned in a similar fashion that transplanted cells for metabolic disorders could be modified with transgenes coding for engagers against CD43, CD45 and or CD148 for optimal engraftment, potentially without the need of lymphoablation.

j. Other Cells Contemplated for Use/Transplantation

Other cells contemplated for use/transplantation herein include: Endoderm derived cells such as: exocrine secretory epithelial cells (Brunner's gland cell in duodenum (enzymes and alkaline mucus), Insulated goblet cell of respiratory and digestive tracts (mucus secretion), stomach, foveolar cell (mucus secretion), chief cell (pepsinogen secretion), parietal cell (hydrochloric acid secretion), pancreatic acinar cell (bicarbonate and digestive enzyme secretion), paneth cell of small intestine (lysozyme secretion), type ii pneumocyte of lung (surfactant secretion), club cell of lung): barrier cells (type I pneumocyte (lung), gall bladder epithelial cell, centroacinar cell (pancreas), intercalated duct cell (pancreas), intestinal brush border cell (with microvilli); Hormone-secreting cells: Enteroendocrine cell, K cell (secretes gastric inhibitory peptide), L cell (secretes glucagon-like peptide-1, peptide YY3-36, oxyntomodulin, and glucagon-like peptide-2), I cell (secretes cholecystokinin (CCK)), G cell (secretes gastrin), Enterochromaffin cell (secretes serotonin), Enterochromaffin-like cell (secretes histamine), N cell (secretes neurotensin), S cell (secretes secretin), D cell (secretes somatostatin), Mo cell (or M cell) (secretes motilin), other hormones secreted: vasoactive intestinal peptide, substance P, alpha and gamma-endorphin, bombesin; Thyroid gland cells, Thyroid epithelial cell, Parafollicular cell, Parathyroid gland cells, Parathyroid chief cell, Oxyphil cell; Pancreatic islets (islets of Langerhans), Alpha cell (secretes glucagon), Beta cell (secretes insulin and amylin), Delta cell (secretes somatostatin), Epsilon cell (secretes ghrelin), PP cell (gamma cell) (secretes pancreatic polypeptide).

Ectoderm derived cells such as exocrine secretory epithelial cells, salivary gland mucous cell, salivary gland serous cell, von Ebner's gland cell in tongue (washes taste buds), mammary gland cell (milk secretion), lacrimal gland cell (tear secretion), ceruminous gland cell in ear (earwax secretion), eccrine sweat gland dark cell (glycoprotein secretion), eccrine sweat gland clear cell (small molecule secretion), apocrine sweat gland cell (odoriferous secretion, sex-hormone sensitive), gland of moll cell in eyelid (specialized sweat gland), sebaceous gland cell (lipid-rich sebum secretion), Bowman's gland cell in nose (washes olfactory epithelium); hormone-secreting cells, Hormone-secreting cells, Anterior/Intermediate pituitary cells, Corticotropes, Gonadotropes, Lactotropes, Melanotropes, Somatotropes, Thyrotropes, Magnocellular neurosecretory cells, secrete oxytocin and vasopressin, Parvocellular neurosecretory cells, secrete thyrotropin-releasing hormone (TRH), corticotropin-releasing hormone (CRH), vasopressin, oxytocin, neurotensin, and prolactin, Chromaffin cells (adrenal gland); Epithelial cells such as Keratinocyte (differentiating epidermal cell); Epidermal basal cell (stem cell); Melanocyte; Trichocyte (gives rise to hair and nail cells) including Medullary hair shaft cell, Cortical hair shaft cell, Cuticular hair shaft cell, Huxley's layer hair root sheath cell, Henle's layer hair root sheath cell, Outer root sheath hair cell; Surface epithelial cell of cornea, tongue, mouth, nasal cavity, distal anal canal, distal urethra, and distal vagina; basal cell (stem cell) of cornea, tongue, mouth, nasal cavity, distal anal canal, distal urethra, and distal vagina; Intercalated duct cell (salivary glands); Striated duct cell (salivary glands); Lactiferous duct cell (mammary glands); Ameloblast (deposit tooth enamel), Oral cells such as Odontoblast (tooth dentin formation), Cementoblast (tooth cementum formation); Sensory transducer cells such as, Auditory inner hair cells of organ of Corti, Auditory outer hair cells of organ of Corti, Basal cells of olfactory epithelium (stem cell for olfactory neurons), Cold-sensitive primary sensory neurons, Heat-sensitive primary sensory neurons, Merkel cells of epidermis, Olfactory receptor neurons, Pain-sensitive primary sensory neurons; Photoreceptor cells of retina in eye, Photoreceptor rod cells, Photoreceptor blue-sensitive cone cells of eye, Photoreceptor green-sensitive cone cells of eye, Photoreceptor red-sensitive cone cells of eye; Proprioceptive primary sensory neurons; Touch-sensitive primary sensory neuronss; Chemoreceptor glomus cells of carotid body cell (blood pH sensor); Outer hair cells of vestibular system of ear (acceleration and gravity); Inner hair cells of vestibular system of ear (acceleration and gravity) Taste receptor cells of taste bud; Autonomic neuron cells such as; Cholinergic neurons (various types); Adrenergic neural cells (various types); Peptidergic neural cells (various types); Sense organ and peripheral neuron supporting cells such as, Inner pillar cells of organ of Corti, Outer pillar cells of organ of Corti, Inner phalangeal cells of organ of Corti, Outer phalangeal cells of organ of Corti, Border cells of organ of Corti, Hensen's cells of organ of Corti, Vestibular apparatus supporting cells, Taste bud supporting cells, Olfactory epithelium supporting cells, Olfactory ensheathing cells, Schwann cells, Satellite glial cells, Enteric glial cells, Central nervous system neurons and glial cells such as Neuron cells (Interneurons, Basket cells, Cartwheel cells, Stellate cells, Golgi cells, Granule cells, Lugaro cells, Unipolar brush cells, Martinotti cells, Chandelier cells, Cajal-Retzius cells, Double-bouquet cells, Neurogliaform cells, Retina horizontal cells, Amacrine cells, Starburst amacrine cells, Spinal interneurons, Renshaw cells); Principal cells (Spindle neurons, Fork neurons, Pyramidal cells, Place cells, Grid cells, Speed cells, Head direction cells, Betz cells, Stellate cells, Boundary cells, Bushy cells, Purkinje cells, Medium spiny neurons); Astrocytes; Oligodendrocytes; Ependymal cells, Tanycytes; Pituicytes; Nervous system cells such as sensory transducer cells, autonomic neuron cells, sense organ and peripheral neuron supporting cells, central nervous system neurons and glial cells; lens cells (Anterior lens epithelial cell, Crystallin-containing lens fiber cell)

Cells derived primarily from mesoderm such as: Metabolism and storage cells (Adipocytes: (White fat cell and Brown fat cell, Liver lipocyte); Secretory cells (Cells of the Adrenal cortex including Cells of the Zona glomerulosa produce mineralocorticoids, Cells of the Zona fasciculata produce glucocorticoids, Cells of the Zona reticularis produce androgens); Theca intema cell of ovarian follicle secreting estrogen; Corpus luteum cell of ruptured ovarian follicle secreting progesterone (Granulosa lutein cells, Theca lutein cells); Leydig cell of testes secreting testosterone; Seminal vesicle cell (secretes seminal fluid components, including fructose for swimming sperm); Prostate gland cell (secretes seminal fluid components); Bulbourethral gland cell (mucus secretion); Bartholin's gland cell (vaginal lubricant secretion); Gland of Littre cell (mucus secretion); Uterus endometrium cell (carbohydrate secretion); Juxtaglomerular cell (renin secretion); Macula densa cell of kidney; Peripolar cell of kidney; Mesangial cell of kidney; Barrier cells Urinary system (Parietal epithelial cell; Podocyte, Proximal tubule brush border cell, Loop of Henle thin segment cell, Kidney distal tubule cell, Kidney collecting duct cell, Principal cell, Intercalated cell, Transitional epithelium (lining urinary bladder); Reproductive system: Duct cell (of seminal vesicle, prostate gland, etc.), Efferent ducts cell, Epididymal principal cell, Epididymal basal cell; Circulatory system: Endothelial cells; Extracellular matrix cells: Planum semilunatum epithelial cell of vestibular system of ear (proteoglycan secretion), Organ of Corti interdental epithelial cell (secreting tectorial membrane covering hair cells), Loose connective tissue fibroblasts, Corneal fibroblasts (corneal keratocytes), Tendon fibroblasts, Bone marrow reticular tissue fibroblasts, Other nonepithelial fibroblasts, Pericyte (Hepatic stellate cell (Ito cell)), Nucleus pulposus cell of intervertebral disc, Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, Stellate cell of perilymphatic space of ear, Pancreatic stellate cell; Contractile cells such as: Skeletal muscle cell (Red skeletal muscle cell (slow twitch), White skeletal muscle cell (fast twitch), Intermediate skeletal muscle cell, Nuclear bag cell of muscle spindle, Nuclear chain cell of muscle spindle, Myosatellite cell (stem cell)), Cardiac muscle cells (Cardiac muscle cell, SA node cell, Purkinje fiber cell); Smooth muscle cell (various types); Myoepithelial cell of iris; Myoepithelial cell of exocrine glands; Blood and immune system cells such as Erythrocyte (red blood cell) and precursor erythroblasts, Megakaryocyte (platelet precursor), Platelets if considered distinct cells, currently there's debate on the subject, Monocyte (white blood cell), Connective tissue macrophage (various types), Epidermal Langerhans cell, Osteoclast (in bone), Dendritic cell (in lymphoid tissues), Microglial cell (in central nervous system), Neutrophil granulocyte and precursors (myeloblast, promyelocyte, myelocyte, metamyelocyte), Eosinophil granulocyte and precursors, Basophil granulocyte and precursors, Mast cell, Helper T cell, Regulatory T cell, Cytotoxic T cell, Natural killer T cell, B cell, Plasma cell, Natural killer cell, Hematopoietic stem cells and committed progenitors for the blood and immune system (various types); Germ cells such as Oogonium/Oocyte, Spermatid, Spermatocyte, Spermatogonium cell (stem cell for spermatocyte), Spermatozoon; Nurse cell, Granulosa cell (in ovaries), Sertoli cell (in testis), Epithelial reticular cell (in thymus; and Interstitial cells: Interstitial kidney cells.

2. Therapeutic Particles

a. Extracellular Vesicles

It can also be envisioned to use the introduction of CD43, CD45 and or CD148 engagers into either extracellular vesicles directly or into the parental cells that is utilized for extracellular vesicle production for either targeted or systemic delivery of CD43, CD45 and or CD148 engagers and/or therapeutic transgene or protein.

b. Adenoviral Gene Delivery

One of the challenges of in vivo viral gene delivery is cellular rejection of gene modified cells due to the immunogenicity of the vector proteins. Co introduction of engagers against CD43, CD45 and/or CD148 proteins may lead to a better gene modification by avoiding cellular responses to in vivo modified cells.

c. Oncolytic Viruses

Despite active research in virotherapy, this apparently safe modality has not achieved widespread success. The immune response to viral infection appears to be an essential factor that determines the efficacy of oncolytic viral therapy. The challenge is determining whether the viral-elicited immune response is a hindrance or a tool for viral treatment. NK cells are a key component of innate immunity that mediates antiviral immunity while also coordinating tumor clearance. Various reports have suggested that the NK response to oncolytic viral therapy is a critical factor in premature viral clearance while also mediating downstream antitumor immunity. As a result, particular attention should be given to the NK cell response to various oncolytic viral vectors and how their antiviral properties can be suppressed while maintaining tumor clearance. In this setup, one can envision that oncolytic viruses can be engineered to include genes coding for engagers against CD43, CD45 and or CD148 whereby the OV infected cells would be saved from NK cell mediated killing.

This strategy can also be utilized for cellular drug delivery where graft cells ferry the drugs to target tissue where these graft cells are co modified with CD43, CD45 and or CD148 engagers.

C. Specific Conditions

1. Control of Chronic Inflammatory Diseases by Repetitive Transient Gene Delivery.

It can also be envisioned that mRNA or DNA coding for CD43, CD45 and or CD148 engagers can be delivered in a local or systemic fashion to patients with chronic inflammatory diseases where cellular cytotoxicity is a part of the disease physiology. This could be done locally or systematically in patients with autoimmune diseases such as multiple sclerosis, inflammatory bowel diseases, Crohn's disease.

2. Wound Healing and Skin Grafts

The present invention can be used in connection with conventional stem cell therapies to produce cells and tissues for treatment of wounds without fear of rejection(3). Kosaric N, Kiwanuka H, Gurtner G C. Stem cell therapies for wound healing. Expert Opin Biol Ther. 2019; 19(6):575-85

3. Inherited Metabolic Disorders

The present invention can be used to treat inherited metabolic disorders such as 17-alpha-hydroxylase deficiency, 17-beta hydroxysteroid dehydrogenase 3 deficiency, 18 Hydroxylase deficiency, 2-Hydroxyglutaric aciduria, 2-methyl-3-hydroxybutyric aciduria, 2-methylbutyryl-CoA dehydrogenase deficiency, 3 Methylcrotonyl-CoA carboxylase 1 deficiency, 3-alpha hydroxyacyl-CoA dehydrogenase deficiency, 3-Hydroxyisobutyric aciduria, 3-methylcrotonyl-CoA carboxylase deficiency, 3-methylglutaconyl-CoA hydratase deficiency (AUH defect), 5-oxoprolinase deficiency, 6-pyruvoyl-tetrahydropterin synthase deficiency, Abdominal obesity metabolic syndrome, Abetalipoproteinemia, Acatalasemia, Aceruloplasminemia, Acetyl CoA acetyltransferase 2 deficiency, Acetyl-camitine deficiency, Acrodermatitis enteropathica, Acromegaly, Acute intermittent porphyria, Adenine phosphoribosyltransferase deficiency, Adenosine deaminase deficiency, Adenosine monophosphate deaminase 1 deficiency, Adenylosuccinase deficiency, Adrenomyeloneuropathy, Adult polyglucosan body disease, Adult-onset citrullinemia type II, Albinism deafness syndrome, Albinism ocular late onset sensorineural deafness, ALG1-CDG (CDG-Ik), ALGI1-CDG (CDG-Ip), ALG12-CDG (CDG-Ig), ALG13-CDG, ALG2-CDG (CDG-Ii), ALG3-CDG (CDG-Id), ALG6-CDG (CDG-Ic), ALG8-CDG (CDG-Ih), ALG9-CDG (CDG-IL), Alkaptonuria, Alpers syndrome, Alpha-1 antitrypsin deficiency, Alpha-ketoglutarate dehydrogenase deficiency, Alpha-mannosidosis, Aminoacylase 1 deficiency, Anemia due to Adenosine triphosphatase deficiency, Anemia sideroblastic and spinocerebellar ataxia, Apparent mineralocorticoid excess, Arginase deficiency, Argininosuccinic aciduria, Aromatic L-amino acid decarboxylase deficiency, Arthrogryposis renal dysfunction cholestasis syndrome, Arts syndrome, Aspartylglycosaminuria, Ataxia with oculomotor apraxia type 1, Ataxia with vitamin E deficiency, Atransferrinemia, Atypical Gaucher disease due to saposin C deficiency (Gaucher disease), Autoimmune polyglandular syndrome type 2, Autosomal dominant neuronal ceroid lipofuscinosis 4B, Autosomal dominant optic atrophy and cataract, Autosomal dominant optic atrophy plus syndrome, Autosomal recessive neuronal ceroid lipofuscinosis 4A (Adult neuronal ceroid lipofuscinosis), Autosomal recessive spastic ataxia 4, Autosomal recessive spinocerebellar ataxia 9, B4GALT1-CDG (CDG-IId), Bantu siderosis, Barth syndrome, Bartter syndrome, Bartter syndrome antenatal type 1, Bartter syndrome antenatal type 2, Bartter syndrome type 3, Bartter syndrome type 4, Beta ketothiolase deficiency, Biotin-thiamine-responsive basal ganglia disease, Biotinidase deficiency, Bjornstad syndrome, Blue diaper syndrome, Carbamoyl phosphate synthetase 1 deficiency, Carnitine palmitoyl transferase 1A deficiency, Carnitine-acylcamitine translocase deficiency, Camosinemia, Central diabetes insipidus, Cerebral folate deficiency, Cerebrotendinous xanthomatosis, Ceroid lipofuscinosis neuronal 1, Chanarin-Dorfman syndrome, Chediak-Higashi syndrome, CHILD syndrome, Childhood hypophosphatasia, Childhood-onset cerebral X-linked adrenoleukodystrophy, Cholesteryl ester storage disease, Chondrocalcinosis 1, Chondrocalcinosis 2, Chondrocalcinosis due to apatite crystal deposition, Chondrodysplasia punctata 1, X-linked recessive, Chronic progressive external ophthalmoplegia, Chylomicron retention disease, Citrulline transport defect, COGI-CDG (CDG-IIg), COG4-CDG (CDG-IIj), COG5-CDG (CDG-IIi), COG7-CDG (CDG-IIe), COG8-CDG (CDG-IIh), Combined oxidative phosphorylation deficiency 16, Congenital bile acid synthesis defect, type 1, Congenital bile acid synthesis defect, type 2, Congenital disorder of glycosylation type I/IIX, Congenital dyserythropoietic anemia type 2, Congenital erythropoietic porphyria, Congenital lactase deficiency, Congenital muscular dystrophy-dystroglycanopathy with or without intellectual disability (type B), Copper deficiency, familial benign, CoQ-responsive OXPHOS deficiency, Crigler Najjar syndrome, type 1, Crigler-Najjar syndrome type 2, Cystinosis, Cystinosis, ocular nonnephropathic, Cytochrome c oxidase deficiency, D-2-hydroxyglutaric aciduria, D-bifunctional protein deficiency, D-glycericacidemia, Danon disease, DCMA syndrome, DDOST-CDG (CDG-Ir), Deafness, dystonia, and cerebral hypomyelination, Dentatorubral-pallidoluysian atrophy, Desmosterolosis, Diamond-Blackfan anemia, Dicarboxylic aminoaciduria, Dihydrolipoamide dehydrogenase deficiency, Dihydropteridine reductase deficiency, Dihydropyrimidinase deficiency, Dihydropyrimidine dehydrogenase deficiency, Dipsogenic diabetes insipidus, DOLK-CDG (CDG-Im), Dopa-responsive dystonia, Dopamine beta hydroxylase deficiency, Dowling-Degos disease, DPAGT1-CDG (CDG-Ij), DPM1-CDG (CDG-Ie), DPM2-CDG, DPM3-CDG (CDG-Io), Dubin-Johnson syndrome, Encephalopathy due to prosaposin deficiency (Sphingolipidosis), Erythropoietic protoporphyria, Erythropoietic uroporphyria associated with myeloid malignancy, Ethylmalonic encephalopathy, Fabry disease, Familial HDL deficiency, Familial hypocalciuric hypercalcemia type 1, Familial hypocalciuric hypercalcemia type 2, Familial hypocalciuric hypercalcemia type 3, Familial LCAT deficiency, Familial partial lipodystrophy type 2, Fanconi Bickel syndrome, Farber's disease, Fatal infantile encephalomyopathy, Fatty acid hydroxylase-associated neurodegeneration, Fish-eye disease, Fructose-1,6-bisphosphatase deficiency, Fucosidosis, Fukuyama type muscular dystrophy, Fumarase deficiency, Galactokinase deficiency, Galactosialidosis, Gamma aminobutyric acid transaminase deficiency, Gamma-cystathionase deficiency, Gaucher disease, Gaucher disease—ophthalmoplegia—cardiovascular calcification (Gaucher disease), Gaucher disease perinatal lethal, Gaucher disease type 1, Gaucher disease type 2, Gaucher disease type 3, Gestational diabetes insipidus, Gilbert syndrome, Gitelman syndrome, Glucose transporter type 1 deficiency syndrome, Glucose-galactose malabsorption, Glutamate formiminotransferase deficiency, Glutamine deficiency, congenital, Glutaric acidemia type I, Glutaric acidemia type II, Glutaric acidemia type III, Glutathione synthetase deficiency, Glutathionuria, Glycine N-methyltransferase deficiency, Glycogen storage disease 8, Glycogen storage disease type 0, liver, Glycogen storage disease type 12, Glycogen storage disease type 13, Glycogen storage disease type 1A, Glycogen storage disease type 1B, Glycogen storage disease type 3, Glycogen storage disease type 5, Glycogen storage disease type 6, Glycogen storage disease type 7, Glycoproteinosis, GM1 gangliosidosis type 1, GM1 gangliosidosis type 2, GM1 gangliosidosis type 3, GM3 synthase deficiency, GRACILE syndrome, Greenberg dysplasia, GTP cyclohydrolase I deficiency, Guanidinoacetate methyltransferase deficiency, Gyrate atrophy of choroid and retina, Haim-Munk syndrome, Hartnup disease, Hawkinsinuria, Hemochromatosis type 2, Hemochromatosis type 3, Hemochromatosis type 4, Hepatic lipase deficiency, Hepatoerythropoietic porphyria, Hereditary amyloidosis, Hereditary coproporphyria, Hereditary folate malabsorption, Hereditary fructose intolerance, Hereditary hyperekplexia, Hereditary multiple osteochondromas, Hereditary sensory and autonomic neuropathy type 1E, Hereditary sensory neuropathy type 1, Hermansky Pudlak syndrome 2, Histidinemia, HMG CoA lyase deficiency, Homocarnosinosis, Homocysteinemia, Homocystinuria due to CBS deficiency, Homocystinuria due to MTHFR deficiency, Hurler syndrome, Hurler-Scheie syndrome, Hydroxykynureninuria, Hyper-IgD syndrome, Hyperbetaalaninemia, Hypercoagulability syndrome due to glycosylphosphatidylinositol deficiency, Hyperglycerolemia, Hyperinsulinism due to glucokinase deficiency, Hyperinsulinism-hyperammonemia syndrome, Hyperlipidemia type 3, Hyperlipoproteinemia type 5, Hyperlysinemia, Hyperphenylalaninemia due to dehydratase deficiency, Hyperprolinemia, Hyperprolinemia type 2, Hypertryptophanemia, Hypolipoproteinemia, Hypophosphatasia, I cell disease, Imerslund-Grasbeck syndrome, Iminoglycinuria, Inclusion body myopathy 2, Inclusion body myopathy 3, Infantile free sialic acid storage disease (Free sialic acid storage disease), Infantile neuroaxonal dystrophy, Infantile onset spinocerebellar ataxia, Insulin-like growth factor I deficiency, Intrinsic factor deficiency, Isobutyryl-CoA dehydrogenase deficiency, Isovaleric acidemia, Kanzaki disease, Keams-Sayre syndrome, Krabbe disease atypical due to Saposin A deficiency, L-2-hydroxyglutaric aciduria, L-arginine:glycine amidinotransferase deficiency, Lactate dehydrogenase A deficiency, Lactate dehydrogenase deficiency, Lathosterolosis, LCHAD deficiency, Leber hereditary optic neuropathy, Leigh syndrome, French Canadian type, Lesch Nyhan syndrome, Leucine-sensitive hypoglycemia of infancy, Leukoencephalopathy—dystonia—motor neuropathy, Leukoencephalopathy with brain stem and spinal cord involvement and lactate elevation, Limb-girdle muscular dystrophy type 21, Limb-girdle muscular dystrophy type 2K, Limb-girdle muscular dystrophy type 2M, Limb-girdle muscular dystrophy type 2N, Limb-girdle muscular dystrophy type 20-, Limb-girdle muscular dystrophy type 2T (Limb-girdle muscular dystrophy), type 2C, Lipase deficiency combined, Lipoic acid synthetase deficiency, Lipoid proteinosis of Urbach and Wiethe, Lowe oculocerebrorenal syndrome, Lysinuric protein intolerance, Malonyl-CoA decarboxylase deficiency, MAN1B1-CDG, Mannose-binding lectin protein deficiency, Mannosidosis, beta A, lysosomal, Maple syrup urine disease type 1A, Maple syrup urine disease type 1B, Maple syrup urine disease type 2, Maternal hyperphenylalaninemia, Maternally inherited diabetes and deafness, Medium-chain acyl-coenzyme A dehydrogenase deficiency, Megaloblastic anemia due to dihydrofolate reductase deficiency, Menkes disease, Metachromatic leukodystrophy, Metachromatic leukodystrophy due to saposin B deficiency, Methionine adenosyltransferase deficiency, Methylcobalamin deficiency cbl G type, Methylmalonic acidemia with homocystinuria type cblC, Methylmalonic acidemia with homocystinuria type cblD, Methylmalonic acidemia with homocystinuria type cblF, Methylmalonic acidemia with homocystinuria type cblJ, Methylmalonic aciduria, cblA type, Methylmalonic aciduria, cblB type, Mevalonic aciduria, MGAT2-CDG (CDG-IIa), Mild phenylketonuria, Mitochondrial complex I deficiency, Mitochondrial complex II deficiency, Mitochondrial complex III deficiency, Mitochondrial DNA depletion syndrome, encephalomyopathic form with methylmalonic aciduria, Mitochondrial DNA-associated Leigh syndrome, Mitochondrial encephalomyopathy lactic acidosis and stroke-like episodes, Mitochondrial myopathy with diabetes, Mitochondrial myopathy with lactic acidosis, Mitochondrial neurogastrointestinal encephalopathy syndrome, Mitochondrial trifunctional protein deficiency, MOGS-CDG (CDG-IIb), Mohr-Tranebjaerg syndrome, Molybdenum cofactor deficiency, Monogenic diabetes, Morquio syndrome B, MPDU1-CDG (CDG-If), MPI-CDG (CDG-Ib), MPV17-related hepatocerebral mitochondrial DNA depletion syndrome, Mucolipidosis III alpha/beta, Mucolipidosis type 4, Mucopolysaccharidosis type II, Mucopolysaccharidosis type III, Mucopolysaccharidosis type IIIA, Mucopolysaccharidosis type IIIB, Mucopolysaccharidosis type IIIC, Mucopolysaccharidosis type HID, Mucopolysaccharidosis type IVA, Mucopolysaccharidosis type VI, Mucopolysaccharidosis type VII, Multiple congenital anomalies-hypotonia-seizures syndrome, Multiple congenital anomalies-hypotonia-seizures syndrome type 2, Multiple endocrine neoplasia type 2B, Multiple sulfatase deficiency, Multiple symmetric lipomatosis, Muscle eye brain disease, Muscular dystrophy, congenital, megaconial type, Muscular phosphorylase kinase deficiency, Musculocontractural Ehlers-Danlos syndrome, Myoclonic epilepsy with ragged red fibers, Myoglobinuria recurrent, N acetyltransferase deficiency, N-acetyl-alpha-D-galactosaminidase deficiency type III, N-acetylglutamate synthase deficiency, NBIA/DYT/PARK-PLA2G6, Neonatal adrenoleukodystrophy, Neonatal hemochromatosis, Neonatal intrahepatic cholestasis caused by citrin deficiency, Nephrogenic diabetes insipidus, Neu Laxova syndrome, Neuroferritinopathy, Neuronal ceroid lipofuscinosis 10, Neuronal ceroid lipofuscinosis 2, Neuronal ceroid lipofuscinosis 3, Neuronal ceroid lipofuscinosis 5, Neuronal ceroid lipofuscinosis 6, Neuronal ceroid lipofuscinosis 7, Neuronal ceroid lipofuscinosis 9, Neuropathy ataxia retinitis pigmentosa syndrome, Neutral lipid storage disease with myopathy, Niemann-Pick disease type A, Niemann-Pick disease type B, Niemann-Pick disease type C1, Niemann-Pick disease type C2, Northern epilepsy, Not otherwise specified 3-MGA-uria type, Occipital horn syndrome, Ocular albinism type 1, Oculocutaneous albinism type 1, Oculocutaneous albinism type 1B, Oculocutaneous albinism type 2, Oculocutaneous albinism type 3, OPA3 defect, Optic atrophy 1, Omithine transcarbamylase deficiency, Ornithine translocase deficiency syndrome, Orotic aciduria type 1, Papillon Lefevre syndrome, Parkinson disease type 9, Paroxysmal nocturnal hemoglobinuria, Pearson syndrome, Pentosuria, Permanent neonatal diabetes mellitus, Peroxisomal biogenesis disorders, Peroxisome disorders, Perrault syndrome, Peters plus syndrome, PGM1-CDG, Phosphoglycerate kinase deficiency, Phosphoglycerate mutase deficiency, Phosphoribosylpyrophosphate synthetase deficiency, PMM2-CDG (CDG-Ia), Pontocerebellar hypoplasia type 6, Porphyria cutanea tarda, Primary camitine deficiency, Primary hyperoxaluria type 1, Primary hyperoxaluria type 2, Primary hyperoxaluria type 3, Primary hypomagnesemia with secondary hypocalcemia, Progressive external ophthalmoplegia, autosomal recessive 1, Progressive familial intrahepatic cholestasis 1, Progressive familial intrahepatic cholestasis type 2, Progressive familial intrahepatic cholestasis type 3, Prolidase deficiency, Propionic acidemia, Pseudocholinesterase deficiency, Pseudoneonatal adrenoleukodystrophy, Purine nucleoside phosphorylase deficiency, Pycnodysostosis, Pyridoxal 5′-phosphate-dependent epilepsy, Pyridoxine-dependent epilepsy, Pyruvate carboxylase deficiency, Pyruvate dehydrogenase complex deficiency, Pyruvate dehydrogenase phosphatase deficiency, Pyruvate kinase deficiency, Refsum disease, Refsum disease with increased pipecolic acidemia, Refsum disease, infantile form, Renal glycosuria, Renal hypomagnesemia 2, Renal hypomagnesemia-6, Renal tubulopathy, diabetes mellitus, and cerebellar ataxia due to duplication of mitochondrial DNA, RFT1-CDG (CDG-In), Rhizomelic chondrodysplasia punctata type 3 (Rhizomelic chondrodysplasia punctata), Rotor syndrome, Saccharopinuria, Salla disease (Free sialic acid storage disease), Sarcosinemia, Scheie syndrome, Schimke immunoosseous dysplasia, Schindler disease type 1, Schneckenbecken dysplasia, SCOT deficiency, Sea-Blue histiocytosis, Sengers syndrome, Sensory ataxic neuropathy, dysarthria, and ophthalmoparesis, Sepiapterin reductase deficiency, Severe combined immunodeficiency, Short-chain acyl-CoA dehydrogenase deficiency, Sialidosis type I, Sialidosis, type II, Sialuria, French type, Sideroblastic anemia and mitochondrial myopathy, Sitosterolemia, Sjogren-Larsson syndrome, SLC35A1-CDG (CDG-IIf), SLC35A2-CDG, SLC35C1-CDG (CDG-IIc), Smith-Lemli-Opitz syndrome, Spastic paraplegia 7, Spinocerebellar ataxia 28, Spinocerebellar ataxia autosomal recessive 3, Spondylocostal dysostosis 1, Spondylocostal dysostosis 2, Spondylocostal dysostosis 3, Spondylocostal dysostosis 4, Spondylocostal dysostosis 5, Spondylocostal dysostosis 6, Spondylodysplastic Ehlers-Danlos syndrome, Spondyloepimetaphyseal dysplasia joint laxity, SRD5A3-CDG (CDG-Iq), SSR4-CDG, Succinic semialdehyde dehydrogenase deficiency, Tangier disease, Tay-Sachs disease, Thiamine responsive megaloblastic anemia syndrome, Thiopurine S methyltranferase deficiency, Tiglic acidemia, TMEM165-CDG (CDG-IIk), Transaldolase deficiency, Transcobalamin 1 deficiency, Transient neonatal diabetes mellitus, Trehalase deficiency, Trimethylaminuria, Triosephosphate isomerase deficiency, Tyrosine hydroxylase deficiency, Tyrosine-oxidase temporary deficiency, Tyrosinemia type 1, Tyrosinemia type 2, Tyrosinemia type 3, Urea cycle disorders, Valinemia, Variegate porphyria, VLCAD deficiency, Walker-Warburg syndrome, Wilson disease, Wolfram syndrome, Wolman disease, Wrinkly skin syndrome, X-linked adrenoleukodystrophy, X-linked Charcot-Marie-Tooth disease type 5, X-linked creatine deficiency, X-linked dominant chondrodysplasia punctata 2, X-linked sideroblastic anemia, Xanthinuria type 1, Xanthinuria type 2 and Zellweger syndrome.

1. A therapeutic agent comprising one or more molecules or cells configured to modulate the ability of CD45, CD148, or CD43 to form a functional immunological synapse with a cytotoxic cell, thereby preventing cytotoxicity.

2.-63. (canceled)

64. The therapeutic agent of claim 1, which comprises a protein, aptamer, peptide nucleic acid (PNA), nanoparticle, or cell which expresses or secretes the one or more molecules.

65. The therapeutic agent of claim 64, which comprises a protein, preferably a protein comprising an antibody, more preferably comprising a single chain antibody or VHH nanobody.

66. The therapeutic agent of claim 1, which comprises a nanoparticle, preferably a lipid nanoparticle (LNP), dendrimer, ribonucleoprotein (RNP), or an extracellular vesicle, preferably an exosome or microvesicle.

67. The therapeutic agent of claim 1, which comprises a component of viral or bacterial origin, preferably ULL or E3/49k, or a fragment thereof.

68. The therapeutic agent of claim 1, which does not comprise an E3/49k protein, or fragment thereof.

69. The therapeutic agent of claim 64, which comprises SEQ ID NO: 1, 3, 5, 64, 66, 68, 71, 73, 220, 223, or 224, or a protein having at least 80% identity to SEQ ID NO: 1, 3, 5, 64, 66, 68, 71, 73, 220, 223, or 224.

70. The therapeutic agent of claim 64, which comprises a cell having one or more molecules expressed on the surface of the cell.

71. The therapeutic agent of claim 70, wherein the one or more molecules expressed on the surface of the cell comprises a transmembrane protein expressed and the cell comprises a graft cell.

72. The therapeutic agent of claim 71, wherein the transmembrane protein is capable of binding to CD45, CD148, or CD43.

73. The therapeutic agent of claim 72, wherein the CD45, CD148, or CD43 is present on the surface of a cytotoxic cell, preferably a T cell or natural killer (NK) cell.

74. The therapeutic agent of claim 73, wherein the transmembrane protein is capable of retaining CD45, CD148, or CD43 in a developing immunological synapse on the surface of the cytotoxic cell, thereby disrupting functional immunological synapse formation.

75. A protein complex capable of preventing cytotoxic cell-induced lysis, which protein complex comprises:
an engager comprising SEQ ID NO: 1, 3, 5, 64, 66, 68, 71, 73, 220, 223, or 224, or a protein having at least 80% identity to SEQ ID NO: 1, 3, 5, 64, 66, 68, 71, 73, 220, 223, or 224; and
a CD45, CD148, or CD43 protein expressed on the surface of a T cell or NK cell.

76. The therapeutic agent of claim 1, which is configured for use in the prevention or treatment of one or more of the following conditions: autoimmune disease, blood cancers, including lymphomas and leukemias; bone marrow failure syndromes, including anemias and cytopenias; inherited immune disorders, including WAS and SCID; hemoglobinopathies, including sickle cell disease (SCD) and thalassemia; neurological disorders, including neuromyelitis optica; graft vs. host disease, psoriasis, and vitiligo.

77. An nonautologous cell comprising an engager on its surface and which is configured to avoid synapse formation with one or more host cytotoxic cells, wherein the engager comprises SEQ ID NO: 1, 3, 5, 64, 66, 68, 71, 73, 220, 223, or 224, or a protein having at least 80% identity to SEQ ID NO: 1, 3, 5, 64, 66, 68, 71, 73, 220, 223, or 224.

78. The nonautologous cell of claim 77, wherein the host cytotoxic cell is a natural killer cell, a T cell, or a macrophage.

79. The nonautologous cell of claim 77, wherein the cytotoxic cell is a T cell, preferably a gamma-delta T cell, a CD8+ T cell, a CD4+ T cell, or a mucosal associated invariant T cell.

80. The nonautologous cell of claim 77, which is free of genetic modification.

81. An recombinant protein comprising: (i) a signal peptide, (ii) a heavy chain of an antibody, (iii) a first linker, (iv) a light chain of an antibody, (v) optionally, a second linker, (vi) a stalk, (vii) a transmembrane region, and (viii) optionally, an intracellular region, which is a single chain antibody, preferably a single chain antibody which binds specifically to CD45, CD148, or CD43.

82. The recombinant protein of claim 81, wherein (i)-(vii) are each present, and are connected in order from amino terminus to carboxyl terminus of the protein.

83. The recombinant protein of claim 81, wherein
the signal peptide is an IL2 signal peptide;
the first linker comprises an SGGGG motif and/or may vary in length from 5-60, preferably 10-50, more preferably 20-45 amino acids,
the second linker, when present, may vary in length from 5 to 60, preferably 5-40, more preferably 7-15 amino acids;
the stalk is at least 8 and no more than 200 amino acids in length, and
the transmembrane region is derived from CD34, CD45, CD28, and/or Cd8a.
